SEQUENCE LISTING (444 sequences)

SEQ ID NO: 1 - DNA, 32 bp, Artificial Sequence
  Synthetic
gatcagggtaccaggagccttaggaggtacgg

SEQ ID NO: 2 - DNA, 33 bp, Artificial Sequence
  Synthetic
gatagcctcgagttatttccaggtcatgagcca

SEQ ID NO: 3 - DNA, 20 bp, Artificial Sequence
  Synthetic
ttccaggtcatgagccatta

SEQ ID NO: 4 - DNA, 21 bp, Artificial Sequence
  Synthetic
cataaggtgtgctctgaagtg

SEQ ID NO: 5 - DNA, 20 bp, Artificial Sequence
  Synthetic
ttactcatggtaatgtcttt

SEQ ID NO: 6 - DNA, 20 bp, Artificial Sequence
  Synthetic
attaaaaacatgtatcactt

SEQ ID NO: 7 - DNA, 21 bp, Artificial Sequence
  Synthetic
ggaacacagaagcaaggtggt

SEQ ID NO: 8 - DNA, 20 bp, Artificial Sequence
  Synthetic
ccgtacctcctaaggctcct

SEQ ID NO: 9 - DNA, 20 bp, Artificial Sequence
  Synthetic
cccatagtgctgtcacaaat

SEQ ID NO: 10 - DNA, 20 bp, Artificial Sequence
  Synthetic
gcacagcagcattcccaagg

SEQ ID NO: 11 - DNA, 20 bp, Artificial Sequence
  Synthetic
ttgcaaattggcatggcagg

SEQ ID NO: 12 - DNA, 20 bp, Artificial Sequence
  Synthetic
tggtatgggctttactcttt

SEQ ID NO: 13 - DNA, 20 bp, Artificial Sequence
  Synthetic
aaaaggttgcccaggaacgg

SEQ ID NO: 14 - DNA, 20 bp, Artificial Sequence
  Synthetic
gggagtcctggagccccctt

SEQ ID NO: 15 - DNA, 20 bp, Artificial Sequence
  Synthetic
ccattaagctgggcttggcc

SEQ ID NO: 16 - DNA, 20 bp, Artificial Sequence
  Synthetic
tgcgagctccccgtacctcc

SEQ ID NO: 17 - DNA, 20 bp, Artificial Sequence
  Synthetic
gcccaagctggcatccgtca

SEQ ID NO: 18 - DNA, 20 bp, Artificial Sequence
  Synthetic
ggattgccaagcccatggtg

SEQ ID NO: 19 - DNA, 20 bp, Artificial Sequence
  Synthetic
ctaagtagtgctagccggga

SEQ ID NO: 20 - DNA, 38 bp, Artificial Sequence
  Synthetic
gatcagggtaccccaaagaaaaagtgatttgtcattgc

SEQ ID NO: 21 - DNA, 35 bp, Artificial Sequence
  Synthetic
gatagcctcgaggataatgaattggctgacaagac

SEQ ID NO: 22 - DNA, 20 bp, Artificial Sequence
  Synthetic
gggtaagactccacttctga

SEQ ID NO: 23 - DNA, 20 bp, Artificial Sequence
  Synthetic
gggtctccaaaggttgtgga

SEQ ID NO: 24 - DNA, 20 bp, Artificial Sequence
  Synthetic
gttcctgggtctccaaaggt

SEQ ID NO: 25 - DNA, 20 bp, Artificial Sequence
  Synthetic
acacacagagattggagggt

SEQ ID NO: 26 - DNA, 20 bp, Artificial Sequence
  Synthetic
gctcacgtagaagaccctcc

SEQ ID NO: 27 - DNA, 20 bp, Artificial Sequence
  Synthetic
ggcagggctgatgacaatcc

SEQ ID NO: 28 - DNA, 20 bp, Artificial Sequence
  Synthetic
tgcaaaacaggcagggctga

SEQ ID NO: 29 - DNA, 20 bp, Artificial Sequence
  Synthetic
agaccagggcacttcccagg

SEQ ID NO: 30 - DNA, 20 bp, Artificial Sequence
  Synthetic
cctgcctccgtgtgtggccc

SEQ ID NO: 31 - DNA, 20 bp, Artificial Sequence
  Synthetic
gaccagccagcaccaagagc

SEQ ID NO: 32 - DNA, 20 bp, Artificial Sequence
  Synthetic
ccacaggacagcgttgccac

SEQ ID NO: 33 - DNA, 20 bp, Artificial Sequence
  Synthetic
ccggttcttgtactcgggcc

SEQ ID NO: 34 - DNA, 20 bp, Artificial Sequence
  Synthetic
ccaaccaggagaggtgaggc

SEQ ID NO: 35 - DNA, 20 bp, Artificial Sequence
  Synthetic
ggcaaagcagtaggtcaggc

SEQ ID NO: 36 - DNA, 20 bp, Artificial Sequence
  Synthetic
gcctcatgatccccacgatc

SEQ ID NO: 37 - DNA, 20 bp, Artificial Sequence
  Synthetic
agtcctactaccagccgcct

SEQ ID NO: 38 - DNA, 20 bp, Artificial Sequence
  Synthetic
tcagggtaagactccacttc

SEQ ID NO: 39 - DNA, 20 bp, Artificial Sequence
  Synthetic
agggtgttcctgggtctcca

SEQ ID NO: 40 - DNA, 20 bp, Artificial Sequence
  Synthetic
ctccgtgtgtggcccatggc

SEQ ID NO: 41 - DNA, 20 bp, Artificial Sequence
  Synthetic
ggatggtgatgttccctgcc

SEQ ID NO: 42 - DNA, 20 bp, Artificial Sequence
  Synthetic
tgagaaagaccagccagcac

SEQ ID NO: 43 - DNA, 20 bp, Artificial Sequence
  Synthetic
gggcgcagagccaggatcac

SEQ ID NO: 44 - DNA, 20 bp, Artificial Sequence
  Synthetic
ggcccaggatgggagcaggt

SEQ ID NO: 45 - DNA, 20 bp, Artificial Sequence
  Synthetic
agggcgtacactttcccttc

SEQ ID NO: 46 - DNA, 20 bp, Artificial Sequence
  Synthetic
cagccccttgcttctgcgga

SEQ ID NO: 47 - DNA, 20 bp, Artificial Sequence
  Synthetic
aaggagagggatgccagcca

SEQ ID NO: 48 - DNA, 22 bp, Artificial Sequence
  Synthetic
ctgttactttacagagggtttg

SEQ ID NO: 49 - DNA, 25 bp, Artificial Sequence
  Synthetic
cttctgttactttacagagggtttg

SEQ ID NO: 50 - DNA, 21 bp, Artificial Sequence
  Synthetic
ctgttactttacagagggttt

SEQ ID NO: 51 - DNA, 20 bp, Artificial Sequence
  Synthetic
gccctcgtcagatgggcgca

SEQ ID NO: 52 - DNA, 20 bp, Artificial Sequence
  Synthetic
agtcctactaccagccgcct

SEQ ID NO: 53 - DNA, 20 bp, Artificial Sequence
  Synthetic
agtaagagtctattgaggta

SEQ ID NO: 54 - DNA, 20 bp, Artificial Sequence
  Synthetic
ggttgagtttcacaacctga

SEQ ID NO: 55 - DNA, 20 bp, Artificial Sequence
  Synthetic
gtccacagaatggaacagag

SEQ ID NO: 56 - DNA, 20 bp, Artificial Sequence
  Synthetic
ggcatccacccggcagatgc

SEQ ID NO: 57 - DNA, 20 bp, Artificial Sequence
  Synthetic
tggatggcatccacccggca

SEQ ID NO: 58 - DNA, 20 bp, Artificial Sequence
  Synthetic
aggcacctcctaggctcaca

SEQ ID NO: 59 - DNA, 20 bp, Artificial Sequence
  Synthetic
gccaatggagcttaggcacc

SEQ ID NO: 60 - DNA, 20 bp, Artificial Sequence
  Synthetic
catgatggggaaagccagga

SEQ ID NO: 61 - DNA, 20 bp, Artificial Sequence
  Synthetic
aattgcaagccatagcttca

SEQ ID NO: 62 - DNA, 20 bp, Artificial Sequence
  Synthetic
cggcaaggcagcaatacctt

SEQ ID NO: 63 - DNA, 20 bp, Artificial Sequence
  Synthetic
cccagcaatgacagacagca

SEQ ID NO: 64 - DNA, 20 bp, Artificial Sequence
  Synthetic
ggtctgaaaggaccaggccc

SEQ ID NO: 65 - DNA, 20 bp, Artificial Sequence
  Synthetic
tgggaaacccccggaagcaa

SEQ ID NO: 66 - DNA, 20 bp, Artificial Sequence
  Synthetic
ggctttgggaaacccccgga

SEQ ID NO: 67 - DNA, 19 bp, Artificial Sequence
  Synthetic
tcagattcaggatctggga

SEQ ID NO: 68 - DNA, 20 bp, Artificial Sequence
  Synthetic
cccaggtgaagtcctctgac

SEQ ID NO: 69 - DNA, 20 bp, Artificial Sequence
  Synthetic
ctgcgccgaatcctgcccca

SEQ ID NO: 70 - DNA, 20 bp, Artificial Sequence
  Synthetic
caggcccgaaggtaaggctg

SEQ ID NO: 71 - DNA, 20 bp, Artificial Sequence
  Synthetic
tcagctagcacggtgctgaa

SEQ ID NO: 72 - DNA, 20 bp, Artificial Sequence
  Synthetic
ggcccagcaaacttgcccgt

SEQ ID NO: 73 - DNA, 20 bp, Artificial Sequence
  Synthetic
ccaccacagtgggctcagcc

SEQ ID NO: 74 - DNA, 19 bp, Artificial Sequence
  Synthetic
ggccatgagggcaatctaa

SEQ ID NO: 75 - DNA, 21 bp, Artificial Sequence
  Synthetic
gtggccatgagggcaatctaa

SEQ ID NO: 76 - DNA, 20 bp, Artificial Sequence
  Synthetic
gatttaacatttggcgccca

SEQ ID NO: 77 - DNA, 20 bp, Artificial Sequence
  Synthetic
aaagttacaacattatatct

SEQ ID NO: 78 - DNA, 20 bp, Artificial Sequence
  Synthetic
agtgcgattctcaaacctac

SEQ ID NO: 79 - DNA, 16 bp, Artificial Sequence
  Synthetic
tatttgcgagctcccc

SEQ ID NO: 80 - DNA, 15 bp, Artificial Sequence
  Synthetic
tatttgcgagctccc

SEQ ID NO: 81 - DNA, 14 bp, Artificial Sequence
  Synthetic
tatttgcgagctcc

SEQ ID NO: 82 - DNA, 20 bp, Artificial Sequence
  Synthetic
cgacagctcctgcgctcctc

SEQ ID NO: 83 - DNA, 16 bp, Artificial Sequence
  Synthetic
agctccccgtacctcc

SEQ ID NO: 84 - DNA, 16 bp, Artificial Sequence
  Synthetic
tgcgagctccccgtac

SEQ ID NO: 85 - DNA, 10 bp, Artificial Sequence
  Synthetic
ctccccgtac

SEQ ID NO: 86 - DNA, 11 bp, Artificial Sequence
  Synthetic
gctccccgtac

SEQ ID NO: 87 - DNA, 12 bp, Artificial Sequence
  Synthetic
agctccccgtac

SEQ ID NO: 88 - DNA, 13 bp, Artificial Sequence
  Synthetic
gagctccccgtac

SEQ ID NO: 89 - DNA, 14 bp, Artificial Sequence
  Synthetic
cgagctccccgtac

SEQ ID NO: 90 - DNA, 15 bp, Artificial Sequence
  Synthetic
gcgagctccccgtac

SEQ ID NO: 91 - DNA, 13 bp, Artificial Sequence
  Synthetic
gcgagctccccgt

SEQ ID NO: 92 - DNA, 15 bp, Artificial Sequence
  Synthetic
gccgccgccaagtct

SEQ ID NO: 93 - DNA, 24 bp, Artificial Sequence
  Synthetic
gagaagcaaagctttcaccctgtg

SEQ ID NO: 94 - DNA, 22 bp, Artificial Sequence
  Synthetic
gaagcaaagctttcaccctgtg

SEQ ID NO: 95 - DNA, 19 bp, Artificial Sequence
  Synthetic
gcaaagctttcaccctgtg

SEQ ID NO: 96 - DNA, 24 bp, Artificial Sequence
  Synthetic
ctccccgtacctcctaaggctcct

SEQ ID NO: 97 - DNA, 22 bp, Artificial Sequence
  Synthetic
ccccgtacctcctaaggctcct

SEQ ID NO: 98 - DNA, 19 bp, Artificial Sequence
  Synthetic
ccgtacctcctaaggctcc

SEQ ID NO: 99 - DNA, 32 bp, Artificial Sequence
  Synthetic
gatcagggtaccaagagtggctcctgtaggca

SEQ ID NO: 100 - DNA, 32 bp, Artificial Sequence
  Synthetic
gatagcctcgaggtagaattccaatcagctga

SEQ ID NO: 101 - DNA, 20 bp, Artificial Sequence
  Synthetic
tgcatcccccaggccaccat

SEQ ID NO: 102 - DNA, 21 bp, Artificial Sequence
  Synthetic
gccgaggtccatgtcgtacgc

SEQ ID NO: 103 - DNA, 20 bp, Artificial Sequence
  Synthetic
acacgtctacaggagtctgg

SEQ ID NO: 104 - DNA, 20 bp, Artificial Sequence
  Synthetic
caagcccatggtgcatctgg

SEQ ID NO: 105 - DNA, 20 bp, Artificial Sequence
  Synthetic
ctggggtccatcgtgggtgc

SEQ ID NO: 106 - DNA, 20 bp, Artificial Sequence
  Synthetic
ccgtgctgcctacaggagcc

SEQ ID NO: 107 - DNA, 20 bp, Artificial Sequence
  Synthetic
ggtgcttccgtaagttctgg

SEQ ID NO: 108 - DNA, 20 bp, Artificial Sequence
  Synthetic
ggattgccaagcccatggtg

SEQ ID NO: 109 - DNA, 20 bp, Artificial Sequence
  Synthetic
ctaagtagtgctagccggga

SEQ ID NO: 110 - DNA, 20 bp, Artificial Sequence
  Synthetic
tgcgtctccacggaaacagc

SEQ ID NO: 111 - DNA, 20 bp, Artificial Sequence
  Synthetic
gtgcggcccaggtacttggc

SEQ ID NO: 112 - DNA, 20 bp, Artificial Sequence
  Synthetic
acaaggaggagggccacagt

SEQ ID NO: 113 - DNA, 20 bp, Artificial Sequence
  Synthetic
tgagaggtttggaggaaatc

SEQ ID NO: 114 - DNA, 20 bp, Artificial Sequence
  Synthetic
gatagtctctctgtcagcgt

SEQ ID NO: 115 - DNA, 20 bp, Artificial Sequence
  Synthetic
gttgctgggcctgctaggct

SEQ ID NO: 116 - DNA, 20 bp, Artificial Sequence
  Synthetic
ctaggtctcgtcgtcggtgg

SEQ ID NO: 117 - DNA, 20 bp, Artificial Sequence
  Synthetic
tctcactgccttcactctgc

SEQ ID NO: 118 - DNA, 21 bp, Artificial Sequence
  Synthetic
gtaccagatgaaggttatcaa

SEQ ID NO: 119 - DNA, 20 bp, Artificial Sequence
  Synthetic
ctttggagattattcgagtt

SEQ ID NO: 120 - DNA, 20 bp, Artificial Sequence
  Synthetic
gcaagtgtaaagccctgagt

SEQ ID NO: 121 - DNA, 20 bp, Artificial Sequence
  Synthetic
agaattccaatcagctgaga

SEQ ID NO: 122 - DNA, 20 bp, Artificial Sequence
  Synthetic
tctgagaaactctgcacttc

SEQ ID NO: 123 - DNA, 20 bp, Artificial Sequence
  Synthetic
tcctcaggctctcactgcct

SEQ ID NO: 124 - DNA, 20 bp, Artificial Sequence
  Synthetic
ggttgttcaagtccgtgctg

SEQ ID NO: 125 - DNA, 21 bp, Artificial Sequence
  Synthetic
gccgaggtccatgtcgtagcc

SEQ ID NO: 126 - DNA, 20 bp, Artificial Sequence
  Synthetic
agactccacttctgagatgt

SEQ ID NO: 127 - DNA, 20 bp, Artificial Sequence
  Synthetic
tgaagaaaaattccactttt

SEQ ID NO: 128 - DNA, 20 bp, Artificial Sequence
  Synthetic
tttagtttcacagcttgctg

SEQ ID NO: 129 - DNA, 20 bp, Artificial Sequence
  Synthetic
tcccaggtgcaaaacaggca

SEQ ID NO: 130 - DNA, 20 bp, Artificial Sequence
  Synthetic
gtgaaagccaacaatttgga

SEQ ID NO: 131 - DNA, 20 bp, Artificial Sequence
  Synthetic
catggcttcagatgcttagg

SEQ ID NO: 132 - DNA, 20 bp, Artificial Sequence
  Synthetic
ttgaggtatggacacttgga

SEQ ID NO: 133 - DNA, 20 bp, Artificial Sequence
  Synthetic
gcgttgccacttctttcact

SEQ ID NO: 134 - DNA, 20 bp, Artificial Sequence
  Synthetic
ttttgccagtagatgcgagt

SEQ ID NO: 135 - DNA, 20 bp, Artificial Sequence
  Synthetic
ggccatatattcatgtcccc

SEQ ID NO: 136 - DNA, 20 bp, Artificial Sequence
  Synthetic
gccaggatcacaatggagag

SEQ ID NO: 137 - DNA, 20 bp, Artificial Sequence
  Synthetic
gtatgtgccctcgtcagatg

SEQ ID NO: 138 - DNA, 20 bp, Artificial Sequence
  Synthetic
ttcagccaggtgttcccgct

SEQ ID NO: 139 - DNA, 20 bp, Artificial Sequence
  Synthetic
ggaagtcagctttgactgat

SEQ ID NO: 140 - DNA, 20 bp, Artificial Sequence
  Synthetic
cctccagaggttgagcaaat

SEQ ID NO: 141 - DNA, 20 bp, Artificial Sequence
  Synthetic
ccaaccaggagaggtgaggc

SEQ ID NO: 142 - DNA, 20 bp, Artificial Sequence
  Synthetic
gaagctgtggttggttgtca

SEQ ID NO: 143 - DNA, 20 bp, Artificial Sequence
  Synthetic
ttgaaggtctgattcactct

SEQ ID NO: 144 - DNA, 20 bp, Artificial Sequence
  Synthetic
aaggtaatggcccaggatgg

SEQ ID NO: 145 - DNA, 20 bp, Artificial Sequence
  Synthetic
aagcagtaggtcaggcagca

SEQ ID NO: 146 - DNA, 20 bp, Artificial Sequence
  Synthetic
ccttgcttctgcggacactg

SEQ ID NO: 147 - DNA, 20 bp, Artificial Sequence
  Synthetic
agccccttgcttctgcggac

SEQ ID NO: 148 - DNA, 20 bp, Artificial Sequence
  Synthetic
tgacggaggctaccttcaga

SEQ ID NO: 149 - DNA, 20 bp, Artificial Sequence
  Synthetic
gtaaaacagcttaaatttgt

SEQ ID NO: 150 - DNA, 20 bp, Artificial Sequence
  Synthetic
agaagaggttacattaagca

SEQ ID NO: 151 - DNA, 20 bp, Artificial Sequence
  Synthetic
agataatgaattggctgaca

SEQ ID NO: 152 - DNA, 20 bp, Artificial Sequence
  Synthetic
gcgtcatcatccgcaccatc

SEQ ID NO: 153 - DNA, 20 bp, Artificial Sequence
  Synthetic
cgttgcttgtgccgacagtg

SEQ ID NO: 154 - DNA, 20 bp, Artificial Sequence
  Synthetic
gctcacgaagaacaccttcc

SEQ ID NO: 155 - DNA, 20 bp, Artificial Sequence
  Synthetic
agagaaactagtaagagtct

SEQ ID NO: 156 - DNA, 20 bp, Artificial Sequence
  Synthetic
tggcatccacccggcagatg

SEQ ID NO: 157 - DNA, 20 bp, Artificial Sequence
  Synthetic
tcgagaaacagagatgtaga

SEQ ID NO: 158 - DNA, 20 bp, Artificial Sequence
  Synthetic
tggagcttaggcacctccta

SEQ ID NO: 159 - DNA, 20 bp, Artificial Sequence
  Synthetic
tggggaaagccaggaatcta

SEQ ID NO: 160 - DNA, 20 bp, Artificial Sequence
  Synthetic
cagcacaaagagaagaatga

SEQ ID NO: 161 - DNA, 20 bp, Artificial Sequence
  Synthetic
atgaggagagttgtaacggc

SEQ ID NO: 162 - DNA, 20 bp, Artificial Sequence
  Synthetic
aagtccggttcttatactcg

SEQ ID NO: 163 - DNA, 20 bp, Artificial Sequence
  Synthetic
gcaggtaatccttttagtgt

SEQ ID NO: 164 - DNA, 20 bp, Artificial Sequence
  Synthetic
gtgaagtcctctgacacgtg

SEQ ID NO: 165 - DNA, 20 bp, Artificial Sequence
  Synthetic
cgaatcctgccccaaagagc

SEQ ID NO: 166 - DNA, 20 bp, Artificial Sequence
  Synthetic
actgcgccgaatcctgcccc

SEQ ID NO: 167 - DNA, 20 bp, Artificial Sequence
  Synthetic
ttgatgatgacaacgatgac

SEQ ID NO: 168 - DNA, 20 bp, Artificial Sequence
  Synthetic
ctgttgtttgtttctctgct

SEQ ID NO: 169 - DNA, 20 bp, Artificial Sequence
  Synthetic
tgttcagctaatgcttcttc

SEQ ID NO: 170 - DNA, 20 bp, Artificial Sequence
  Synthetic
gttaactctatcttgtgtca

SEQ ID NO: 171 - DNA, 20 bp, Artificial Sequence
  Synthetic
tccacttcagtcatcaagca

SEQ ID NO: 172 - DNA, 20 bp, Artificial Sequence
  Synthetic
tgctcaatactctcttttta

SEQ ID NO: 173 - DNA, 20 bp, Artificial Sequence
  Synthetic
aggcccagcaaacttgcccg

SEQ ID NO: 174 - DNA, 20 bp, Artificial Sequence
  Synthetic
aacggcaaggcagcaatacc

SEQ ID NO: 175 - DNA, 20 bp, Artificial Sequence
  Synthetic
cagaagcaaggtggtaagaa

SEQ ID NO: 176 - DNA, 20 bp, Artificial Sequence
  Synthetic
gcctgtccactgtagctcca

SEQ ID NO: 177 - DNA, 20 bp, Artificial Sequence
  Synthetic
agaatgttactcagtcccat

SEQ ID NO: 178 - DNA, 20 bp, Artificial Sequence
  Synthetic
tcagaggagcagcaccagag

SEQ ID NO: 179 - DNA, 20 bp, Artificial Sequence
  Synthetic
tggcatggcaggtctgcagt

SEQ ID NO: 180 - DNA, 20 bp, Artificial Sequence
  Synthetic
agctcactcaggctttggtt

SEQ ID NO: 181 - DNA, 20 bp, Artificial Sequence
  Synthetic
tgcctaagtatacctcattc

SEQ ID NO: 182 - DNA, 20 bp, Artificial Sequence
  Synthetic
ctgtcaaatttctctttgcc

SEQ ID NO: 183 - DNA, 20 bp, Artificial Sequence
  Synthetic
catatacttggaatgaacac

SEQ ID NO: 184 - DNA, 20 bp, Artificial Sequence
  Synthetic
ggtccaactgtccgaatcaa

SEQ ID NO: 185 - DNA, 20 bp, Artificial Sequence
  Synthetic
tgatctgaagattgtgaagt

SEQ ID NO: 186 - DNA, 20 bp, Artificial Sequence
  Synthetic
aagcccttgtccttgatctg

SEQ ID NO: 187 - DNA, 20 bp, Artificial Sequence
  Synthetic
tgtgatggatgatacattga

SEQ ID NO: 188 - DNA, 20 bp, Artificial Sequence
  Synthetic
tcaggttgactgaagttagc

SEQ ID NO: 189 - DNA, 20 bp, Artificial Sequence
  Synthetic
gtgtatagatgagcaggtca

SEQ ID NO: 190 - DNA, 20 bp, Artificial Sequence
  Synthetic
tctgtgacattatcttgaga

SEQ ID NO: 191 - DNA, 20 bp, Artificial Sequence
  Synthetic
aagataaaagccgcgtcttg

SEQ ID NO: 192 - DNA, 20 bp, Artificial Sequence
  Synthetic
agaaaaccatcacacatata

SEQ ID NO: 193 - DNA, 20 bp, Artificial Sequence
  Synthetic
agagttgcgaggccgcttct

SEQ ID NO: 194 - DNA, 20 bp, Artificial Sequence
  Synthetic
tccctctccattgtgttggt

SEQ ID NO: 195 - DNA, 20 bp, Artificial Sequence
  Synthetic
catcagatctttcaggtata

SEQ ID NO: 196 - DNA, 20 bp, Artificial Sequence
  Synthetic
ggctttactctttaattaaa

SEQ ID NO: 197 - DNA, 20 bp, Artificial Sequence
  Synthetic
gaaatcaaaaaggttgccca

SEQ ID NO: 198 - DNA, 20 bp, Artificial Sequence
  Synthetic
ggagtcctggagccccctta

SEQ ID NO: 199 - DNA, 20 bp, Artificial Sequence
  Synthetic
ttggcatacggagcagagct

SEQ ID NO: 200 - DNA, 20 bp, Artificial Sequence
  Synthetic
tgtgctctgaagtgaaaaga

SEQ ID NO: 201 - DNA, 20 bp, Artificial Sequence
  Synthetic
ggcttggcccataagtgtgc

SEQ ID NO: 202 - DNA, 20 bp, Artificial Sequence
  Synthetic
cctaaattttatttccaggt

SEQ ID NO: 203 - DNA, 20 bp, Artificial Sequence
  Synthetic
gctccaagtgtcccaatgaa

SEQ ID NO: 204 - DNA, 20 bp, Artificial Sequence
  Synthetic
agtatgtttctcactccgat

SEQ ID NO: 205 - DNA, 20 bp, Artificial Sequence
  control oligonucleotide
tgccagcacccggtacgtcc

SEQ ID NO: 206 - DNA, 20 bp, Artificial Sequence
  Synthetic
gctgcctacaggagccactc

SEQ ID NO: 207 - DNA, 20 bp, Artificial Sequence
  Synthetic
tcaagtccgtgctgcctaca

SEQ ID NO: 208 - DNA, 20 bp, Artificial Sequence
  Synthetic
gtctacaggagtctggttgt

SEQ ID NO: 209 - DNA, 20 bp, Artificial Sequence
  Synthetic
agcttgcgtctccacggaaa

SEQ ID NO: 210 - DNA, 20 bp, Artificial Sequence
  Synthetic
tcacactatcaagtttctct

SEQ ID NO: 211 - DNA, 20 bp, Artificial Sequence
  Synthetic
gtcaaagctcgtgcggccca

SEQ ID NO: 212 - DNA, 20 bp, Artificial Sequence
  Synthetic
gtgaagtcgtagagtccagt

SEQ ID NO: 213 - DNA, 20 bp, Artificial Sequence
  Synthetic
gtgaccttgcttagacgtgc

SEQ ID NO: 214 - DNA, 20 bp, Artificial Sequence
  Synthetic
catcttcttaggtttcgggt

SEQ ID NO: 215 - DNA, 20 bp, Artificial Sequence
  Synthetic
ggctgttggagatactgaac

SEQ ID NO: 216 - DNA, 20 bp, Artificial Sequence
  Synthetic
gggaatgaaagagagaggct

SEQ ID NO: 217 - DNA, 20 bp, Artificial Sequence
  Synthetic
acatacaatgatgagcagca

SEQ ID NO: 218 - DNA, 20 bp, Artificial Sequence
  Synthetic
gtctctctgtcagcgttact

SEQ ID NO: 219 - DNA, 20 bp, Artificial Sequence
  Synthetic
tgccaagcccatggtgcatc

SEQ ID NO: 220 - DNA, 20 bp, Artificial Sequence
  Synthetic
gcaatttggggttcaagttc

SEQ ID NO: 221 - DNA, 20 bp, Artificial Sequence
  Synthetic
caatcagctgagaacatttt

SEQ ID NO: 222 - DNA, 20 bp, Artificial Sequence
  Synthetic
ttttgtataaaacaatcata

SEQ ID NO: 223 - DNA, 20 bp, Artificial Sequence
  Synthetic
ccttcactctgcatttggtt

SEQ ID NO: 224 - DNA, 20 bp, Artificial Sequence
  Synthetic
tgcatgttatcaccatactc

SEQ ID NO: 225 - DNA, 20 bp, Artificial Sequence
  Synthetic
ccctccagtgatgtttacaa

SEQ ID NO: 226 - DNA, 20 bp, Artificial Sequence
  Synthetic
gaagaccctccagtgatgtt

SEQ ID NO: 227 - DNA, 20 bp, Artificial Sequence
  Synthetic
cgtagaagaccctccagtga

SEQ ID NO: 228 - DNA, 20 bp, Artificial Sequence
  Synthetic
ttcccaggtgcaaaacaggc

SEQ ID NO: 229 - DNA, 20 bp, Artificial Sequence
  Synthetic
tggcttcagatgcttagggt

SEQ ID NO: 230 - DNA, 20 bp, Artificial Sequence
  Synthetic
cctccgtgtgtggcccatgg

SEQ ID NO: 231 - DNA, 20 bp, Artificial Sequence
  Synthetic
ggtgatgttccctgcctccg

SEQ ID NO: 232 - DNA, 20 bp, Artificial Sequence
  Synthetic
gatggtgatgttccctgcct

SEQ ID NO: 233 - DNA, 20 bp, Artificial Sequence
  Synthetic
aggtatggacacttggatgg

SEQ ID NO: 234 - DNA, 20 bp, Artificial Sequence
  Synthetic
gaaagaccagccagcaccaa

SEQ ID NO: 235 - DNA, 20 bp, Artificial Sequence
  Synthetic
cagcgttgccacttctttca

SEQ ID NO: 236 - DNA, 20 bp, Artificial Sequence
  Synthetic
gtgaccacaggacagcgttg

SEQ ID NO: 237 - DNA, 20 bp, Artificial Sequence
  Synthetic
agatgcgagtttgtgccagc

SEQ ID NO: 238 - DNA, 20 bp, Artificial Sequence
  Synthetic
ccttttgccagtagatgcga

SEQ ID NO: 239 - DNA, 20 bp, Artificial Sequence
  Synthetic
cggttcttgtactcgggcca

SEQ ID NO: 240 - DNA, 20 bp, Artificial Sequence
  Synthetic
cgcagagccaggatcacaat

SEQ ID NO: 241 - DNA, 20 bp, Artificial Sequence
  Synthetic
cttcagccaggtgttcccgc

SEQ ID NO: 242 - DNA, 20 bp, Artificial Sequence
  Synthetic
taacgtcacttcagccaggt

SEQ ID NO: 243 - DNA, 20 bp, Artificial Sequence
  Synthetic
ttctccattttccaaccagg

SEQ ID NO: 244 - DNA, 20 bp, Artificial Sequence
  Synthetic
ctgttgtgttgatggcattt

SEQ ID NO: 245 - DNA, 20 bp, Artificial Sequence
  Synthetic
catgaagctgtggttggttg

SEQ ID NO: 246 - DNA, 20 bp, Artificial Sequence
  Synthetic
aggaaaatgctcttgcttgg

SEQ ID NO: 247 - DNA, 20 bp, Artificial Sequence
  Synthetic
tgggagcaggttatcaggaa

SEQ ID NO: 248 - DNA, 20 bp, Artificial Sequence
  Synthetic
taaggtaatggcccaggatg

SEQ ID NO: 249 - DNA, 20 bp, Artificial Sequence
  Synthetic
ggtcaggcagcatatcacaa

SEQ ID NO: 250 - DNA, 20 bp, Artificial Sequence
  Synthetic
gccccttgcttgtgcggaca

SEQ ID NO: 251 - DNA, 20 bp, Artificial Sequence
  Synthetic
agatcttttcagccccttgc

SEQ ID NO: 252 - DNA, 20 bp, Artificial Sequence
  Synthetic
tttgttaagggaagaatgcc

SEQ ID NO: 253 - DNA, 20 bp, Artificial Sequence
  Synthetic
aaaggagagggatgccagcc

SEQ ID NO: 254 - DNA, 20 bp, Artificial Sequence
  Synthetic
caagacaattcaagatggca

SEQ ID NO: 255 - DNA, 20 bp, Artificial Sequence
  Synthetic
cgtgtgtctgtgctagtccc

SEQ ID NO: 256 - DNA, 20 bp, Artificial Sequence
  Synthetic
gctgcttctgctgtgaccta

SEQ ID NO: 257 - DNA, 20 bp, Artificial Sequence
  Synthetic
tatttgcgagctccccgtac

SEQ ID NO: 258 - DNA, 20 bp, Artificial Sequence
  Synthetic
gcataagcacagcagcattc

SEQ ID NO: 259 - DNA, 20 bp, Artificial Sequence
  Synthetic
tccaaaaagagaccagatgc

SEQ ID NO: 260 - DNA, 20 bp, Artificial Sequence
  Synthetic
aaatgcctgtccactgtagc

SEQ ID NO: 261 - DNA, 20 bp, Artificial Sequence
  Synthetic
cttcagaggagcagcaccag

SEQ ID NO: 262 - DNA, 20 bp, Artificial Sequence
  Synthetic
gaatcttcagaggagcagca

SEQ ID NO: 263 - DNA, 20 bp, Artificial Sequence
  Synthetic
caaattggcatggcaggtct

SEQ ID NO: 264 - DNA, 20 bp, Artificial Sequence
  Synthetic
gctttggttttgagagtttg

SEQ ID NO: 265 - DNA, 20 bp, Artificial Sequence
  Synthetic
aggctttggttttgagagtt

SEQ ID NO: 266 - DNA, 20 bp, Artificial Sequence
  Synthetic
gctcactcaggctttggttt

SEQ ID NO: 267 - DNA, 20 bp, Artificial Sequence
  Synthetic
ggtcctgccaaaatactact

SEQ ID NO: 268 - DNA, 20 bp, Artificial Sequence
  Synthetic
agcccttgtccttgatctga

SEQ ID NO: 269 - DNA, 20 bp, Artificial Sequence
  Synthetic
tgtgggctttttgtgatgga

SEQ ID NO: 270 - DNA, 20 bp, Artificial Sequence
  Synthetic
aatcattcctgtgggctttt

SEQ ID NO: 271 - DNA, 20 bp, Artificial Sequence
  Synthetic
ccgtgtatagatgagcaggt

SEQ ID NO: 272 - DNA, 20 bp, Artificial Sequence
  Synthetic
accgtgtatagatgagcagg

SEQ ID NO: 273 - DNA, 20 bp, Artificial Sequence
  Synthetic
tcatcttcttaggttctggg

SEQ ID NO: 274 - DNA, 20 bp, Artificial Sequence
  Synthetic
acaagctgatggaaacgtcg

SEQ ID NO: 275 - DNA, 20 bp, Artificial Sequence
  Synthetic
tgctcgtaacatcagggaat

SEQ ID NO: 276 - DNA, 20 bp, Artificial Sequence
  Synthetic
aagatggtcatattgctcgt

SEQ ID NO: 277 - DNA, 20 bp, Artificial Sequence
  Synthetic
cgcgtcttgtcagtttccag

SEQ ID NO: 278 - DNA, 20 bp, Artificial Sequence
  Synthetic
cagctgtaatccaaggaatg

SEQ ID NO: 279 - DNA, 20 bp, Artificial Sequence
  Synthetic
gggcttcatcagatctttca

SEQ ID NO: 280 - DNA, 20 bp, Artificial Sequence
  Synthetic
catgtatcacttttgtcgca

SEQ ID NO: 281 - DNA, 20 bp, Artificial Sequence
  Synthetic
agcccccttattactcatgg

SEQ ID NO: 282 - DNA, 20 bp, Artificial Sequence
  Synthetic
ggagttacagccaggctatt

SEQ ID NO: 283 - DNA, 20 bp, Artificial Sequence
  Synthetic
agtctcctcttggcatacgg

SEQ ID NO: 284 - DNA, 20 bp, Artificial Sequence
  Synthetic
cccataagtgtgctctgaag

SEQ ID NO: 285 - DNA, 20 bp, Artificial Sequence
  Synthetic
tccgtcatcgctcctcaggg

SEQ ID NO: 286 - DNA, 20 bp, Artificial Sequence
  Synthetic
gctcagcctttccacttcag

SEQ ID NO: 287 - DNA, 20 bp, Artificial Sequence
  Synthetic
gctcagcctttccacttcag

SEQ ID NO: 288 - DNA, 19 bp, Artificial Sequence
  Synthetic
ggccctcctccttgtgatg

SEQ ID NO: 289 - DNA, 29 bp, Artificial Sequence
  Synthetic
tgctcatcattgtatgtcacaagaagccg

SEQ ID NO: 290 - DNA, 19 bp, Artificial Sequence
  Synthetic
ctgggcctgctaggctgat

SEQ ID NO: 291 - DNA, 20 bp, Artificial Sequence
  Synthetic
caggaagctacgggcaagtt

SEQ ID NO: 292 - DNA, 27 bp, Artificial Sequence
  Synthetic
tgggcctttgattgcttgatgactgaa

SEQ ID NO: 293 - DNA, 18 bp, Artificial Sequence
  Synthetic
gtgggctcagcctttcca

SEQ ID NO: 294 - DNA, 20 bp, Artificial Sequence
  Synthetic
tcaagtccttccacacccaa

SEQ ID NO: 295 - DNA, 1424 bp, Homo sapiens
  CDS (148)...(1119)
aggagccttaggaggtacggggagctcgcaaatactccttttggtttattcttaccaccttgcttctgtgttccttgggaatgctgctgtgcttatgcatctggtctctttttggagctacagtggacaggcatttgtgacagcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaataaaaaaaaaaaaaaaaaaaa

SEQ ID NO: 296 - DNA, 2781 bp, Homo sapiens
  CDS (117)...(1106)
ggaaggcttgcacagggtgaaagctttgcttctctgctgctgtaacagggactagcacagacacacggatgagtggggtcatttccagatattaggtcacagcagaagcagccaaaatggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaaacccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgtttttttttaaatagacctctcaatttctggaaaactgccttttatctgcccagaattctaagctggtgccccactgaatcttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcaaaaaaaaaaaaaaaaaaaaaaaaaaaaaaaaaaaaaaaaaaaaaaaaaaaaaaaaaaaaaaaaaaaaaaaaaaaaa

SEQ ID NO: 297 - DNA, 68001 bp, Homo sapiens
gctgtcttggtggtggtatttctgttgcagttgttgttttcttgcctgcttggtgacatatttctattgacttgacacttaactggcatcttatctaggtagataatgctaattcaaaattctgcagatattgttctgttgttttttgccatttagggttgagtaagatgccaagttgttttttgtttctctgtagtcattctgttttcattttgtttttagctttgcctttggaatttaaaatgttcaaaatgatttgtctggatgagaatcgattttcataacttttgctttgatacactaaacagtttgagtttctagatgatgcccattttaattcatacgaggaaatatcttctagtatagtttctgcttgattaattctatgtttgtctcttagggacatctattaattttataatgctgcctttttttcagacttctgtttcagaatattcgctttcatcaatgtaatccttggctatagtaggaatgaaataataaaagcagtagcttctgtctgccctccttggttatgcagtccttacagaacatctccccatctcccatccccccaccccagctcagtgaaactctccacactttggttgtggaaattggcagggttaggtggctactcactcccaatccacatccacaataaatcactttttattatcttatcaaaatctgtagaatgcctctttattctattttgttgctgcggaggtttgttttctctttctaattattttattttctaggttttttgagggaatttcaagaggggagattttttattcaggctcatcttaacgtcatgtctggaactcaagctactgaattatatattctttaatacatatagacctacgtcaatgagtttaaactgcaaggaaagggttaaatttcttcctcaagtgtggtcaaaatctgtagagaaaagaggaacagcttctcttaaagaaagttagctgggtaggtatacagtcattgccgaggaaggcttgcacagggtgaaagctttgcttctctgctgctgtaacagggactagcacagacacacggatgagtggggtcatttccagatattaggtcacagcagaagcagccaaaatggatccccagtggtgagtaataattcttattctttgcagagaagttatgagttgtgactgcagtgaaaggctgaggttgaagatggtgctttgatgtgtgtccttcacttagttcctaagtggagaagctttctttttctacaaaagatctttggcacataaaggcaagaattatttgcaatgcccaaagcagttcattggtggtagttatatatatttttaggtgcctaattttggttttgtaaatctgttattcaaatactgaatgttacagtcattgattttagtgaagaatcaggaatttttaaaatatctgcataagaatgacaaataacagggaatatgttttttgtctaccagggtcagtttggtctgagggtggaggaatgagatagagaaggtagagggagagagatcaagaaaaagaaagagaaaaaagaggtataaggagaaaatgcaaaactcagttaatatgtcataatcaggccatgggagattctgggcagggttgggtagtggaaggaggtagagtgattaaattagttaccatgtattgaacatgcatgatgtgctgggtactttactagtgctatttcattgaattttattcttcacaatgacttttggaaagacatcatcattctttttgacagatggggtaactgtggcttaaaaaacttgcccaagttcacactattcataaggggtagagctaaaatctttcctgcccgcttcgtggtgcgccagaaggtttctccatgctgtggagacttcctggaaggagtcacacccgcccttctcttgggtggtggcagctggcgccagtcactatgtatttatttatttttaattatttatttttgaaacagagtctcgctctgtcgccaagctggagtgcagtggcgcaatctcagctcactgcaacctccgcctctcggattcaaacgattctccttcctcagtctcctgagtagctgggactacaggcgcccaccaccacgcccggctaatttttgtatttttggtagagacggggtccaccatgttggccaggatggtctcgatctcttgaccttgtgatctggccacctcggcctcccaaagtgctgggattacaggcgtgagccaccgcgcacccggccgtcactatgtatttataattactgttctttgaaaatcgaagtaactttcatctacccagtgcttactggtttgagaaaaagctttgttgcttttatttcagaagattaaaatttaattttccagtaaagattccttttgctccagtggaattttgaagcgttatacttgtatgaagaaaaaaagaatttcaaaatttataatttttgtggtaccatagaggggatactacttaattatgctagcactgtctgcagaggtctaaaaaaccataggctgctgtctatattgaacttgttaagattccttttgtttcacagtgcctgaagattggtcatgaaccagtaatagccattaaacaatgtctgttctcataagagatgaaataaatacaaattaaaacaacagtgaagtatcattttttctctatcaaagagataaatattaagttttaaaaagcaagcaatcaagaaccctctatcttgctaagaagggaggattatttgtaccctagttgcgcactagtggtgatatcatctttctggacaataatctagtgatacatatcaaaagcctttaaaatgtatatgccctttaaccaagcaattcaccttttaggaatttattctaagatataataatacatgtttgtaaagttttagtgatggatatttttcttgctattgtttctaataaggaaaatcttagaaacaatttacgtgtttaaaaacaagtgattggatgattatggaacatccataatggaataccatgtaattatttaaaattccactgtagaacaattcataatgtgtttagttaaaggggaaaaatgcagaacgaacagtgctatcacttttatacatgatacatgtatacaaacaatattaatcagaaatataggtagtttgtattttctactgtgtgcttttcaatttgaatcccatctgtaggcagaaaaataaagttaaatatttaagatttaaaaaaaacaatagctggtttttttcagagaagtaaccatctagtgatgtgtgataatttataagttgtgagattccatagttagggctttagccctttgcatttatctttcttcatctcttgaatcctcttcaaaatacacccactctacccataactcattagatcttgaaaggcatgttctgatagaattttatatttagaacaggctgcagcactcttcccttattttacagaagtgattgcatggctctctagggtgagttgcatattgagagggaggacagtgcagtggctaagtggccgcacattgaagccaggctgctggggtttgaataccagttccccaacttcctagctatgtgattttggacaagttgcttaaccattgtaattcccagtttcttgtctgtaaaatgggagtatggtaatatgtcgtaaagtagttgtgagattttaatgagataatccatatcctgctaagtactcaggaattgttagtagtttttattactattactgtttggattaagaaacagaggaaaagtgatttgtccaagattatacaaccacttaatggcattactaagaacagaatggaggaaggttttttccagcagaaatgttcagtatcctctgtgcctggcaggacaaccccaagttgtgcttttgggatggaggagctgatctaaaacaagcagtacccaggacaaggccagcctccagggagtgactgatgacagtgggaagccaaatggtagaaaggcaggtgaagttaaggaaactgagagtcaacttaggagcaggaatgaagcctggagcaaagagctagtgcaagagagagcagatgactcagagctactggggcttttgtaggccaccagctatgggcttcacgggaggttaatgtggtttaaagtctcaagacctgggtaattaaatagacaaatggggtctaggtgcatgggggaattttaagtatagctttgagaagatgcctaatggggagtaataatagagaaagaagctgggtggggccagaacaaggagcccacagtccaggcatccagctatgatcatcctaaaggaacagcctaagtttgaagaatcataaacaaatagagacatgataaaatttactttaaaaaaaatcccttggcagaagaatgaaaactgatttggagtgacaaggagatcatttaggagactattggagtaagccaaggagaaatggcgagggcatgaaaccagggcagttatggtgggatcagactggagaggatgcatttaagagatattttagcaccagaattgacagaatttggtttttgacagatgtagacactgggagaggaggagagatctaagtgcatagtggcatccttcacaaaatggaaggtataggaagcagagtgggtttgcgccaggcagagagaaaagacagatggtgaattccttctctaacatgttgagtttgaggtgcctgtggagcagctggatagagatgtccaagcagacaagtagatatttaggtgcaagttcaaaaaagagggatggcctggaatgcacatgaagagtcttctgcataagtatggttgacagttgaaattctcattgtgggtcaattcagtagcagagaggttggaggattgagaggaagccaaggacagaacctggaaaaccttgacatctaaggagggagatgaggaagaagaatctacaatagatactaaggaggggctagagagactggagcagcccaggagaaaagtggtgtcatagaaatcaagtgggcctgtagtcccagctacttgggagactgaggcacgagaatagcttgaacccaggaggcagaggttgcagtgagctgagatagcaccacggcactccagcctgggtgacagagtgagactccgtctcaaaaaaaaaaaaaaaagaaagaaatcaagtggggagatggatgcaagaaagaggagaatgcattcatggaaagagcatatttaccaagctttccatgttgaacacatgagatgtgacatgaaaggtaactgtagtgactacatgttaagcgttgaattgtggctcccttaaaattcatatgttgaaatcctaactcccagggcttcaggatttgatcatatttggagatagggtctttacagagataataaaatttaaatgaggtcattagggtgggtcctaatccaagacaattgttgtgcttattgtaaggaagggaaacacacagcggaaaagcggtgtgaagacacagggcgaagacggccatccacatacaagccagagagaaaggctcgcaacagattctttcctcacaaccttcagaaagaaccaaccctgtggaaaccctaagtttggactcctggcttccagaaaaaaaataaattttttttgtttaagccaccccagtttgtggtactttgttaccacagccccagcaaactaatacacttggtgagagtgcatacagccagagaaagaagctgtgaataagtgcatcagggaggagggaaagaaaccaaaacaggattacatcacttaaattaagagtagaaacatttcacaaagcagagtgtagtcacaggtcaaattctgcagaaaggcgaagtaggagaatgactgaaaatgtcagtccagaggccctcagtgaccttgacctttgcagaacactcccagttgagtggaggcagtagactttggagagcttggaaaatggaggcagcacagatggtctcctgcagaaagtctggtgataaaatgagacctcctcactggagtaaactcacctctgctgtcggtggaaataatctggagtcaggccagccagacccagagttcttccttcctccattttaaaggttaaacagagctgaggtcaatggctcatgcctgtaatcccagtgactcaggaggcggaggtgggaggatggcttgaggcgaggagttccgttcaagaccagtctgagcaacatagcgagactcccattcctaaaaaaattttaatttaaattaaaaaaaaaggctaaccaaaaataaaatccaatactttatttttcccacccaaaactagtttgggaaggatttctggaagaaaataatttttgcagtcattttacatgttggattttgagtgcacataacatacagattctattctgtattatcagttcagaggcaagttgagatttgaggcttcgcagaggtaaagcctctggtgaatctggtgagataaagaagaaaacaagcccaagaggaatttcagggatcatttataatttacatcaataaacagaatgggaaaaaaaacccattagagtttggaatagagaagtattaaaacactttcttagaaagctttgagtcaaaattaatctttctgtagtggcaggaatatgataagccaaacaaccctaatgtcacagctctatattattaggtgtcgaatcagatttgcactaaaacatcaagtaaaaataaaaggaatgaacatttggttaagtgaaccaattagtcaatacacgccagaaaatggtaaaactggataaacctaaaatactcaactacctagattaatcaaggccaacctagattatcaccccaatattacaactattttcaaccaactaaacaataaatctttatcaagagcctgatagtttaaggtactgtgatgaatacaaatgaaattgctgatactttttttcaagtctatttagaaatagaaacccacaattatgaaatgacaaaaacaattaatgcagttaataattcagtaacttttaaaaagaaataaacatgacaaaaagttcattctcaccaaatactaaagaaatgccaattcaaacaccaatgagatattttcctgtatcagattagacagtaaaacaaacaatccaatcagaaaaatgggcaaaagatataaaaagacatttccccaaagaaaatatacagatggtgaacaaccatataagagagtcaacatcatttgcctttatgaaaaaattaaaccactacctacctataaaaatggttaaaataataaaagataatgacaacaccaaatgatggcaggatgcggagaaactggatcatgcatacattgcttgttgggaatgtacaatggtcaagccactctagaaaacagtttggcagtttcttataaaaccaaacatgcatttagtatatgacccagcaactgcattcttgggttttgatcccagagaaataaaagcctatgctcctgcaaaaatcagtatatgaatatttataccagctttattcataatagtaaaaaactggggaaaaaagtccctcagtgggtgaatcgtaacacaaactgtgtgtgcaagatgttaccactgaaggaagctgggtgaaggtacacaggacttccctgtacattttttcaacttcttttgaatcaataattatttaaaaatgaaaagtttaaaaagtaaaaaaaaaaaacaaaaactaaaaatgttcatcttcactaaatattaaaaaaaatgccaattcaaacacaagatattctccttttacaaattaacaatttatatggattttgggagggttgggagtaatcatgctaataagtatgcaataagaggatactttcgtatactactgattgtgggaatatgaatggggagaacatttctggaaagcaatatgtcaacaatatcaagagtcttaaaaatggttgtacaagcagacacccattctgggcactgccaatttctccatgtccttagtacattttttttcagttcattcagcatctttgttccaggcactgtgctaaacattaaaaatacaccaaagatgagtatgagtaaacatgatttctgttctcaagaatttcagttttgtggtaaatatatcaaaggtgattttttataagagttttttataacagggtgtgacgtttcataggagcatgaaggtagctgtttcctatttgtctgtaggcagtatgatgtcttagataaatgccagggttttgagctagtttggttggtatcaaataataagtagttaataaatcatcttctatttattagtggtatcactttgggaagcctattagcttcctgaacttcagtgtcctctgtaagatgaggctactaagcacttgccaatgccatgaggaatataacaatttataatggacaggaagtcctatggatataagatattttaggactcacattctttgctttaaaatctattatttcctatatttttaattgtcagagttctttagctctgccttttctgattgatttccagcagatggactcttacctataacctagaagttgctatagtagacctcctaactatagataagagaagggcatgccaaatgcagttgaatcaggtgaaagtcaagcaacaaagctgcctaaaataaattttatgtaaggtagggtgccaaaatcattaaaataaaattctattctataactgtaatcacgtaagtgctttcatgaagttgtctatgaaaactttctttttcgctttctggacttcaaatattttaagtttgcttttcatttacaaagatttttttgctcattagtaatcatgaactgtattcaaacttacacttctaattctagaagatatataaactaccattttttaattataaaaatgtttatatatcttgctttaataatttcacctctagggatctagctagttaaataacaagagctacggaaacatatttgtgcaccaaaatgtttattacattatttaatgtaatgaaaaataagaatcaacctaaataagtagaagaataggtaagtaagttaaaaatgaagttaataatgactccttaatgagagaagacaacataaggctacatacagaattgtaaagaaaataatccacagaatgtgtgttttattttggtaaatggttcctaaaactaagtaagtacatagaaagatttttttttttttttttttaaagacagagtctcactcacgttgtcacctaggctggagtgcagtagtgcaatctcagttcactgcagccttcacctcccgggttcaggacacgccaccacacccagctaattttcttgtatttttagtagagacacagtttcatcatggggctggtctcaaactcctgacctcaagtgatctgcccaccttggccttctaaagtgctgtattataggtgtgacccaccgcacctggcctaggaagaattttagaaagaaagctccatatcaggaattgagaagccggtgttttaattgagaatatatttgcacagaaaaatcttggcataaatattggtttacaaaacaaacaaacaaagtaatgtcctttaatcttggatcaggagctgcccaacaactccaaaaagtcagctcatgcaaaaccatccaaggacagatgaatcagccaaacaagagagaaaggggaagggaaagtgtcttttcacaggcagcttttgaggcagtgcataaaccatgcctctgcaccatccagaccagacagttgtgacacagggttgacaaagcaggacaacgaagggtagctgctcctaaggtggggatgatgctggagcaagggggagcaccaagaggaaaaaaaaaaagcataaaaataagatagcatagtaaaaaataagatagtcatagtagttacctcttgattgatgggattatggaaatagggttatttctttgactaaaattgccagatcttcagtacaattacattgctctgctgagcaggatgaaatcaagttgaaaagtaatctagtagtgaggtacagcccgtatgctgcaaatggccaacatagatcctcagatgacagaagtgagtgatgcaggcctgtggtttacgtacagctccatgacgtatgaatggcagaagctgtgtatgtccacaggcgagccccatttcaagaagtgcttctggtcaccactctgttgtcctgtgtataaggatgtggttcagaacagccaagtcttatatttcaagaggagccagaaagacaaacttaccagtgaaatcctaatatttatataatagctcaatatctggaggggctggtttggtataaatcaccagttttacctctgacctctgttaatgcatcagaggttcaggagagagtgagaaaattgtaaatggaatattttaggactattatattggggcccaggctttagtgagtcagagcgagaaagtagggggctgaaactatttgactattaatttattcaataaagttttatttaatgttggaaagatgaagatgaatcagatagaaatcctgtttagaagcttctatgggaagagatatcaccatagctaatatgtcttagcctctaaaaggaattatggcaaactatattgtatcatatatgatctcatttaatcatcataactgcaggaggtaagtagtattatccctgattttccataagtgaaaactgagtctctcttagttacatagctgacaacacaaccaggattccaaatgccagtctgatcccagagccaagctatgaacaaccatgctatatattatggcagatcagggaaggaagacattacttctagcagcaagaaacatttgtggatgaaaagatttgagcttggagagagctgtgttgagccatgtccaaaacaatttttggcaaccatacgataaaggtaaaagccagagattgaaaagtaaaagctggtggactaaaaatggtccatagacagagagtcatcaaaacaacaaaacaaaacaaaaaccttaatcataattaattgccaatatttcaaaagtctggagaattcacataagtttagatttccagctcttctggagaattagaatatctagtaacagtgtgctaataatcccacatggtaacaactggtgaatctggcagaggctgcccagcttccaatggtgtataagctctcctcttcctatgcaacctgcctgcctcatttatatgagctgcttggatgctgcagatgcttgaatctaagagctttggtctggaaagtgaggccctaacaaggaagaaggaataacctatggatcaaggagaactgggagtccagaaatgaaggttatttgtaagtctggacccagccattccaactcctttgaggaaataagattctaaggaaaaggccgtttgcattctgctctccaagatctctgggtgttggaagaaactgaattgggggagagggggaaacttgactgggggctcaatacagacatgtaaatttgaaggaaacagagagtttagatgacaggcagtagaaaagttaatgtgtccattctatggctgacccaagattctgtttccagaagacctctctggcttgttaagtgttcatggttgcaggggaaaagttagaaaaaaagaaaaacaagccaaaacccagctccttaaatgtttctaattttattttcaaacaatcaggcagagtaatccctttacacactcttcaggcattggctgagggtcccagtcaagaaccattcagttttgggggccttaagaaaaatatttcctatgattaaaggaactttggacaggttattaccttctttgagcctcagtttctttgtcacttagaaggttgaatggtttccacattctgagggtaaggaatacgagagtgaatgaagaaatatcaagtgcatagctcagagtaggaagaaagaagtgaccagggacaaggctaagaacttactcaaaaggtgcccagctgctcagcattctgtccaaaaaagggacactgacatctctccagcattctaacagcagtcacatagcattatcagctagaaatgaaaacagattcaattctatatcctgctaaaagcttgagggtcacactagctgtgtgatctttggcagctggccaaaaccctctgaaaggcagtttcctcacctataaattttttaaaaaatatttattgtgaagattaaatgaagtaatgcatttaaaatacttagggtgccttagagttccagcacagttcgtagctcatagtaatcagttaatagatattgactttaataaatagatacttaactgagcctccaccctgggcctgttactatgctaagtaccaggagtgcaaagggaaatgaaacacgttccccaaacttgtggaactcagagcaggccaactatatgagtagtagatttttaacacgatgaggaaactgttataatggaaaaataaatagtgtgaagagggactaaggaagataaagatatggtgaaaaaaagagggttcaggctccaactgtggcatgatctagatctgcaaggaagagtagagaattgcagtagagaaagcagggaagacattccaggcagaagaaacagcttccacagaggtaaagaaggcaaaacgttttcaaaggttctaggggtgggggatggtggggaagatggtcacggaatagtgaatggttcaagtgaaactgttgtatgggttgtgagggcagttttatgggttgggtgataaaatcagaagagaaaaattgaattcacgctttgaaaggccttgtgtgtcttgctgagaagcgtgaacactaactcatgaggagtaaggcgcgggtctttaaaggaaggaagtttcataatcaaattgttttccatgaaagataatgaagggagccagccacaaggctgttgcaatcaatcgtccaaacaagaggtgacaaagactctaaaactggccaccaatgaattgaaagtggatggtgaggaagaaagaatacagggtgacataaacacaatatgtaaaatccaaggtatgaaaatggcctggtcccttcgtccttaccacagtcatcccaagagaccaaaacaaaaacacagaaaatctcttttaaaaataatctcttttgtttgtatgcaaataggccatccacagtgaaaatgcaactcaaatgcaatattttatctgcagtccaccaaatgcaaagatcaaattggtttacaaatgctgtccttcttaaaaattccaactcctcacattaaaactgtagccagctagtgcaagttaagattgtttgcaatcttaaataagatttgagtaaagctgaaattgagacacttttcaaaagaggccatcccttactcacattgctgaagagtagaaagattgacaccttcttttatcagaaaatttctttcagggagtaatgcctcttgtgtggtggcagacccttcaagtcttccagataaagcatgtgatggaagtagcagggagctgcaaaaattgcaactctatgattctgcatcaccgacttgaaaactacaagcccaggttgacaaatgtacattttaagtgttcagagaagtcttaagtgccttgctttggtcacaaagttgccacagggaagtaagtttttgaatgtgcagtgccccgtccccagctctgttgtgaaatggaaactttaaaaaaaaaatcactgatttaaaaaaccactggttttgttttttaacaagtttagtcatattgcctgtgttctatataccccaatctattttattttactttgtagtgtacatttaatttatactcaaataaatattttacataaggtgctttcacggaccttcatgcttccctaaatattaaatatgctgcccatttgttaaaatgtgttagttttgttatgtattatatcccttcccctgcacacatagaaaaaaaaaatatgaacttaaccttcaagcaagttttacgtttagaatattacctcattttatttcttctaactgaggttataaagaaaatggcaaatgtcatgtgctttgtaaagaaatgacagtttctcagaatacgtaaatgcacacccttagacaaataggccacttaaattcagaatcacagtcttttgaatattggtttaatattctcagataaagattgaaaggataacaagttttggaatcaggtgtttactttgagttctgaaaggtgacatcacaggtgttggtagtttattgatatttaatttttaaatgtgctgctgtatatatttcatttgattagaaatatgttacatagttatgttatttgttaaataatagaccatcttttgtataacctatcagaagaagttctgagattgtaagattagattgtaaatcctattgcataactagaatacagaattattaaattggaaaggaagttaaatcacttagaccgtccttcccccaggaagtcctctctacagtgttatccatgacaaatgtttatgcaggcaccctaaccatttaaaatacgaagagaaggagaagagatatatcgagaaaactattcctgagacaagagaccaggggaaaacttttcttagttgtaacctacacaactagctagaatctacctcatgctcttaagatggtgtttaatcatgaggatatcatacataactgaaattgtataatgttgaacatcttttggtggcaagtaccttgactttattaaatcagtgtaggtctcctgcattcaactatggtctctaggagggcagggaccacacttcttagctcaccttggatctcaagaccagtccaaggccttcattcagtcagagctagttaagcatttgttaattgaatgaaggaaattaaaaaaaaaaaaactagaaattccaaattgtgcaattacatctgtgaattttaaggtgttattggaaaatgggaaaaattactgagattcgaagatggataggaacaaaggtaaagcatgaaaatgtgcatatttgtgtgttgctagagtcaatagtgattgccagttcctatgcagccctggcaccacctctgaaaccttggccaacccctgccattaggtgttgagatacacgacaggcaggtgaggaaggaggggtgctctggatgtagtcttgccgctagactgtggtctttagtgtccatgtccatggggtgagttgtgagcccatcagtttcccacactacagtccttctctggcttagccttccttccctgtcctgtggtccaggttacccctggcccctgtggccttcctccagagatggaccttcctccacacacttctcaggagctcctgattctaggcatgccccagaacaccgcaactccactctgccctgtctccatcagaccagagacctcacttagaccctgggtatggggttgttggttctccacttgctgcggttgtcactagcctgagctctatcctgagctctgtccatccttctcatcttcctcattgccctttctgctaaagcaactgcacatctgaaggctatacttatccctagtacaatggtactttttctaaaatgcatacaccctaatgcttacccttttgacaattttttcctgaacctaccttttaatataagcaaattcagtcttcaattcaaataagtgtattttgctgctgaagccaccatgtgattttgagagatagtgaagacagacagtcttctgcattcacttgtagaatcctgaaaatacctcttggtgtcccttgcctttctgacttcttgcttgaagacatctagacaaaaatgtgtccctgggtcctagtttctgggttcagaaattgatcgaatgcaagaaaacaatacacattgcctcttccttagcatgcatgaatggtaggtgtgaatttgcatcatgagaaagtaaataaaagaagattccccagggcagctagggaggcagaaaaagccagcctaggatgggagtggaggacatgttagaggttatgagagtgagggtccatcctaccccacctcggtaactcactggtatggtataaatgcaaaattttggctcacacaaagaaaaatactcaacttctaatgcttaactatgtaaaatttgcttttaaagtacaagttaaaattgtatcgcccctcaaagaaacaagaaactcattacatttctaagaatgttccttcagaaacatggaactgaaagctatttttaaaaattgatctggcccttagaaaactggggccttttctttaatttacctaaggaattgacataaaagtctagggttctgcaccagaaaaatgcagaaagtgtcaaaataaaaggcagaaatacaaaaggagactttttgcagcaacgttctatgtatagcattgattccaagggtgcaacatagggaagtgaacatgtggactgtgaaattgatgctaattttctttcccactagtctagcagccctctaaaatgtcacattattaatttagttactttaccagaaatccgtgtatgtggttagcatgtgtgtttttttttaattaacagactttacttatttttagaacagttttagattttcaaaaaattgagcacatagtacgagaattcccatctactccctttgtggaacacaatttcccctatttttaccatcttgcattagtgtgatgtatttcttacgatcaagccattgttgcttcattattattattatttaaagcccataatttacattaagtttctctctttgggttgtacagttctatggattttgacaaatacacaatgtcatgtatccaccattatagtatcatacagaatagcttcactgccctaagaatcccctgtgctctgcctgttgacccttcccacccctccccaacccctggaaaccactgatctttttactgtctccacagttttgccttttccagaatgttctatctttggaatcatacagtatgtacccttttcagattgacttctttcattaagcaatatgaatttaagttttctccatgtcttttcacatcttgatggctcatttctatttattaccacataatattccattgtctggatataccacagctttaccaactgaggggcatcttagttgctaattatgaataaagtggctatacatattcacgtgtaggttttgtgtggacataagtcttcaattcaattgagtaaatatacctagaagtgtgactgctggatcatatggtaagagtatatttacttttttaagaaactgctaaactatatcccaaagtagttttaccattttgcattcttttctttattttttttttattatactttaagttttagggtatgtgtgcacaatgtgcaggttagttacatatgtatacatgtgccatgttggtgtgctgcacccattaactcatcatttagcattaggaggtaaatctcctaatgctatccctcccccctccccccaccccacaacagtccccagagtgtgatgttccccttcctgtgtccatgtgttctcattgttcaactcctatctatgagtgagaacatgcggtgtttggttttttgtccttgcggtagtttactgagaatgatgatttccaatttcatccatgtccctacaaaggacatgaactcatcattttttatggctgcatagtattccatggtgtatatgtgccatattttcttaatccagtctatcattgttggacatttgggttggttccaagtctttgctattgtgaatagtgccacaataaacatacgtgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacattctctccagcacctgttgtttcctgactttttaatgattgccattctaactggtgtgagatggtatctcattgtggttttgatttgcatttgtctgatggccagtgatggtgagcattttttcatgtgtcttttggctgcataaatgtcttcttttgagaagtgtctgttcatatcccttgcccactttttgatggggttgtttgtttttttcttgtaaatttgtttgagttcattgtagattctggatattagccctttgtcagatgagtaggttgcaaaaattttttcccattttgtaggttgcctgttcactctgatggtattttcttttgctgtgcagaagctctttagtttaattagatcccatttgtcaattttggctttggttgccattgcttttggtgttttagacatgaagtccttgcccatgcctatgtcctgaatggtaatgtctaggttttcttctagggtttttatggttttaggtctaatgtttaagtctttaatccatcttgaattgatttttgtataaggtgtaaggaagggatccagtttcagctttctgcatatggctagccagttttcccagcaccatttattaaacagggaatcctttccccattgcttgtttttctcaggtttgtcaaagatcagatagttgtagatatgcggccttatttctgagggctctgtcactatacatctactagaatgggtaaaatccaaaaatctgacaataccaaatgctgctgaggatgtggagcaacaggaagcctcattgctccacatcctcagcagcatttggtattgtcagatttttggattttagccattctactagatgtgtagtggtatcttactgttttaatttgcaattctctaatgaggtatgatgctgaccaccttttcatatgcttatttgctgtccgtgtaccttctttggtgaggtatatgttcagatcttttgctccttattaaattgggctgtttgttcttttatctttgagttataagagttcattgtgtattttggataccagccctttatcagatatatcttttgcaaatattttttccccaatctttggcgtgtctttttattcatataatggttgatacatgtttctgctttaaggaggaagggttttaaaaatacaatttacagtagcagtaaaaataaaaatttattgcaaatgtcttatgttcactctcaggtgatgtcagggaactatggacccagcagggtttaattaaaggggagtgtcaagtcctgggggctgtggttgacaatcctcctttattggcaattgtgcagcagggctgggagtaagaagacaacccagtcctgagctgcatcacttctaaattaagaataattcaggaactgtgtttacggtgaaatcctggcccttctcacatagattatattatgcataggatatgaatttctgtccatgaatccaagtatatatgaaatcatcactttgaaaatttcctttaactcaacttaatcccactggtgagcctcaatcctgccagttgaaaaagagactgtaactgggtcatgcaggagtctccttcctttcctgcagcccagtcagaattcaagaagttcacctggtaactggaaaatgatggaagggcctcaagtccctagtctgtcctggttgccattggcacccttactatctgagcccatagtggtctgtgaagtccggcagctccctgcccccatgccacagtggggaatgagaatatctactgatgctgggccccatgagcaaagcatgctgcctttctaggcatgagccatcacacctgaggttgctaccccctcgggagcactgatggaggggcagttgggtttctactgctcacaggacccagacaaccatcccctgccctcccttcttcttgcacttcaaaagcactctcttcctctcttttcacctctaagccaccggtatcatctcttccatgggctttcacaaaagtctggatgaacctttgaacttgtatctcttctgctttcccccttgcatcaagaaagcttagaaaacaaacactaattaacgtttcaataaataatgctgctctaattatttgtggaaactattctgtattagaactaccatcagcaccgcctcctagagtgcttttagacttgaccactggccgcaggaggccacttccatataacaacaaacagatggctgaaattggaaaactcagctaaatgttcagatgtttctagactcccacgggtttctggctctggcacatggagtagatcctgactgtgtggtcctcaggggactctctctggtgaagtttggtgaggtcaacttccacacccacacacaccagctactgtgtgtagcctgtcctcctctggttgcttctacttgcagccttggcctcttcagtcctgagagcgttggagaatgaggcagtggaggaagcagccccacacagaaagcagtttctgaagtaacctcagcaacttcctcctcaccaaacacaaggaactgatcttctccactgggctcggcctctggtcagccaaggacaacactgttgaccaccatcacggttggccccactccacccttggctctgatgacatatgtgggagtcagaggaattttgattggctgactgctggcctgtcacacaaacaagatggggcaagggggttgcgatatgacttgacatgtgaaaaaaaaaaaagccgtggtcagcaaccccctgcaactgttgaaaggctaattcaatctctgactctttaacaaaagtgatcttgttcactgcctgttctgccctgagagccttctctgctaggaggtaggttgactgactcagggagaagggtgctggtggcagagctgccaatgggtgagggtcctagagactatcgacatgaggggcagttgagaacactgtagtatttagctgagaggagagactattaataaaatttacaaaatcagctttcagctatttggaagggtttatataaaaggataaaataatatgttctggtagttctagaaaacaggacagagacaagtagctggtacttatggattggaggagtgagtggcagtagtttggggattatttataaaaaagacatttttctgttaactctcttttctaatagtgaatttcccaacctgacaagtaagaaagcacaggctagacacgcatctgtcatgacactgaaggggtccttgcttgagtgagagactggaatgatgagttttgaggtcccttacagtccagcaactctagctaagttggagaataagagaattccatgacaccatatcaccccctcatttctgctgcctgcctcaccattcatctctctttactccttttaatatcattctacgttacagcattggaggaggctgctctaaataggaactgaaataagtagattaaagaagtgctatggaagggaaaacaataaaacaacttgttttttaagagcctactattgccaggatctgtgctaagcaccatatatatgccatgttatttaaccgtcatgacatgcctatgagatatttagtattacttctgtgaggaagccaaagctcagagaggttaaataactgccccaagaacacacaggcattaagtagtggagcagggtttgaacacaggtctctatgactccaaagtgcagtgtgatatgttatttttactgatctgtttatggaaaatgatactgctttctaatttagtattaacacaaagatttttttctaaatagatttacttaaagtatgttataaaaatactatataaataatgaaacagattttacatgagtatgaagtggtactagtagctagaatgatgaaagtttggggaatactactccaaatattttgatagctagcctttcaatttagcctgtcttatatttggactgctgagtacaaggaaaagaaggaaacatgaaaattaagtgaaatatgagttacttcccctgtgctctgataggtgggtaattgatcatatgtcacaataagaaaatcaaatgaaccctttcaaacaacagcaaaatctgtgattgtaaaatccagaggaaaaccccaggtgggatctatctgtatgaaggatgaaatttccaaggtctgaacatagaatggctgagaggaagtgatgaccctgtgagtcaagaccctggaccctgggggagccctgtgggtttgagaagccctgggtgaaaggtgaagggttttacaggcctgtttacagacctctgtagtgacagaagggagatctttgtgcaaaggtcaaagtaagaattgggaaagtctgaaaagaaaacaggaaagtaataatgaagatgaaataactacttggcatactctgccacatgatttacaggcaaggtttcctttgtttttcacaacaatgcagcaaagaagtgattatgagtcacatttcataagtgagaagactgacattcaaacatgttcaataactggcccagggtccagtggtcaagccaggactggacttcggaccaccagttccaaacccacaccccttcccttgcaccacacgcttttgtgtggatgagcctccccaaccctgtcaacaacaaactgtcactttgtcacttttaatgtctcctgcttcacaggacacagctagcctccaagagatcagggaggcatgcccagagggtgctgcttctctcttttgaagctcaagtgccacagacctcagaggcacataaatgtcccccacactgagcagaggactttgcagtgcctgatcagggcagaaaaaggaggcatgcacctgggggaggatcacatacgagtgaaacctgtccccgctgaagcactaggtttggagaaatctactgggcatttacacacctttcccacttctgcttatgacttgtagccaaactcaagagtaccacccacttccaggaatagtgtaccaaggtaacagaaacattctagattcatacaattggggttagattaggatcatctgaaaatgaaggttgtgtatgtcaattgccttctaacaggatgggtggagagatgtacttaatgaatgattttggggaagggctagaagtgaagcacatggcctctctgccctcactcattgaaggctgtcttctgaagccccgtggagctcagtgcctgtcacatggttgcccacatttgttgaactgaactgcattttcatctatgggcttcaaaggctgtgtgtactctgggatctctgggaatctgtcagggaaggtgtctttgtcatgtttgtggatggggctccctttgggggtttcccagggctttacactcatgctccgagggtacgtttgtagtcattctcatcagtggaaatgcccacctgccggcagaagttatttggaaccaagcaagagcactgtccctggctgtggtgttgtttctctagtcagttcccctttctgtatttgagttctaccgtcagtcctggcattatttctctctctacaaggagccttaggaggtacggggagctcgcaaatactccttttggtttattcttaccaccttgcttctgtgttccttgggaatgctgctgtgcttatgcatctggtctctttttggagctacagtggacaggcatttgtgacaggtatgtttgtggaggctcagacgcctagggagtggcatgagataaagctgcaagctgcatctggggcagaaatgctgatgtgctaatggccggccagagaatgagtaaaagggattgcagagagcatgcttaaaacctctgaccatcaggtttgcttctcagattgactacattggaggtgggatattacaaaaatctgtctcttcctgccagatcccttcatctgtttttcgtgagctaagagacaaaataggcaggaaatagaaggtgccacttaccaaataattggcagctgttcttggctttggggtgctggggtctccgagcagcctctgctctagaagaagcagtccaaagatgtcagctcgcctcgcctgagtcccctgtgccagtgggaaatccagagaagggggatttcctcctcttgcagcctctctgcaatggacttacttggctttcctgtttgacctttcccttctctggtccagagacccttccccaatatttcttcccatccaagtgccccatcccaatattagccccacttggcaccagagaccaagatctaatttaaaaagaaatattcttgggtcaaaaaagagcccaagcaagtgattgaacataatgtgtttcacatacggtgaacctatttgcatttgcatttgcaaacgggcttaaaatatcatctctattaatagcaatttaaggttctggagagccaggtgaaaatagtttttgacaaagggaacttcctactccccttaaactgtaataatgaaggaaatgaactgtttatcttacatgtaacctcaatcttgggactaaggccctgtactaaaatgcgtctatttatgtgctcagacttgcagttcgtgttatgtctgctgctgcagataccgttaatattatttatgtgagctatcctgtgtataatggaagcttttataaatctctatttatttattcctaatatagttattaagtgcttgctatgttccaggtactagggacttaacaggtagcataaaagacataaggaaaagctgcactcttgttttctagcctagtggggaaatcacattaatttaatcacactaaacatgactacatagcaatagtgctttaaagggaaggaaattgttctatgtgactatatcagctgattaattaccaagcctttgcatttgatattttggttagtctattcttcttgaatttcatatgcctcttcctgggtgggggtgaggatgggattttatggagttgaggctagggcaggtagggagaaaacatgagaaagatgaagagataagccaagccagattcttcagcagaaaaatcaaggttgaaataccatgtttcaaaaatcagactgaggtgggagttgaggttaggggtccctaggccaggggattgaagcttcaaagagataaaactagagcaaaagcaagcacagagagtggcagagaggtccctgggcatttttccacagtccattctagtgctggcaatccacctttcatggccaggcaggtaagagtatttgtggggtgggagaaaggacagggccataggctgggcacacagccctttactggcccttatctctcctctcttctcctatacagtgctgtttccgaactgtacattggcttacactcgggctgaggtttgggaaataggcgccattttgaatatgtgtggaggaagaaaagtgtgtcttcagcactttccacctccccatcacggccctgagacctcaacaccgggaagcatctcgttccctatcggtcctcctttattcatggacggatatgattcctttctaagttccatgtcctttttagataaattaacttgaacctaatgcctaatggcttaaaaacaaacaaaaaaaaccctcttccttccagctagcatttgcattttaacaggggctttcaaaaaatgccttagcccaaggaatgagtaatgtgggaattccaagcagcagggtaggactggtgcacagtatggggagagaaggcccctcaagttgtggccctgaaatgttggcttcctctctttgaccatgatgctgtttctgagaaaacaagaatcaggctaccttaggggaccaggatgggcatggctcccttttagtgagttctatgagcctcatacctgacagtcagagccctcgagtggatgagcacagactagaagaagcactgtgaaactttgcatgatccttacctttttggcaaaaaggaaaaaaaatcgttctcaaattcatcaatagtttgaaatagggtgtgccttgattcagaaagtttcgattctagatacaactcggagaactaggcgtgtcttgtacacagatttgctcttgggggaccggaaaagctaaatgctatcgccatgctatgctccttcttctaggccagtgaggggaacgcattcttcattttaatatttcagttgcctacaatattggaaggtggataaaagcaccctctgctccttctaaatctgcgaagacatttcttctctgcacctactcatccttgatgcagctctcctcatgtctgtatggaaacactgtgctctcaaatgagtttcagaaagaacaactcacgaaagaaaacaagcattcggtcagaaaaatctccacaaatggggaataagggggatttgctccaaggagagactggaaaccaagtcagacataaaatccagcctaagctagaaggagacatggctggtgggagcttgaggaaaacagagctcaggatggaggacgtctccacctccagtcatgtcctctgtccaccagacaccaagaagtgttcatgttccatcgaggcagccctcacacccatcccttcctcatcatgccgactgcctctttactgcttcaggctcaccatctcaagtcgacgagcctgtaatactggctttcttgatcaccctgataccagccgtcacctcttgacaggcttattttctttaagctgtcattacaccatttttctgctcccaaactattaattccaaacttccaattttctgttaaattaaatatgaattccttatttgactttccatgccctattaggctatcttgctccttgctttacttatagaaactaatctcccattatttatccaaagacaacctctgctgcaggccagtcagcttttcttactgtcctgtaaaaattccatggtcactcctccatttccatgtgtccttaaaaactgttatttgattgtgtctcagaaagtcgtcaaagaatatataccaatgaaaagcatcaaaaaggttatacttgatgttatgtgtgtatcaaaaatatggctgaaatatttatccagtgaaactcaatcaacactaaaaagtggttctttcggaagcatcagttctttgagacccattaaacagatgcctcggatgcagggttatatattatcaggaatctgtctagggaagaattattggaagcttgcaaagcctttcaaggacagaggacgatagctaccacgttgagttctaggaaattaaccattgttattgttaaaggaagacagcgtttctcagaggaagactgttaaacagtgcagtggcccaggctaacagccctcataagtgggagtatcagaatgagtggacttaattacttaaaaccaatacagggtggaacttcatctgctataacagaaatcaactcgtgcaagttctaacatgcagggtacagttctgagaccaagtctgactcacctgtcaaagctcagctcaactattaccacctttacaccacccttccaagctgtaggagtgcttgctgttctccatgtcttctgaagccctggatcacttgtagccagctcagcagactctacccagacagggatcctttaaatgtaccatattgtctactgtgttaaaaatgagaggaactgactcagggtgagagcgatggagtgtccagatgttctcctttatttctccttattcctggaaatgtaatgagaatcttagaggtgaactgaaaagttatgagttcaaccacttactcaattcgagattcgctcctaaaatgtctcttctgtgttatcacccccactttggtttgaatagtacttgtgacagggagcttatcacctcacaagaaaatccagtcattgcttgtagctctctattaaaagttttccatcatctggaactgaaatctggctccctgtaacttttagttattggaactacttgcccttcagcaacagtgtatgtatcctcccatggaagggcccttacatatttgcagacacccagcatatacttgcaatcttttcttcttcaggttcattaccctagtccttttagttgttcttcatttgacataatttcattattcactagtgaaccttgctgcccttccccttgataaaccgaatttgtcagtgtcattcaagtataactgacctcacagaacgtgataccacaagcgatgtggtctgattagcacagagttcagtgaatgaatcctacactaggattggatgaaatttacttagccataccacactaacacttatgtgatttttatgtttactatggatagactatttctcctgtgtccacttcttcctcttacacagttgttatttcaaaactgaagtacagattcttacacttaccctcaggagattcatcatgttagtattagtctctcttttcaggctttatgaatgttaattcagctaactcatttttgagctatctgtctcattttgtgccatctgcacagcataagtttgatttctgttgcttttattagtagttttactaaatacataaaagtgaaatagtgaaacacagagtcttgtagcatccactgtgggatcagtcttttagacaagaatgatgcagttgctgagtcaaatgaataaatgaataaatcaaacaatactttgtcctcatttcccatattgatctatcaccatatcctgttaattataattctaaatatttcttgatctatccacttttcccttacttcacctgctactatcccagaccaaacagccatcttctttcactcaaacaattgcagtagccaactgattggtcttcctgcatctgtcctggcttccctatcatccatttgctacacagaaaccatggtcatcttttcaaaatgcaaatctgatgatatcagtctcagctctaatttctttggtggttcacatataaagactgaaatctttaactgaccaataacacacgtgtgatctggcccctgctcacctcttcagccttgtctttcacctgtctcttcattttggccacagggacctcctcgtaccttctctcacgtgccctcctgcctcagcgcctttgcatatgctgttccctttgccgagaactcttcctgtcaactcccaagcccttcacctacttagcacctacctattcaatctgttctgtttgcctcttggtatgttacaaactgtctccaaacttagcagcttagaacaatgaatcctttaccctctctcacaatgtttggggtcaggaatttgagcgggccttggctgatttttctgttcctcatgccatcaattgatatcacctgatgttattaagctgatggatgggctgatctggagatgcactgtccagtttggtagccactggttacctgaaatgcagccagtcctaattgagatgtgctataactataaaacacccacatgattattgaagatttggtgccaccaaaaaatttaaaatattcgttaataatttgtattctgattacatgttgagattataatatttcacatacatcagataacataaaatgtcattaaaattaatgtcacctatttctttttaatttctttaatgtgactactacaagttttcaaattatatctgtggcttgtaattgtggcttgtattgtattctttttttctgagatggagtcttactctgttgcccaggctggagtgcagtggcgagatctctgctcatcgcaagctctgcctcccaggttcaagtgattctcctgcctcagcctcctgagtagctgaaattacaggtgcccgccactatgcccagctaatttttgtatttttagtagagacggggtttccccataatggccaggctggtctcaaactcctgacctcaggtaatctgcccacctcggcctcccaaagtgctgggattacaagcatgagccaccacacctggcctgttttatattcttactggacagtgctgatctagagcaggagtcaagcagttttttctatgaaaggccacatagaaaatgttttcagctttgcaggccatgcagtctccatcatagctgttcaactcttccattgcactgcaaaagcagccatagataataatttacaatagacatagcagtgttccagtacaactattaataaaaataggtggtagccagatttggcctacaggctgtagtttgctgacccctgatctagaagatccaagattttattcatatgtctggtggcttggcagggataggtggaaggctcagctgggaccattgacccaaacagctatacagtcctctccagcatgatggtctcggggtagtgggacatcttacgtggtggctcagaactccagataaggtactcccagagagacaggtagaagctgtgaggcttcttatgaccaagctctcgaagtcccagaatatcccttgtactgtattctatggtcaaacaggtcactcaggctagcccagattcaaagagaggagatccaactctacctcttcatgggaggaggagtagccaaggatatgtgtttctttttaatctattatatcattcttcagatctcagtttaggctggtcctgttatgggctctcaaagtaccatgaacctctcttttgtagcacttgtcatagctagttttacatttctctgtatgattacttgatcactatcttgcttttctactaaactgtaggcaaccacgtgaagaggaactgtttctggttttgctcattatattcctagcaccaaacacaatgcttggttcaataaatatttgtggaagaaacgaatgaatgaatgaaccaatagcaaatgaatgaatgagtaataactgtatcaatattaatcctacatttctccatattgctgtcacgtatatcataagatactctgtcagaagccttgctaaaattcaaatatatttgattcccagtaaccttcttattttgtagttcagaaactttataaagaaggaaataagcctatcttactcttcccagtatctcaaagagggtttctgccctgagctgctcaagagggtttctgccctgagctgctgttcattctgcaaacactgctcgaatacccactgtgtgccaggtacagagagttcttctctgctgtaatctggacaggcaccagcttcccagcgtgggtttaggcttcaggtgcacactactgtgtaccgtctaagccacacctagaagagctctggggaaatatgactacttgggcagaaaaggaaggaactaagaagaggtatctttgtgtctgaggtctgaaggagcgtgtgggctcttgttcaggcaaagggcaggatgaggggaggtggggtggcagcagccagtaatggggtgggacagcggaatgcagaggatgaaacttcaggtcctggtgctctgagaagtaacgctgtgcagcatgtcacacccagaggcaaaccaaggccccagggagctgatgttgcactggagctctactctcctctcagcgagctggtgacgtgccagtccagcaggcctggcttatccaaccacaagtatgaatcggcagaaggcaatgagctgggccctgagtgctgctgggctgaggccgacctaatccttcctccacagagactgtggtgtcccctgctttgctcagggtaagaactcttgtatacctcacaagaagccaaggactacctaccaccttccacactggccctggagcctgcattgtagttatttgtggacactttttcttctctttagtgccaggtgggggaccaaggcctacatgtctttacaacccctcaatctctagaacaagtctgacactgagtagatgtagcaaatgtttgcctgaaagactacctcaataaataaccttctgaggcaccagcaaacttctcagcatttttcctgatactccggttaccactaacattctacacaaagttgtgaaataagtctttttctttgttgctctccaacatctactgtggacccctcctctcacttcctgtttcatcctctctgcactcccctgtcccaccccattactggctgctgccattccacctccctcatcctgccctttgcctgaatgagagcccacatgctccttcagacctcagatacaaagataccccttcttagttccttccttttctgcccaagtagtcatatttccccagagctcttctagatatggcttagatggtccacagtagtgtgcacctgaagcctaaatccacgctgggaagctggtgcctgtccaggttaaagtggagaagtactctctgtacctggcacacagtgggtattcgagcagtgtttgcagaatgaacagcagctcagggcagaaaccctcttgatgcaaagggatactttggggccccttcttctcccaccccagtctgtctctctgagagtcctctcgattccaggagccaccatcacacctggccctaggctgtgctgctcccgtctgtctcagaggctagataacatcagagtcctttccactggctcctgtggcagagcaaaaactggttggcatttttaaacgtgctacaccagtgtgtgaaagaaacacaggctgcatgggtttaaatctcagctgtaccatttactagctgggcagcctagggcaagtactgtgacctctctgagactccattccttcatctgtaacatggggacaaataatctcaccctgttgtgagcagtaataatatgattaatcatttagccaactcttattcatgttctctgatgggccagacatacaaagtaagtgaaagtggattacggcaggtgctcttcttggtttctggagtgaacctccatttacatggaggctcctctttttagatttctgactagttcacccaccttattcatagaccttattctgtgcttagctgacagaaatctcctctcagagaatccccccggtaaattcttaggttctttcctcttccattcccctttttgctctctccctccgaaggcaagagtttccactttacaggcccactggagaaagttatggcttctggttgtggttggaggttcattcctgagggagtggggacatttctacacttcttcacggccaatgacattggagaaactggcttcctaacccagcccacaccctcgcacacacacatcacacatcatggctagaatggagagaaattcttcatatggggcacttgtacttcatgaaagaaaatcatatcaatcttgagtattttaacatcctattacagcagggtcactgataaactaagtgtccagagtgttttctaggatggtgtgtggtctccaaattaacattagtgaagcttactggaaggattgttactcctgggccaggccaggattttgaggagagatgtgtttgctgtcaccaaatccttgacagactttggcagaagtgtgttaggcttactctggatagcttcagaggacaaaactagtattgacggaaggaaggtaaggagaagcagcttctaacccaggggaagagagagtttccaaactgagaaatcaaaaatggtactgattccttgtcagggtcagtgcttctccccactgtgtgaattacaggggccatttgtccaagattccttagagcaatactgatttcatgtaattatttgaatgaaaggtgatttgttaaatttatagtaaaatataatttgatttgtgtccctgtttgtcatgccaccccagaagaaaaattgtctttggttaggtcgaacataatggttttttggtttgcaaaccatgagcgattcccatattaggtgggagttcagattcaaagggccctcttttttttttttttttttttgtagtagccagcctaatgagtaggaagttgttctcactgtcattttatattgaatttcttttattttgagtatgaccatcttttcaaatgtatgagatagttatttccagttccacatactatctgtacatttcttttgcccgcttttagtttgggtctttggcctttttcttattgatttatagaagctcttttatacatagaaaattaatactttgtgactagttgcaaatattttcagttgctgaaatacacagtaggtgttccatgtaagagctgaacagctggttcctgattgctgtctccctcccttccagccaatagatttcagagtttgggcattacctattgagccaaagctgacaccacacaagcgcagagtatgggaacagagttctctgtctgattcctgtgagcttcctcatactaaatcaccaacagcaacctacttatcacagaatatgagaattgaacaagtgttggcaaggatgtggagaaattggagctcttgttccagttgtcgatgggaatgtaaagtgatgtcgctgctatggaaaatagtgtagcagttcctcagaaaattaaaaatagaatgaccacatgatctagcaattccccttctgggtatatacccaaaagaactgaaagcagagtcttaaagagatattcatacagccttgttcataccagcattatgcacaatagccaaaaggtggaagcaactcaaatgtccatcaaaaatgaatggataaacaaaatgtagtatgtacatacagtggaatatcatttagtcttagaaagaaaggaaattcaaacacatgctacaatgtggatggcccttgaatacattatactaagtgaaataagccagtcacaaaaagacaaatactgtatgagtttacttataccctaagcagtcaaattcatggaaacagaaggtggaatggtggttggcaagagctgagaggaggagagaaagaagagttattgtttaataggtatagaggcttagttttgcaagatgaaagagttctgaagatggatgtagtgatgactgtacaacaatgtgaatgtatttcataccactgtacactcaaaaggtgaagatggcaaattttatgtgtattatgccacaactaataaagatttctaaaacttatgagatctaatttcaccgtttcctattgctaaagatcacaaattagaaaacacgttggcaaaaggtacatgaaaataagcactcttgtgttgatcagagcataaacgtataatctcataaactaataaagatttctaaataacaaagatttctaaaacttatgagatgtaatttcaccatttcctattgctaaagatcacaaattagaaaacatgttggcaaaaggtacatgaaaataagcactcttgtgttgatcagagcataaacgtataatctcaggggagaacaatttgcaactattcttcaaccctttggtcaaacgattctgcttctaggaatatagcttactcccacctgtgtgatatggcatataatcaaggttttccattgcaacaaaagattggaaacaacgttaagtatccatcactagtggtctggaaatatatatatattattgtcatccaatagaatacaatagactaatatgcaacttttagcatgaggatactcgttacatgctgatacagaataatctccaaggtagtcatatgtgtgcaaaaccgtacatagtatgctaccatttgtgcttaaaaataaaaagaaaacagaatatgggtcaatgtttttgtttagttttgtctaaagtaactttaagtagaggcaagaaactggtaacatgtaacagtgatcacccctgttacctctgtggaagaaaactagacagctaagggacaaggctgggaggcagacttgctttccactatttatcacctttatctttcaaatttagtaccatctacatttagtaccatgatctattcaaaaatatttattaaaaaaagaaaaggtatagtctagaaggaaaaaaaacataacagacacttctagcccaatgtcctgcactgggtgctatgagagcagaggaaagaaacacatatggcttctagacaacaccgtctggggcatacatttctgctattcgatcaagaatagttgtgcatcttttcctggaaagaattgatttgtttttatcaacagacctatgaatttagtggacagacctgtgaattaattcactggttaggttttcctttttacattggctgttaaaaagctataagccaaatttatgtccccctcagtgcaaattgggcagatttctagggcaagcatttagcactggccttgtccttggctctgtatcatattcctgtatttggtttgcttttccacctgtttctcatgttggtcatctttcctgtgtatggccataccatcctgaatgtgcctgatcgcatctaatgttggtcacctctccttattctttgcttccttataagccactaagcagcctttttggtgctagttagggtaagtgcgtgggtagtgaaggagggaggagggagaggaagaaagaagatagaggttataaagcaaagcatatcctttttcttggcttcatcatgtagattaagtgaattgctctcaaagcgtggtccttaggccggcagcattgtcatcaccttatgttgttaaacataaaaattcatgggtttcatcccaacttactaagccagactttctgtggttgaggcccaggaaactctccaggtgatttttactcacattcaagtttgagaaccacaggaaaacaaaaggaaggcagatttctaagcgtaaatgcaatactaaccgattgcccccatcatgcctgttatgttggtcaagataaataatactagctactgcaataatcaatccctcaaattttattttttgccaatatcacaatccattgtagatcagttgtgggagaggtgtaaagagagctgctttattagtttattaagcaaaccagatctcttccattgtgagactttgcgattttctaggcccttggacatttcctctggatcccctgctgctaagaaggcaggagagggaggaaagagaagagactttagcagccagatctggaagaaacatcttttctgcccacaattccattggctagaagccagtctcatggcctgtataactgcaggggaggctgggaaatgtgacctatcgatggagctaagagcaaaaggaaatggctttgatgaagccctggcattgtctctgcacacccgagaacccaagtgaatcccaaactccacgtccaggtcatgttttggtgaacatcggttttcagtttccttttctaatcaagttttacctttttttttctcgactctagcactatgggactgagtaacattctctttgtgatggccttcctgctctctggtaagaacctttcagctttgttaagtcctggaatcctactgtctcctgatgagtctgaccacagcaagcccaggcctgagacttggtgggttttactcactttctactgagcattgtacaagaccacatgcaaaaaagactttcctggagaagaaggaagtgttatgattgagagcagctgatggcaggcagctgggatggagctctcccccccgtgtgcttcttcctcctctgcagtctcacatcagtgagcctagatgctcagagtagggtagcctggcccatcccatggggatgggggaaggctgctgcactgaggcccctgagacttgactcttttgttccacacatattctcttctggtcttctctgaccctgtttctgtctttctcaggctcctaggaaacaactgacagaattccaaaagtctcccttcattcggagcactggctttcacgtccctgacttccctaccctctctcactcccttccctacagcccatgcacatacctcatggttgccacggcttcctgacaactatggatgttcagctaattgtgtcagctgatttatagtggagccaatgaagctgaagcttcagagccctccatttgcacaaccctttctaaatccccctcaagaccctgtgaagggccccctagcagtgtggtcacctgtcttatgctttggtaaaatttgaataagtaagatattgtaaccacaataagttatgaccactgtctccttcctctgcaacttttccctccatgccattctcctgtctggtggtgttagcagtcaggggcattttgtatttgaattctacattctttttcttaactatccaccacctcccctcaaaattttaacagcatccagcctcacaaaactcagatcttccctgtttacagttccactttgagtttcagtttcttcatctataaacaggagttggctgcggtccctgccatgtatcctgtgactcagtgtctcgtagttactcctggcccaccccttcctgctgctccttgtctccacctgcaggcctgagagggaagccaccccactaagacagggaggtgaactgagcctgaagtttggctacagcacccacaggccaccagccatgagttcacctcctccagatggccacacaccaggcccttggccactgtccccatgtctgctgtggatgatgaggagtcagggaactacaaagagatggtccctcagatccatgctggctgggataagccttttcagatttctgtttttctgcttagcaccttgagcttgtggagtccttgagtgcaaggtctgtagatgtgccagctgatcactgacttaggtaacaacagcagcttccaacccccagggcccatgacctgctaccttagctcctggggatgtgggaggtatgtgtgtgtcagagagcaaggcaagaagactctagagaacattatccagtaagattcccttctcatcccacttcttatttatttattttatttattttattttttgagacagcatctttctctgtcacccaggctggagtacagtggcacagtcacagctcactgtggcctcgattacctgggctcaagcaattctcccacctcagcctccccaagtgctagaattatatgcatgagccatcgcacatgacttattttatttatttgataaatgcatatatacacacagtcatgaatcgtttaacaacaggggtacgttctgagaaacacattattaggcgattttgtcattgtataatcatcatagggtgtccttacacaaaactagatagcatagcctgctccatacttaggctacctggcacagcctattgctcctaggctacaagcctgcacagcatgttactgtgctgaatactgtaggtgttgtaacacaatggtatgtatttttgtatctgaacatatctaagcatagaaaagatacagtaaaaatatggtgttataatcttatgggaccaccattgtatatgactgaaatgtggctgtgcaatacatgacagtatatgcatatatatatatatcccttactttgtgcctggtactgttctaagtacctcataaatattaactcatttgagcctcacaataactctctgctttaggtcttgttgttatttcccattttaagatgtggacactaaagcccagagagatgaagtaatttacccaagatcgacagagctactaagtggcagagcttggattcacacccagcaatgtagatttagcattcgttcacttgactcttctcctaactcttgtggtaaaccatgaataagtggtaagacttcttccatggggcctgaacagctttggtggataatatagcttctgcctcatccgtgttcatccagtgcctcctccccatcacctgcagctgacacctcagttgacccaagagcttgggcccaagcccttctcatcaaagtgaccagcccagctctcaagatctgggagagaaggaagaaaaatgccctggaaacacatttccagaaaacactaaactggaacaccatttcccaccaaattttctgactccgcacactgaaagtgagaaagtaaagccgagacactctatgaaaactgagttcaggtgtcacttttgcccttgatttgccattgacacttcttagaagtttcttagctcctgagaaaagagttaccaatattgaaagcaacaacctcaaatggtaaccgtttaagttttatggtggtgagagaataagtgactatatttttggcagtacaattttaaagtggaatagaaagcccatgacatcagatcagaaaataacattgccagtaattcacacacgatgaaaagcaacaaaaaatcagattctatttgaattctttcttctcagggcacacctctgcttactgggctggtgaacagtgacctagccacagggccggcttccaaagggagaaaggagatgcaattggcccacataatccaccctcaaaatgtagagctgaataattcatttcatggcatagaaatagcaatacagtgaagcaattctgtttaacttttccctccctatattttgtgtcctctgtcatggaaatttgacacagtagtatttgctgcccctgctcttgaggataaaattggatgggagtttaagactgaaacgggcacctgtggccttgcagaattaggttacagtttgtgccttgtatttacaaagcgaaaggaattcctagtgccacctgcagaggcacttctaactttcaagctctgtttgccactgtcctggcacctccatcacacttttaggctggagccagagaggtttttgaaaaatcagtagctcccacatcaggaggaagtatctttccagtttgagttttggtagctgctctctttttgtctgagggttctctgggtcctagggctttctcatttctcttgaacaacacctctagttaatttcatgtacctggagtggtagttggaatatttcttcactttaagattttttttttttttttttgagatggagtctcactctgttgcccaggctaaagtgcaatggcatgatcttggctcacggcaacccccgcctcccaggttcaagtgattctcttgcctcagcctcccaagtagctgggattacacctaccaccacaaaatacaaaaatacacaaataatttttgtatttttggtagagacggggtttcaccatgttggccatgctagtctcgaactcctgacctcaggtgatctgcccgcctcgacctcccaaagtgctgggattacagacaggcatgagccactgcgcccggcccaccttaagatttatgtaagattggctcaaaagctcattcctgtggaaaggtccactgttttcctcccaagatttttgcagatatctgcgtgggtggttacttttgactcccatttcctgctgttgttgatagccctcattaaaaccatcacctggaggtgaatagacagtcgagacctatcattcccaaagaattgtcatggagcctaatagttctattggattcacccctttatgttaagccaccatttcagtgtttttcaaaatagatatatgttatctagtagggagtatcttacccccaaattagttgattgtttcaggagggcttttagtgggttccagagaaaatgagcaatcagacaagttgatttagtggaagacagtcactgaataggatgtgtatagggttgtttgggagcaagagtgaaattggtatggaacagagaggctcccaaggcaagcagacattttttttggaagaagcaagtgtttgagagactgtggcttatttttcctttgtgagaggggagttttaataccatttccaaaatatgtaacctggtattttgtccccagaagtactgttgagatttatggaagcaaaaaactctgtcacccaggctagaggagtgcagtggtgctatcaaagcttactgcagcctctaattcccaggctcaagagatgtttctgcctcagccacctgaatagctggcactataagtacatgccaccatgcctggctagttttttttgttgttgttttgttttgctttagagacggggtctcgctttgtgcccaggctggtcttgaactccttttaagtgattatctcttctcagcttcttaaagtcctgggattataggcatggcctatctatttttatgttttataatttcttgtactttttgatgttacttcaaatatctttttaagtatcctaaatatacttatttaaattttttttgagtaaatttatctataaattattgattttatgtcgatagacattgttctctatcattaataatgttaaaaataaataaaaaaacaaaaacaagtaaatcaattaatgcttaccacaggccagtatttgatccaacactaactcaaatattcatttctttaatcctcacaacaaacctatgaggtaggtaccattattgttcctgctttttgcaagaggaaactgagacacagggaagttaagtaatttgcctatggtaacacaggcagtgagtagttgagctgagattgaactcacgctgtccagaatccatgctattagttataatagtgtactgccctatagctttctgtttcacagctacatggcattactttgtatggatgtatcattatttgttaaaccatttaacttatttccagtgtattgttcttataaacaatgaatacctgtgtacctctaattttgtgcacatgtatctttttgtagaatgaattcttaagaaattgagttgctaagtcaatgcttaagcccataattaattttcttacatattaccaactgtcctccaaaaaggttgtaccaatttagaattttaccagcagtaaattcagcagttaggacccattttcctaacactctcgcggacactgggtattaccagtattttttttaatacgtgccaatcaaatgggcaaaaagaatggtttctcactgaggtttaaattgcatttccctagttattcttgagatttttcctttcctttcttcaacaattacttattgagtgcttcatatttgtaagggacaattgcaggtactggaaatgtcacagtgaggaaaagtgacaaagcccctgctgtcatggagcttattctaatgggagatgtcaggtgctcagctgagctgggagagagagagctgagttgtcaggtgtcagaggagccaattatagcagcaaaacaaaaataaaatagttcagcttttaatctcttactacgacggtataatcaagaggctaaaatgggaggaagggcagactctgcctgttccatttccccacatagagtgagtataccagtcgagggtcaggtaatcagtgcagacttagggggtcgccttaccattgaagaagccccaaatgaaaggctctagcagttttatggacctgggggtggaggaatccaagggtggggagaattcatgaggaaaatgaggtgagagggctaggagtggaaaagtacaaagtactgagttagcgtggggaatagtgtctttagggctaggagtggaaaaaatactaggtactgagtcagagtggaaaacagtgtcttcaaggcagggagtggaaaagtgctaggtactgagtccgagtggagaaaagtgtcttctctatgatgaggaggcttcagcagaggtgcctgaagacctcaccccagagcctcagataaagagacctaagaatgagggtgcctgggctaagattgcaagtatgtgaaaaagcatgactggcgggaggctgagatcttgattgcagcccccttcagagactgccatgcactgactgtgcaccaagtctgctgtagaaagggcaacttcctcagcaaggcttgtcagattaagcctctttaattgcctgtggtcaggtctgaaaaatcacacatagatttttaatcagaacccagacatctcaggagagacagacaataaccaaacataccgtgtcatgtcatgtcatgataagtaccacaataaatataagtcagcatgagggacagaatgcccaggatgctatcttcaatagaatggttagagaaatctccctgggaggtagcatttaatgaaagacctacatgaagtgaaggagaagctatgagactgtctggaggaagaaccttctggacagagggaacaacatgagaagaggacttgagacagagtgtgtgatcttttggaggaatgtcaagggaggcagtgtggctggggagagtaagcaggggaaagaggcctgataggtactggggacccaattacatgaggtcttgtaaggccaggggaaggactttggatgtagttctcagtgtgaggggaagggatctggatatatttttcagtttggtggaaggcatcagaggcttctgaacaggaggattatgtgattggagctgtatttttaagggatcattttggcttgagaaactagacccggggacaaggacggagcaggcagatgagttaggagacaattacattagtctcctctacccttttcttaacatattggagttcagctctggctgtagtagttctagatctcctcagacacacttgtgtagagcctctgttgggtattttgggtacacaaatgattcatcttggttatacagatgatttagatgattgtagacagaagagggttgtctggtcattcccagacaggggagcattccttgagatagagtagaggaaggctgaaggggaggaagacagtacctgttgctatctagatagagacatccagcaggaagttgaatacaggtatctgaaactctagtgaaagttataggctggcaataagcacctgggagttattagcttttacttgacagttgaatccgtggggctagaggagaaaaaccaggaaagtatggagaataagaagaccaagaacatgcactcaaggttaccaaaattaaagagtgatttgagaaaattaacaaggaaatcagagattgggaaagaatagagcatttcaatgaggagagatgccaacacttgcatttgacacagcggtcaaatgagttgagatctgaaaagagctcaagccttggccatggtgtgaagtcaccaacaacctttgtcagggagtttcagtagagaggtgggggtgggaggctgggaataaaggcagcaattgctgcttactctttcagggagtttgactccaagggaaagagaaactaaaagcagtagcacaaggtttgtgtttgaagtaatggaggtgaaccaggtgaatagcctggaggccgagtgaagtgagacaggacactgcagatttggaatgtcaccagtccgcacaactgaataatttcctccagaactgctcaattgcccagttgtaagaacagatatgtagaccaaaagtagagtgtccccagggtaaattttatagagacaaaggggtgtgtttattgaagttgtggaaaggaataattacaaagacatactattgttgcattgtccaatataataaccactagccatatgtgactacttaaatttcaattaattaaaattaaataagattaaaaattcatcttctcagtcatactagctatgtatcaattgctcaatagccacaggggctggtggctatcatattgttcagcacagagacagagcatttccattatcactaagagttcttgtggaaaacactgcactacagggtctggataaagctgaggtcttgattaagttgaacaacagttgtagaaggagtaagcaagagcaaaacctggatgaataggaggttgtggacggagattagtatattgagattaagattctagggactgagctgctccaggtgaaaagtttcagggttatgtcataagaaggtggggggcagctgctgaaatagtctgcgggtgtagacctgtggagttgacaagatcaaagaaatttgaggcaaggttgttagactcattcatgaagaagtcacccaaattgttagcaagaccttgcatctaatgccaaaatcctcatttagcaaggtggtagtgacttagtagctacaagcaatgagaaagtcagacacctcaaaaggggaaggtgttgctcaaagtccccacaaagtgtgataaaacaaacagtagctggggctggagcaagtggcttcctttgggtgaagccagatttcactgaaataataacctcagggaaacagtcaatgaaggggttaaagatgtgggagagtttccttgtagtaagtaatggaatgaggctttcaaagggccaagtaaaactttggaggaagtttagtaaaagaaggaattttttttagtacagataagcataggaacataaagaagagataattcttaaacatataagatatgcatttggggatagcagccagggaacactgaagtcccagtggggtcagagacttcataaggctagcaaattacagtttttgagtggcattccaacagtagagtgtattgctcaggaagtccttaattatcctttgaaacaaattccttcagctgattacgaaggcatctagctggattcttgagcgacttgttcctgacatcatagcaacccattgtaactagacttcgaccattcctcttacccaagtgctggggaagggagagattctcaatgcttacccacctatggaatcccagtaagtccagttgctaggtggcttgaggtctggggtcataaaatggaaggcctgaagtcatttggtgatcacagaccttgagccaaactttccccatttagtcagagaaaggattagcagcatcccccatgcctggctctgtgtgagatcatggaagccagtggttggtgaggtgctatggagtataaattgcaaaatactttcagttccactcagaatggatttcaaagtgatttccaccccatggggaggagagggagtctgaggagggatggatggaaaaaaaattttcatgtcattttctgtgatccactctggagacagaggcagagattctctacaacagctgctcaaactatagctcttgttaaaatggaggttctgaatcagtaagtcttgggtggggccagagattccgtgtttcagaccagcccacatgtgacgtgaatctcattggtccatacatcacactttcagttgctaggtgaagaagggagcactcgatgagtggaagagaaagccgttgtaatctttgggagaaggggcctgggtcagcggagttagactggtctgtgagtggacagaatggatgggaaggaaagaagatactgtgaggctctacagaaaaaaaaaaaaaaaaaaatatatatatatatatatatatatatatgtaaatcaagaagacagaagcagctaaagacgaagtcatttccaggtccagaaggcacaactgacagctgagtaataacataacattgactgttaattggcagaatttttaactgtgtgtttggtttctccatcaggtcatctgtcctatattacatgacaatttagactaaaaccagtatttcctcagagacaatgctagaagcttttacagtagggggcactcttgcattacattaagagctcagcaaagaagatgcagaagcctcaggtttgccttgtaaggtgattcataaacacactaaatcttccttaggtctccctttcactgtcagggtacgcatatagattttccttcctccctccaataccggtacgcatcctctacaggtggtgcattttatacctcaagtacttcacagggtcctagtgagtgtagtgaaataggcagtgattcatatttgtgcaaactcccactgatgcctgctgtctgcttccctaagagttcaagaccaccaccaaccccttgattatgtgttctcactgggccactctgtacacagtttagtttgacaagtgcatgtcactgttatctgtccttctattccctctttcaagagaaaccacatcaatttaattactcccccacttagaactcttcaaatgaagctcctctcatctctctcatcaacccatctcctccctttcctcctcaatgtcaacatgccttcacataaatcctgaatgatgaaattttatttagaacttacactaacttcctctccaaggtggcatctaacttcatattaagtaagaaacagccttcccactctccacccccgcacttctcacccaccactgcttactttttttttttttttttttttttttttgccaagtctcaagtaattctgtaacctagaaaaggtcctacacaaaccccgtgatcattcacatttaagtagttgggtggcccacatccttcccacaaaccccaaagtgtcctcaaggactaaagcctttctctcaacccttccagcatgatgtctatggttgtaaaattgtccagggtcagtgcatactgggagcagcaagtttgtggtgcctggggtttccccaatactcccaaagcacatcctcacctgcccatctatgattcattttcagcatttcactcatgtgccttaaatggtcattgaccaccacaatccgaaaacagccatcaaatttgcccagttctctttctgatctctgaaagagcttagagaggtcactgaaaataaaggccttggttcactatcgaagtcatttctaaagcatttgacatccttggaagtgctggccatgggagcagcagtcataggggaagttctgtaaagggagctatttgaatttcaaagatgttactcaacgtgattccccaactaatgaagtataataaaggggggctataatttattaccattatcagcaatcttttcaccatagcagaccaaggaatatgtggatgggaggggaggggaaagcttttggtgatggtgtagaagttatggaacctgtaacagctacagtgatgaaaactaaaattaaggttataggaaggtaactggtgggtgaatgggttgtctaactctactggtttttccctgtcttgcaatttaaattcacagaaccacagtactagaaagacccttggaacatttagtcaaccacttcattaatcagatgaggaaactgaggctcataaagattgcagtttgtacaaggccacacatttagtcagcggtgaagcaaggacaaaggtcctaatctccagatgccaagcagatgtgcacagttccagagcttaatatcttattcttcagcatgattactgataagatagtatctgggtattgtataaagagaaatggaggttttttcccctttcctcttgtttctccctccctaatccttaaccttcttttttaggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttggtatgtggtcaatggtgtgtgttcagattcttagccttctcagatgagactgcaaatgagttagaaaaacactggagggggacttgaggggcccaggggaaaaggggggtctatagagagaaggcagaggacagccacttctgggaagtgcatttgaagggagtgtagagtctgggagtagggaactgaaagtcttttgtactttttatagtctgcttctgaaggatcagtaaaaatctgctttggggaaaaaatagagctaattgaacaaagataatatggctaattacctatagtaaaaaccatggataatttggccatcacaaagtttatataaccataaaggcctcagatgtcttacattcattttttccttgggtccaagatttttcacctactaaatctttgcctggagctcctagcaaagcggacagctgacacatttgggttttcccttcagcctcctctaggttgcttatgagttgtttgctgccacaaccatgagcctggtagacagaagggaaaaaaacccaacaaacataacccacaaacttacaaaccagctcctctgcttcacgagaccttggaaggcctaaatgccactacagatttttttaaaactatcacacagtaaaattatttttttttgttttgatatactgttctactgattgtatagatcttgtatagatttaggtaaccgccacaggacatagagcatttctatcaccctaaaaatttccctcaggctgtcccttcatagagtcataccctgtctgcactcataacccttgttgggcatcctatagttttgtctttttgacagtgtcacataagtgaagccacacagtatgtaaccttttaagcctggcttctttcgtttagcgcgccttcgagattcacccaagttgttgcacatatcgagcttgtccctttttattgctgagtagcattttattgtttatccattcaactcagtaaaagacattgggttgtttctggtttggggctcttatgaataaggctgctgtaaacgttcatgtacaggtttttgtgtgaacataagttctcagttctctagaggaaatacccaggtgtggtattactggatccaggttaatttttgatgaaacttgaaaaggcagatcaacacctattctaaaaccatagagtaaaacagaagcaaaagtaaaaatagaatggagagctgctccctttgaaccctgtgtgatttaaactaggctgcagggctttaggaatagttaaccaagtgctaaatccgtgttttcaaaatgtggtcaggtaccattggaaatgttttaggtgggacacagataagcattttgaaaagccatgttgtatttgttttaatgtatattagaaaaactctaacttacgcaacatgtgatttcacagatcttgttaatgaagctaaacacggtctggcaattcaccttctacaggccacatagactccaagaagactgctcaaatagtacactgatatagcaaaacttataaagatgacatgcaaatgacagaccttttagtaagaatacactaaattataaattagtttgtagaacctgcaaactacctagtaactataaaagaacaagggattttttctgacagaaggcacatgacacaggtctagggactccatgccagtgatcctgaacagccagaaaagtgagaatggcaaaggcaagagaaacactgtgtttattaagatcatgtatttttccctaaaatagctggatttggccttcttcttagagtatgttatgaagacactttgatgctcatgccaaaaatcagtgttctgaatttcgaattccaaaatatccacccactcacttaccacaatcctgcttgggtttctgaaagatatgacgcagggcatctcagcaccatgaactctgtcagttcctggtgagactccagctcaattccttcctgctctcttagtctggggagctggaatgtgccccatgggacacctgggccctagagtcagaccacttctccttccaaagactctactccctggaaacagtggcttcattgtaaatctttggtgactcaattacagccctcctgtcacttagagagcacccctttgatttggataagcaggaagtaagcatggctgcaaactctattgttgaaaaataaacatgaagtcattatgtggcactcaccttgggctgagggtcacattttagacaccctgaggctcccaggtgtgccccaatgagccccagatcaagtacccagttatttgctattccctcctagatacatctaaacttagattgatttttttttatctctcttctgctttcagctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctataggtaaagctgttttccaagactatttctttcagcaggtattatacacaaatgcttaaggcagatcatccaatgtccccgacttgctaggaaacctccaactgggccattttatgacgctgttaggaaggacccagatggaggtctcctgcttctcctgagtgatgcagggtccaggaggctacgagcctatgttgcacttgaagaaatatgcttttagccctgaaactgactcagtctcttggtttacctttggatggaggattctgaagttttgatttaaaaatacaggattcctccaggctagaattctttctttgattacaacacatacatgcgcttgcacacacacacacacacacacacacacacaccatgcatacatgcagacatacaaatgatatttattgtgagtatagaaccatttgggacattattggtcacaggagtgaaaacaaaaagatatgacaccccctctgcccttgaggaccttccaatagaatcagaaccctgtaatgtgcacacatgaaaaactggatttttaaaaggttgaattggaatctaaattttattccatggaaatatctgactaaatttaaaataaaagtgactggtaatgagatttatgggcattcagaggtaggcaagatccctgagggtcagggaatggttcctaaaggaaggggtaccttgtaacatgtaaaataaattattggggttaataaatgtggtgaggaggggagggcattctggatgacaggttcccaaaactgtggtgacttccgtagctgaaaaaatttgagacagtatctgggctaagcaggtgagaggaccacagtggatcagctgtatctgacgtaagtgcaggaggtatgtcaaagaaagccttggaggcagaaatgcttgtgtgttcacaagtattcttcagggacaagttcagtggaggaaaggattgaaactaagcagtagccactaataggagcctgacattttaaagtcctggctttacccaggagggcatgtgtctatatttgactcctcttttaagaagctgtaactgcaagattccctcctggaataaaggtggtctgcatctaccctgtcccatcactgcctgtgctgaccttgacacccacatctgccttcttcttaccttgaccccttctccagcggtgatttcttggcttgccccctccagtgacatccatccaactccttgctccataccctggctttgtcacctcctttctcccagtgtcttgttgttcagatataacttggtctgtgaacagcccacggggccagtccccatgaaccaactttacaactgggccaatctcatctcctgctactgacttcttcctattcagacacttcagcctctgagaatccagtaaatggtggagccaactcgtcctgtcccagttgcttctcctgtatcctctcttggccagatagaagcctctccaagctatgcctgaagttcagtacctccttcaatgtgtaattagtttgattggtggccacaagatggccatatatgacatgccccagggccctctgttacggctcccatagtctacaaattaacaggggcttgccaccactataacctcatcatggctcaccttcctgctgcttctcaactactgttctgccaaacttcaacaggtacccccatcttcagaaatgtttcagctctagctgcctcaggaagatggggcttgcctctctgggtttcccattctatcgcttgatcagagataggttagaccctgagtcaaggggccttttttgcatgttaaaaggtagcagcctccacgttagtaagtataacccctaaccccctttactgggagtgccaaactggctcaagtggaatagactgggacagactcaaaagggattaaatatggcctgcaatgccaacaacttcttaacatcccagaaacagggcatgtgtctacaaattatagctaagctaatagatcagctggtcctaattttcctgaaatttgggattagctaccagaactgttcccaaaaatgtctttaaagtgggcgactccgttctaagttttccccacaaagcctgttttccaactccccagaaacttaggagttctcatgtaaggaagtagttcctgaaggcgtgaaggttcctcaaggcatgaagaaacatcaaaggtttttcagtagatgagatatgctgaaagccatgcagaggaaacctgctgtgacctcagtaggaaaaaactaaacaaacaagcaaatgaaaactagaggtaggggcctgtggaagctgttccatttgtccaagtgagaggtgtctggagattatagtggacagaagaatcatcacgagaggaacttcagggcctgggaactgactgcagaggggggcaggatagcaggcacggcacaaatgactgcacgtgcagagcctcagcacagacacctcacccagattccagaatcacgggccaggctgaccctcttcttcctgatcatggtcggtgttatccccacctccatgaaggcatggcagctcagtccaggcatttggccagaggcatgggctcgattcttaggtcgctgctgaggccctgagcctgggactttctatggcctcctattgtggatttcaggcttctctggccttagagccctggggagaggctggcaggtaaataaagagaagagcagctagcagaaaccttttgtaaatgactctcctggctgattgaaaatttgtggtcatttgtagagcttgaggaccctcagcctcccccagaccacattccttggattacagctgtacttccaacagttattatatgtgtgatggttttctgtctaattctatggaaatggaagaagaagaagcggcctcgcaactcttataaatgtggtgagtgagtccttgtcctccccacagactgtcactttgcacctacttcccaatcggctggctgccttccggagcttgttggctgagcctagactggcaaaaagtcaggaagttgttgggaaaaaaggttttcccttggagttttgagcctatacagactggcagtagcagataatgctgctcttggacttcaaagaaaggcgacatttctaacctctggtttacaaatgtacttctggtttccagggaaaactgattattacttgctttatctacctcacttcatgaggttactgtgacatatacataaagtaaaatggtgaaaccactcctaaatgttaaagattgtggacctggtggtgtttaagcagggatatttgctaaatgaccacaagaatcagcttctcgtctctaaaaaaatctaggtttcttatgaaataagttagatgaattattgcccattgacttataacaaacaatattaactttaactaatttctaagtaatacatatccattatcatatataccaaaaataaaataatctataactccactaataagaaaaaatgattacacaaatatttttggtgcctatctttaagatttttctgtgtatcaatctatgttgttttccataattaggattatcataagggttatttttcacaatttggataatatatgtactgtgttctaattttgttatactaaatgtagcaagacaattttcaatgtcataaatatcattctacagcatcatttttaatggctgcaagatattcccttttgtggatacaccataatttatttatttaaccaacctcattttttggacacttgagttagtccaatagttttgttattataaacaccctccccactgacttctgttataaaaatgtttcatggggacaaagtggtccctaactttataataatgccatgcctttttgtagtttggtctggttctaagctaagattggactttatctcagtaattgcctccagtagtaattagtttgattggtgctaataattaaggtaaccttctaactcacttatggtagaaagcacaagatgagtattgcctctggccagcatcttgtttttcagtatactgattttaaaatctaactagaaaatagatggatgacattagcagtcattcaatgcatcctgctgtactttaaaaataagaaattggggagcaacgatcgaatttaaataaattaacacaaagcatgtggcagagccattcaaactgccaatgtatggagtgtgctgcgagatttctatgatataaaagtataaaattcctagcacagatgtaaagacatatcatgcttgtccaggctttgacttttcaaggtgagagttttgagcttcactttctttcaacctcattgccatttaaaattagtcaaatatgaagaagtgacttacatcttgggaataagctgtttgctagatttttcttcacattagaatgatcagcttacaaatgaaacaaagaagggttggagaaaaagattaaggatgtttcttcctccatgaggcaatcagaaaaaaatcaggagactagataggggagataaagaggatatgtgtgttcacatgagagaagttagaaggtggttaaataagctctgtaggtacagatgagatggtcagattgggctgagtggcacatacatgacccctaagaatgtaatgaagaatattggtaagaaaaagttatttattcagacagtcatccatgccactgagtttgatcaaagagagaagccttgctatcactgtagggagggaggtgcaacaggtataactatgccattatagatatgatatatttgtaaatttggattctgtaacttcagcaatatctgccattgctttgtgggtactcctggcattggctatgtgataggtaaaataatgccccccacaagacgtccacctcctatactccagaacctgtaatatgttatcttacatggcaaaaggaacttcacataggtgattaaggcaccaagcttgagatggtgagattaacctggattatccaggtgggcccaatgtaatcacatgagtcagagaacctttcctagctgggatggagaaatgaactggaagaaggagagatctgaaacttgagaagctcaacccagcatttctagctttgaagatggaaggaggaagccatgagccaaggaatgtaagtagcttctagaagctggaagtggctctcagttgacagccagccattaaggaaattaggatctcagttctgcaactataaggagctgaattctgccaagagaccaatgtggaaacagcagatccctccacagagacacaagcttactgataactggtaggaatttctccaaaagtggagcttcctcctactccagtgttaatccctttctcagaggagacggtcctcaaactaactaacttggcaccaaaagtcctatccagtgttttctcattatagtttttctatgcctcaactgtatatatttacccagtttaggctgtttaaatgaataaaaaggaaatgccatagttattctagccagtttccaatctctcttctctttttttgttttgtcaaatagggcagataaggcatgagaatttataactatgaattactgtcttttcccaaacagaaatcaccctatcagcttacccattgggagaaaaactaaaatagctccccctgaaattttacttcctcatttgggtcttgtgtgactgaaatctgtatacaatgccctagcaacaacggtttttacagcttgcctccctagaacaaacctaggagtctcagctgtttcaggaatgatttcttaaaggtaaagtgcctttttcaaaagaaattattattatttttttttaattttttttttgtgtgtgtgtgagacagagcctcactctgtcaccaggctggagtgcagtggcacgatctcagcacactgcaacctctgcctcccaggttcaagcgattctcctgcctcagcctcccaagtagctgggactacaggcacgtgccaccaagcccaggtaatttttgtattttcagtagagatgggttttcaccatgttggccaggatggtctcgatctcttgacctcgtgatccgtttttaaccaacatttaaacagaaatattcacaggcttaaagactgaaagttagtgatatcatcacatttccccttcaaaatgctgaatttgtaagcaaatttaaaagtttagaatctaccttttaattgtctgctttcatttttttgacagtggctttttttgatatggtgactattttgtcatgggtataaaaggataattcattttgtgttaatctgaagacatctgaaatactgtattcaactataagtacctttttttacatttataagattctttttcaaaatttttatttgaatagttttttgggaactactgaactaaactaggtggtttttggttacatggataagttatttagtggtgatttctgagactttggtgccacctgtcactcgagcagtgtacactgcaccagtgtgtagtcttttatctctcacccctcccactctttcctctgagtccccaaagtccattatattattcttatgtctttgcatcctcatagtttagctcccacttatcagtgaaaacatacaatatttgtttctccattcttgagttacttcacttagaataatggtctctggttccatcaaagttgctgcaaatgccattattttgtttctttttatggctgagtaatattccatgagggatatttaccacattttccttatccactcatgggttgatggacatttaggttggttccttatttttggaattgcaaattgtgctgctataaacatgcgtgtgcatgtgtctttttcatataatgaattattttcctttgggtatatacccagtagtaggattgctgaattaaatagtagagttctacttttagttctttaaggaatctccatactgttttccatagtgtttgtactagtttacattcccaccagcagtgtaaacatgttcccttttcaccacatccatgccaacatctattattttttgattttttaataatggccattcttgcaggagtaaggtggtatctcatggtggttttaatttgcatttccctgatagttagtgatattgaactttttttcatgtttgttggccatttgtatattttcttttcagaattgtctattcatgtccttataaacaccattatttttaagaagaaactttacaaaaatagaacataaccagatttataaagcatctgggaactcagtcaattaagaaatagctcaagtaactgatgatgcttcacctgaaagaaggcctggagagaacagagatactgtcttcaaatatctgaagagctaccatgggatgcaaagattgagcttgatggtatgactctgaagggcatctctatgaatgaaggttatgagagggtataaggaattaagagagacttttctaacaattaaaaggtcttttaggccaggggtggtggctcacacctgtaatcccagcacttttggaggctgaggcaggcagatcaccttagatcaggagttcgagacccgcctggccaacatggtgaaaccccatttctactaaacatacaaaaattagctgggtgtggtggcaggcacctgtaatcccagctacttgggaggctgagagaggagaatcgcttgaacctgggaggcagaggttgcagtgagccaagatcacaccactgcactccagcctgggtgacagaagatcaagattccgtcttaaaaaatataaataaataaataaataaataaatagtctttaaaattgtatagaagaagtagacttctgcttcctccaacaaaggattaactgctataggaattgccctctttccataaacaactagaaagcagacaaaatatatgaaacaactgttttcagagatcggatgacagacagcagaaaactgtagtccctgagtgaaggaaagaaaaaatgagataagccctatgattgctctagtttgctgcctggagccagtgtccaggcccctctgaaggcaggggagccctgatactgaactaggaaaagacattgcaagaaaagaaaactacaaacatctctcgtgaaatgcttaacaaaattagcaactaaaatctagcaatatgttaaaagtataatacatcatgatcaagtggggtttattcaagaaacacaggtaagctcaacattcaaaaatcaggcaataacctttactacataaataaactaaaaagaaaaaaacatatgatcatgtcaatggatacaggaaaaacttttgacaaaattaatacccattcatagttttaaatggaaagaaaagctctcataaaaataggaatacaagatgacttcctcaacctgacaaaggacatctaccaaaaattcttctgttagcataatatttcatgatagaagactgattgcttttaccttaagatggcgaatgtggggaggatgtctactctctctacttttgttccacattgtactggaggtcatagccagagaaacaagactagaaaaagaaataaaagacatacagattggaaaggaagtaaaactgtcttttttcacagataatgatcatgcttgtagaaaatcctgaggaatctatcaaaaacctattaaaactgataagtgagtgtagcaaagacacaggatacaaagtcaatacacaaaatcaattatttctatatactaacaaaagcaattgtacattgaaaaaaattaatagcatttataatagcatcaaataatattaaaaacttggaaataaatttaacaaaacaagtacaaggtctatatactgaaaactatacaatattactactggagaaattaaagtaaaccaaaataaatggagacataggccatgtttatgaatcagaagactagatgttaagataaccattctctccaagttgatctatggattaaatgtaatcacaatcaaaatcctggtaagctctctaatagatactaaaaatcttactcgaaaagttatagggaaatgcaaagaatctacaattgccaaaacaattctgaaaaataagaacaaaggttaaaaatacaaaattagccaggcatggtggcgcatgcctgtaatcccagctactctggaggctgaggcaggagaattgcttgaacccgggaggcagaggttgctgtgagctgagatcgtgccattgcactccagcctgggcaacaagagtgaaactccctctcaaaaaaaaaaaaaaaaaaaaaaaaaaagaacaaaggtggacttaacctacctaatttcaatatttactatatatagtaattaatacagtgtgatattggtaaaaggacagacatatcagtcaatggaacaaaatagagagtcaaaaatagattcacactgttgacaaagctaccaaggtaattccatgcagaaaggatagtattttcaacaaatagtgttgggacaattagatatccacatggaaaaagtatgaacctagacacacacaaagtaacttatatattaagaattaaaatgaaaggacttccaaaagaaaacagaggagaaaatctttgtaaccttaagttaggcaagtcttcttagataggacacagaaagcaaaaaccatatcataaaaagataaaatggatgtcatcaatatggaaaacttttgttctttgactttgtttaaaaaacgaaaagtcaaaccacagacagggagaaaacgtttgcaaaatatatatctgataaaggacttgtatccagtatataattacatattgctactcattagtaagaagacaatccatttaataaaaggcaagaagaagagacttgaacagatacataacagaagaagatatacagatggccgatgagcacagtcacaacatcattagtcatcagggaagtacaaattaaaacgataatgagataccactgcacaccctctagaatggctaaaattaaaaggtctgataaacatcaagtgttggagaggatatgaagcaactgaaactctcatatactgctatacaacccagaaatcctagacatttaccaaacagaaattttaaaaaatttaaaaatatataaagactcatacacaaatgttcatagcagcttgcttcataataccaaacctggcattctaaattttcatcagttggcggtggtatatttatacaatgaaatactgcaaagctatagaaaggaatggactactaataatacacaagaacatagataaatttcaaaagcattatgctaagtgaaacaatccaggcacaagaagaatacacattatacaatttcatgtatatgaaatttgagaaaaagcaaaactattttaagtagattcatggttatccatgggatgggggaaaggaatcagctgaaaagcgaactattttggcttataaaaatgttctcgatcttgattgtggtggtggttacgtgactatatatattcgttaaaatcaccaaactctaaactgaaaatgattgggttttattatttattaattatacctccataaagctgattgtttttatcttttatttttattttatttcaatagtttttggggaacagatggttttcggttacatggatgagttctttagtggtgatttctgagattttgatgcacctgtcacccgagcaatgtccactgtacccaatgtgtagtcttttatccttcatccacctctctctcactcttccccccaagtacccaagtccattatatcattcttatgactttgtggcctcataaaagctgattgtttttaaatacacacatacacacataaaagagaacttccagtgacaggaagtgttcaagaatgctctatttagtaaagacagaatcacaaaaccatcagaggtattgttgagtggattcttgtggtctataaatacctccatggacacccaggttagcaacctgttggagtttacgtgggacaatagcatcatcacaacagtcagcctagagaaatttacatcccaagttgtgtcagtagcaagtccctatcaatagcaactcaggctttgtgaggtctagctggctagaaatttcccacttggccttgcccatgcaacattgtgtaatattcttagcaccatctggctagccgatttaggcatcaacatcttcaagacttcttctcctcctccttataaaccttgctttcagaaaaggattagaaactcttccaatcacaaaatgattgctaaaactaaatatattacccctcccaatggtattttttggttagccaggatagagatataagtgaaaaatctatttccagtgttagaatttaaggcacagtgagaaagggaaggcatatactttttgaatgcaagaaacttcttcccaatccccctgaaattgcatcatttgagtaactatctcttccatatataaagtcacaacaatttctctctcagtcccagaactttgaagccttttcaaactttccttcttttggtatctaggaggaatacatttttgaagattgttcttggtgtctttcaggaaccaacacaatggagagggaagagagtgaacagaccaagaaaaggtaaatcctgaccctgagacattgatgagagagaggtataatccccagagtgcctgttacttgaataggcttatgcctaacatatgttgagacctcagcaaacctgaactaatggagagggagaggaaaataaaactagttaagaactggaagaaaataacctgataatggatgacagggtatccaatgcacaatgcccagaaagcatgacaagctctgtcatggtcaagtaaaagtcaataccaaagacttcagaggtggtgaacatgggcttcatcttatctgccacagtaaccccagtacctggcacagtgcctagattagtgggcatcctacatgtgtggaatgaataaatgaagaagtggggaatgataacatgtttgcttcagcctgagcatcttagtatttgctatggccctgtttagatgttcttctgccacttctttacctcattcttcagatcttgcctcaagcagcactttcttaaaaaccctttcccaaactagaaaatgtcaacttgttacagtgtcatgtggatcccttggctttttcttaataacaccagattatgcttacatatttgtgtaattatcttattaaactctataaactagacttaactaaatcctatgaagagcagagaccataccagttaagctcatcattgtgctgctagcacttagcatggtgcctggcatatagcaggttctcaataaatgttgaaagaatgattgatgcatgatgaatacataaaagttcgtggtgatcagtcctttcacaacgtgaagctatcagatagtctgtacctctatccctcctgagaaattaagctctcaggaatatcaaggctctgactgcatacccataggatcaaagcaaccctcagtcacaagcctggtttcagagatagggtcataacccccagggtgcagagacaaccgagagtacccagcactaatccagatataccagccactgtgattctagcaacaaaactaataattccgggcacccttggacaatgagaaagggtgctgaaatcctgcctaccctgtcacactcagtttcagaaatggtctggaagagcctgcagagggcaggcagcagagaaccggcagagggcatgggaagggccaggcagaaataaagggtagctcttgaagcatagatgacagtgtagaccgtggttcttttctcttgctttctccacctttctcttcaatagtttgtttctcctcattgctgttccaatggcaacctctattctgccctatcattgaaatctagaaaaagaaagtagctcaaatgtgaaatatcacctaatcttttcttctatttctccagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcatttaaaagtctacattttctgctttggcttcaagagtactactcaacccttgtggtctgatgttccctgctctgtcctctgaatgtacttcctttctctttacatctctatggctagaagcctctcacgcatcctgtatcttctcctcctcccttttccctaccattatttgagaaaggaggcttgtatacttctatatgtttatctcagtaataagtcataaaaaatcaagtaagaatggttgtttttgaggacaactaagaaatctggaataaggaagggaagcttacttttgagtttgtaacctgtagtgtgtaattttttaattatgtacttacatgtacattaaacaaaagcttaatgtaaaaatattccttgaaaacaccatgattataaaataaatgcatatatacacatacagcatgtgagaggagccaggaaaactctggaaaaaagaaaattacctagactctgtgagggcaggaatgtgtttaatttctctccaatggatcctcagacaactaagatagttgtctattctattgtccatctttttgtcttttgttgtatttcttaaagattccctcaactttatcttctaacttctgttgtatttttatttctgctatcatgtattcttttcagaattcttttttgttctctcaaaacatatctgtttaaagattgaatgaaatattaacatgccctttggtgagaacatccctcctttgtatattaaattctctgaactgctgtattctaagactaggggaaagaaaaagaaggttgaaagaggtcattaggcagaatagtactagctaacattatttcacatttaccatatacccgtcactcatctaaacctttaaactcattatcctatttaatcctcacaatgaccctgtgacgtaggtaatggaatattatgcccattatgctgatgagaaaatataaacacagagataagtcagagtaatttacccaacattgttaactttgtaagtggcagagctttgtaacaggcagaggttggaacagtttggagggctcagaagaagacaggaagatgtaggaaagtttggaacttcccagagccttgttgaatggctttgaccaaaatgctgatagtaatatggacaatgaaatacaggctgaggtggtctcagatagagaagaggaacttgttgggaactggaataaaggtgactcttgctatgttttagcaaagacactggtgg

SEQ ID NO: 298 - DNA, 20 bp, Artificial Sequence
  Antisense Oligonucleotide
accaaaaggagtatttgcga

SEQ ID NO: 299 - DNA, 20 bp, Artificial Sequence
  Antisense Oligonucleotide
cattcccaaggaacacagaa

SEQ ID NO: 300 - DNA, 20 bp, Artificial Sequence
  Antisense Oligonucleotide
actgtagctccaaaaagaga

SEQ ID NO: 301 - DNA, 20 bp, Artificial Sequence
  Antisense Oligonucleotide
ctgtcacaaatgcctgtcca

SEQ ID NO: 302 - DNA, 20 bp, Artificial Sequence
  Antisense Oligonucleotide
tcagtcccatagtgctgtca

SEQ ID NO: 303 - DNA, 20 bp, Artificial Sequence
  Antisense Oligonucleotide
ctgttacagcagcagagaag

SEQ ID NO: 304 - DNA, 20 bp, Artificial Sequence
  Antisense Oligonucleotide
tccctgttacagcagcagag

SEQ ID NO: 305 - DNA, 20 bp, Artificial Sequence
  Antisense Oligonucleotide
atctggaaatgaccccactc

SEQ ID NO: 306 - DNA, 20 bp, Artificial Sequence
  Antisense Oligonucleotide
gtgacctaatatctggaaat

SEQ ID NO: 307 - DNA, 20 bp, Artificial Sequence
  Antisense Oligonucleotide
cattttggctgcttctgctg

SEQ ID NO: 308 - DNA, 20 bp, Artificial Sequence
  Antisense Oligonucleotide
ggaacttacaaaggaaaggg

SEQ ID NO: 309 - DNA, 20 bp, Artificial Sequence
  Antisense Oligonucleotide
aaaaaggttgcccaggaact

SEQ ID NO: 310 - DNA, 20 bp, Artificial Sequence
  Antisense Oligonucleotide
tgccttctggaagaaatcaa

SEQ ID NO: 311 - DNA, 20 bp, Artificial Sequence
  Antisense Oligonucleotide
tttttgccttctggaagaaa

SEQ ID NO: 312 - DNA, 20 bp, Artificial Sequence
  Antisense Oligonucleotide
ctattccacttagagggagt

SEQ ID NO: 313 - DNA, 20 bp, Artificial Sequence
  Antisense Oligonucleotide
tctgatctggaggaggtatt

SEQ ID NO: 314 - DNA, 20 bp, Artificial Sequence
  Antisense Oligonucleotide
agaaattgagaggtctattt

SEQ ID NO: 315 - DNA, 20 bp, Artificial Sequence
  Antisense Oligonucleotide
caccagcttagaattctggg

SEQ ID NO: 316 - DNA, 20 bp, Artificial Sequence
  Antisense Oligonucleotide
aggtagttgtttagtcacag

SEQ ID NO: 317 - DNA, 20 bp, Artificial Sequence
  Antisense Oligonucleotide
ccagactgaggaggtagttg

SEQ ID NO: 318 - DNA, 20 bp, Artificial Sequence
  Antisense Oligonucleotide
cagtacatagatctctatgt

SEQ ID NO: 319 - DNA, 20 bp, Artificial Sequence
  Antisense Oligonucleotide
ttacagtacatagatctcta

SEQ ID NO: 320 - DNA, 20 bp, Artificial Sequence
  Antisense Oligonucleotide
gatgagaactccttagcagg

SEQ ID NO: 321 - DNA, 20 bp, Artificial Sequence
  Antisense Oligonucleotide
tagcaacagcccagatagaa

SEQ ID NO: 322 - DNA, 20 bp, Artificial Sequence
  Antisense Oligonucleotide
tctgttgcttgtttcaagac

SEQ ID NO: 323 - DNA, 20 bp, Artificial Sequence
  Antisense Oligonucleotide
tccatttggacagactatcc

SEQ ID NO: 324 - DNA, 20 bp, Artificial Sequence
  Antisense Oligonucleotide
gggaaactgctgtctgtctt

SEQ ID NO: 325 - DNA, 20 bp, Artificial Sequence
  Antisense Oligonucleotide
tgcttccaggaagatgacat

SEQ ID NO: 326 - DNA, 20 bp, Artificial Sequence
  Antisense Oligonucleotide
attcatcccattatcaaggt

SEQ ID NO: 327 - DNA, 20 bp, Artificial Sequence
  Antisense Oligonucleotide
agccaggagtggaaagtcct

SEQ ID NO: 328 - DNA, 20 bp, Artificial Sequence
  Antisense Oligonucleotide
cttcctaattccgttgcagc

SEQ ID NO: 329 - DNA, 20 bp, Artificial Sequence
  Antisense Oligonucleotide
catctgtaggctaagtaagc

SEQ ID NO: 330 - DNA, 20 bp, Artificial Sequence
  Antisense Oligonucleotide
cccgtaggacatctgtaggc

SEQ ID NO: 331 - DNA, 20 bp, Artificial Sequence
  Antisense Oligonucleotide
gccctatgctgggccagccc

SEQ ID NO: 332 - DNA, 20 bp, Artificial Sequence
  Antisense Oligonucleotide
gtctctgtatgcaagtttcc

SEQ ID NO: 333 - DNA, 20 bp, Artificial Sequence
  Antisense Oligonucleotide
ccagtatatctgtctctgta

SEQ ID NO: 334 - DNA, 20 bp, Artificial Sequence
  Antisense Oligonucleotide
ccaggttttcaaagtcattt

SEQ ID NO: 335 - DNA, 20 bp, Artificial Sequence
  Antisense Oligonucleotide
agccaggttttcaaagtcat

SEQ ID NO: 336 - DNA, 20 bp, Artificial Sequence
  Antisense Oligonucleotide
cccttagtgatcccacctta

SEQ ID NO: 337 - DNA, 20 bp, Artificial Sequence
  Antisense Oligonucleotide
ctgccccatcccttagtgat

SEQ ID NO: 338 - DNA, 20 bp, Artificial Sequence
  Antisense Oligonucleotide
tttatgtttgggcagagact

SEQ ID NO: 339 - DNA, 20 bp, Artificial Sequence
  Antisense Oligonucleotide
catggcagtctataaccctt

SEQ ID NO: 340 - DNA, 20 bp, Artificial Sequence
  Antisense Oligonucleotide
tagcatggcagtctataacc

SEQ ID NO: 341 - DNA, 20 bp, Artificial Sequence
  Antisense Oligonucleotide
tctagcatggcagtctataa

SEQ ID NO: 342 - DNA, 20 bp, Artificial Sequence
  Antisense Oligonucleotide
ttgtctagcatggcagtcta

SEQ ID NO: 343 - DNA, 20 bp, Artificial Sequence
  Antisense Oligonucleotide
aagcttgtctagcatggcag

SEQ ID NO: 344 - DNA, 20 bp, Artificial Sequence
  Antisense Oligonucleotide
acatggacaagcttgtctag

SEQ ID NO: 345 - DNA, 20 bp, Artificial Sequence
  Antisense Oligonucleotide
ttacatggacaagcttgtct

SEQ ID NO: 346 - DNA, 20 bp, Artificial Sequence
  Antisense Oligonucleotide
gaatattacatggacaagct

SEQ ID NO: 347 - DNA, 20 bp, Artificial Sequence
  Antisense Oligonucleotide
aactagccaggtgctaggag

SEQ ID NO: 348 - DNA, 20 bp, Artificial Sequence
  Antisense Oligonucleotide
aattattactcaccactggg

SEQ ID NO: 349 - DNA, 20 bp, Artificial Sequence
  Antisense Oligonucleotide
taatatttagggaagcatga

SEQ ID NO: 350 - DNA, 20 bp, Artificial Sequence
  Antisense Oligonucleotide
ggaccctgggccagttattg

SEQ ID NO: 351 - DNA, 20 bp, Artificial Sequence
  Antisense Oligonucleotide
caaacatacctgtcacaaat

SEQ ID NO: 352 - DNA, 20 bp, Artificial Sequence
  Antisense Oligonucleotide
gtgatatcaattgatggcat

SEQ ID NO: 353 - DNA, 20 bp, Artificial Sequence
  Antisense Oligonucleotide
tgctacatctactcagtgtc

SEQ ID NO: 354 - DNA, 20 bp, Artificial Sequence
  Antisense Oligonucleotide
tggaaactcttgccttcgga

SEQ ID NO: 355 - DNA, 20 bp, Artificial Sequence
  Antisense Oligonucleotide
ccatccacattgtagcatgt

SEQ ID NO: 356 - DNA, 20 bp, Artificial Sequence
  Antisense Oligonucleotide
tcaggatggtatggccatac

SEQ ID NO: 357 - DNA, 20 bp, Artificial Sequence
  Antisense Oligonucleotide
tcccatagtgctagagtcga

SEQ ID NO: 358 - DNA, 20 bp, Artificial Sequence
  Antisense Oligonucleotide
aggttcttaccagagagcag

SEQ ID NO: 359 - DNA, 20 bp, Artificial Sequence
  Antisense Oligonucleotide
cagaggagcagcacctaaaa

SEQ ID NO: 360 - DNA, 20 bp, Artificial Sequence
  Antisense Oligonucleotide
gaccacataccaagcactga

SEQ ID NO: 361 - DNA, 20 bp, Artificial Sequence
  Antisense Oligonucleotide
atctttcagaaacccaagca

SEQ ID NO: 362 - DNA, 20 bp, Artificial Sequence
  Antisense Oligonucleotide
gagtcaccaaagatttacaa

SEQ ID NO: 363 - DNA, 20 bp, Artificial Sequence
  Antisense Oligonucleotide
ctgaagttagctgaaagcag

SEQ ID NO: 364 - DNA, 20 bp, Artificial Sequence
  Antisense Oligonucleotide
acagctttacctatagagaa

SEQ ID NO: 365 - DNA, 20 bp, Artificial Sequence
  Antisense Oligonucleotide
tcctcaagctctacaaatga

SEQ ID NO: 366 - DNA, 20 bp, Artificial Sequence
  Antisense Oligonucleotide
gactcactcaccacatttat

SEQ ID NO: 367 - DNA, 20 bp, Artificial Sequence
  Antisense Oligonucleotide
agtgatagcaaggcttctct

SEQ ID NO: 368 - DNA, 20 bp, Artificial Sequence
  Antisense Oligonucleotide
cttggagagaatggttatct

SEQ ID NO: 369 - DNA, 20 bp, Artificial Sequence
  Antisense Oligonucleotide
gaagatgttgatgcctaaat

SEQ ID NO: 370 - DNA, 20 bp, Artificial Sequence
  Antisense Oligonucleotide
gtgttggttcctgaaagaca

SEQ ID NO: 371 - DNA, 20 bp, Artificial Sequence
  Antisense Oligonucleotide
caggatttaccttttcttgg

SEQ ID NO: 372 - DNA, 20 bp, Artificial Sequence
  Antisense Oligonucleotide
agggcagaatagaggttgcc

SEQ ID NO: 373 - DNA, 20 bp, Artificial Sequence
  Antisense Oligonucleotide
tttttctctggagaaataga

SEQ ID NO: 374 - DNA, 20 bp, Artificial Sequence
  Antisense Oligonucleotide
gttactcagtcccatagtgc

SEQ ID NO: 375 - DNA, 20 bp, Artificial Sequence
  Antisense Oligonucleotide
caaagagaatgttactcagt

SEQ ID NO: 376 - DNA, 20 bp, Artificial Sequence
  Antisense Oligonucleotide
ccatcacaaagagaatgtta

SEQ ID NO: 377 - DNA, 20 bp, Artificial Sequence
  Antisense Oligonucleotide
ggaaggccatcacaaagaga

SEQ ID NO: 378 - DNA, 20 bp, Artificial Sequence
  Antisense Oligonucleotide
gagcaggaaggccatcacaa

SEQ ID NO: 379 - DNA, 20 bp, Artificial Sequence
  Antisense Oligonucleotide
ccagagagcaggaaggccat

SEQ ID NO: 380 - DNA, 20 bp, Artificial Sequence
  Antisense Oligonucleotide
aaataagcttgaatcttcag

SEQ ID NO: 381 - DNA, 20 bp, Artificial Sequence
  Antisense Oligonucleotide
agtctcattgaaataagctt

SEQ ID NO: 382 - DNA, 20 bp, Artificial Sequence
  Antisense Oligonucleotide
aggtctgcagtctcattgaa

SEQ ID NO: 383 - DNA, 20 bp, Artificial Sequence
  Antisense Oligonucleotide
ctactagctcactcaggctt

SEQ ID NO: 384 - DNA, 20 bp, Artificial Sequence
  Antisense Oligonucleotide
aaatactactagctcactca

SEQ ID NO: 385 - DNA, 20 bp, Artificial Sequence
  Antisense Oligonucleotide
ctgccaaaatactactagct

SEQ ID NO: 386 - DNA, 20 bp, Artificial Sequence
  Antisense Oligonucleotide
ttcagaaccaagttttcctg

SEQ ID NO: 387 - DNA, 20 bp, Artificial Sequence
  Antisense Oligonucleotide
cctcattcagaaccaagttt

SEQ ID NO: 388 - DNA, 20 bp, Artificial Sequence
  Antisense Oligonucleotide
gtatacctcattcagaacca

SEQ ID NO: 389 - DNA, 20 bp, Artificial Sequence
  Antisense Oligonucleotide
gcctaagtatacctcattca

SEQ ID NO: 390 - DNA, 20 bp, Artificial Sequence
  Antisense Oligonucleotide
ctctttgcctaagtatacct

SEQ ID NO: 391 - DNA, 20 bp, Artificial Sequence
  Antisense Oligonucleotide
cccatatacttggaatgaac

SEQ ID NO: 392 - DNA, 20 bp, Artificial Sequence
  Antisense Oligonucleotide
cttgtgcggcccatatactt

SEQ ID NO: 393 - DNA, 20 bp, Artificial Sequence
  Antisense Oligonucleotide
atcaaaacttgtgcggccca

SEQ ID NO: 394 - DNA, 20 bp, Artificial Sequence
  Antisense Oligonucleotide
cccttgtccttgatctgaag

SEQ ID NO: 395 - DNA, 20 bp, Artificial Sequence
  Antisense Oligonucleotide
acaagcccttgtccttgatc

SEQ ID NO: 396 - DNA, 20 bp, Artificial Sequence
  Antisense Oligonucleotide
ttgatacaagcccttgtcct

SEQ ID NO: 397 - DNA, 20 bp, Artificial Sequence
  Antisense Oligonucleotide
atacattgatacaagccctt

SEQ ID NO: 398 - DNA, 20 bp, Artificial Sequence
  Antisense Oligonucleotide
tggatgatacattgatacaa

SEQ ID NO: 399 - DNA, 20 bp, Artificial Sequence
  Antisense Oligonucleotide
gaattcatctggtggatgcg

SEQ ID NO: 400 - DNA, 20 bp, Artificial Sequence
  Antisense Oligonucleotide
gttcagaattcatctggtgg

SEQ ID NO: 401 - DNA, 20 bp, Artificial Sequence
  Antisense Oligonucleotide
tgacagttcagaattcatct

SEQ ID NO: 402 - DNA, 20 bp, Artificial Sequence
  Antisense Oligonucleotide
agcactgacagttcagaatt

SEQ ID NO: 403 - DNA, 20 bp, Artificial Sequence
  Antisense Oligonucleotide
tagcaagcactgacagttca

SEQ ID NO: 404 - DNA, 20 bp, Artificial Sequence
  Antisense Oligonucleotide
tgaagttagcaagcactgac

SEQ ID NO: 405 - DNA, 20 bp, Artificial Sequence
  Antisense Oligonucleotide
ttgactgaagttagcaagca

SEQ ID NO: 406 - DNA, 20 bp, Artificial Sequence
  Antisense Oligonucleotide
ctatttcaggttgactgaag

SEQ ID NO: 407 - DNA, 20 bp, Artificial Sequence
  Antisense Oligonucleotide
tctgttatattagaaattgg

SEQ ID NO: 408 - DNA, 20 bp, Artificial Sequence
  Antisense Oligonucleotide
gcaggtcaaatttatgtaca

SEQ ID NO: 409 - DNA, 20 bp, Artificial Sequence
  Antisense Oligonucleotide
gtatagatgagcaggtcaaa

SEQ ID NO: 410 - DNA, 20 bp, Artificial Sequence
  Antisense Oligonucleotide
gggtaaccgtgtatagatga

SEQ ID NO: 411 - DNA, 20 bp, Artificial Sequence
  Antisense Oligonucleotide
aggttctgggtaaccgtgta

SEQ ID NO: 412 - DNA, 20 bp, Artificial Sequence
  Antisense Oligonucleotide
tagcaaaacactcatcttct

SEQ ID NO: 413 - DNA, 20 bp, Artificial Sequence
  Antisense Oligonucleotide
gttcttagcaaaacactcat

SEQ ID NO: 414 - DNA, 20 bp, Artificial Sequence
  Antisense Oligonucleotide
attcttggttcttagcaaaa

SEQ ID NO: 415 - DNA, 20 bp, Artificial Sequence
  Antisense Oligonucleotide
gatagttgaattcttggttc

SEQ ID NO: 416 - DNA, 20 bp, Artificial Sequence
  Antisense Oligonucleotide
accatcatactcgatagttg

SEQ ID NO: 417 - DNA, 20 bp, Artificial Sequence
  Antisense Oligonucleotide
atcttgagatttctgcataa

SEQ ID NO: 418 - DNA, 20 bp, Artificial Sequence
  Antisense Oligonucleotide
acattatcttgagatttctg

SEQ ID NO: 419 - DNA, 20 bp, Artificial Sequence
  Antisense Oligonucleotide
cgtacagttctgtgacatta

SEQ ID NO: 420 - DNA, 20 bp, Artificial Sequence
  Antisense Oligonucleotide
agacaagctgatggaaacgt

SEQ ID NO: 421 - DNA, 20 bp, Artificial Sequence
  Antisense Oligonucleotide
gaaacagacaagctgatgga

SEQ ID NO: 422 - DNA, 20 bp, Artificial Sequence
  Antisense Oligonucleotide
ggaatgaaacagacaagctg

SEQ ID NO: 423 - DNA, 20 bp, Artificial Sequence
  Antisense Oligonucleotide
catcagggaatgaaacagac

SEQ ID NO: 424 - DNA, 20 bp, Artificial Sequence
  Antisense Oligonucleotide
cgtaacatcagggaatgaaa

SEQ ID NO: 425 - DNA, 20 bp, Artificial Sequence
  Antisense Oligonucleotide
agctctatagagaaaggtga

SEQ ID NO: 426 - DNA, 20 bp, Artificial Sequence
  Antisense Oligonucleotide
cctcaagctctatagagaaa

SEQ ID NO: 427 - DNA, 20 bp, Artificial Sequence
  Antisense Oligonucleotide
ggaggctgagggtcctcaag

SEQ ID NO: 428 - DNA, 20 bp, Artificial Sequence
  Antisense Oligonucleotide
agtacagctgtaatccaagg

SEQ ID NO: 429 - DNA, 20 bp, Artificial Sequence
  Antisense Oligonucleotide
ttggaagtacagctgtaatc

SEQ ID NO: 430 - DNA, 20 bp, Artificial Sequence
  Antisense Oligonucleotide
ataataactgttggaagtac

SEQ ID NO: 431 - DNA, 20 bp, Artificial Sequence
  Antisense Oligonucleotide
catcacacatataataactg

SEQ ID NO: 432 - DNA, 20 bp, Artificial Sequence
  Antisense Oligonucleotide
tccatttccatagaattaga

SEQ ID NO: 433 - DNA, 20 bp, Artificial Sequence
  Antisense Oligonucleotide
tcttcttccatttccataga

SEQ ID NO: 434 - DNA, 20 bp, Artificial Sequence
  Antisense Oligonucleotide
atttataagagttgcgaggc

SEQ ID NO: 435 - DNA, 20 bp, Artificial Sequence
  Antisense Oligonucleotide
ttggttccacatttataaga

SEQ ID NO: 436 - DNA, 20 bp, Artificial Sequence
  Antisense Oligonucleotide
ctctccattgtgttggttcc

SEQ ID NO: 437 - DNA, 20 bp, Artificial Sequence
  Antisense Oligonucleotide
cttccctctccattgtgttg

SEQ ID NO: 438 - DNA, 20 bp, Artificial Sequence
  Antisense Oligonucleotide
tggtctgttcactctcttcc

SEQ ID NO: 439 - DNA, 20 bp, Artificial Sequence
  Antisense Oligonucleotide
ttcatcagatctttcaggta

SEQ ID NO: 440 - DNA, 20 bp, Artificial Sequence
  Antisense Oligonucleotide
atcacttttgtcgcatgaag

SEQ ID NO: 441 - DNA, 20 bp, Artificial Sequence
  Antisense Oligonucleotide
gctttactctttaattaaaa

SEQ ID NO: 442 - DNA, 20 bp, Artificial Sequence
  Antisense Oligonucleotide
gtatgggctttactctttaa

SEQ ID NO: 443 - DNA, 20 bp, Artificial Sequence
  Antisense Oligonucleotide
atacttgtatgggctttact

SEQ ID NO: 444 - DNA, 20 bp, Artificial Sequence
  Antisense Oligonucleotide
aatgaatacttgtatgggct

(19) United States
(12) Patent Application Publication
(10) Pub. No.: US 2004/0023917 A1
(43) Pub. Date: Feb. 5, 2004

(54) Oligonucleotide compositions and methods for the modulation of the expression of B7 protein

(76) Inventors: C. Frank Bennett, Carlsbad, CA (US); Timothy A. Vickers, Oceanside, CA (US); James G. Karras, San Marcos, CA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/444,206

(22) Filed: May 23, 2003

Related U.S. Application Data
(63) Continuation-in-part of application No. 09/851,871, filed on May 9, 2001.
Application No. 09/851,871 is a continuation-in-part of application No. PCT/US00/14471, filed on May 25, 2000.
Application No. PCT/US00/14471 is a continuation-in-part of application No. 09/326,186, filed on Jun. 4, 1999, now Pat. No. 6,319,906.
Application No. 09/326,186 is a continuation-in-part of application No. 08/777,266, filed on Dec. 31, 1996, now Pat. No. 6,077,833.

(51) Int. Cl. A61K 48/00

(52) U.S. Cl. 514/44

(57) ABSTRACT

Compositions and methods for the treatment of asthma with oligonucleotides which specifically hybridize with nucleic acids encoding B7 proteins.

### INTRODUCTION

This is a continuation-in-part of U.S. application Ser. No. 09/851,871, filed May 9, 2001, which is a continuation-in-part of International Patent Application No. PCT/US00/14471, which is a continuation-in-part of U.S. application Ser. No. 09/326,186, filed Jun. 4, 1999, which is a continuation-in-part of U.S. application Ser. No. 08/777,266, filed Dec. 31, 1996, the entire disclosures of which are incorporated herein by reference.

### FIELD OF THE INVENTION

This invention relates to diagnostics, research reagents and therapeutics for disease states which respond to modulation of T cell activation. In particular, this invention relates to antisense oligonucleotide interactions with certain messenger ribonucleic acids (mRNAs) or DNAs involved in the synthesis of proteins that modulate T cell activation. Antisense oligonucleotides designed to hybridize to nucleic acids encoding B7 proteins are provided. These oligonucleotides have been found to lead to the modulation of the activity of the RNA or DNA, and thus to the modulation of T cell activation. Palliative, therapeutic and prophylactic effects result.

### BACKGROUND OF THE INVENTION

Inflammation is a localized protective response mounted by tissues in response to injury, infection, or tissue destruction resulting in the destruction of the infectious or injurious agent and isolation of the injured tissue. A typical inflammatory response proceeds as follows: recognition of an antigen as foreign or recognition of tissue damage, synthesis and release of soluble inflammatory mediators, recruitment of inflammatory cells to the site of infection or tissue damage, destruction and removal of the invading organism or damaged tissue, and deactivation of the system once the invading organism or damage has been resolved. In many human diseases with an inflammatory component, the normal, homeostatic mechanisms which attenuate the inflammatory responses are defective, resulting in damage and destruction of normal tissue.

Cell-cell interactions are involved in the activation of the immune response at each of the stages described above. One of the earliest detectable events in a normal inflammatory response is adhesion of leukocytes to the vascular endothelium, followed by migration of leukocytes out of the vasculature to the site of infection or injury. In general, the first inflammatory cells to appear at the site of inflammation are neutrophils, followed by monocytes and lymphocytes. Cell-cell interactions are also critical for activation of both B-lymphocytes (B cells) and T-lymphocytes (T cells) with resulting enhanced humoral and cellular immune responses, respectively.

The hallmark of the immune system is its ability to distinguish between self (host) and nonself (foreign invaders). This remarkable specificity exhibited by the immune system is mediated primarily by T cells. T cells participate in the host's defense against infection but also mediate organ damage of transplanted tissues and contribute to cell attack in graft-versus-host disease (GVHD) and some autoimmune diseases. In order to induce an antigen-specific immune response, a T cell must receive signals delivered by an antigen-presenting cell (APC). T cell-APC interactions can be divided into three stages: cellular adhesion, T cell receptor (TCR) recognition, and costimulation. At least two discrete signals are required from an APC for induction of T cell activation. The first signal is antigen-specific and is provided when the TCR interacts with an antigen in the context of a major histocompatibility complex (MHC) protein, or an MHC-related CD1 protein, expressed on the surface of an APC (“CD,” standing for “cluster of differentiation,” is a term used to denote different T cell surface molecules). The second (costimulatory) signal involves the interaction of the T cell surface antigen, CD28, with its ligand on the APC, which is a member of the B7 family of proteins.

CD28, a disulfide-linked homodimer of a 44 kilodalton polypeptide and a member of the immunoglobulin superfamily, is one of the major costimulatory signal receptors on the surface of a resting T cell for T cell activation and cytokine production (Allison, Curr. Opin. Immunol., 1994, 6, 414; Linsley and Ledbetter, Annu. Rev. Immunol., 1993, 11, 191; June et al., Immunol. Today, 1994, 15, 321). Signal transduction through CD28 acts synergistically with TCR signal transduction to augment both interleukin-2 (IL-2) production and proliferation of naive T cells. B7-1 (also known as CD80) was the first ligand identified for CD28 (Liu and Linsley, Curr. Opin. Immunol., 1992, 4, 265). B7-1 is normally expressed at low levels on APCs, however, it is upregulated following activation by cytokines or ligation of cell surface molecules such as CD40 (Lenschow et al., Proc. Natl. Acad. Sci. U.S.A., 1993, 90, 11054; Nabavi et al., Nature, 1992, 360, 266). Initial studies suggested that B7-1 was the CD28 ligand that mediated costimulation (Reiser et al., Proc. Natl. Acad. Sci. U.S.A., 1992, 89, 271; Wu et al., J. Exp. Med., 1993, 178, 1789; Harlan et al., Proc. Natl. Acad. Sci. U.S.A., 1994, 91, 3137). However, the subsequent demonstration that anti-B7-1 monoclonal antibodies (mabs) had minimal effects on primary mixed lymphocyte reactions and that B7-1-deficient mice responded normally to antigens (Lenschow et al., Proc. Natl. Acad. Sci. U.S.A., 1993, 90, 11054; Freeman et al., Science, 1993, 262, 909) resulted in the discovery of a second ligand for the CD28 receptor, B7-2 (also known as CD86). In contrast with anti-B7-1 mAbs, anti-B7-2 mabs are potent inhibitors of T cell proliferation and cytokine production (Wu et al., J. Exp. Med., 1993, 178, 1789; Chen et al., J. Immunol., 1994, 152, 2105; Lenschow et al., Proc. Natl. Acad. Sci. U.S.A., 1993, 90, 11054). B7:CD28 signaling may be a necessary component of other T cell costimulatory pathways, such as CD40:CD40L (CD40 ligand) signaling (Yang et al., Science, 1996, 273, 1862).

In addition to binding CD28, B7-1 and B7-2 bind the cytolytic T-lymphocyte associated protein CTLA4. CTLA4 is a protein that is structurally related to CD28 but is expressed on T cells only after activation (Linsley et al., J. Exp. Med., 1991, 174, 561). A soluble recombinant form of CTLA4, CTLA4-Ig, has been determined to be a more efficient inhibitor of the B7:CD28 interaction than monoclonal antibodies directed against CD28 or a B7 protein. In vivo treatment with CTLA4-Ig results in the inhibition of antibody formation to sheep red blood cells or soluble antigen (Linsley et al., Science, 1992, 257, 792), prolongation of cardiac allograft and pancreatic islet xenograft survival (Lin et al., J. Exp. Med., 1993, 178, 1801; Lenschow et al., 1992, Science, 257, 789; Lenschow et al., Curr. Opin. Immunol., 1991, 9, 243), and significant suppression of immune responses in GVHD (Hakim et al., J. Immun., 1995, 155, 1760). It has been proposed that CD28 and CTLA4, although both acting through common B7 receptors, serve opposing costimulatory and inhibitory functions, respectively (Allison et al., Science, 1995, 270, 932). CTLA41 g, which binds both B7-1 and B7-2 molecules on antigen-presenting cells, has been shown to block T-cell costimulation in patients with stable psoriasis vulgaris, and to cause a 50% or greater sustained improvement in clinical disease activity in 46% of the patients to which it was administered. This result was dose-dependent. Abrams et al., J. Clin. Invest., 1999, 9, 1243-1225.

European Patent Application No. EP 0 600 591 discloses a method of inhibiting tumor cell growth in which tumor cells from a patient are recombinantly engineered ex vivo to express a B7-1 protein and then reintroduced into a patient. As a result, an immunologic response is stimulated against both B7-transfected and nontransfected tumor cells.

International Publication No. WO95/03408 discloses nucleic acids encoding novel CTLA4/CD28 ligands which costimulate T cell activation, including B7-2 proteins. Also disclosed are antibodies to B7-2 proteins and methods of producing B7-2 proteins.

International Publication No. WO95/05464 discloses a polypeptide, other than B7-1, that binds to CTLA4, CD28 or CTLA4-Ig. Also disclosed are methods for obtaining a nucleic acid encoding such a polypeptide.

International Publication No. WO 95/06738 discloses nucleic acids encoding B7-2 (also known as B70) proteins. Also disclosed are antibodies to B7-2 proteins and methods of producing B7-2 proteins.

European Patent Application No. EP 0 643 077 discloses a monoclonal antibody which specifically binds a B7-2 (also known as B70) protein. Also disclosed are methods of producing monoclonal antibodies which specifically bind a B7-2 protein.

U.S. Pat. No. 5,434,131 discloses the CTLA4 protein as a ligand for B7 proteins. Also disclosed are methods of producing CTLA4 fusion proteins (e.g., CTLA4-Ig) and methods of regulating immune responses using antibodies to B7 proteins or CTLA4 proteins.

International Publication No. WO95/22619 discloses antibodies specific to B7-1 proteins which do not bind to B7-2 proteins. Also disclosed are methods of regulating immune responses using antibodies to B7-1 proteins.

International Publication No. WO95/34320 discloses methods for inhibiting T cell responses using a first agent which inhibits a costimulatory agent, such as an CTLA4-Ig fusion protein, and a second agent which inhibits cellular adhesion, such as an anti-LFA-1 antibody. Such methods are indicated to be particularly useful for inhibiting the rejection of transplanted tissues or organs.

International Publication No. WO95/32734 discloses Fc RII bridging agents which either prevent the upregulation of B7 molecules or impair the expression of ICAM-3 on antigen presenting cells. Such FcRII bridging agents include proteins such as aggregated human IgG molecules or aggregated Fc fragments of human IgG molecules.

International Publication No. WO96/11279 discloses recombinant viruses comprising genetic sequences encoding (1) one or more immunostimulatory agents, including B7-1 and B7-2, and (2) and antigens from a disease causing agent. Also disclosed are methods of treating diseases using such recombinant viruses.

To date, there are no known therapeutic agents which effectively regulate and prevent the expression of B7 proteins such as B7-1 and B7-2. Thus, there is a long-felt need for compounds and methods which effectively modulate critical costimulatory molecules such as the B7 proteins.

### SUMMARY OF THE INVENTION

In accordance with the present invention, oligonucleotides are provided which specifically hybridize with nucleic acids encoding B7-1 or B7-2. Certain oligonucleotides of the invention are designed to bind either directly to mRNA transcribed from, or to a selected DNA portion of, the B7-1 or B7-2 gene, thereby modulating the amount of protein translated from a B7-1 or B7-2 mRNA or the amount of mRNA transcribed from a B7-1 or B7-2 gene, respectively.

Oligonucleotides may comprise nucleotide sequences sufficient in identity and number to effect specific hybridization with a particular nucleic acid. Such oligonucleotides are commonly described as “antisense.” Antisense oligonucleotides are commonly used as research reagents, diagnostic aids, and therapeutic agents.

It has been discovered that the B7-1 and B7-2 genes, encoding B7-1 and B7-2 proteins, respectively, are particularly amenable to this approach. As a consequence of the association between B7 expression and T cell activation and proliferation, inhibition of the expression of B7-1 or B7-2 leads to inhibition of the synthesis of B7-1 or B7-2, respectively, and thereby inhibition of T cell activation and proliferation. Additionally, the oligonucleotides of the invention may be used to inhibit the expression of one of several alternatively spliced mRNAs of a B7 transcript, resulting in the enhanced expression of other alternatively spliced B7 mRNAs. Such modulation is desirable for treating various inflammatory or autoimmune disorders or diseases, or disorders or diseases with an inflammatory component such as asthma, juvenile diabetes mellitus, myasthenia gravis, Graves' disease, rheumatoid arthritis, allograft rejection, inflammatory bowel disease, multiple sclerosis, psoriasis, lupus erythematosus, systemic lupus erythematosus, diabetes, multiple sclerosis, contact dermatitis, rhinitis, various allergies, and cancers and their metastases. Such modulation is further desirable for preventing or modulating the development of such diseases or disorders in an animal suspected of being, or known to be, prone to such diseases or disorders.

In one embodiment, the invention provides methods of inhibiting the expression of a nucleic acid molecule encoding B7-1 or B7-2 in an individual, comprising the step of administering to said individual a compound of the invention targeted to a nucleic acid molecule encoding B7-1 or B7-2, wherein said compound specifically hybridizes with and inhibits the expression of a nucleic acid molecule encoding B7-1 or B7-2.

The invention further provides methods of inhibiting expression of a nucleic acid molecule encoding B7-1 or B7-2 in an individual, comprising the step of administering to an individual a compound of the invention which specifically hybridizes with at least an 8-nucleobase portion of an active site on a nucleic acid molecule encoding B7-1 or B7-2. Regions in the nucleic acid which when hybridized to a compound of the invention effect significantly lower B7-1 or B7-2 expression compared to a control, are referred to as active sites.

The invention also provides methods of inhibiting expression of a nucleic acid molecule encoding B7-1 or B7-2 in an individual, comprising the step of administering a compound of the invention targeted to a nucleic acid molecule encoding B7-1 or B7-2, wherein the compound specifically hybridizes with the nucleic acid and inhibits expression of B7-1 or B7-2.

In another aspect the invention provides methods of inhibiting expression of a nucleic acid molecule encoding B7-1 or B7-2 in an individual, comprising the step of administering a compound of the invention targeted to a nucleic acid molecule encoding B7-1 or B7-2, wherein the compound specifically hybridizes with the nucleic acid and inhibits expression of B7-1 or B7-2, said compound comprising at least 8 contiguous nucleobases of any one of the compounds of the invention.

The invention also provides methods of inhibiting the expression of a nucleic acid molecule encoding B7-1 or B7-2 in an individual, comprising the step of administering a compound of the invention targeted to a nucleic acid molecule encoding B7-1 or B7-2, wherein the compound specifically hybridizes with an active site in the nucleic acid and inhibits expression of B7-1 or B7-2, and the compound comprises at least 8 contiguous nucleobases of any one of the compounds of the invention.

In another aspect the invention provides methods of inhibiting expression of a nucleic acid molecule encoding B7-1 or B7-2 in an individual, comprising the step of administering an oligonucleotide mimetic compound targeted to a nucleic acid molecule encoding B7-1 or B7-2, wherein the compound specifically hybridizes with the nucleic acid and inhibits expression of B7-1 or B7-2, and the compound comprises at least 8 contiguous nucleobases of a compound of the invention.

In another aspect, the invention provides methods of inhibiting the expression of a nucleic acid molecule encoding B7-1 or B7-2 in an individual comprising the step of administering a compound of the invention target to a nucleic acid encoding B7-1 or B7-2, wherein the compound inhibits B7-1 or B7-2 mRNA expression by at least 5% in 80% confluent HepG2 cells in culture at an optimum concentration compared to a control. In yet another aspect, the compounds inhibit expression of mRNA encoding B7-1 or B7-2 in 80% confluent HepG2 cells in culture at an optimum concentration by at least 10%, at least 15%, at least 20%, at least 25%, at least 30%, at least 35%, at least 40%, or at least 50%, compared to a control.

The invention also relates to pharmaceutical compositions which comprise an antisense oligonucleotide to a B7 protein in combination with a second anti-inflammatory agent, such as a second antisense oligonucleotide to a protein which mediates intercellular interactions, e.g., an intercellular adhesion molecule (ICAM) protein.

Methods comprising contacting animals with oligonucleotides specifically hybridizable with nucleic acids encoding B7 proteins are herein provided. These methods are useful as tools, for example, in the detection and determination of the role of B7 protein expression in various cell functions and physiological processes and conditions, and for the diagnosis of conditions associated with such expression. Such methods can be used to detect the expression of 17 genes (i.e., B7-1 or B7-2) and are thus believed to be useful both therapeutically and diagnostically. Methods of modulating the expression of B7 proteins comprising contacting animals with oligonucleotides specifically hybridizable with a B7 gene are herein provided. These methods are believed to be useful both therapeutically and diagnostically as a consequence of the association between B7 expression and T cell activation and proliferation. The present invention also comprises methods of inhibiting B7-associated activation of T cells using the oligonucleotides of the invention. Methods of treating conditions in which abnormal or excessive T cell activation and proliferation occurs are also provided. These methods employ the oligonucleotides of the invention and are believed to be useful both therapeutically and as clinical research and diagnostic tools. The oligonucleotides of the present invention may also be used for research purposes. Thus, the specific hybridization exhibited by the oligonucleotides of the present invention may be used for assays, purifications, cellular product preparations and in other methodologies which may be appreciated by persons of ordinary skill in the art.

The methods disclosed herein are also useful, for example, as clinical research tools in the detection and determination of the role of B7-1 or B7-2 expression in various immune system functions and physiological processes and conditions, and for the diagnosis of conditions associated with their expression. The specific hybridization exhibited by the oligonucleotides of the present invention may be used for assays, purifications, cellular product preparations and in other methodologies which may be appreciated by persons of ordinary skill in the art. For example, because the oligonucleotides of this invention specifically hybridize to nucleic acids encoding B7 proteins, sandwich and other assays can easily be constructed to exploit this fact. Detection of specific hybridization of an oligonucleotide of the invention with a nucleic acid encoding a B7 protein present in a sample can routinely be accomplished. Such detection may include detectably labeling an oligonucleotide of the invention by enzyme conjugation, radiolabeling or any other suitable detection system. A number of assays may be formulated employing the present invention, which assays will commonly comprise contacting a tissue or cell sample with a detectably labeled oligonucleotide of the present invention under conditions selected to permit hybridization and measuring such hybridization by detection of the label, as is appreciated by those of ordinary skill in the art.

### BRIEF DESCRIPTION OF THE DRAWINGS

FIG. 1 is a bar graph showing the inhibitory effect of the indicated oligonucleotides on B7-1 protein expression in COS-7 cells.

FIG. 2 is a dose-response curve showing the inhibitory effect of oligonucleotides on cell surface expression of B7-1 protein. Solid line, ISIS 13812; dashed line, ISIS 13800; dotted line, ISIS 13805.

FIG. 3 is a bar graph showing the inhibitory effect of the indicated oligonucleotides on cell surface expression of B7-2 in COS-7 cells.

FIG. 4 is a bar graph showing the inhibitory effect of the indicated oligonucleotides, including ISIS 10373 (a 20-mer) and ISIS 10996 (a 15-mer) on cell surface expression of B7-2 in COS-7 cells.

FIG. 5 is a bar graph showing the specificity of inhibition of B7-1 or B7-2 protein expression by oligonucleotides. Cross-hatched bars, B7-1 levels; striped bars, B7-2 levels.

FIG. 6 is a dose-response curve showing the inhibitory effect of oligonucleotides having antisense sequences to ICAM-1 (ISIS 2302) or B7-2 (ISIS 10373) on cell surface expression of the ICAM-1 and B7-2 proteins. Solid line with X's, levels of B7-1 protein on cells treated with ISIS 10373; dashed line with asterisks, levels of ICAM-1 protein on cells treated with ISIS 10373; solid line with triangles, levels of B7-1 protein on cells treated with ISIS 2302; solid line with squares, levels of ICAM-1 protein on cells treated with ISIS 10373.

FIG. 7 is a bar graph showing the effect of the indicated oligonucleotides on T cell proliferation.

FIG. 8 is a dose-response curve showing the inhibitory effect of oligonucleotides on murine B7-2 protein expression in COS-7 cells. Solid line with asterisks, ISIS 11696; dashed line with triangles, ISIS 11866.

FIG. 9 is a bar graph showing the effect of oligonucleotides ISIS 11696 and ISIS 11866 on cell surface expression of murine B7-2 protein in IC-21 cells. Left (black) bars, no oligonucleotide; middle bars, 3 μM indicated oligonucleotide; right bars, 10/i M indicated oligonucleotide.

FIG. 10 is a graph showing the effect of ISIS 17456 on severity of EAE at various doses.

FIGS. 11A-B are graphs showing the detection of B7.2 mRNA (FIG. 11A) and B7.1 mRNA (FIG. 11B) during the development of ovalbumin-induced asthma in a mouse model.

FIG. 12 is a graph showing that intratracheal administration of ISIS 121874, an antisense oligonucleotide targeted to mouse B7.2, following allergen challenge, reduces the airway response to methacholine.

FIG. 13 is a graph showing the dose-dependent inhibition of the Penh response to 50 mg/ml methacholine by ISIS 121874. Penh is a dimensionless parameter that is a function of total pulmonary airflow in mice (i.e., the sum of the airflow in the upper and lower respiratory tracts) during the respiratory cycle of the animal. The lower the PENH, the greater the airflow. The dose of ISIS 121874 is shown on the x-axis.

FIG. 14 is a graph showing the inhibition of allergen-induced eosinophilia by ISIS 121874. The dose of ISIS 121874 is shown on the x-axis.

FIG. 15 is a graph showing the lung concentration-dose relationship for ISIS 121874 delivered by intratracheal administration.

FIG. 16 is a graph showing the retention of ISIS 121874 in lung tissue following single dose (0.3 mg/kg) intratracheal instillation in the ovalbumin-induced mouse model of asthma.

FIG. 17 is a graph showing the effects of ISIS 121874, a 7 base pair mismatched control oligonucleotide (ISIS 131906) and a gap ablated control oligonucleotide which does not promote cleavage by RNase H (ISIS 306058).

FIGS. 18A-B is a graph showing the effect of ISIS 121874 on B7.2 (FIG. 18A) and B7.1 (FIG. 18B) mRNA in lung tissue of ovalbumin-challenged mice.

FIGS. 19A-B is a graph showing the effect of ISIS 121874 on B7.2 (FIG. 19A) and B7.1 (FIG. 19B) mRNA in draining lymph nodes of ovalbumin-challenged mice.

FIG. 20 is a graph showing that treatment with an antisense oligonucleotide targeted to B7.1 (ISIS 121844) reduces allergen-induced eosinophilia in the ovalbumin-induced mouse model of asthma.

FIGS. 21A-B are graphs showing that treatment with ISIS 121844 reduces the levels of B7.1 mRNA (FIG. 21A) and B7.2 mRNA (FIG. 21B) in the mouse lung.

### DETAILED DESCRIPTION OF THE INVENTION

The present invention employs oligonucleotides for use in antisense inhibition of the function of RNA and DNA encoding B7 proteins including B7-1 and B7-2. The present invention also employs oligonucleotides which are designed to be specifically hybridizable to DNA or messenger RNA (mRNA) encoding such proteins and ultimately to modulate the amount of such proteins transcribed from their respective genes. Such hybridization with mRNA interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, and possibly even independent catalytic activity which may be engaged in by the RNA. The overall effect of such interference with mRNA function is modulation of the expression of a B7 protein, wherein “modulation” means either an increase (stimulation) or a decrease (inhibition) in the expression of a B7 protein. In the context of the present invention, inhibition is the preferred form of modulation of gene expression.

Oligonucleotides may comprise nucleotide sequences sufficient in identity and number to effect specific hybridization with a particular nucleic acid. Such oligonucleotides which specifically hybridize to a portion of the sense strand of a gene are commonly described as “antisense.” Antisense oligonucleotides are commonly used as research reagents, diagnostic aids, and therapeutic agents. For example, antisense oligonucleotides, which are able to inhibit gene expression with exquisite specificity, are often used by those of ordinary skill to elucidate the function of particular genes, for example to distinguish between the functions of various members of a biological pathway. This specific inhibitory effect has, therefore, been harnessed by those skilled in the art for research uses.

“Hybridization”, in the context of this invention, means hydrogen bonding, which may be Watson-Crick, Hoogsteen or reversed Hoogsteen hydrogen bonding, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” and “complementary” are terms which are used to indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of activity, and there is a sufficient degree of complementarity to avoid non-specific binding of the oligonucleotide to non-target nucleic acid sequences under conditions in which specific binding is desired, i.e., under physiological conditions in the case of in vivo assays or therapeutic treatment or, in the case of in vitro assays, under conditions in which the assays are conducted.

It is understood in the art that the sequence of the oligomeric compound need not be 100% complementary to that of its target nucleic acid to be specifically hybridizable. Moreover, an oligomeric compound may hybridize over one or more segments such that intervening or adjacent segments are not involved in the hybridization event (e.g., a loop structure or hairpin structure). It is preferred that the oligomeric compounds of the present invention comprise at least 70% sequence complementarity to a target region within the target nucleic acid, more preferably that they comprise 90% sequence complementarity and even more preferably comprise 95% sequence complementarity to the target region within the target nucleic acid sequence to which they are targeted. For example, an oligomeric compound in which 18 of 20 nucleobases of the oligomeric compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

In the present invention the phrase “stringent hybridization conditions” or “stringent conditions” refers to conditions under which an oligomeric compound of the invention will hybridize to its target sequence, but to a minimal number of other sequences. Stringent conditions are sequence-dependent-and will vary with different circumstances and in the context of this invention; “stringent conditions” under which oligomeric compounds hybridize to a target sequence are determined by the nature and composition of the oligomeric compounds and the assays in which they are being investigated.

The specificity and sensitivity of oligonucleotides is also harnessed by those of skill in the art for therapeutic uses. For example, the following U.S. patents demonstrate palliative, therapeutic and other methods utilizing antisense oligonucleotides. U.S. Pat. No. 5,135,917 provides antisense oligonucleotides that inhibit human interleukin-1 receptor expression. U.S. Pat. No. 5,098,890 is directed to antisense oligonucleotides complementary to the c-myb oncogene and antisense oligonucleotide therapies for certain cancerous conditions. U.S. Pat. No. 5,087,617 provides methods for treating cancer patients with antisense oligonucleotides. U.S. Pat. No. 5,166,195 provides oligonucleotide inhibitors of HIV. U.S. Pat. No. 5,004,810 provides oligomers capable of hybridizing to herpes simplex virus Vmw65 mRNA and inhibiting replication. U.S. Pat. No. 5,194,428 provides antisense oligonucleotides having antiviral activity against influenza virus. U.S. Pat. No. 4,806,463 provides antisense oligonucleotides and methods using them to inhibit HTLV-III replication. U.S. Pat. No. 5,286,717 provides oligonucleotides having a complementary base sequence to a portion of an oncogene. U.S. Pat. No. 5,276,019 and U.S. Pat. No. 5,264,423 are directed to phosphorothioate oligonucleotide analogs used to prevent replication of foreign nucleic acids in cells. U.S. Pat. No. 4,689,320 is directed to antisense oligonucleotides as antiviral agents specific to CMV. U.S. Pat. No. 5,098,890 provides oligonucleotides complementary to at least a portion of the mRNA transcript of the human c-myb gene. U.S. Pat. No. 5,242,906 provides antisense oligonucleotides useful in the treatment of latent EBV infections.

Oligonucleotides capable of modulating the expression of B7 proteins represent a novel therapeutic class of anti-inflammatory agents with activity towards a variety of inflammatory or autoimmune diseases, or disorders or diseases with an inflammatory component such as asthma, juvenile diabetes mellitus, myasthenia gravis, Graves' disease, rheumatoid arthritis, allograft rejection, inflammatory bowel disease, multiple sclerosis, psoriasis, lupus erythematosus, systemic lupus erythematosus, diabetes, multiple sclerosis, contact dermatitis, eczema, atopic dermatitis, seborrheic dermatitis, nummular dermatitis, generalized exfoliative dermatitis, rhinitis and various allergies. In addition, oligonucleotides capable of modulating the expression of B7 proteins provide a novel means of manipulating the ex vivo proliferation of T cells.

It is preferred to target specific genes for antisense attack. “Targeting” an oligonucleotide to the associated nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a foreign nucleic acid from an infectious agent. In the present invention, the target is a cellular gene associated with several immune system disorders and diseases (such as inflammation and autoimmune diseases), as well as with ostensibly “normal” immune reactions (such as a host animal's rejection of transplanted tissue), for which modulation is desired in certain instances. The targeting process also includes determination of a region (or regions) within this gene for the oligonucleotide interaction to occur such that the desired effect, either detection or modulation of expression of the protein, will result. Once the target region have been identified, oligonucleotides are chosen which are sufficiently complementary to the target, i.e., hybridize sufficiently well and with sufficient specificity to give the desired effect.

Generally, there are five regions of a gene that may be targeted for antisense modulation: the 5′ untranslated region (hereinafter, the “5′-UTR”), the translation initiation codon region (hereinafter, the “tIR”), the open reading frame (hereinafter, the “ORF”), the translation termination codon region (hereinafter, the “tTR”) and the 3′ untranslated region (hereinafter, the “3“-UTR”). As is known in the art, these regions are arranged in a typical messenger RNA molecule in the following order (left to right, 5′ to 3′): 5′-UTR, tIR, ORF, tTR, 3′-UTR. As is known in the art, although some eukaryotic transcripts are directly translated, many ORFs contain one or more sequences, known as “introns,” which are excised from a transcript before it is translated; the expressed (unexcised) portions of the ORF are referred to as “exons” (Alberts et al., Molecular Biology of the Cell, 1983, Garland Publishing Inc., New York, pp. 411-415). Furthermore, because many eukaryotic ORFs are a thousand nucleotides or more in length, it is often convenient to subdivide the ORF into, e.g., the 5′ ORF region, the central ORF region, and the 3′ ORF region. In some instances, an ORF contains one or more sites that may be targeted due to some functional significance in vivo. Examples of the latter types of sites include intragenic stem-loop structures (see, e.g., U.S. Pat. No. 5,512,438) and, in unprocessed mRNA molecules, intron/exon splice sites. Within the context of the present invention, one preferred intragenic site is the region encompassing the translation initiation codon of the open reading frame (ORF) of the gene. Because, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Furthermore, 5′-UUU functions as a translation initiation codon in vitro (Brigstock et al., Growth Factors, 1990, 4, 45; Gelbert et al., Somat. Cell. Mol. Genet., 1990, 16, 173; Gold and Stormo, in: Escherichia coli and Salmonella typhimurium: Cellular and Molecular Biology, Vol. 2, 1987, Neidhardt et al., eds., American Society for Microbiology, Washington, D.C., p. 1303). Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions, in order to generate related polypeptides having different amino terminal sequences (Markussen et al., Development, 1995, 121, 3723; Gao et al., Cancer Res., 1995, 55, 743; McDermott et al., Gene, 1992, 117, 193; Perri et al., J. Biol. Chem., 1991, 266, 12536; French et al., J. Virol., 1989, 63, 3270; Pushpa-Rekha et al., J. Biol. Chem., 1995, 270, 26993; Monaco et al., J. Biol. Chem., 1994, 269, 347; DeVirgilio et al., Yeast, 1992, 8, 1043; Kanagasundaram et al., Biochim. Biophys. Acta, 1992, 1171, 198; Olsen et al., Mol. Endocrinol., 1991, 5, 1246; Saul et al., Appl. Environ. Microbiol., 1990, 56, 3117; Yaoita et al., Proc. Natl. Acad. Sci. USA, 1990, 87, 7090; Rogers et al., EMBO J., 1990, 9, 2273). In the context of the invention, “start codon” and “translation initiation codon” refer to the codon or codons that are used in vivo to initiate translation of an mRNA molecule transcribed from a gene encoding a B7 protein, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination region” refer to a portion of such an mRNA or gene that encompasses from about 25 to about 50 contiguous nucleotides in either direction (i.e., 5′ or 3′) from a translation termination codon.

In the context of this invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620).

Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

Oligomer and Monomer Modifications

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside linkage or in conjunction with the sugar ring the backbone of the oligonucleotide. The normal internucleoside linkage that makes up the backbone of RNA and DNA is a 3′ to 5′ phosphodiester linkage.

Modified Internucleoside Linkages

Specific examples of preferred antisense oligomeric compounds useful in this invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

In the C. elegans system, modification of the internucleotide linkage (phosphorothioate) did not significantly interfere with RNAi activity. Based on this observation, it is suggested that certain preferred oligomeric compounds of the invention can also have one or more modified internucleoside linkages. A preferred phosphorus containing modified internucleoside linkage is the phosphorothioate internucleoside linkage.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In more preferred embodiments of the invention, oligomeric compounds have one or more phosphorothioate and/or heteroatom internucleoside linkages, in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃) —N(CH₃) —CH₂— and —O—N(CH₃)—CH₂—CH₂— [wherein the native phosphodiester internucleotide linkage is represented as —O—P(═O)(OH)—O—CH₂—]. The MMI type internucleoside linkages are disclosed in the above referenced U.S. Pat. No. 5,489,677. Preferred amide internucleoside linkages are disclosed in the above referenced U.S. Pat. No. 5,602,240.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Oligomer Mimetics

Another preferred group of oligomeric compounds amenable to the present invention includes oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid. One such oligomeric compound, an oligonucleotide mimetic that has been shown to have excellent hybridization properties, is referred to as a peptide nucleic acid (PNA). In PNA oligomeric compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United. States patents that teach the preparation of PNA oligomeric compounds include, but are not limited to, U.S. Pat. Nos. 5,539,082; 5,714,331; and 5,719,262, each of which is herein incorporated by reference. Further teaching of PNA oligomeric compounds can be found in Nielsen et al., Science, 1991, 254, 1497-1500.

One oligonucleotide mimetic that has been reported to have excellent hybridization properties is peptide nucleic acids (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

PNA has been modified to incorporate numerous modifications since the basic PNA structure was first prepared. The basic structure is shown below:

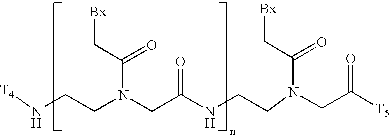

wherein

Bx is a heterocyclic base moiety;

T₄ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₅ is —OH, —N(Z₁)Z₂, R₅, D or L α-amino acid linked via the α-amino group or optionally through the β-amino group when the amino acid is lysine or ornithine or a peptide derived from D, L or mixed D and L amino acids linked through an amino group, a chemical functional group, a reporter group or a conjugate group;

Z₁ is hydrogen, C₁-C₆ alkyl, or an amino protecting group;

Z₂ is hydrogen, C₁-C₆ alkyl, an amino protecting group, —C(═O)—(CH₂)ₙ-J-Z₃, a D or L α-amino acid linked via the α-carboxyl group or optionally through the o-carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, —C(═O)—CH₃, benzyl, benzoyl, or —(CH₂)ₙ—N(H)Z₁;

each J is O, S or NH;

R₅ is a carbonyl protecting group; and

n is from 2 to about 50.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. A preferred class of linking groups have been selected to give a non-ionic oligomeric compound. The non-ionic morpholino-based oligomeric compounds are less likely to have undesired interactions with cellular proteins. Morpholino-based oligomeric compounds are non-ionic mimics of oligonucleotides which are less likely to form undesired interactions with cellular proteins (Dwaine A. Braasch and David R. Corey, Biochemistry, 2002, 41(14), 4503-4510). Morpholino-based oligomeric compounds are disclosed in U.S. Pat. No. 5,034,506, issued Jul. 23, 1991. The morpholino class of oligomeric compounds have been prepared having a variety of different linking groups joining the monomeric subunits.

Morpholino nucleic acids have been prepared having a variety of different linking groups (L₂) joining the monomeric subunits. The basic formula is shown below:

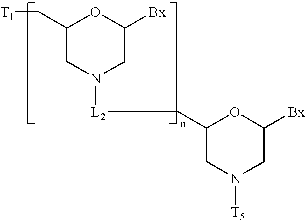

wherein

T₁ is hydroxyl or a protected hydroxyl;

T₅ is hydrogen or a phosphate or phosphate derivative;

L₂ is a linking group; and

n is from 2 to about 50.

A further class of oligonucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. Coli RNase resulting in cleavage of the target RNA strand.

The general formula of CeNA is shown below:

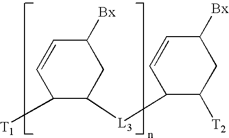

wherein

each Bx is a heterocyclic base moiety;

T₁ is hydroxyl or a protected hydroxyl; and

T₂ is hydroxyl or a protected hydroxyl.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566) and would have the general formula:

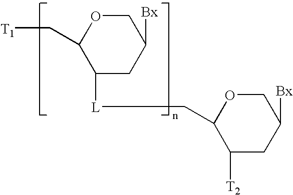

A further preferred modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage is preferably a methylene (—CH₂—)ₙ group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10 C), stability towards 3′-exonucleolytic degradation and good solubility properties. The basic structure of LNA showing the bicyclic ring system is shown below:

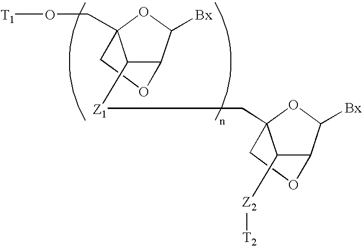

The conformations of LNAs determined by 2D NMR spectroscopy have shown that the locked orientation of the LNA nucleotides, both in single-stranded LNA and in duplexes, constrains the phosphate backbone in such a way as to introduce a higher population of the N-type conformation (Petersen et al., J. Mol. Recognit., 2000, 13, 44-53). These conformations are associated with improved stacking of the nucleobases (Wengel et al., Nucleosides Nucleotides, 1999, 18, 1365-1370).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands.

Novel types of LNA-oligomeric compounds, as well as the LNAs, are useful in a wide range of diagnostic and therapeutic applications. Among these are antisense applications, PCR applications, strand-displacement oligomers, substrates for nucleic acid polymerases and generally as nucleotide based drugs. Potent and nontoxic antisense oligonucleotides containing LNAs have been described (Wahlestedt et al., Proc. Natl. Acad. Sci. U.S.A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

The first analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-Amino- and 2‘-methylamino-LNA’s have been prepared and the thermal stability of their duplexes with complementary RNA and DNA strands has been previously reported.

Further oligonucleotide mimetics have been prepared to include bicyclic and tricyclic nucleoside analogs having the formulas (amidite monomers shown):

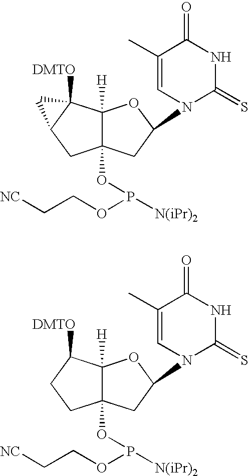

(see Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids incorporate a phosphorus group in a backbone the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

The general formula (for definitions of Markush variables see: U.S. Pat. Nos. 5,874,553 and 6,127,346 herein incorporated by reference in their entirety) is shown below.

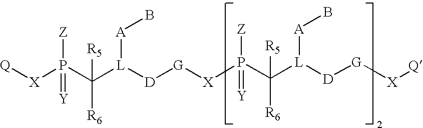

Another oligonucleotide mimetic has been reported wherein the furanosyl ring has been replaced by a cyclobutyl moiety.

Modified Sugars

Oligomeric compounds of the invention may also contain one or more substituted sugar moieties. Preferred oligomeric compounds comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃] ₂, where n and m are from 1 to about 10. Other preferred oligonucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃) 2

Other preferred sugar substituent groups include methoxy (—O—CH₃), aminopropoxy (—OCH₂CH₂CH₂NH₂), allyl (—CH₂—CH═CH₂), —O-allyl (—O—CH₂—CH═CH₂) and fluoro (F). 2′-Sugar substituent groups may be in the arabino (up) position or ribo (down) position. A preferred 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Further representative sugar substituent groups include groups of formula Iₐ or IIₐ:

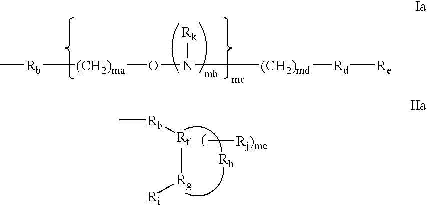

wherein:

R_{b} is O, S or NH;

R_{d} is a single bond, O, S or C(═O);

Rₑ is C₁-C₁₀ alkyl, N(Rₖ)(Rₘ), N(Rₖ)(Rₙ), N═C (Rₚ) (R_{q}), N═C(Rₚ)(Rᵣ) or has formula IIIₐ;

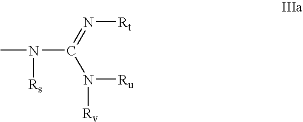

Rₚ and R_{q} are each independently hydrogen or C₁-C₁₀ alkyl;

Rᵣ is —Rₓ—R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, a nitrogen protecting group or —Rₓ—R_{y};

Rₚ is hydrogen, a nitrogen protecting group or —Rₓ—R_{y};

Rₓ is a bond or a linking moiety;

R_{y} is a chemical functional group, a conjugate group or a solid support medium;

each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ) (Rᵥ), guanidino and acyl where said acyl is an acid amide or an ester;

or Rₘ and Rₙ, together, are a nitrogen protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;

mₐ is 1 to about 10;

each m_{b} is, independently, 0 or 1;

m_{c} is 0 or an integer from 1 to 10;

m_{d} is an integer from 1 to 10;

mₑ is from 0, 1 or 2; and

provided that when m_{c} is 0, m_{d} is greater than 1.

Representative substituents groups of Formula I are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic substituent groups of Formula II are disclosed in U.S. patent application Ser. No. 09/123,108, filed Jul. 27, 1998, entitled “RNA Targeted 2′-Oligomeric compounds that are Conformationally Preorganized,” hereby incorporated by reference in its entirety.

Particularly preferred sugar substituent groups include O[(CH₂)ₙO]ₘCH₃, O(CH₂)ₙOCH₃, O(CH₂)ₙNH₂, (CH₂)ₙCH₃, O(CH₂)ₙONH₂, and O(CH₂)ₙON[(CH₂)ₙCH₃)]₂, where n and m are from 1 to about 10.

Representative guanidino substituent groups that are shown in formula III and IV are disclosed in co-owned U.S. patent application Ser. No. 09/349,040, entitled “Functionalized Oligomers”, filed Jul. 7, 1999, hereby incorporated by reference in its entirety.

Representative acetamido substituent groups are disclosed in U.S. Pat. No. 6,147,200 which is hereby incorporated by reference in its entirety.

Representative dimethylaminoethyloxyethyl substituent groups are disclosed in International Patent Application PCT/US99/17895, entitled “2′-O-Dimethylaminoethyloxyethyl-Oligomeric compounds”, filed Aug. 6, 1999, hereby incorporated by reference in its entirety.

Modified Nucleobases/Naturally Occurring Nucleobases

Oligomeric compounds may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine-(5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research: and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

In one aspect of the present invention oligomeric compounds are prepared having polycyclic heterocyclic compounds in place of one or more heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

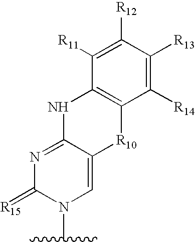

Representative cytosine analogs that make 3 hydrogen bonds with a guanosine in a second strand include 1,3-diazaphenoxazine-2-one (R₁₀₌O, R₁₁-R₁₄=H) [Kurchavov, et al., Nucleosides and Nucleotides, 1997, 16, 1837-1846], 1,3-diazaphenothiazine-2-one (R₁₀=S, R₁₁-R₁₄=H), [Lin, K.-Y.; Jones, R. J.; Matteucci, M. J. Am. Chem. Soc. 1995, 117, 3873-3874] and 6,7,8,9-tetrafluoro-1,3-diazaphenoxazine-2-one (R₁₀=O, R₁₁-R₁₄=F) [Wang, J.; Lin, K.-Y., Matteucci, M. Tetrahedron Lett. 1998, 39, 8385-8388]. Incorporated into oligonucleotides these base modifications were shown to hybridize with complementary guanine and the latter was also shown to hybridize with adenine and to enhance helical thermal stability by extended stacking interactions (also see U.S. Patent Application entitled “Modified Peptide Nucleic Acids” filed May 24, 2002, Ser. No. 10/155,920; and U.S. Patent Application entitled “Nuclease Resistant Chimeric Oligonucleotides” filed May 24, 2002, Ser. No. 10/013,295, both of which are commonly owned with this application and are herein incorporated by reference in their entirety).

Further helix-stabilizing properties have been observed when a cytosine analog/substitute has an aminoethoxy moiety attached to the rigid 1,3-diazaphenoxazine-2-one scaffold (R₁₀₌O, R₁₁=—O—(CH₂)₂—NH₂, R₁₂₋₁₄=H) [Lin, K.-Y., Matteucci, M. J. Am. Chem. Soc. 1998, 120, 8531-8532]. Binding studies demonstrated that a single incorporation could enhance the binding affinity of a model oligonucleotide to its complementary target DNA or RNA with a ΔTₘ of up to 180 relative to 5-methyl cytosine (dC5^{me}), which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. No. 6,028,183, which issued on May 22, 2000, and U.S. Pat. No. 6,007,992, which issued on Dec. 28, 1999, the contents of both are commonly assigned with this application and are incorporated herein in their entirety.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity [Lin, K-Y; Matteucci, M. J. Am. Chem. Soc. 1998, 120, 8531-8532]. The activity enhancement was even more pronounced in case of G-clamp, as a single substitution was shown to significantly improve the in vitro potency of a 20mer 2′-deoxyphosphorothioate oligonucleotides [Flanagan, W.-M.; Wolf, J. J.; Olson, P.; Grant, D.; Lin, K.-Y.; Wagner, R. W.; Matteucci, M. Proc. Natl. Acad. Sci. USA, 1999, 96, 3513-3518]. Nevertheless, to optimize oligonucleotide design and to better understand the impact of these heterocyclic modifications on the biological activity, it is important to evaluate their effect on the nuclease stability of the oligomers.

Further modified polycyclic heterocyclic compounds useful as heterocyclcic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. patent application Ser. No. 09/996,292 filed Nov. 28, 2001, certain of which are commonly owned with the instant application, and each of which is herein incorporated by reference.

The oligonucleotides of the present invention also include variants in which a different base is present at one or more of the nucleotide positions in the oligonucleotide. For example, if the first nucleotide is an adenosine, variants may be produced which contain thymidine, guanosine or cytidine at this position. This may be done at any of the positions of the oligonucleotide. Thus, a 20-mer may comprise 60 variations (20 positions×3 alternates at each position) in which the original nucleotide is substituted with any of the three alternate nucleotides. These oligonucleotides are then tested using the methods described herein to determine their ability to inhibit expression of HCV mRNA and/or HCV replication.

Conjugates

A further preferred substitution that can be appended to the oligomeric compounds of the invention involves the linkage of one or more moieties or conjugates which enhance the activity, cellular distribution or cellular uptake of the resulting oligomeric compounds. In one embodiment such modified oligomeric compounds are prepared by covalently attaching conjugate groups to functional groups such as hydroxyl or amino groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-o-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

The oligomeric compounds of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Chimeric Oligomeric Compounds

It is not necessary for all positions in an oligomeric compound to be uniformly modified, and in fact more than one of the aforementioned modifications may be incorporated in a single oligomeric compound or even at a single monomeric subunit such as a nucleoside within a oligomeric compound. The present invention also includes oligomeric compounds which are chimeric oligomeric compounds. “Chimeric” oligomeric compounds or “chimeras,” in the context of this invention, are oligomeric compounds that contain two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, oligonucleotide analogs, oligonucleosides and/or oligonucleotide mimetics as described above. Such oligomeric compounds have also been referred to in the art as hybrids hemimers, gapmers or inverted gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

3′-Endo Modifications

In one aspect of the present invention oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. There is an apparent preference for an RNA type duplex (A form helix, predominantly 3′-endo) as a requirement (e.g. trigger) of RNA interference which is supported in part by the fact that duplexes composed of 2α-deoxy-2′-F-nucleosides appears efficient in triggering RNAi response in the C. elegans system. Properties that are enhanced by using more stable 3′-endo nucleosides include but aren't limited to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. The present invention provides oligomeric triggers of RNAi having one or more nucleosides modified in such a way as to favor a C3′-endo type conformation.

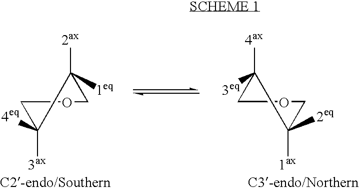

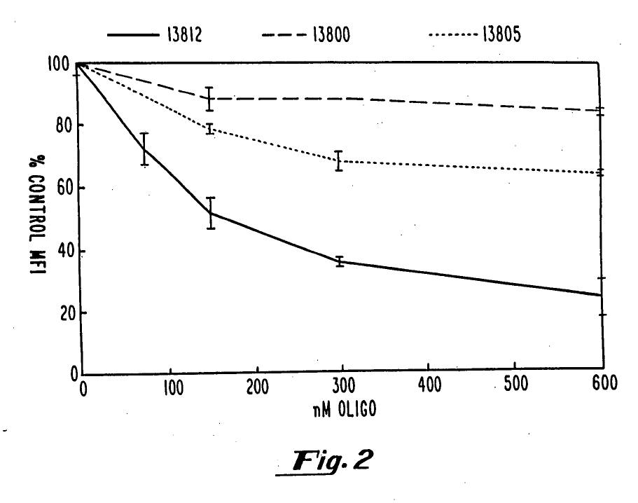

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′′-endo conformation can be achieved while maintaining the 2′-OH as a recognition element, as illustrated in FIG. 2, below (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2° F.-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, oligomeric triggers of RNAi response might be composed of one or more nucleosides modified in such a way that conformation is locked into a C3′-endo type conformation, i.e. Locked Nucleic Acid (LNA, Singh et al, Chem. Commun. (1998), 4, 455-456), and ethylene bridged Nucleic Acids (ENA, Morita et al, Bioorganic & Medicinal Chemistry Letters (2002), 12, 73-76.) Examples of modified nucleosides amenable to the present invention are shown below in Table I. These examples are meant to be representative and not exhaustive.

TABLE I

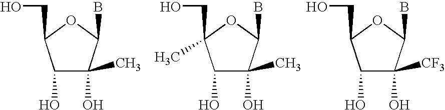

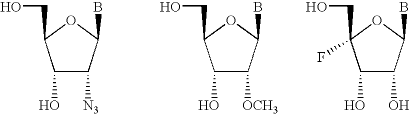

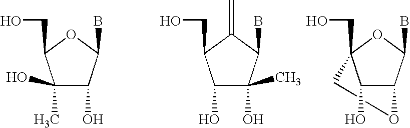

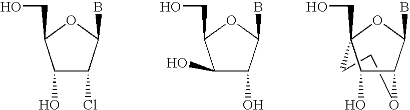

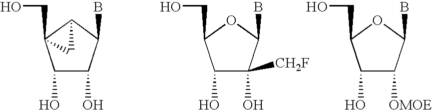

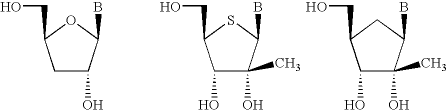

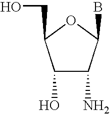

The preferred conformation of modified nucleosides and their oligomers can be estimated by various methods such as molecular dynamics calculations, nuclear magnetic resonance spectroscopy and CD measurements. Hence, modifications predicted to induce RNA like conformations, A-form duplex geometry in an oligomeric context, are selected for use in the modified oligoncleotides of the present invention. The synthesis of numerous of the modified nucleosides amenable to the present invention are known in the art (see for example, Chemistry of Nucleosides and Nucleotides Vol 1-3, ed. Leroy B. Townsend, 1988, Plenum press., and the examples section below.) Nucleosides known to be inhibitors/substrates for-RNA dependent RNA polymerases (for example HCV NS5B).

In one aspect, the present invention is directed to oligonucleotides that are prepared having enhanced properties compared to native RNA against nucleic acid targets. A target is identified and an oligonucleotide is selected having an effective length and sequence that is complementary to a portion of the target sequence. Each nucleoside of the selected sequence is scrutinized for possible enhancing modifications. A preferred modification would be the replacement of one or more RNA nucleosides with nucleosides that have the same 3′-endo conformational geometry. Such modifications can enhance chemical and nuclease stability relative to native RNA while at the same time being much cheaper and easier to synthesize and/or incorporate into an oligonulceotide. The selected sequence can be further divided into regions and the nucleosides of each region evaluated for enhancing modifications that can be the result of a chimeric configuration. Consideration is also given to the 5′ and 3′-termini as there are often advantageous modifications that can be made to one or more of the terminal nucleosides. The oligomeric compounds of the present invention include at least one 5′-modified phosphate group on a single strand or on at least one 5′-position of a double stranded sequence or sequences. Further modifications are also considered such as internucleoside linkages, conjugate groups, substitute sugars or bases, substitution of one or more nucleosides with nucleoside mimetics and any other modification that can enhance the selected sequence for its intended target. The terms used to describe the conformational geometry of homoduplex nucleic acids are “A Form” for RNA and “B Form” for DNA. The respective conformational geometry for RNA and DNA duplexes was determined from X-ray diffraction analysis of nucleic acid fibers (Arnott and Hukins, Biochem. Biophys. Res. Comm., 1970, 47, 1504.) In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). As used herein, B-form geometry is inclusive of both C2′-endo pucker and 04′-endo pucker. This is consistent with Berger, et. al., Nucleic Acids Research, 1998, 26, 2473-2480, who pointed out that in considering the furanose conformations which give rise to B-form duplexes consideration should also be given to a 04′-endo pucker contribution.

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 21)5, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as but not limited to antisense and RNA interference as these mechanisms require the binding of a synthetic oligonucleotide strand to an RNA target strand. In the case of antisense, effective inhibition of the mRNA requires that the antisense DNA have a very high binding affinity with the mRNA. Otherwise the desired interaction between the synthetic oligonucleotide strand and target mRNA strand will occur infrequently, resulting in decreased efficacy.

One routinely used method of modifying the sugar puckering is the substitution of the sugar at the 2′-position with a substituent group that influences the sugar geometry. The influence on ring conformation is dependant on the nature of the substituent at the 2′-position. A number of different substituents have been studied to determine their sugar puckering effect. For example, 2′-halogens have been studied showing that the 2′-fluoro derivative exhibits the largest population (65%) of the C3′-endo form, and the 2′-iodo exhibits the lowest population (7%). The populations of adenosine (2′-OH) versus deoxyadenosine (2′-H) are 36% and 19%, respectively. Furthermore, the effect of the 2′-fluoro group of adenosine dimers (2′-deoxy-2′-fluoroadenosine-2′-deoxy-2′-fluoro-adenosine) is further correlated to the stabilization of the stacked conformation.

As expected, the relative duplex stability can be enhanced by replacement of 2′-OH groups with 2′-F groups thereby increasing the C3′-endo population. It is assumed that the highly polar nature of the 2′-F bond and the extreme preference for C3′-endo puckering may stabilize the stacked conformation in an A-form duplex. Data from UV hypochromicity, circular dichroism, and ¹H NMR also indicate that the degree of stacking decreases as the electronegativity of the halo substituent decreases. Furthermore, steric bulk at the 2′-position of the sugar moiety is better accommodated in an A-form duplex than a B-form duplex. Thus, a 2′-substituent on the 3′-terminus of a dinucleoside monophosphate is thought to exert a number of effects on the stacking conformation steric repulsion, furanose puckering preference, electrostatic repulsion, hydrophobic attraction, and hydrogen bonding capabilities. These substituent effects are thought to be determined by the molecular size, electronegativity, and hydrophobicity of the substituent. Melting temperatures of complementary strands is also increased with the 2′-substituted adenosine diphosphates. It is not clear whether the 3′-endo preference of the conformation or the presence of the substituent is responsible for the increased binding. However, greater overlap of adjacent bases (stacking) can be achieved with the 3′-endo conformation.

One synthetic 2′-modification that imparts increased nuclease resistance and a very high binding affinity to nucleotides is the 2-methoxyethoxy (2′-MOE, 2′-OCH₂CH₂OCH₃) side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-O-methoxyethyl substituent also have been shown to be antisense inhibitors of gene expression with promising features for in vivo use (Martin, P., Helv. Chim. Acta, 1995, 78, 486-504; Altmann et al., Chimia, 1996, 50, 168-7176; Altmann et al., Biochem. Soc. Trans., 1996, 24, 630-637; and Altmann et al., Nucleosides Nucleotides, 1997, 16, 917-926). Relative to DNA, the oligonucleotides having the 2′-MOE modification displayed improved RNA affinity and higher nuclease resistance. Chimeric oligonucleotides having 2′-MOE substituents in the wing nucleosides and an internal region of deoxy-phosphorothioate nucleotides (also termed a gapped oligonucleotide or gapmer) have shown effective reduction in the growth of tumors in animal models at low doses. 2′-MOE substituted oligonucleotides have also shown outstanding promise as antisense agents in several disease states. One such MOE substituted oligonucleotide is presently being investigated in clinical trials for the treatment of CMV retinitis.

Chemistries Defined

Unless otherwise defined herein, alkyl means C₁-C₁₂, preferably C₁-C₈, and more preferably C₁-C₆, straight or (where possible) branched chain aliphatic hydrocarbyl.

Unless otherwise defined herein, heteroalkyl means C₁-C₁₂, preferably C₁-C₈, and more preferably C₁-C₆, straight or (where possible) branched chain aliphatic hydrocarbyl containing at least one, and preferably about 1 to about 3, hetero atoms in the chain, including the terminal portion of the chain. Preferred heteroatoms include N, O and S.

Unless otherwise defined herein, cycloalkyl means C₃-C₁₂, preferably C₃-C₈, and more preferably C₃-C₆, aliphatic hydrocarbyl ring.

Unless otherwise defined herein, alkenyl means C₂-C₁₂, preferably C₂-C₈, and more preferably C₂-C₆ alkenyl, which may be straight or (where possible) branched hydrocarbyl moiety, which contains at least one carbon-carbon double bond.

Unless otherwise defined herein, alkynyl means C₂-C₁₂, preferably C₂-C₈, and more preferably C₂-C₆ alkynyl, which may be straight or (where possible) branched hydrocarbyl moiety, which contains at least one carbon-carbon triple bond.

Unless otherwise defined herein, heterocycloalkyl means a ring moiety containing at least three ring members, at least one of which is carbon, and of which 1, 2 or three ring members are other than carbon. Preferably the number of carbon atoms varies from 1 to about 12, preferably 1 to about 6, and the total number of ring members varies from three to about 15, preferably from about 3 to about 8. Preferred ring heteroatoms are N, O and S. Preferred heterocycloalkyl groups include morpholino, thiomorpholino, piperidinyl, piperazinyl, homopiperidinyl, homopiperazinyl, homomorpholino, homothiomorpholino, pyrrolodinyl, tetrahydrooxazolyl, tetrahydroimidazolyl, tetrahydrothiazolyl, tetrahydroisoxazolyl, tetrahydropyrrazolyl, furanyl, pyranyl, and tetrahydroisothiazolyl.

Unless otherwise defined herein, aryl means any hydrocarbon ring structure containing at least one aryl ring. Preferred aryl rings have about 6 to about 20 ring carbons. Especially preferred aryl rings include phenyl, napthyl, anthracenyl, and phenanthrenyl.

Unless otherwise defined herein, hetaryl means a ring moiety containing at least one fully unsaturated ring, the ring consisting of carbon and non-carbon atoms. Preferably the ring system contains about 1 to about 4 rings. Preferably the number of carbon atoms varies from 1 to about 12, preferably 1 to about 6, and the total number of ring members varies from three to about 15, preferably from about 3 to about 8. Preferred ring heteroatoms are N, O and S. Preferred hetaryl moieties include pyrazolyl, thiophenyl, pyridyl, imidazolyl, tetrazolyl, pyridyl, pyrimidinyl, purinyl, quinazolinyl, quinoxalinyl, benzimidazolyl, benzothiophenyl, etc.

Unless otherwise defined herein, where a moiety is defined as a compound moiety, such as hetarylalkyl (hetaryl and alkyl), aralkyl (aryl and alkyl), etc., each of the sub-moieties is as defined herein.

Unless otherwise defined herein, an electron withdrawing group is a group, such as the cyano or isocyanato group that draws electronic charge away from the carbon to which it is attached. Other electron withdrawing groups of note include those whose electronegativities exceed that of carbon, for example halogen, nitro, or phenyl substituted in the ortho- or para-position with one or more cyano, isothiocyanato, nitro or halo groups.

Unless otherwise defined herein, the terms halogen and halo have their ordinary meanings. Preferred halo (halogen) substituents are Cl, Br, and I.

The aforementioned optional substituents are, unless otherwise herein defined, suitable substituents depending upon desired properties. Included are halogens (Cl, Br, I), alkyl, alkenyl, and alkynyl moieties, NO₂, NH₃ (substituted and unsubstituted), acid moieties (e.g. —CO₂H, —OSO₃H₂, etc.), heterocycloalkyl moieties, hetaryl moieties, aryl moieties, etc.

In all the preceding formulae, the squiggle (˜) indicates a bond to an oxygen or sulfur of the 5′-phosphate.

Phosphate protecting groups include those described in U.S. Pat. No. US 5,760,209, US 5,614,621, US 6,051,699, US 6,020,475, US 6,326,478, US 6,169,177, US 6,121,437, US 6,465,628 each of which is expressly incorporated herein by reference in its entirety.

The oligonucleotides in accordance with this invention (single stranded or double stranded) preferably comprise from about 8 to about 80 nucleotides, more preferably from about 12-50 nucleotides and most preferably from about 15 to 30 nucleotides. As is known in the art, a nucleotide is a base-sugar combination suitably bound to an adjacent nucleotide through a phosphodiester, phosphorothioate or other covalent linkage.

The oligonucleotides of the present invention also include variants in which a different base is present at one or more of the nucleotide positions in the oligonucleotide. For example, if the first nucleotide is an adenosine, variants may be produced which contain thymidine, guanosine or cytidine at this position. This may be done at any of the positions of the oligonucleotide. Thus, a 20-mer may comprise 60 variations (20 positions×3 alternates at each position) in which the original nucleotide is substituted with any of the three alternate nucleotides. These oligonucleotides are then tested using the methods described herein to determine their ability to inhibit expression of B7.1 or B7.2 mRNA.

The oligonucleotides used in accordance with this invention may be conveniently and routinely made through the well-known technique of solid phase synthesis. Equipment for such synthesis is sold by-several vendors including, for example, Applied Biosystems (Foster City, Calif.). Any other means for such synthesis known in the art may additionally or alternatively be employed. It is also known to use similar techniques to prepare other oligonucleotides such as the phosphorothioates and alkylated derivatives.

The oligonucleotides of the present invention can be utilized as therapeutic compounds, diagnostic tools and as research reagents and kits. The term “therapeutic uses” is intended to encompass prophylactic, palliative and curative uses wherein the oligonucleotides of the invention are contacted with animal cells either in vivo or ex vivo. When contacted with animal cells ex vivo, a therapeutic use includes incorporating such cells into an animal after treatment with one or more oligonucleotides of the invention. While not intending to be bound to a particular utility, the ex vivo modulation of, e.g., T cell proliferation by the oligonucleotides of the invention can be employed in, for example, potential therapeutic modalities wherein it is desired to modulate the expression of a B7 protein in APCs.

As an example, oligonucleotides that inhibit the expression of B7-1 proteins are expected to enhance the availability of B7-2 proteins on the surface of APCs, thus increasing the costimulatory effect of B7-2 on T cells ex vivo (Levine et al., Science, 1996, 272, 1939).

For therapeutic uses, an animal suspected of having a disease or disorder which can be treated or prevented by modulating the expression or activity of a B7 protein is, for example, treated by administering oligonucleotides in accordance with this invention. The oligonucleotides of the invention can be utilized in pharmaceutical compositions by adding an effective amount of an oligonucleotide to a suitable pharmaceutically acceptable diluent or carrier. Workers in the field have identified antisense, triplex and other oligonucleotide compositions which are capable of modulating expression of genes implicated in viral, fungal and metabolic diseases. Antisense oligonucleotides have been safely administered to humans and several clinical trials are presently underway. It is thus established that oligonucleotides can be useful therapeutic instrumentalities that can be configured to be useful in treatment regimes for treatment of cells, tissues and animals, especially humans.

The oligonucleotides of the present invention can be further used to detect the presence of B7-specific nucleic acids in a cell or tissue sample. For example, radiolabeled oligonucleotides can be prepared by ³²P labeling at the 5′ end with polynucleotide kinase (Sambrook et al., Molecular Cloning. A Laboratory Manual, Cold Spring Harbor Laboratory Press, 1989, Volume 2, pg. 10.59). Radiolabeled oligonucleotides are then contacted with cell or tissue samples suspected of containing B7 message RNAs (and thus B7 proteins), and the samples are washed to remove unbound oligonucleotide. Radioactivity remaining in the sample indicates the presence of bound oligonucleotide, which in turn indicates the presence of nucleic acids complementary to the oligonucleotide, and can be quantitated using a scintillation counter or other routine means. Expression of nucleic acids encoding these proteins is thus detected.

Radiolabeled oligonucleotides of the present invention can also be used to perform autoradiography of tissues to determine the localization, distribution and quantitation of B7 proteins for research, diagnostic or therapeutic purposes. In such studies, tissue sections are; treated with radiolabeled oligonucleotide and washed as described above, then exposed to photographic emulsion according to routine autoradiography procedures. The emulsion, when developed, yields an image of silver grains over the regions expressing a B7 gene. Quantitation of the silver grains permits detection of the expression of mRNA molecules encoding these proteins and permits targeting of oligonucleotides to these areas.

Analogous assays for fluorescent detection of expression of B7 nucleic acids can be developed using oligonucleotides of the present invention which are conjugated with fluorescein or other fluorescent tags instead of radiolabeling. Such conjugations are routinely accomplished during solid phase synthesis using fluorescently-labeled amidites or controlled pore glass (CPG) columns. Fluorescein-labeled amidites and CPG are available from, e.g., Glen Research, Sterling Va.

The present invention employs oligonucleotides targeted to nucleic acids encoding B7 proteins and oligonucleotides targeted to nucleic acids encoding such proteins. Kits for detecting the presence or absence of expression of a B7 protein may also be prepared. Such kits include an oligonucleotide targeted to an appropriate gene, i.e., a gene encoding a B7 protein. Appropriate kit and assay formats, such as, e.g., “sandwich” assays, are known in the art and can easily be adapted for use with the oligonucleotides of the invention. Hybridization of the oligonucleotides of the invention with a nucleic acid encoding a B7 protein can be detected by means known in the art. Such means may include conjugation of an enzyme to the oligonucleotide, radiolabelling of the oligonucleotide or any other suitable detection systems. Kits for detecting the presence or absence of a B7 protein may also be prepared.

In the context of this invention, “hybridization” means hydrogen bonding, which may be Watson-Crick, Hoogsteen or reversed Hoogsteen hydrogen bonding, between complementary nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that an oligonucleotide need not be 100% complementary to its target DNA sequence to be specifically hybridizable. An oligonucleotide is specifically hybridizable when binding of the oligonucleotide to the target DNA or RNA molecule interferes with the normal function of the target DNA or RNA to cause a decrease or loss of function, and there is a sufficient degree of complementarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed.

The formulation of therapeutic compositions and their subsequent administration is believed to be within the skill of those in the art. In general, for therapeutics, a patient in need of such therapy is administered an oligonucleotide in accordance with the invention, commonly in a pharmaceutically acceptable carrier, in doses ranging from 0.01 μg to 100 g per kg of body weight depending on the age of the patient and the severity of the disorder or disease state being treated. Further, the treatment regimen may last for a period of time which will vary depending upon the nature of the particular disease or disorder, its severity and the overall condition of the patient, and may extend from once daily to once every 20 years. Following treatment, the patient is monitored for changes in his/her condition and for alleviation of the symptoms of the disorder or disease state. The dosage of the oligonucleotide may either be increased in the event the patient does not respond significantly to current dosage levels, or the dose may be decreased if an alleviation of the symptoms of the disorder or disease state is observed, or if the disorder or disease state has been ablated.

In some cases, it may be more effective to treat a patient with an oligonucleotide of the invention in conjunction with other therapeutic modalities in order to increase the efficacy of a treatment regimen. In the context of the invention, the term “treatment regimen” is meant to encompass therapeutic, palliative and prophylactic modalities. In a preferred embodiment, the oligonucleotides of the invention are used in conjunction with an anti-inflammatory and/or immunosuppressive agent, preferably one or more antisense oligonucleotides targeted to an intercellular adhesion molecule (ICAM), preferably to ICAM-1. Other anti-inflammatory and/or immunosuppressive agents that may be used in combination with the oligonucleotides of the invention include, but are not limited to, soluble ICAM proteins (e.g., sICAM-1), antibody-toxin conjugates, prednisone, methylprednisolone, azathioprine, cyclophosphamide, cyclosporine, interferons, sympathomimetics, conventional antihistamines (histamine H₁ receptor antagonists, including, for example, brompheniramine maleate, chlorpheniramine maleate, dexchlorpheniramine maleate, tripolidine HCl, carbinoxamine maleate, clemastine fumarate, dimenhydrinate, diphenhydramine HCl, diphenylpyraline HCl, doxylamine succinate, tripelennamine citrate, tripelennamine HCl, cyclizine HCl, hydroxyzine HCl, meclizine HCl, methdilazine HCl, promethazine HCl, trimeprazine tartrate, azatadine maleate, cyproheptadine HCl, terfenadine, etc.), histamine H₂ receptor antagonists (e.g., ranitidine). See, generally, The Merck Manual of Diagnosis and Therapy, 15th Ed., Berkow et al., eds., 1987, Rahway, N.J., pages 302-336 and 2516-2522). When used with the compounds of the invention, such agents may be used individually, sequentially, or in combination with one or more other such agents.

In another preferred embodiment of the invention, an antisense oligonucleotide targeted to one B7 mRNA species (e.g., B7-1) is used in combination with an antisense oligonucleotide targeted to a second B7 mRNA species (e.g., B7-2) in order to inhibit the costimulatory effect of B7 molecules to a more extensive degree than can be achieved with either oligonucleotide used individually. In a related version of this embodiment, two or more oligonucleotides of the invention, each targeted to an alternatively spliced B7-1 or B7-2 mRNA, are combined with each other in order to inhibit expression of both forms of the alternatively spliced mRNAs. It is known in the art that, depending on the specificity of the modulating agent employed, inhibition of one form of an alternatively spliced mRNA may not result in a sufficient reduction of expression for a given condition to be manifest. Thus, such combinations may, in some instances, be desired to inhibit the expression of a particular B7 gene to an extent necessary to practice one of the methods of the invention. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years. In the case of in individual known or suspected of being prone to an autoimmune or inflammatory condition, prophylactic effects may be achieved by administration of preventative doses, ranging from 0.01 μg to 100 g per kg of body weight, once or more daily, to once every 20 years. In like fashion, an individual may be made less susceptible to an inflammatory condition that is expected to occur as a result of some medical treatment, e.g., graft versus host disease resulting from the transplantation of cells, tissue or an organ into the individual.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer or metered dose inhaler; intratracheal, intranasal, epidermal and transdermal, oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavoring agents, diluents, emulsifiers, dispersing aids or binders may be desirable. Compositions for oral administration also include pulsatile delivery compositions and bioadhesive composition as described in copending U.S. patent application Ser. No. 09/944,493, filed Aug. 22, 2001, and Ser. No. 09/935,316, filed Aug. 22, 2001, the entire disclosures of which are incorporated herein by reference.

Compositions for parenteral, intrathecal or intraventricular administration may include sterile aqueous, solutions which may also contain buffers, diluents and other suitable additives.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, and may be given once or more daily, weekly, monthly or yearly, or even once every 2 to 20 years.

The following examples illustrate the invention and are not intended to limit the same. Those skilled in the art will recognize, or be able to ascertain through routine experimentation, numerous equivalents to the specific substances and procedures described herein. Such equivalents are considered to be within the scope of the present invention.

The following examples are provided for illustrative purposes only and are not intended to limit the invention.

### EXAMPLES

### Example 1

### Synthesis of Nucleic Acids Oligonucleotides

Oligonucleotides were synthesized on an automated DNA synthesizer using standard phosphoramidite chemistry with oxidation using iodine. β-Cyanoethyldiisopropyl phosphoramidites were purchased from Applied Biosystems (Foster City, Calif.). For phosphorothioate oligonucleotides, the standard oxidation bottle was replaced by a 0.2 M solution of 3H-1,2-benzodithiole-3-one-1,1-dioxide in acetonitrile for the stepwise thiation of the phosphite linkages. The thiation cycle wait step was increased to 68 seconds and was followed by the capping step.

The 2′-fluoro phosphorothioate oligonucleotides of the invention were synthesized using 5′-dimethoxytrityl-3′-phosphoramidites and prepared as disclosed in U.S. patent application Ser. No. 463,358, filed Jan. 11, 1990, and Ser. No. 566,977, filed Aug. 13, 1990, which are assigned to the same assignee as the instant application and which are incorporated by reference herein. The 2′-fluoro oligonucleotides were prepared using phosphoramidite chemistry and a slight modification of the standard DNA synthesis protocol: deprotection was effected using methanolic ammonia at room temperature.

The 2′-methoxy (2′-O-methyl) oligonucleotides of the invention were synthesized using 2′-methoxy β-cyanoethyldiisopropyl-phosphoramidites (Chemgenes, Needham Mass.) and the standard cycle for unmodified oligonucleotides, except the wait step after pulse delivery of tetrazole and base is increased to 360 seconds. Other 2′-alkoxy oligonucleotides are synthesized by a modification of this method, using appropriate 2′-modified amidites such as those available from Glen Research, Inc., Sterling, Va. The 3′-base used to start the synthesis was a 2′-deoxyribonucleotide. The 2′-O-propyl oligonucleotides of the invention are prepared by a slight modification of this procedure.

The 2′ methoxyethoxy (2′-O—CH₂CH₂OCH₃) oligonucleotides of the invention were synthesized according to the method of Martin, Helv. Chim. Acta 1995, 78, 486. For ease of synthesis, the last nucleotide was a deoxynucleotide. All 2′-O—CH₂CH₂OCH₃ cytosines were 5-methyl cytosines, which were synthesized according to the following procedures.

Synthesis of 5-Methyl Cytosine Monomers: 2,2′-Anhydro[1-(β-D-arabinofuranosyl)-5-methyluridine]

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60° C. at 1 mm Hg for 24 h) to give a solid which was crushed to a light tan powder (57 g, 85% crude yield). The material was used as is for further reactions.

2′-O-Methoxyethyl-5-methyluridine

2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160° C. After heating for 48 hours at 155-160° C., the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃CN (600 mL) and evaporated. A silica gel column (3 kg) was packed in CH₂Cl₂/acetone/MeOH (20:5:3) containing 0.5% Et₃NH. The residue was dissolved in CH₂Cl₂ (250 mL) and adsorbed onto silica (150 g) prior to loading onto the column. The product was eluted with the packing solvent to give 160 g (63%) of product.

2′-O-Methoxyethyl-5′-O-dimethoxytrityl-5-methyluridine 2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH₃CN (200 mL). The residue was dissolved in CHCl₃ (1.5 L) and extracted with 2×500 mL of saturated NaHCO₃ and 2×500 mL of saturated NaCl. The organic phase was dried over Na₂SO₄, filtered and evaporated. 275 g of residue was obtained. The residue was purified on a 3.5 kg silica gel column, packed and eluted with EtOAc/Hexane/Acetone (5:5:1) containing 0.5% Et₃NH. The pure fractions were evaporated to give 164 g of product. Approximately 20 g additional was obtained from the impure fractions to give a total yield of 183 g (57%).

3′-O-Acetyl-2′-O-methoxyethyl-5′-O-dimethoxytrityl-5-methyluridine

2′-O-Methoxyethyl-5′-O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by tic by first quenching the tic sample with the addition of MeOH. Upon completion of the reaction, as judged by tic, MeOH (50 mL) was added and the mixture evaporated at 35° C. The residue was dissolved in CHCl₃ (800 mL) and extracted with 2×200 mL of saturated sodium bicarbonate and 2×200 mL of saturated NaCl. The water layers were back extracted with 200 mL of CHCl₃. The combined organics were dried with sodium sulfate and evaporated to give 122 g of residue (approx. 90% product). The residue was purified on a 3.5 kg silica gel column and eluted using EtOAc/Hexane (4:1Y. Pure product fractions were evaporated to yield 96 g (84%).

3 t-O-Acetyl-2′-O-methoxyethyl-5′-O-dimethoxytrityl-5-methyl-4-triazoleuridine

A first solution was prepared by dissolving 3′-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) was added to a solution of triazole (90 g, 1.3 M) in CH₃CN (1 L), cooled to −5° C. and stirred for 0.5 h using an overhead stirrer. POCl₃ was added dropwise, over a 30 minute period, to the stirred solution maintained at 0-10° C., and the resulting mixture stirred for an additional 2 hours. The first solution was added to the later solution dropwise, over a 45 minute period. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with 1×300 mL of NaHCO₃ and 2×300 mL of saturated NaCl, dried over sodium sulfate and evaporated. The residue was triturated with EtOAc to give the title compound.

2′-O-Methoxyethyl-5′-O-dimethoxytrityl-5-methylcytidine

A solution of 3′-O-acetyl-2′-O-methoxyethyl-5′-O-dimethoxytrityl-5-methyl-4-triazoleuridine (103 g, 0.141 M) in dioxane (500 mL) and NH₄OH (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. MeOH (400 mL) saturated with NH₃ gas was added and the vessel heated to 100° C. for 2 hours (tlc showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound.

N⁴-Benzoyl-2′-O-methoxyethyl-5′-O-dimethoxytrityl-5-methylcytidine

2′-O-Methoxyethyl-5′-O-dimethoxytrityl-5-methylcytidine (85 g, 0.134 M) was dissolved in DMF (800 mL) and benzoic anhydride (37.2 g, 0.165 M) was added with stirring. After stirring for 3 hours, tlc showed the reaction to be approximately 95% complete. The solvent was evaporated and the residue azeotroped with MeOH (200 mL). The residue was dissolved in CHCl₃ (700 mL) and extracted with saturated NaHCO₃ (2×300 mL) and saturated NaCl (2×300 mL), dried over MgSO₄ and evaporated to give a residue (96 g). The residue was chromatographed on a 1.5 kg silica column using EtOAc/Hexane (1:1) containing 0.5% Et₃NH as the eluting solvent. The pure product fractions were evaporated to give 90 g (90%) of the title compound.

N⁴-Benzoyl-21-O-methoxyethyl-5′-O-dimethoxytrityl-5-methylcytidine-3′-amidite

N⁴-Benzoyl-2′-O-methoxyethyl-5′-O-dimethoxytrityl-5-methylcytidine (74 g, 0.10 M) was dissolved in CH₂Cl₂ (1 L) Tetrazole diisopropylamine (7.1 g) and-2-cyanoethoxy-tetra-(isopropyl)phosphite (40.5 mL, 0.123 M) were added with stirring, under a nitrogen atmosphere. The resulting mixture was stirred for 20 hours at room temperature (tlc showed the reaction to be 95% complete). The reaction mixture was extracted with saturated NaHCO₃ (1×300 mL) and saturated NaCl (3×300 mL). The aqueous washes were back-extracted with CH₂Cl₂ (300 mL), and the extracts were combined, dried over MgSO₄; and concentrated. The residue obtained was chromatographed on a 1.5 kg silica column using EtOAc\Hexane (3:1) as the eluting solvent. The pure fractions were combined to give 90.6 g. (87%) of the title compound.

2′-O-(Aminooxyethyl) Nucleoside Amidites and 2′-O-(dimethylaminooxyethyl) Nucleoside Amidites: 2′-(Dimethylaminooxyethoxy) Nucleoside Amidites

2′-(Dimethylaminooxyethoxy) nucleoside amidites [also known in the art as 2′-O-(dimethylaminooxyethyl) nucleoside amidites] are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

5′-O-tert-Butyldiphenylsilyl-O₂-2′-anhydro-5-methyluridine

O²-2′-anhydro-5-methyluridine (Pro. Bio. Sint., Varese, Italy, 100.0 g, 0.416 mmol), dimethylaminopyridine (0.669, 0.013 eq, 0.0054 mmol) were dissolved in dry pyridine (500 ml) at ambient temperature under an argon atmosphere and with mechanical stirring. tert-Butyldiphenylchlorosilane (125.8 g, 119.0 mL, 1.1 eq, 0.458 mmol) was added in one portion. The reaction was stirred for 16 h at ambient temperature. TLC (Rf 0.22, ethyl acetate) indicated a complete reaction. The solution was concentrated under reduced pressure to a thick oil. This was partitioned between dichloromethane (1 L) and saturated sodium bicarbonate (2×1 L) and brine (1 L). The organic layer was dried over sodium sulfate and concentrated under reduced pressure to a thick oil. The oil was dissolved in a 1:1 mixture of ethyl acetate and ethyl ether (600 mL) and the solution was cooled to −10° C. The resulting crystalline product was collected by filtration, washed with ethyl ether (3×200 mL) and dried (40° C., 1 mm Hg, 24 h) to 149 g (74.8%) of white solid. TLC and NMR were consistent with pure product.

5′-O-tert-Butyldiphenylsilyl-2′-O-(2-hydroxyethyl)-5-methyluridine

In a 2 L stainless steel, unstirred pressure reactor was added borane in tetrahydrofuran (1.0 M, 2.0 eq, 622 mL). In the fume hood and with manual stirring, ethylene glycol (350 mL, excess) was added cautiously at first until the evolution of hydrogen gas subsided. 5′-O-tert-Butyldiphenylsilyl-O²-2′-anhydro-5-methyluridine (149 g, 0.311 mol) and sodium bicarbonate (0.074 g, 0.003 eq) were added with manual stirring. The reactor was sealed and heated in an oil bath until an internal temperature of 160° C. was reached and then maintained for 16 h (pressure<100 psig). The reaction vessel was cooled to ambient and opened. TLC (Rf 0.67 for desired product and Rf 0.82 for are-T side product, ethyl acetate) indicated about 70% conversion to the product. In order to avoid additional side product formation, the reaction was stopped, concentrated under reduced pressure (10 to 1 mm Hg) in a warm water bath (40-100° C.) with the more extreme conditions used to remove the ethylene glycol. [Alternatively, once the low boiling solvent is gone, the remaining solution can be partitioned between ethyl acetate and water. The product will be in the organic phase.] The residue was purified by column chromatography (2 kg silica gel, ethyl acetate-hexanes gradient 1:1 to 4:1). The appropriate fractions were combined, stripped and dried to product as a white crisp foam (84 g, 50%), contaminated starting material (17.4 g) and pure reusable starting material 20 g. The yield based on starting material less pure recovered starting material was 58%. TLC and NMR were consistent with 99% pure product.

2′-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol). It was then dried over P₂O₅ under high vacuum for two days at 40° C. The reaction mixture was flushed with argon and dry THF (369.8 mL, Aldrich, sure seal bottle) was added to get a clear solution. Diethylazodicarboxylate (6.98 mL, 44.36 mmol) was added dropwise to the reaction mixture. The rate of addition is maintained such that resulting deep red coloration is just discharged before adding the next drop. After the addition was complete, the reaction was stirred for 4 hrs. By that time TLC showed the completion of the reaction (ethylacetate:hexane, 60:40). The solvent was evaporated in vacuum. Residue obtained was placed on a flash column and eluted with ethyl acetate:hexane (6.0:40), to get 2′([2-phthalimidoxy)ethyl]-5′-t-butyldiphenylsilyl-5-methyluridine as white foam (21.819 g, 86%).

5′-O-tert-butyldiphenylsilyl-2′-O-[(2-formadoximinooxy)ethyl]-5-methyluridine

2′-O-(.[2-phthalimidoxy)ethyl]-5′-t-butyldiphenylsilyl-5-methyluridine (3.1 g, 4.5 mmol) was dissolved in dry CH₂Cl₂ (4.5 mL) and methylhydrazine (300 mL, 4.64 mmol) was added dropwise at −10° C. to 0° C. After 1 h the mixture was filtered, the filtrate was washed with ice cold CH₂Cl₂ and the combined organic phase was washed with water, brine and dried over anhydrous Na₂SO₄. The solution was concentrated to get 2′-O-(aminooxyethyl) thymidine, which was then dissolved in MeOH (67.5 mL). To this formaldehyde (20% aqueous solution, w/w, 1.1 eq.) was added and the resulting mixture was strirred for 1 h. Solvent was removed under vacuum; residue chromatographed to get 5′-O-tert-butyldiphenylsilyl-2′-[(2-formadoximinooxy) ethyl]-5-methyluridine as white foam (1.95 g, 78%).

5′-O-tert-Butyldiphenylsilyl-2′-O-[N,N-dimethylaminooxyethyl]-5

5′-O-tert-butyldiphenylsilyl-2′-O-[(2-formadoximinooxy)ethyl]-5-methyluridine (1.77 g, 3.12 mmol) was dissolved in a solution of 1M pyridinium p-toluenesulfonate (PPTS) in dry MeOH (30.6 mL). Sodium cyanoborohydride (0.39 g, 6.13 mmol) was added to this solution at 100 C under inert atmosphere. The reaction mixture was stirred for 10 minutes at 10C. After that the reaction vessel was removed from the ice bath and stirred at room temperature for 2 h, the reaction monitored by TLC (5% MeOH in CH₂Cl₂). Aqueous NaHCO₃ solution (5%, 10 mL) was added and extracted with ethyl acetate (2×20 mL). Ethyl acetate phase was dried over anhydrous Na₂SO₄, evaporated to dryness. Residue was dissolved in a solution of 1M PPTS in MeOH (30.6 mL). Formaldehyde (20% w/w, 30 mL, 3.37 mmol) was added and the reaction mixture was stirred at room temperature for 10 minutes. Reaction mixture cooled to 10° C. in an ice bath, sodium cyanoborohydride (0.39 g, 6.13 mmol) was added and reaction mixture stirred at 10° C. for 10 minutes. After 10 minutes, the reaction mixture was removed from the ice bath and stirred at room temperature for 2 hrs. To the reaction mixture 5% NaHCO₃ (25 mL) solution was added and extracted with ethyl acetate (2×25 mL). Ethyl acetate layer was dried over anhydrous Na2SO4 and evaporated to dryness. The residue obtained was purified by flash column chromatography and eluted with 5% MeOH in CH₂Cl₂ to get 5′-O-tert-butyldiphenylsilyl-2′-O-[N,N-dimethylaminooxyethyl]-5-methyluridine as a white foam (14.6 g, 80%).

2′-O-(dimethylaminooxyethyl)-5-methyluridine

Triethylamine trihydrofluoride (3.91 mL, 24.0 mmol) was dissolved in dry THF and triethylamine (1.67 mL, 12 mmol, dry, kept over KOH). This mixture of triethylamine-2HF was then added to 5′-O-tert-butyldiphenylsilyl-2′-O-[N,N-dimethylaminooxyethyl]-5-methyluridine (1.40 g, 2.4 mmol) and stirred at room temperature for 24 hrs. Reaction was monitored by TLC (5% MeOH in CH₂Cl₂).<Solvent was removed under vacuum and the residue placed on a flash column and eluted with 10% MeOH in CH₂Cl₂ to get 2′-O-(dimethylaminooxyethyl)-5-methyluridine (766 mg, 92.5%).

5′-O-DMT-2′-O-(dimethylaminooxyethyl)-5-methyluridine

2′-O-(dimethylaminooxyethyl)-5-methyluridine (750 mg, 2.17 mmol) was dried over P₂O₅ under high vacuum overnight at 40° C. It was then co-evaporated with anhydrous pyridine (20 mL). The residue obtained was dissolved in pyridine (11 mL) under argon atmosphere. 4-dimethylaminopyridine (26.5 mg; 2.60 mmol), 4,4′-dimethoxytrityl chloride (880 mg, 2.60 mmol) was added to the mixture and the reaction mixture was stirred at room temperature until all of the starting material disappeared. Pyridine was removed under vacuum and the residue chromatographed and eluted with 10% MeOH in CH₂Cl₂ (containing a few drops of pyridine) to get 5′-O-DMT-2′-O-(dimethylamino-oxyethyl)-5-methyluridine (1.13 g, 80%).

5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) was co-evaporated with toluene (20 mL). To the residue N,N-diisopropylamine tetrazonide (0.29 g, 1.67 mmol) was added and dried over P205 under high vacuum overnight at 40° C. Then the reaction mixture was dissolved in anhydrous acetonitrile (8.4 mL) and 2-cyanoethyl-N,N,N1,N1-tetraisopropylphosphoramidite (2.12 mL, 6.08 mmol) was added. The reaction mixture was stirred at ambient temperature for 4 hrs under inert atmosphere. The progress of the reaction was monitored by TLC (hexane:ethyl acetate 1:1). The solvent was evaporated, then the residue was dissolved in ethyl acetate (70 mL) and washed with 5% aqueous NaHCO3 (40 mL). Ethyl acetate layer was dried over anhydrous Na2SO4 and concentrated. Residue obtained was chromatographed (ethyl acetate as eluent) to get 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%).

2′-(Aminooxyethoxy) Nucleoside Amidites

2′-(Aminooxyethoxy) nucleoside amidites [also known in the art as 2′-O-(aminooxyethyl) nucleoside amidites] are prepared as described in the following paragraphs. Adenosine, cytidine and thymidine nucleoside amidites are prepared similarly.

N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl) diaminopurine riboside along with a minor amount of the 3′-O-isomer. 2′-α-(2-ethylacetyl) diaminopurine riboside may be resolved and converted to 2′-O-(2-ethylacetyl)guanosine by treatment with adenosine deaminase. (PCT WO94/02501). Standard protection procedures should afford 2′-O-(2-ethylacetyl)-5′-O-(4,1-4′-dimethoxytrityl)guanosine and 2-N-isobutyryl-6-O-diphenylcarbamoyl-2′-O-(2-ethylacetyl)-5′-O-(4,4′-dimethoxytrityl)guanosine which may be reduced to provide 2-N-isobutyryl-6-O-diphenylcarbamoyl-2′-O-(2-ethylacetyl)-5′-O-(4,4′-dimethoxytrityl)guanosine. As before the hydroxyl group may be displaced by N-hydroxyphthalimide via a Mitsunobu reaction, and the protected nucleoside may phosphitylated as usual to yield 2-N-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite].

2′-dimethylaminoethoxyethoxy (2′-DMAEOE) Nucleoside Amidites

2′-dimethylaminoethoxyethoxy nucleoside amidites (also known in the art as 2′-O-dimethylaminoethoxyethyl, i.e., 2′-O—CH₂—O—CH₂—N(CH₂)₂, or 2′-DMAEOE nucleoside amidites) are prepared as follows. Other nucleoside amidites are prepared similarly.

2′-O-[2(2-N,N-dimethylaminoethoxy)ethyl]-5-methyl Uridine

2[2-(Dimethylamino)ethoxy]ethanol (Aldrich, 6.66 g, 50 mmol) is slowly added to a solution of borane in tetrahydrofuran (1 M, 10 mL, 10 mmol) with stirring in a 100 mL bomb. Hydrogen gas evolves as the solid dissolves. O2-,2′-anhydro-5-methyluridine (1.2 g, 5 mmol), and sodium bicarbonate (2.5 mg) are added and the bomb is sealed, placed in an oil bath and heated to 155 C for 26 hours. The bomb is cooled to room temperature and opened. The crude solution is concentrated and the residue partitioned between water (200 mL) and hexanes (200 mL). The excess phenol is extracted into the hexane layer. The aqueous layer is extracted with ethyl acetate (3×200 mL) and the combined organic layers are washed once with water, dried over anhydrous sodium sulfate and concentrated. The residue is columned on silica gel using methanol/methylene chloride 1:20 (which has 2% triethylamine) as the eluent. As the column fractions are concentrated a colorless solid forms which is collected to give the title compound as a white solid.

5′-O-dimethoxytrityl-2′-O-[2(2-N,N-dimethylaminoethoxy)ethyl)]-5-methyl Uridine

To 0.5 g (1.3 mmol) of 2′-O-[2(2-N,N-dimethylaminoethoxy)ethyl)]-5-methyl uridine in anhydrous pyridine (8 mL), triethylamine (0.36 mL) and dimethoxytrityl chloride (DMT-Cl, 0.87 g, 2 eq.) are added and stirred for 1 hour. The reaction mixture is poured into water (200 mL) and extracted with CH₂Cl₂ (2×200 mL). The combined CH₂Cl₂ layers are washed with saturated NaHCO3 solution, followed by saturated NaCl solution and dried over anhydrous sodium sulfate. Evaporation of the solvent followed by silica gel chromatography using MeOH:CH₂Cl₂:Et3N (20:1, v/v, with 1% triethylamine) gives the title compound.

5′-O-Dimethoxytrityl-2′-O-[2(2-N,N-dimethylaminoethoxy)-ethyl)]-5-methyl uridine-3′-O— (cyanoethyl-N,N-diisopropyl)phosphoramidite

Diisopropylaminotetrazolide (0.6 g) and 2-cyanoethoxy-N,N-diisopropyl phosphoramidite (1.1 mL, 2 eq.) are added to a solution of 5′-O-dimethoxytrityl-2′-O-[2(2-N,N-dimethylaminoethoxy)ethyl)]-5-methyluridine (2.17 g, 3 mmol) dissolved in CH₂Cl₂ (20 mL) under an atmosphere of argon. The reaction mixture is stirred overnight and the solvent evaporated. The resulting residue is purified by silica gel flash column chromatography with ethyl acetate as the eluent to give the title compound.

Purification:

After cleavage from the controlled pore glass column (Applied Biosystems) and deblocking in concentrated ammonium hydroxide at 55° C. for 18 hours, the oligonucleotides were purified by precipitation twice out of 0.5 M NaCl with 2.5 volumes ethanol. Analytical gel electrophoresis was accomplished in 20% acrylamide, 8 M urea, 45 mM Tris-borate buffer, pH 7.0. Oligodeoxynucleotides and their phosphorothioate analogs were judged from electrophoresis to be greater than 80% full length material.

B7 Antisense Oligonucleotides

A series of oligonucleotides with sequences designed to hybridize to the published human B7-1 (hB7-1) and murine (mB7-1) mRNA sequences (Freeman et al., J. Immunol., 1989, 143, 2714, and Freeman et al., J. Exp. Med., 1991, 174, 625 respectively). The sequences of and modifications to these oligonucleotides, and the location of each of their target sites on the hB7-1 mRNA, are given in Tables 1 and 2. Similarly, a series of oligonucleotides with sequences designed to hybridize to the human B7-2 (hB7-2) and murine B7-2 (mB7-2) mRNA published sequences (respectively, Azuma et al., Nature, 1993, 366, 76; Chen et al., J. Immunol., 1994, 152, 4929 were synthesized. The sequences of and modifications to these oligonucleotides and the location of each of their target sites on the hB7-2 mRNA are described in Tables 3 and 4. Antisense oligonucleotides targeted to ICAM-1, including ISIS 2302 (SEQ ID NO: 17), have been described in U.S. Pat. No. 5,514,788, which issued May 7, 1996, hereby incorporated by reference. ISIS 1082 (SEQ ID NO: 102) and ISIS 3082 (SEQ ID NO: 101) have been previously described (Stepkowski et al., J. Immunol., 1994, 153, 5336).

Subsequent to their initial cloning, alternative splicing events of B7 transcripts have been reported. The reported alternative splicing for B7-1 is relatively simple, in that it results in messages extended 5′ relative to the 5′ terminus of the human and murine B7-1 cDNA sequences originally reported (Borriello et al., J. Immunol., 1994, 153, 5038; Inobe et al., J. Immunol., 1996, 157, 588). In order to retain the numbering of the B7-1 sequences found in the references initially reporting B7-1 sequences, positions within these 5′ extensions of the initially reported sequences have been given negative numbers (beginning with position −1, the most 3′ base of the 5′ extension) in Tables 1 and 2. The processing of murine B7-2 transcripts is considerably more complex than that so far reported for B7-1; for example, at least five distinct murine B7-2 mRNAs, and at least two distinct human B7-2 mRNAs, can be produced by alternative splicing events (Borriello et al., J. Immunol., 1995, 155, 5490; Freeman et al., WO 95/03408, published Feb. 2, 1995; see also Jellis et al., Immunogenet., 1995, 42, 85). The nature of these splicing events is such that different 5′ exons are used to produce distinct B7-2 mRNAs, each of which has a unique 5′ sequence but which share a 3′ portion consisting of some or all of the B7-2 sequence initially reported. As a result, positions within the 5′ extensions of B7-2 messages cannot be uniquely related to a position within the sequence initially reported. Accordingly, in Table 3, a different set of coordinates (corresponding to those of SEQ ID NO: 1 of WO 95/03408) and, in Table 4, the exon number (as given in Borriello et al., J. Immunol., 1995, 155, 5490) is used to specify the location of targeted sequences which are not included in the initially reported B7-2 sequence. Furthermore, although these 5′ extended messages contain potential in-frame start codons upstream from the ones indicated in the initially published sequences, for simplicity's sake, such additional potential start codons are not indicated in the description of target sites in Tables 1-4.

In Tables 1-4, the following abbreviations are used: UTR, untranslated region; ORF, open reading frame; tIR, translation initiation region; tTR, translation termination region; FITC, fluorescein isothiocyanate. Chemical modifications are indicated as follows. Residues having 2′fluoro (2° F.), 2′-methoxy (2′MO) or 2′-methoxyethoxy (2′ME) modification are emboldened, with the type of modification being indicated by the respective abbreviations. Unless otherwise indicated, interresidue linkages are phosphodiester linkages; phosphorothioate linkages are indicated by an “S” in the superscript position (e.g., T^{S}A). Target positions are numbered according to Freeman et al., J. Immunol., 1989, 143:2714 (human B7-1 cDNA sequence; Table l), Freeman et al., J. Exp. Med., 1991, 174, 625 (murine B7-1 cDNA sequence; Table 2), Azuma et al., Nature, 1993, 366:76 (human B7-2 cDNA sequence; Table 3) and Chen et al., J. Immunol., 1994, 152:4929 (murine B7-2 cDNA sequence; Table 4). Nucleotide base codes are as given in 37 C.F.R. § 1.822(b)(1).

TABLE 1

Sequences of Oligonucleotides Targeted to Human B7-1 mRNA

| | | | |
|---|---|---|---|
| | | | SEQ |
| | Target Position; Site | Oligonucleotide Sequence(5′−>3′) and | ID |
| ISIS # | (and/or Description) | Chemical Modifications | NO: |

| | | | |
|---|---|---|---|
| 13797 | 0053-0072; 5′ UTR | G^{S}G^{S}G^{S}T^{S}A^{S}A^{S}G^{S}A^{S}C^{S}T^{S}C^{S}C^{S}A^{S}C^{S}T^{S}T^{S}C^{S}T^{S}G^{S}A | 22 |
| 13798 | 0132-0151; 5′ UTR | G^{S}G^{S}G^{S}T^{S}C^{S}T^{S}C^{S}C^{S}A^{S}A^{S}A^{S}G^{S}G^{S}T^{S}T^{S}G^{S}T^{S}G^{S}G^{S}A | 23 |
| 13799 | 0138-0157; 5′ UTR | G^{S}T^{S}T^{S}C^{S}C^{S}T^{S}G^{S}G^{S}G^{S}T^{S}C^{S}T^{S}C^{S}C^{S}A^{S}A^{S}A^{S}G^{S}G^{S}T | 24 |
| 13800 | 0158-0177; 5′ UTR | A^{S}C^{S}A^{S}C^{S}A^{S}C^{S}A^{S}G^{S}A^{S}G^{S}A^{S}T^{S}T^{S}G^{S}G^{S}A^{S}G^{S}G^{S}G^{S}T | 25 |
| 13801 | 0193-0212; 5′ UTR | G^{S}C^{S}T^{S}C^{S}A^{S}C^{S}G^{S}T^{S}A^{S}G^{S}A^{S}G^{S}A^{S}C^{S}C^{S}C^{S}T^{S}C^{S}C | 26 |
| 13802 | 0217-0236; 5′ UTR | G^{S}G^{S}C^{S}A^{S}G^{S}G^{S}G^{S}C^{S}T^{S}G^{S}A^{S}T^{S}G^{S}A^{S}C^{S}A^{S}A^{S}T^{S}C^{S}C | 27 |
| 13803 | 0226-0245; 5′ UTR | T^{S}G^{S}C^{S}A^{S}A^{S}A^{S}A^{S}C^{S}A^{S}G^{S}G^{S}C^{S}A^{S}G^{S}G^{S}G^{S}C^{S}T^{S}G^{S}A | 28 |
| 13804 | 0246-0265; 5′ UTR | A^{S}G^{S}A^{S}C^{S}C^{S}A^{S}G^{S}G^{S}G^{S}C^{S}A^{S}C^{S}T^{S}T^{S}C^{S}C^{S}C^{S}A^{S}G^{S}G | 29 |
| 13805 | 0320-0339;tIR | C^{S}C^{S}T^{S}G^{S}C^{S}C^{S}T^{S}C^{S}C^{S}G^{S}T^{S}G^{S}T^{S}G^{S}T^{S}G^{S}G^{S}C^{S}C^{S}C | 30 |
| 13806 | 0380-0399; 5′ ORF | G^{S}A^{S}C^{S}C^{S}A^{S}G^{S}C^{S}C^{S}A^{S}G^{S}C^{S}A^{S}C^{S}C^{S}A^{S}A^{S}G^{S}A^{S}G^{S}C | 31 |
| 13807 | 0450-0469; 5′ ORF | C^{S}C^{S}A^{S}C^{S}A^{S}G^{S}G^{S}A^{S}C^{S}A^{S}G^{S}C^{S}G^{S}T^{S}T^{S}G^{S}C^{S}C^{S}A^{S}C | 32 |
| 13808 | 0568-0587; 5′ ORF | C^{S}C^{S}G^{S}G^{S}T^{S}T^{S}C^{S}T^{S}T^{S}G^{S}T^{S}A^{S}C^{S}T^{S}C^{S}G^{S}G^{S}G^{S}C^{S}C | 33 |
| 13809 | 0634-0653; central ORF | G^{S}C^{S}C^{S}C^{S}T^{S}C^{S}G^{S}T^{S}C^{S}A^{S}G^{S}A^{S}T^{S}G^{S}G^{S}G^{S}C^{S}G^{S}C^{S}A | 51 |
| 13810 | 0829-0848; central ORF | C^{S}C^{S}A^{S}A^{S}C^{S}C^{S}A^{S}G^{S}G^{S}A^{S}G^{S}A^{S}G^{S}G^{S}T^{S}G^{S}A^{S}G^{S}G^{S}C | 34 |
| 13811 | 1102-1121; 3′ORF | G^{S}G^{S}C^{S}A^{S}A^{S}A^{S}G^{S}C^{S}A^{S}G^{S}T^{S}A^{S}G^{S}G^{S}T^{S}C^{S}A^{S}G^{S}C | 35 |
| 13812 | 1254-1273; 3′-UTR | G^{S}C^{S}C^{S}T^{S}C^{S}A^{S}T^{S}G^{S}A^{S}T^{S}C^{S}C^{S}C^{S}C^{S}A^{S}C^{S}G^{S}A^{S}T^{S}C | 36 |
| 13872 | (scrambled#13812) | A^{S}G^{S}T^{S}C^{S}C^{S}T^{S}A^{S}C^{S}T^{S}A^{S}C^{S}C^{S}A^{S}G^{S}C^{S}C^{S}C^{S}C^{S}C^{S}T | 52 |
| 12361 | 0056-0075; 5′ UTR | T^{S}C^{S}A^{S}G^{S}G^{S}G^{S}T^{S}A^{S}A^{S}G^{S}A^{S}C^{S}T^{S}C^{S}C^{S}A^{S}C^{S}T^{S}T^{S}C | 38 |
| 12348 | 0056-0075; 5′ UTR | T C A G G G^{S}T^{S}A^{S}A^{S}G^{S}A^{S}C^{S}T^{S}C^{S}C A C T T C | 38 |
| | | (2′ME) | |
| 12473 | 0056-0075; 5′ UTR | T^{S}C^{S}A^{S}G^{S}G^{S}G^{S}T^{S}A^{S}A^{S}G^{S}A^{S}C^{S}T^{S}C^{S}C^{S}A^{S}C^{S}T^{S}T^{S}C | 38 |
| | | (2′ F1) | |
| 12362 | 0143-0162; 5′ UTR | A^{S}G^{S}G^{S}G^{S}T^{S}G^{S}T^{S}T^{S}C^{S}C^{S}T^{S}G^{S}G^{S}G^{S}T^{S}C^{S}T^{S}C^{S}C^{S}A | 39 |
| 12349 | 0143-0162; 5′ UTR | A G G G T G^{S}T^{S}T^{S}C^{S}C^{S}T^{S}G^{S}G^{S}G^{S}T C T C C A | 39 |
| | | (2′ ME) | |
| 12474 | 0143-0162; 5′ UTR | A^{S}G^{S}G^{S}G^{S}T^{S}G^{S}T^{S}T^{S}C^{S}C^{S}T^{S}G^{S}G^{S}G^{S}T^{S}C^{S}T^{S}C^{S}C^{S}A | 39 |
| | | (2′ F1) | |
| 12363 | 0315-0334;tIR | C^{S}T^{S}C^{S}C^{S}G^{S}T^{S}G^{S}T^{S}G^{S}T^{S}G^{S}G^{S}C^{S}C^{S}C^{S}A^{S}T^{S}G^{S}G^{S}C | 40 |
| 12350 | 0315-0334; tIR | C T C C G T^{S}G^{S}T^{S}G^{S}T^{S}G^{S}G^{S}C^{S}C C A T G G C | 40 |
| | | (2′ ME) | |
| 12475 | 0315-0334; tIR | C^{S}T^{S}C^{S}C^{S}G^{S}T^{S}G^{S}T^{S}G^{S}G^{S}C^{S}C^{S}C^{S}A^{S}T^{S}G^{S}G^{S}C | 40 |
| | | (2′ F1) | |
| 12364 | 0334-0353; 5′ ORF | G^{S}G^{S}A^{S}T^{S}G^{S}G^{S}T^{S}G^{S}A^{S}T^{S}G^{S}T^{S}T^{S}C^{S}C^{S}C^{S}T^{S}G^{S}C^{S}C | 41 |
| 12351 | 0334-0353; 5′ ORF | G G A T G G^{S}T^{S}G^{S}A^{S}T^{S}G^{S}T^{S}T^{S}C C C T G C C | 41 |
| | | (2′ ME) | |
| 12476 | 0334-0353; 5′ ORF | G^{S}G^{S}A^{S}T^{S}G^{S}G^{S}T^{S}G^{S}A^{S}T^{S}G^{S}T^{S}T^{S}C^{S}C^{S}C^{S}T^{S}G^{S}C^{S}C | 41 |
| | | (2′ F1) | |
| 12365 | 0387-0406; 5′ ORF | T^{S}G^{S}A^{S}G^{S}A^{S}A^{S}A^{S}G^{S}A^{S}C^{S}C^{S}A^{S}G^{S}C^{S}C^{S}A^{S}G^{S}C^{S}A^{S}C | 42 |
| 12352 | 0387-0406; 5′ ORF | T G A G A A^{S}A^{S}G^{S}A^{S}C^{S}C^{S}A^{S}G^{S}C^{S}C A G C A C | 42 |
| | | (2′ ME) | |
| 12477 | 0387-0406; 5′ ORF | T^{S}G^{S}A^{S}G^{S}A^{S}A^{S}A^{S}G^{S}A^{S}C^{S}C^{S}A^{S}G^{S}C^{S}C^{S}A^{S}G^{S}C^{S}A^{S}C | 42 |
| | | (2′ F1) | |
| 12366 | 0621-0640; central ORF | G^{S}G^{S}G^{S}C^{S}G^{S}C^{S}A^{S}G^{S}A^{S}G^{S}C^{S}C^{S}A^{S}G^{S}G^{S}A^{S}T^{S}C^{S}A^{S}C | 43 |
| 12353 | 0621-0640; central ORF | G^{S}G^{S}G^{S}C^{S}G^{S}C^{S}A^{S}G^{S}A^{S}G^{S}C^{S}C^{S}A^{S}G^{S}G^{S}A^{S}T^{S}C^{S}A^{S}C | 43 |
| | | (2′ ME) | |
| 12478 | 0621-0640; central ORF | G^{S}G^{S}G^{S}C^{S}G^{S}C^{S}A^{S}G^{S}A^{S}G^{S}C^{S}C^{S}A^{S}G^{S}G^{S}A^{S}T^{S}C^{S}A^{S}C | 43 |
| | | (2′ F1) | |
| 12367 | 1042-1061; 3′ORF | G^{S}G^{S}C^{S}C^{S}C^{S}A^{S}G^{S}G^{S}A^{S}T^{S}G^{S}G^{S}G^{S}A^{S}G^{S}C^{S}A^{S}G^{S}G^{S}T | 44 |
| 12354 | 1042-1061; 3′ORF | G G C C C A^{S}G^{S}G^{S}A^{S}T^{S}G^{S}G^{S}G^{S}A G C A G G T | 44 |
| | | (2′ ME) | |
| 12479 | 1042-1061; 3′ORF | G^{S}G^{S}C^{S}C^{S}C^{S}A^{S}G^{S}G^{S}A^{S}T^{S}G^{S}G^{S}G^{S}A^{S}G C A G G T | 44 |
| | | (2′ F1) | |
| 12368 | 1069-1088; tTR | A^{S}G^{S}G^{S}G^{S}C^{S}G^{S}T^{S}A^{S}C^{S}A^{S}C^{S}T^{S}T^{S}T^{S}C^{S}C^{S}C^{S}T^{S}T^{S}C | 45 |
| 12355 | 1069-1088; tTR | A G G G C G^{S}T^{S}A^{S}C^{S}A^{S}C^{S}T^{S}T^{S}T C C C T T C | 45 |
| | | (2′ ME) | |
| 12480 | 1069-1088; tTR | A^{S}G^{S}G^{S}G^{S}C^{S}G^{S}T^{S}A^{S}C^{S}A^{S}C^{S}T^{S}T^{S}T^{S}C^{S}C^{S}C^{S}T^{S}T^{S}C | 45 |
| | | (2′ F1) | |
| 12369 | 1100-1209; tTR | C^{S}A^{S}G^{S}C^{S}C^{S}C^{S}C^{S}T^{S}T^{S}G^{S}C^{S}T^{S}T^{S}C^{S}T^{S}G^{S}C^{S}G^{S}G^{S}A | 46 |
| 12356 | 1100-1209; tTR | C A G C C C^{S}C^{S}T^{S}T^{S}G^{S}C^{S}T^{S}T^{S}C^{S}T G C G G A | 46 |
| | | (2′ ME) | |
| 12481 | 1100-1209; tTR | C^{S}A^{S}G^{S}C^{S}C^{S}C^{S}C^{S}T^{S}T^{S}G^{S}C^{S}T^{S}T^{S}C^{S}T^{S}G^{S}C^{S}G^{S}G^{S}A | 46 |
| | | (2′ F1) | |
| 12370 | 1360-1380; 3′ UTR | A^{S}A^{S}G^{S}G^{S}A^{S}G^{S}A^{S}G^{S}G^{S}G^{S}A^{S}T^{S}G^{S}C^{S}C^{S}A^{S}G^{S}C^{S}C^{S}A | 47 |
| 12357 | 1360-1380; 3′ UTR | AAGGAGSASGSGSGSASTSOSCCAGCCA | 47 |
| | | (2′ ME) | |
| 12482 | 1360-1380; 3′ UTR | A^{S}A^{S}G^{S}G^{S}A^{S}G^{S}A^{S}G^{S}G^{S}G^{S}A^{S}T^{S}G^{S}C^{S}C^{A}G^{S}C^{S}C^{S}A | 47 |
| | | (2′ F1) | |
| 12914 | (−0038 to −0059; 5′ | C^{S}T^{S}G^{S}T^{S}T^{S}A^{S}C^{S}T^{S}T^{S}T^{S}A^{S}C^{S}A^{S}G^{S}A^{S}G^{S}G^{S}G^{S}T^{S}T^{S}T^{S}G | 48 |
| | UTR of alternative | (2′ MO) | |
| | mRNA) | | |
| 12915 | (−0035 to −0059; 5′ | C^{S}T^{S}T^{S}C^{S}T^{S}G^{S}T^{S}T^{S}A^{S}C^{S}T^{S}T^{S}T^{S}A^{S}C^{S}A^{S}G^{S}A^{S}G^{S}G^{S}G^{S}T^{S} | 49 |
| | UTR of alternative | T^{S}T^{S}G | |
| | mRNA) | (2′ ME) | |
| 13498 | (−0038 to −0058; 5′ | C^{S}T^{S}G^{S}T^{S}T^{S}A^{S}C^{S}T^{S}T^{S}T^{S}A^{S}C^{S}A^{S}G^{S}A^{S}G^{S}G^{S}G^{S}T^{S}T^{S}T | 50 |
| | UTR of alternative | (2′ ME) | |
| | mRNA) | | |
| 13499 | (−0038 to −0058; 5′ | C T G T T A C T T T A C A G A G G G T T T | 50 |
| | UTR of alternative | (2′ ME) | |
| | mRNA) | | |

TABLE 2

Sequences of Oligonucleotides Targeted to Murine B7-1 mRNA

| | | | |
|---|---|---|---|
| | | | SEQ |
| | | Oligonucleotide Sequence (5′−>3′) | ID |
| ISIS # | Target Position; Site | and Chemical Modifications | NO: |

| | | | |
|---|---|---|---|
| 14419 | 0009-0028; 5′ UTR | A^{S}G^{S}T^{S}A^{S}A^{S}G^{S}A^{S}G^{S}T^{S}C^{S}T^{S}A^{S}T^{S}T^{S}G^{S}A^{S}G^{S}G^{S}T^{S}A | 53 |
| 14420 | 0041-0060; 5′ UTR | G^{S}G^{S}T^{S}T^{S}G^{S}A^{S}G^{S}T^{S}T^{S}T^{S}C^{S}A^{S}C^{S}A^{S}A^{S}C^{S}C^{S}T^{S}G^{S}A | 54 |
| 14421 | 0071-0091; 5′ UTR | G^{S}T^{S}C^{S}C^{S}A^{S}C^{S}A^{S}G^{S}A^{S}A^{S}T^{S}G^{S}G^{S}A^{S}A^{S}C^{S}A^{S}G^{S}A^{S}G | 55 |
| 14422 | 0109-0128; 5′ UTR | G^{S}G^{S}C^{S}A^{S}T^{S}C^{S}C^{S}A^{S}C^{S}C^{S}C^{S}G^{S}G^{S}C^{S}A^{S}G^{S}A^{S}T^{S}G^{S}C | 56 |
| 14423 | 0114-0133; 5′ UTR | T^{S}G^{S}G^{S}A^{S}T^{S}G^{S}G^{S}C^{S}A^{S}T^{S}C^{S}C^{S}A^{S}C^{S}C^{S}C^{S}G^{S}G^{S}C^{S}A | 57 |
| 14424 | 0168-0187; 5′ UTR | A^{S}G^{S}G^{S}C^{S}A^{S}C^{S}C^{S}T^{S}C^{S}C^{S}T^{S}A^{S}G^{S}G^{S}C^{S}T^{S}C^{S}A^{S}C^{S}A | 58 |
| 14425 | 0181-0200; 5′ UTR | G^{S}C^{S}C^{S}A^{S}A^{S}T^{S}G^{S}G^{S}A^{S}G^{S}C^{S}T^{S}T^{S}A^{S}G^{S}G^{S}C^{S}A^{S}C^{S}C | 59 |
| 14426 | 0208-0217; 5′ UTR | C^{S}A^{S}T^{S}G^{S}A^{S}T^{S}G^{S}G^{S}G^{S}G^{S}A^{S}A^{S}A^{S}G^{S}C^{S}C^{S}A^{S}G^{S}G^{S}A | 60 |
| 14427 | 0242-0261; tIR | A^{S}A^{S}T^{S}T^{S}G^{S}C^{S}A^{S}A^{S}G^{S}C^{S}C^{S}A^{S}T^{S}A^{S}G^{S}C^{S}T^{S}T^{S}C^{S}A | 61 |
| 14428 | 0393-0412; 5′ ORF | C^{S}G^{S}G^{S}C^{S}A^{S}A^{S}G^{S}G^{S}C^{S}A^{S}G^{S}C^{S}A^{S}A^{S}T^{S}A^{S}C^{S}C^{S}T^{S}T | 62 |
| 14909 | 0478-0497; 5′ ORF | C^{S}C^{S}C^{S}A^{S}G^{S}C^{S}A^{S}A^{S}T^{S}G^{S}A^{S}C^{S}A^{S}G^{S}A^{S}C^{S}A^{S}G^{S}C^{S}A | 63 |
| 14910 | 0569-0588; central ORF | G^{S}G^{S}T^{S}C^{S}T^{S}G^{S}A^{S}A^{S}A^{S}G^{S}G^{S}A^{S}C^{S}C^{S}A^{S}G^{S}G^{S}C^{S}C^{S}C | 64 |
| 14911 | 0745-0764; central ORF | T^{S}G^{S}G^{S}G^{S}A^{S}A^{S}A^{S}C^{S}C^{S}C^{S}C^{S}C^{S}G^{S}G^{S}A^{S}A^{S}C^{S}C^{S}A^{S}A | 65 |
| 14912 | 0750-0769; central ORF | G^{S}G^{S}C^{S}T^{S}T^{S}T^{S}G^{S}G^{S}G^{S}A^{S}A^{S}A^{S}C^{S}C^{S}C^{S}C^{S}C^{S}G^{S}G^{S}A | 66 |
| 14913 | 0825-0844; 3′ ORF | T^{S}C^{S}A^{S}G^{S}A^{S}T^{S}T^{S}C^{S}A^{S}G^{S}G^{S}A^{S}T^{S}C^{S}C^{S}T^{S}G^{S}G^{S}G^{S}A | 67 |
| 14914 | 0932-0951; 3′ ORF | C^{S}C^{S}C^{S}A^{S}G^{S}G^{S}T^{S}G^{S}A^{S}A^{S}G^{S}T^{S}C^{S}C^{S}T^{S}C^{S}T^{S}G^{S}A^{S}C | 68 |
| 14915 | 1001-1020; 3′ ORF | C^{S}T^{S}G^{S}C^{S}G^{S}C^{S}C^{S}G^{S}A^{S}A^{S}T^{S}C^{S}C^{S}T^{S}G^{S}C^{S}C^{S}C^{S}C^{S}A | 69 |
| 14916 | 1125-1144; tTR | C^{S}A^{S}G^{S}C^{S}C^{S}C^{S}G^{S}A^{S}A^{S}G^{S}G^{S}T^{S}A^{S}A^{S}G^{S}G^{S}C^{S}T^{S}G | 70 |
| 14917 | 1229-1248; 3′ UTR | T^{S}C^{S}A^{S}G^{S}C^{S}T^{S}A^{S}G^{S}C^{S}A^{S}C^{S}G^{S}G^{S}T^{S}G^{S}C^{S}T^{S}G^{S}A^{S}A | 71 |
| 14918 | 1329-1348; 3′ UTR | G^{S}G^{S}C^{S}C^{S}C^{S}A^{S}G^{S}C^{S}A^{S}A^{S}A^{S}C^{S}T^{S}T^{S}G^{S}C^{S}C^{S}C^{S}G^{S}T | 72 |
| 14919 | 1377-1393; 3′ UTR | C^{S}C^{S}A^{S}C^{S}C^{S}A^{S}C^{S}A^{S}G^{S}G^{S}G^{S}G^{S}C^{S}T^{S}C^{S}A^{S}G^{S}C^{S}C | 73 |
| 12912 | −0067 to −0049; 5′ UTR | G^{S}G^{S}C^{S}C^{S}A^{S}T^{S}G^{S}A^{S}G^{S}G^{S}G^{S}C^{S}A^{S}A^{S}T^{S}C^{S}T^{S}A^{S}A | 74 |
| | | (2′MO) | |
| 12913 | −0067 to −0047; 5′ UTR | G^{S}T^{S}G^{S}G^{S}C^{S}C^{S}A^{S}T^{S}G^{S}A^{S}G^{S}G^{S}G^{S}C^{S}A^{S}A^{S}T^{S}C^{S}T^{S}A^{S} | 75 |
| | | A (2′MO) | |
| 13496 | −0067 to −0047; 5′ UTR | G^{S}T^{S}G^{S}G^{S}C^{S}C^{S}A^{S}T^{S}G^{S}A^{S}G^{S}G^{S}G^{S}C^{S}A^{S}A^{S}T^{S}C^{S}T^{S}A^{S} | 75 |
| | | A (2′ME) | |
| 13497 | −0067 to −0047; 5′ UTR | G T G G C C A T G A G G G C A A T C T A | 75 |
| | | A (2′ME) | |

TABLE 3

Sequences of Oligonucleotides Targeted to Human B7-2 mRNA

| | | | |
|---|---|---|---|
| | | | SEQ |
| | | | ID |
| ISIS # | Target Position*; Site** | Oligonucleotide Sequence (5′−>3′) | NO: |

| | | | | |
|---|---|---|---|---|
| 9133 | 1367-1386; 3′-UTR | T^{S}T^{S}C^{S}C^{S}A^{S}G^{S}G^{S}T^{S}C^{S}A^{S}T^{S}G^{S}A^{S}G^{S}C^{S}C^{S}A^{S}T^{S}T^{S}A | 3 | |
| 10715 | scrambled control of #9133 | G^{S}A^{S}T^{S}T^{S}T^{S}A^{S}A^{S}C^{S}A^{S}T^{S}T^{S}T^{S}G^{S}G^{S}C^{S}G^{S}C^{S}C^{S}C^{S}A | 76 | |
| 9134 | 1333-1352; 3′-UTR | C^{S}A^{S}T^{S}A^{S}A^{S}G^{S}G^{S}T^{S}G^{S}T^{S}G^{S}C^{S}T^{S}C^{S}T^{S}G^{S}A^{S}A^{S}G^{S}T^{S}G | 4 | |
| 9135 | 1211-1230; 3′-UTR | T^{S}T^{S}A^{S}C^{S}T^{S}C^{S}A^{S}T^{S}G^{S}G^{S}T^{S}A^{S}A^{S}T^{S}G^{S}T^{S}C^{S}T^{S}T^{S}T^{S} | 5 | |
| 9136 | 1101-1120; tTR | A^{S}T^{S}T^{S}A^{S}A^{S}A^{S}A^{S}A^{S}C^{S}A^{S}T^{S}G^{S}T^{S}A^{S}T^{S}C^{S}A^{S}C^{S}T^{S}T^{S} | 6 | |
| 10716 | (scrambled#9136) | A^{S}A^{S}A^{S}G^{S}T^{S}T^{S}A^{S}C^{S}A^{S}A^{S}C^{S}A^{S}T^{S}T^{S}A^{S}T^{S}A^{S}T^{S}C^{S}T | 77 | |
| 9137 | 0054-0074; 5′-UTR | G^{S}G^{S}A^{S}A^{S}C^{S}A^{S}C^{S}A^{S}G^{S}A^{S}A^{S}G^{S}C^{S}A^{S}A^{S}G^{S}G^{S}T^{S}G^{S}C^{S}T | 7 | |
| 9138 | 0001-0020; 5′-UTR | C^{S}C^{S}G^{S}T^{S}A^{S}C^{S}C^{S}T^{S}C^{S}C^{S}T^{S}A^{S}A^{S}G^{S}G^{S}C^{S}T^{S}C^{S}C^{S}T | 8 | |
| 9139 | 0133-0152; tIR | C^{S}C^{S}A^{S}T^{S}A^{S}G^{S}T^{S}G^{S}C^{S}T^{S}G^{S}T^{S}C^{S}A^{S}C^{S}A^{S}A^{S}A^{S}T | 9 | |
| 10877 | (scrambled #9139) | A^{S}G^{S}T^{S}G^{S}C^{S}G^{S}A^{S}T^{S}T^{S}C^{S}T^{S}C^{S}A^{S}A^{S}A^{S}C^{S}C^{S}T^{S}A^{S}C^{S} | 78 | |
| 10367 | 0073-0092; 5′-UTR | G^{S}C^{S}A^{S}C^{S}A^{S}G^{S}C^{S}A^{S}G^{S}C^{S}A^{S}T^{S}T^{S}C^{S}C^{S}C^{S}A^{S}A^{S}G^{S}G^{S}G | 10 | |
| 10368 | 0240-0259; 5′ORF | T^{S}T^{S}G^{S}C^{S}A^{S}A^{S}A^{S}T^{S}T^{S}G^{S}G^{S}C^{S}A^{S}T^{S}G^{S}G^{S}C^{S}A^{S}G^{S}G | 11 | |
| 10369 | 1122-1141; 3′-UTR | T^{S}G^{S}G^{S}T^{S}A^{S}T^{S}G^{S}G^{S}G^{S}C^{S}T^{S}T^{S}T^{S}A^{S}C^{S}T^{S}C^{S}T^{S}T^{S}T | 12 | |
| 10370 | 1171-1190; 3′-UTR | A^{S}A^{S}A^{S}A^{S}G^{S}G^{S}T^{S}T^{S}G^{S}C^{S}C^{S}C^{S}A^{S}G^{S}G^{S}A^{S}A^{S}C^{S}G^{S}G | 13 | |
| 10371 | 1233-1252; 3′-UTR | G^{S}G^{S}G^{S}A^{S}G^{S}T^{S}C^{S}C^{S}T^{S}G^{S}G^{S}A^{S}G^{S}C^{S}C^{S}C^{S}C^{S}C^{S}T^{S}T | 14 | |
| 10372 | 1353-1372; 3′-UTR | C^{S}A^{S}T^{S}T^{S}A^{S}A^{S}G^{S}C^{S}T^{S}G^{S}G^{S}G^{S}C^{S}T^{S}T^{S}G^{S}G^{S}C^{S}C | 15 | |
| 11149 | 0019-0034; 5′-UTR | T^{S}A^{S}T^{S}T^{S}T^{S}G^{S}C^{S}G^{S}A^{S}G^{S}C^{S}T^{S}C^{S}C^{S}C^{S}C | 79 | |
| 11151 | 0020-0034; 5′-UTR | T^{S}A^{S}T^{S}T^{S}T^{S}G^{S}C^{S}G^{S}A^{S}G^{S}C^{S}T^{S}C^{S}C^{S}C | 80 | |
| 11150 | 0021-0034; 5′-UTR | T^{S}A^{S}T^{S}T^{S}T^{S}G^{S}C^{S}G^{S}A^{S}G^{S}C^{S}T^{S}C^{S}; C | 81 | |
| 10373 | 0011-0030; 5′-UTR | T^{S}G^{S}C^{S}G^{S}A^{S}G^{S}C^{S}T^{S}C^{S}C^{S}C^{S}C^{S}G^{S}T^{S}A^{S}C^{S}C^{S}T^{S}C^{S}C | 16 | |
| 10721 | (scrambled #10373) | C^{S}G^{S}A^{S}C^{S}A^{S}G^{S}G^{S}C^{S}T^{S}C^{S}C^{S}T^{S}G^{S}C^{S}T^{S}C^{S}C^{S}T^{S}C | 82 | |
| 10729 | (5′FITC#10373) | T^{S}G^{S}C^{S}G^{S}A^{S}G^{S}C^{S}T^{S}C^{S}C^{S}C^{S}C^{S}G^{S}T^{S}A^{S}C^{S}C^{S}T^{S}C^{S}C | 16 | |
| 10782 | (5′cholesterol#10373) | T^{S}G^{S}C^{S}G^{S}A^{S}G^{S}C^{S}T^{S}C^{S}C^{S}C^{S}C^{S}G^{S}T^{S}A^{S}C^{S}C^{S}T^{S}C^{S}C | 16 | |
| | #10373 Deletion | | | |
| | Derivatives: | | | |
| 10373 | 0011-0030; 5′-UTR | T^{S}G^{S}C^{S}G^{S}A^{S}G^{S}C^{S}T^{S}C^{S}C^{S}C^{S}C^{S}G^{S}T^{S}A^{S}C^{S}C^{S}T^{S}C^{S}C | 16 | |
| 10888 | 0011-0026; 5′-UTR | A^{S}G^{S}C^{S}T^{S}C^{S}C^{S}C^{S}C^{S}G^{S}T^{S}A^{S}C^{S}C^{S}T^{S}C^{S}C | 83 | |
| 10889 | 0015-0030; 5′-UTR | T^{S}G^{S}C^{S}G^{S}A^{S}G^{S}C^{S}T^{S}C^{S}C^{S}C^{S}C^{S}G^{S}T^{S}A^{S}C | 84 | |
| 10991 | 0015-0024; 5′-UTR | C^{S}T^{S}C^{S}C^{S}C^{S}G^{S}G^{S}T^{S}A^{S}C | 85 | |
| 10992 | 0015-0025; 5′-UTR | G^{S}C^{S}T^{S}C^{S}C^{S}C^{S}C^{S}G^{S}T^{S}A^{S}C | 86 | |
| 10993 | 0015-0026; 5′-UTR | A^{S}G^{S}C^{S}T^{S}C^{S}C^{S}C^{S}C^{S}G^{S}T^{S}A^{S}C | 87 | |
| 10994 | 0015-0027; 5′-UTR | G^{S}A^{S}G^{S}C^{S}T^{S}C^{S}C^{S}C^{S}C^{S}G^{S}T^{S}A^{S}C | 88 | |
| 10995 | 0015-0028; 5′-UTR | C^{S}G^{S}A^{S}G^{S}C^{S}T^{S}C^{S}C^{S}C^{S}C^{S}G^{S}T^{S}A^{S}C | 89 | |
| 10996 | 0015-0029; 5′-UTR | G^{S}C^{S}G^{S}A^{S}G^{S}C^{S}T^{S}C^{S}C^{S}C^{S}C^{S}G^{S}T^{S}A^{S}C | 90 | |
| 11232 | 0017-0029; 5′-UTR | G^{S}C^{S}G^{S}A^{S}G^{S}C^{S}T^{S}C^{S}C^{S}C^{S}C^{S}G^{S}T | 92 | |
| | #10996 Derivatives: | | | |
| 10996 | 0015-0029; 5′-UTR | G^{S}C^{S}G^{S}A^{S}G^{S}C^{S}T^{S}C^{S}C^{S}C^{S}C^{S}G^{S}T^{S}A^{S}C | 90 | |
| 11806 | (scrambled#10996) | G^{S}C^{S}C^{S}G^{S}C^{S}C^{S}G^{S}C^{S}C^{S}A^{S}A^{S}G^{S}T^{S}C^{S}T | 92 | |
| 11539 | (fully 2′ MO #10996) | G^{S}C^{S}G^{S}A^{S}G^{S}C^{S}T^{S}C^{S}C^{S}C^{S}C^{S}G^{S}T^{S}A^{S}C (2′MO) | 90 | |
| 11540 | (control for #11539) | G^{S}C^{S}C^{S}G^{S}C^{S}C^{S}G^{S}C^{S}C^{S}A^{S}A^{S}G^{S}T^{S}C^{S}T (2′MO) | 92 | |
| 11541 | (#10996 7-base “gapmer”) | G^{S}C^{S}G^{S}A^{S}G^{S}C^{S}T^{S}C^{S}C^{S}C^{S}C^{S}G^{S}T^{S}A^{S}C (2′MO) | 90 | |
| 11542 | (control for #11541) | G^{S}C^{S}C^{S}G^{S}C^{S}C^{S}G^{S}C^{S}C^{S}A^{S}A^{S}G^{S}T^{S}C^{S}T | (2′MO) | 92 |
| 11543 | (#10996 9-base “gapmer”) | G^{S}C^{S}G^{S}A^{S}G^{S}C^{S}T^{S}C^{S}C^{S}C^{S}C^{S}G^{S}T^{S}A^{S}C (2′MO) | 90 | |
| 11544 | (control for #11543) | G^{S}C^{S}C^{S}G^{S}C^{S}C^{S}G^{S}C^{S}C^{S}A^{S}A^{S}G^{S}T^{S}C^{S}T (2′MO) | 92 | |
| 12892 | 0001-0020 | C^{S}C^{S}G^{S}T^{S}A^{S}C^{S}C^{S}T^{S}C^{S}C^{S}T^{S}A^{S}A^{S}G^{S}G^{S}T^{S}C^{S}C | 98 | |
| | | (2′MO) | | |

TABLE 4

Sequences of oligonucleotides Targeted to Murine B7-2 mRNA

| | | | |
|---|---|---|---|
| ISIS # | Target Position; Site | Oligonucleotide Sequence (5′−>3′) | SEQ ID NO: |

| | | | |
|---|---|---|---|
| 11347 | 1094-1113; 3′ UTR | A^{S}A^{S}A^{S}T^{S}T^{S}C^{S}C^{S}A^{S}A^{S}T^{S}C^{S}A^{S}G^{S}C^{S}T^{S}G^{S}A^{S}G^{S}A | 121 |
| 11348 | 1062-1081; 3′ UTR | T^{S}C^{S}T^{S}G^{S}A^{S}G^{S}A^{S}A^{S}A^{S}C^{S}T^{S}C^{S}T^{S}G^{S}A^{S}T^{S}T^{S}C | 122 |
| 11349 | 1012-1031; 3′ UTR | T^{S}C^{S}C^{S}T^{S}C^{S}A^{S}G^{S}G^{S}C^{S}T^{S}C^{S}T^{S}C^{S}A^{S}C^{S}T^{S}G^{S}C^{S}C^{S}T | 123 |
| 11350 | 0019-1138; 5′ UTR | G^{S}G^{S}T^{S}T^{S}G^{S}T^{S}T^{S}C^{S}A^{S}A^{S}G^{S}T^{S}C^{S}C^{S}G^{S}T^{S}G^{S}C^{S}T^{S}G | 124 |
| 11351 | 0037-0056; 5′ UTR | A^{S}C^{S}A^{S}C^{S}G^{S}T^{S}C^{S}A^{S}C^{S}A^{S}G^{S}G^{S}A^{S}G^{S}T^{S}C^{S}T^{S}G^{S}G | 103 |
| 11352 | 0089-0108; tIR | C^{S}A^{S}A^{S}G^{S}C^{S}C^{S}C^{S}A^{S}T^{S}G^{S}G^{S}T^{S}G^{S}C^{S}A^{S}T^{S}C^{S}T^{S}G^{S}G | 104 |
| 11353 | 0073-0092; tIR | C^{S}T^{S}G^{S}G^{S}G^{S}G^{S}T^{S}C^{S}C^{S}A^{S}T^{S}C^{S}G^{S}T^{S}G^{S}G^{S}G^{S}T^{S}G^{S}C | 105 |
| 11354 | 0007-0026; 5′ UTR | C^{S}C^{S}G^{S}T^{S}G^{S}C^{S}T^{S}G^{S}C^{S}C^{S}T^{S}A^{S}C^{S}A^{S}G^{S}G^{S}A^{S}G^{S}C^{S}C | 106 |
| 11695 | 0058-0077; 5′ UTR | G^{S}G^{S}T^{S}G^{S}C^{S}T^{S}T^{S}C^{S}C^{S}G^{S}T^{S}A^{S}A^{S}G^{S}T^{S}T^{S}C^{S}T^{S}G^{S}G | 107 |
| 11696 | 0096-0117; tTR | G^{S}G^{S}A^{S}T^{S}T^{S}G^{S}C^{S}C^{S}A^{S}A^{S}G^{S}C^{S}C^{S}C^{S}A^{S}T^{S}G^{S}G^{S}T^{S}G | 108 |
| 11866 | (scrambled #11696) | C^{S}T^{S}A^{S}A^{S}G^{S}T^{S}A^{S}G^{S}T^{S}G^{S}C^{S}T^{S}A^{S}G^{S}C^{S}C^{S}G^{S}G^{S}G^{S}A | 109 |
| 11697 | 0148-0167; 5′ ORF | T^{S}G^{S}C^{S}G^{S}T^{S}C^{S}T^{S}C^{S}C^{S}A^{S}C^{S}G^{S}G^{S}A^{S}A^{S}A^{S}C^{S}A^{S}G^{S}C | 110 |
| 11698 | 0319-0338; 5′ ORF | G^{S}T^{S}G^{S}C^{S}G^{S}G^{S}C^{S}C^{S}C^{S}A^{S}G^{S}G^{S}T^{S}A^{S}C^{S}T^{S}T^{S}G^{S}G^{S}C | 111 |
| 11699 | 0832-0851; 3′ ORF | A^{S}C^{S}A^{S}A^{S}G^{S}G^{S}A^{S}G^{S}G^{S}A^{S}G^{S}G^{S}G^{S}C^{S}C^{S}A^{S}C^{S}A^{S}G^{S}T | 112 |
| 11700 | 0753-0772; 3′ ORF | T^{S}G^{S}A^{S}G^{S}A^{S}G^{S}G^{S}T^{S}T^{S}T^{S}G^{S}G^{S}A^{S}G^{S}G^{S}A^{S}A^{S}A^{S}T^{S}C | 113 |
| 11701 | 0938-0957; 3′ ORF | G^{S}A^{S}T^{S}A^{S}G^{S}T^{S}C^{S}T^{S}C^{S}T^{S}C^{S}T^{S}G^{S}T^{S}C^{S}A^{S}G^{S}C^{S}G^{S}T | 114 |
| 11702 | 0890-0909; 3′ ORF | G^{S}T^{S}T^{S}G^{S}C^{S}T^{S}G^{S}G^{S}G^{S}C^{S}C^{S}T^{S}G^{S}C^{S}T^{S}A^{S}G^{S}G^{S}C^{S}T | 115 |
| 11865 | (scrambled #11702) | C^{S}T^{S}A^{S}G^{S}G^{S}T^{S}C^{S}T^{S}C^{S}G^{S}T^{S}C^{S}G^{S}T^{S}C^{S}G^{S}G^{S}T^{S}G^{S}G | 116 |
| 11703 | 1003-1022; tIR | T^{S}C^{S}T^{S}C^{S}C^{S}A^{S}C^{S}T^{S}G^{S}C^{S}C^{S}T^{S}T^{S}C^{S}A^{S}C^{S}C^{S}T^{S}C^{S}T^{S}G^{S}C | 117 |
| 13100 | Exon 1 (Borriello et al., J. | G^{S}T^{S}A^{S}C^{S}C^{S}A^{S}G^{S}A^{S}T^{S}G^{S}A^{S}A^{S}G^{S}G^{S}T^{S}T^{S}A^{S}T^{S}C^{S}A^{S}A | 118 |
| | Immun., 1995, 155, 5490; | (2′MO) | |
| | 5′ UTR of alternative mRNA) | | |
| 13101 | Exon 4 (Borriello et al.; | C^{S}T^{S}T^{S}T^{S}G^{S}G^{S}A^{S}G^{S}A^{S}T^{S}T^{S}A^{S}T^{S}T^{S}C^{S}G^{S}A^{S}G^{S}T^{S}T | 119 |
| | 5′ UTR of alternative mRNA) | (2′MO) | |
| 13102 | Exon 5 (Borriello et al.; | G^{S}C^{S}A^{S}A^{S}G^{S}T^{S}G^{S}T^{S}A^{S}A^{S}A^{S}G^{S}C^{S}C^{S}C^{S}T^{S}G^{S}A^{S}G^{S}T | 120 |
| | 5′ UTR of alternative mRNA) | (2′MO) | |

cDNA Clones:

A cDNA encoding the sequence for human B7-1 was isolated by using the reverse transcription/polymerase chain reaction (RT-PCR). Poly A+ RNA from Daudi cells (ATCC accession No. CCL 213) was reverse transcribed using oligo-dT primer under standard conditions. Following a 30 minute reaction at 42° C. and heat inactivation, the reaction mixture (20 μL) was brought to 100 μL with water. A 10 μL aliquot from the RT reaction was then amplified in a 50 μL PCR reaction using the 5′ primer,

| | |
|---|---|
| 5′-GAT-CAG-GGT-ACC-CCA-AAG-AAA-AAG-TGA-TTT-GTC- | (sense, SEQ ID NO: 20) |
| ATT-GC-3′, and the 3′ primer, | |
| 5′-GAT-AGC-CTC-GAG-OAT--AAT-GAA-TTG-GCT-GAC-AAG- | (antisense, SEQ ID NO: 21) |
| AC-3′ | |

The primers included unique restriction sites for subcloning of the PCR product into the vector pcDNA-3 (Invitrogen, San Diego, Calif.). The 5′ primer was designed to have identity with bases 1 to 26 of the published human B7-1 sequence (Freeman et al., J. Immunol., 1989, 143, 2714; positions 13-38 of the primer) and includes a Kpn I restriction site (positions 7-12 of the primer) for use in cloning. The 3′ primer was designed to be complementary to bases 1450 to 1471 of the published sequence for B7-1 (positions 14-35 of the primer) and includes a Xho I restriction site (positions 7-12 of the primer). Following PCR, the reaction was extracted with phenol and precipitated using ethanol. The product was digested with the appropriate restriction enzymes and the full-length fragment purified by agarose gel and ligated into the vector pcDNA-3 (Invitrogen, San Diego, Calif.) prepared by digesting with the same enzymes. The resultant construct, pcB7-1, was confirmed by restriction mapping and DNA sequence analysis using standard procedures. A mouse B7-1 clone, pcmB7-1, was isolated in a similar manner by RT-PCR of RNA isolated from a murine B-lymphocyte cell line, 70Z3.

A cDNA encoding the sequence for human B7-2, position 1 to 1391, was also isolated by RT-PCR. Poly A+ RNA from Daudi cells (ATCC accession No. CCL 213) was reverse transcribed using oligo-dT primer under standard conditions. Following a 30 minute reaction at 42° C. and heat inactivation, the reaction mixture (20 μL) was brought to 100 μL with water. A 10 μL aliquot from the RT reaction was then amplified in a 50 μL PCR reaction using the 5′ primer,

| | |
|---|---|
| 5′-GAT-CAG-GGT-ACC-AGG-AGC-CTT-AGG-AGG-TAC-GG-3′, | (sense, ^{S}EQ ID NO: 1) |
| and the 3′ primer, | |
| 5′-GAT-AGC-CTC-GAG-TTA-TTT-CCA-GGT-CAT-GAG-CCA-3′. | (antisense, ^{S}EQ ID NO: 2) |

The 5′ primer was designed to have identity with bases 1-20 of the published B7-2 sequence (Azuma et al., Nature, 1993, 366, 76 and Genbank Accession No. L25259; positions 13-32 of the primer) and includes a Kpn I site (positions 7-12 of the primer) for use in cloning. The 3′ primer was designed to have complementarity to bases 1370-1391 of the published sequence for B7-2 (positions 13-33 of the primer) and includes an Xho I restriction site (positions 7-12 of the primer). Following PCR, the reaction was extracted with phenol and precipitated using ethanol. The product was digested with Xho I and Kpn I, and the full-length: fragment purified by agarose gel and ligated into the vector pcDNA-3 (Invitrogen, San Diego, Calif.) prepared by digesting with the same enzymes. The resultant construct, pcB7-2, was confirmed by restriction mapping and DNA sequence analysis using standard procedures.

A mouse B7-2 clone, pcmB7-2, was isolated in a similar manner by RT-PCR of RNA isolated from P388D1 cells using the 5′ primer,

| | |
|---|---|
| 5′-GAT-CAG-GGT-ACC-AAG-AGT-GGC-TCC-TGT-AGG-CA, | (sense, ^{S}EQ ID NO: 99) |
| and the 3′ primer, | |
| 5′-GAT-AGC-CTC-GAG-GTA-GAA-TTC-CAA-TCA-GCT-GA. | (antisense, ^{S}EQ ID NO: 100) |

The 5′ primer has identity with bases 1-20, whereas the 3′ primer is complementary to bases 1096-1115, of the published murine B7-2 sequence (Chen et al., J. Immun., 1994, 152, 4929). Both primers incorporate the respective restriction enzyme sites found in the other 5′ and 3′ primers used to prepare cDNA clones. The RT-PCR product was restricted with Xho I and Kpn I and ligated into pcDNA-3 (Invitrogen, Carlsbad, Calif.).

Other cDNA clones, corresponding to mRNAs resulting from alternative splicing events, are cloned in like fashion, using primers containing the appropriate restriction sites and having identity with (5′ primers), or complementarity to (3′ primers), the selected B7 mRNA.

### Example 2

### Modulation of hB7-1 Expression by Oligonucleotides

The ability of oligonucleotides to inhibit B7-1 expression was evaluated by measuring the cell surface expression of B7-1 in transfected COS-7 cells by flow cytometry.

Methods:

A T-175 flask was seeded at 75% confluency with COS-7 cells (ATCC accession No. CRL 1651). The plasmid pcB7-1 was introduced into cells by standard calcium phosphate transfection. Following a 4 hour transfection, the cells were trypsinized and seeded in 12-well dishes at 80% confluency. The cells were allowed to adhere to the plastic for 1 hour and were then washed with phosphate-buffered saline (PBS). OptiMEM™ (GIBCO-BRL, Gaithersburg, Md.) medium was added along with 15 μg/mL of Lipofectin™ (GIBCO-BRL, Gaithersburg, Md.) and oligonucleotide at the indicated concentrations. After four additional hours, the cells were washed with phosphate buffered saline (PBS) and incubated with fresh oligonucleotide at the same concentration in DMEM (Dulbecco et al., Virol., 1959, 8, 396; Smith et al., Virol., 1960, 12, 185) with 10% fetal calf sera (FCS).

In order to monitor the effects of oligonucleotides on cell surface expression of B7-1, treated COS-7 cells were harvested by brief trypsinization 24-48 hours after oligonucleotide treatment. The cells were washed with PBS, then resuspended in 100 μL of staining buffer (PBS, 0.2% BSA, 0.1% azide) with 5 μL conjugated anti-B7-1-antibody (i.e., anti-hCD80-FITC, Ancell, Bayport, Minn., FITC: fluorescein isothiocyanate). The cells were stained for 30 minutes at 4° C., washed with PBS, resuspended in 300 μL containing 0.5% paraformaldehyde. Cells were harvested and the fluorescence profiles were determined using a flow cytometer.

Results:

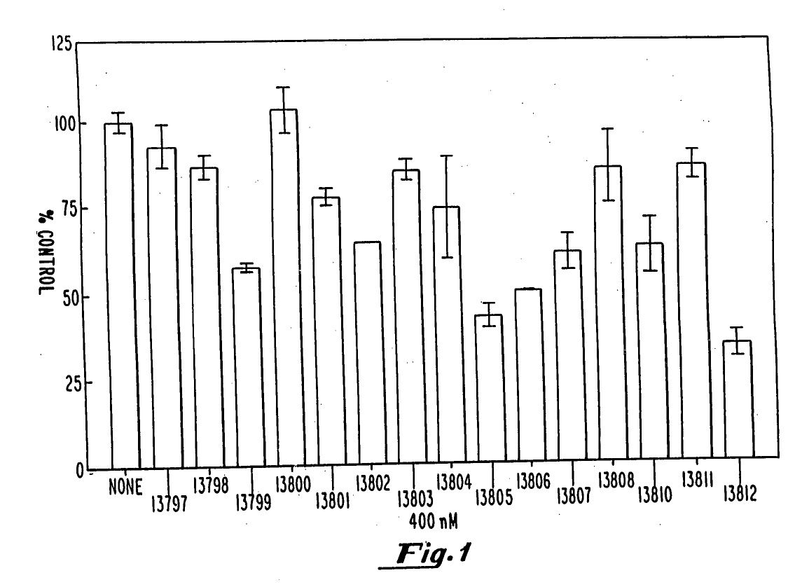

The oligonucleotides shown in Table 1 were evaluated, in COS-7 cells transiently expressing B7-1 cDNA, for their ability to inhibit B7-1 expression. The results (FIG. 1) identified ISIS 13805, targeted to the translation initiation codon region, and ISIS 13812, targeted to the 3′ untranslated region (UTR), as the most active oligonucleotides with greater than 50% inhibition of B7-1 expression. These oligonucleotides are thus highly preferred. ISIS 13799.(targeted to the 5′ untranslated region), ISIS 13802 (targeted to the 5′ untranslated region), ISIS 13806 and 13807 (both targeted to the 5′ region of the ORF), and ISIS 13810 (targeted to the central portion of the ORF) demonstrated 35% to 50% inhibition of B7-1 expression. These sequences are therefore also preferred. Oligonucleotide ISIS 13800, which showed essentially no inhibition of B7-1 expression in the flow cytometry assay, and ISIS Nos. 13805 and 13812 were then evaluated for their ability to inhibit cell surface expression of B7-1 at various concentrations of oligonucleotide. The results of these assays are shown in FIG. 2. ISIS 13812 was a superior inhibitor of B7-1 expression with an IC₅₀ of approximately 150 nM. ISIS 13800, targeted to the 5′ UTR, was essentially inactive.

### Example 3

### Modulation of hB7-2 Protein by Oligonucleotides

In an initial screen, the ability of hB7-2 oligonucleotides to inhibit B7-2 expression was evaluated by measuring the cell surface expression of B7-2 in transfected COS-7 cells by flow cytometry. The methods used were similar to those given in Example 2, with the exceptions that (1) COS-7 cells were transfected with the plasmids pbcB7-2 or BBG-58, a human ICAM-1 (CD54) expression vector (R&D Systems, Minneapolis, Minn.) introduced into cells by standard calcium phosphate transfection, (2) the oligonucleotides used were those described in Table 2, and (3) a conjugated anti-B7-2 antibody (i.e., anti-hCD86-FITC or anti-CD86-PE, PharMingen, San Diego, Calif.; PE: phycoerythrin) was used during flow cytometry.

Results:

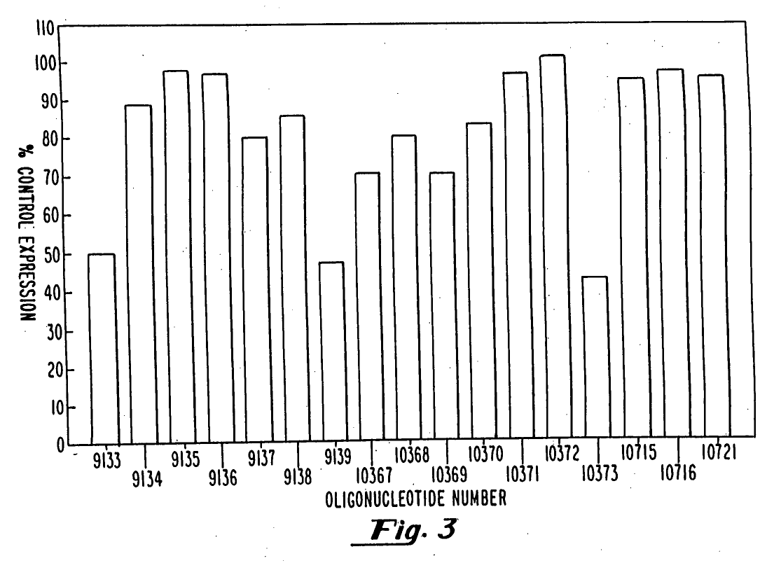

The results are shown in FIG. 3. At a concentration of 200 nM, ISIS 9133, ISIS 9139 and ISIS 10373 exhibited inhibitory activity of 50% or better and are therefore highly preferred. These oligonucleotides are targeted to the 3′ untranslated region (ISIS 9133), the translation initiation codon region (ISIS 9139) and the 5′ untranslated region (ISIS 10373). At the same concentration, ISIS 10715, ISIS 10716 and ISIS 10721, which are scrambled controls for ISIS 9133, ISIS 9139 and ISIS 10373, respectively, showed no inhibitory activity. Treatment with ISIS 10367 and ISIS 10369 resulted in greater than 25% inhibition, and these oligonucleotides are thus also preferred. These oligonucleotides are targeted to the 5′ (ISIS 10367) and 3′ (ISIS 10369) untranslated regions.

### Example 4

### Modulation of hB7-2 mRNA by Oligonucleotides

Methods:

For ribonuclease protection assays, cells were harvested 18 hours after completion of oligonucleotide treatment using a Totally RNA™ kit (Ambion, Austin, Tex.). The probes for the assay were generated from plasmids pcB7-2 (linearized by digestion with Bgl II) and pTR1-b-actin (Ambion Inc., Austin, Tex.). In vitro transcription of the linearized plasmid from the SP6 promoter was performed in the presence of a-32P-UTP (800 Ci/mmole) yielding an antisense RNA complementary to the 3′ end of B7-2 (position 1044-1391). The probe was gel-purified after treatment with DNase I to remove DNA template. Ribonuclease protection assays were carried out using an RPA II™ kit (Ambion) according to the manufacturer's directions. Total RNA (5 μg) was hybridized overnight, at 42° C., with 105 cpm of the B7-2 probe or a control beta-actin probe. The hybridization reaction was then treated, at 37⁹C for 30 minutes, with 0.4 units of RNase A and 2 units of RNase T1. Protected RNA was precipitated, resuspended in 10 μL of gel loading buffer and electrophoresed on a 6% acrylamide gel with 50% w/v urea at 20 W. The gel was then exposed and the lanes quantitated using a PhosphorImager (Molecular Dynamics, Sunnyvale, Calif.) essentially according to the manufacturer's instructions.

Results:

The extent of oligonucleotide-mediated hB7-2 mRNA modulation generally paralleled the effects seen for hB7-2 protein (Table 5). As with the protein expression (flow cytometry assays, the most active oligonucleotides were ISIS 9133, ISIS 9139 and 10373. None of the oligonucleotides tested had an inhibitory effect on the expression of of b-actin mRNA in the same cells.

TABLE 5
Activities of Oligonucleotides Targeted to hB7-2 mRNA

| | | % Control | % Control RNA |
|---|---|---|---|
| ISIS NO. | SEQ ID NO. | Protein | Expression |
| 9133 | 3 | 70.2 | 46.0 |
| 9134 | 4 | 88.8 | 94.5 |
| 9135 | 5 | 98.2 | 83.4 |
| 9136 | 6 | 97.1 | 103.1 |
| 9137 | 7 | 80.5 | 78.1 |
| 9138 | 8 | 86.4 | 65.9 |
| 9139 | 9 | 47.9 | 32.6 |
| 10367 | 10 | 71.3 | 52.5 |
| 10368 | 11 | 81.0 | 84.5 |
| 10369 | 12 | 71.3 | 81.5 |
| 10370 | 13 | 84.3 | 83.2 |
| 10371 | 14 | 97.3 | 92.9 |
| 10372 | 15 | 101.7 | 82.5 |
| 10373 | 16 | 43.5 | 32.7 |

### Example 5

### Additional hB7-1 and hB7-2 Oligonucleotides

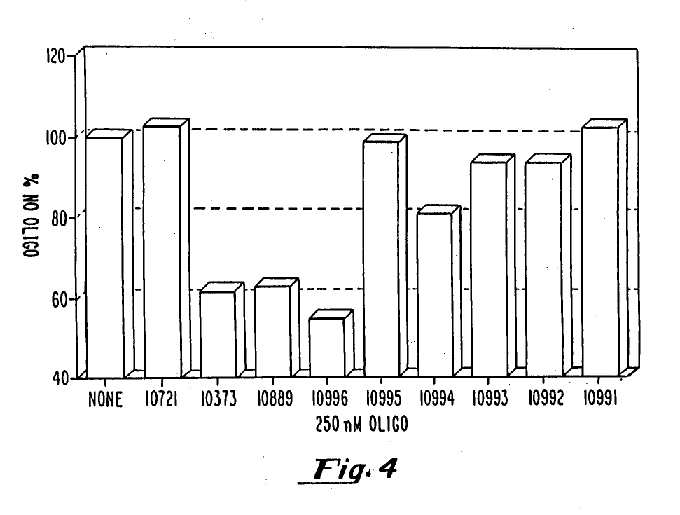

Oligonucleotides having structures and/or sequences that were modified relative to the oligonucleotides identified during the initial screening were prepared. These oligonucleotides were evaluated for their ability to modulate human B7-2 expression using the methods described in the previous examples. ISIS 10996, an oligonucleotide having a 15 nucleotide sequence derived from the 20 nucleotide sequence of ISIS 10373, was also prepared and evaluated. ISIS 10996 comprises 15 nucleotides, 51-GCG-AGC-TCC-CCG-TAC (SEQ ID NO: 90) contained within the sequence of ISIS 10373. Both ISIS 10373 and 10996 overlap a potential stem-loop structure located within the B7-2 message comprising bases 1-67 of the sequence of hB7-2 presented by Azuma et al. (Nature, 1993, 366, 76). While not intending to be bound by any particular theory regarding their mode(s) of action, ISIS 10373 and ISIS 10996 have the potential to bind as loop 1 pseudo-half-knots at a secondary structure within the target RNA. U.S. Pat. No. 5,5152,438, the contents of which are hereby incorporated by reference, describes methods for modulating gene expression by the formation of pseudo-half-knots. Regardless of their mode(s) of action, despite having a shorter length than ISIS 10373, the 15-mer ISIS 10996 is as (or more) active in the B7-2 protein expression assay than the 20-mer from which it is derived (FIG. 4; ISIS 10721 is a scrambled control for ISIS 10373). A related 16-mer, ISIS 10889, was also active in the B7-2 protein expression assay. However, a structurally related 14-mer (ISIS 10995), 13-mer (ISIS 10994), 12-mer (ISIS 10993), 11-mer (ISIS 10992) and 10-mer (ISIS 10991) exhibited little or no activity in this assay. ISIS 10996was further derivatized in the following ways.

ISIS 10996 derivatives having 2′ methoxethoxy substitutions were prepared, including a fully substituted derivative (ISIS 11539), “gapmers” (ISIS 11541 and 11543) and “wingmers” (ISIS 11545 and 11547). As explained in Example 5, the 2′ methoxyethoxy substitution prevents the action of some nucleases (e.g., RNase H) but enhances the affinity of the modified oligonucleotide for its target RNA molecule. These oligonucleotides are tested for their ability to modulate hB7-2 message or function according to the methods of Examples 3, 4, 7 and 8.

ISIS 10996 derivatives were prepared in order to be evaluated for their ability to recruit RNase L to a target RNA molecule, e.g., hB7-2 message. RNase L binds to, and is activated by, (2′-5′)(A)ₙ, which is in turn produced from ATP by (2′-5′)(A)ₙ synthetase upon activation by, e.g., interferon. RNase L has been implicated in antiviral mechanisms and in the regulation of cell growth as well (Sawai, Chemica Scripta, 1986, 21, 169; Charachon et al., Biochemistry 1990, 29, 2550). The combination of anti-B7 oligonucleotides conjugated to (2′-5′) (A)ₙ is expected to result in the activation of RNase L and its targeting to the B7 message complementary to the oligonucleotide sequence. The following oligonucleotides have identical sequences (i.e., that of ISIS 10996) and identical (2′-5′)(A)₄ “caps” on their 5′ termini: ISIS 12492, 12495, 12496 and 13107. The adenosyl residues have 3′ hydroxyl groups and are linked to each other by phosphorothioate linkages. The (3′-5′) portion of the oligonucleotide, which has a sequence complementary to a portion of the human B7-2 RNA, is conjugated to the (2′-5′)(A)₄ “cap” via a phosphorothioate linkage from the 5′ residue of the (3′-5′) portion of the oligonucleotide to an n-aminohexyl linker which is bonded to the cap” via another phosphorothioate linkage. In order to test a variety of chemically diverse oligonucleotides of this type for their ability to recruit RNase L to a specific message, different chemical modifications were made to this set of four oligonucleotides as follows. ISIS 12496 consists of unmodified oligonucleotides in the (3′-5′) portion of the oligonucleotide. In ISIS 13107, phosphorothioate linkages replace the phosphate linkages found in naturally occurring nucleic acids. Phosphorothioate linkages are also employed in ISIS 12492 and 12495, which additionally have 2′-methoxyethoxy substitutions. These oligonucleotides are tested for their ability to modulate hB7-2 message or function according to the methods of Examples 3, 4, 7 and 8.

Derivatives of ISIS 109:96 having modifications at the 2′ position were prepared and evaluated. The modified oligonucleotides included ISIS 11539 (fully 2′-O-methyl), ISIS 11541 (having 2′-o-methyl “wings” and a central 7-base “gap”), ISIS 11543 (2′-O-methyl wings with a 9-base gap), ISIS 11545 (having a 5′ 2′-O-methyl wing) and ISIS 11547 (having a 3′ 2′-O-methyl wing). The results of assays of 2′-O-methyl oligonucleotides were as follows. ISIS 11539, the fully 2-O-methyl version of ISIS 10996, was not active at all in the protein expression assay. The gapped and winged oligonucleotides (ISIS 11541, 11543, 11545 and 11547) each showed some activity at 200 nM (i.e., from 0.60 to 70% expression relative to untreated cells), but less than that demonstrated by the parent compound, ISIS 10996 (i.e., about 50% expression). Similar results were seen in RNA expression assays.

ISIS 10782, a derivative of ISIS 10373 to which cholesterol has been conjugated via a 51 n-aminohexyl linker, was prepared. Lipophilic moieties such as cholesterol have been reported to enhance the uptake by cells of oligonucleotides in some instances, although the extent to which uptake is enhanced, if any, remains unpredictable. ISIS 10782, and other oligonucleotides comprising lipophilic moieties, are tested for their ability to modulate B7-2 message or function according to the methods of Examples 3, 4, 7 and 8.

A series of 2′-methoxyethoxy (herein, 2“ME”) and 2′-fluoride (herein, “2′F”) “gapmer” derivatives of the hB7-1 oligonucleotides ISIS 12361 (ISIS Nos. 12348 and 12473, respectively), ISIS 12362 (ISIS Nos. 12349 and 12474), ISIS 12363 (ISIS Nos. 12350 and 12475), ISIS 12364 (ISIS Nos. 12351 and 12476), ISIS 12365 (ISIS Nos. 12352 and 12477), ISIS 12366 (ISIS Nos. 12353 and 12478), ISIS 12367 (ISIS Nos. 12354 and 12479), ISIS 12368. (ISIS Nos. 12355 and 12480), ISIS 12369 (ISIS Nos. 12356 and 12481) and ISIS 12370 (ISIS Nos. 12357 and, 12482)-were prepared. The central, non-2′-modified portions (Agaps@) of these derivatives support RNase H activity when the oligonucleotide is bound to its target RNA, even though the 2′-modified portions do not. However, the 2′-modified “wings” of these oligonucleotides enhance their affinity to their target RNA molecules (Cook, Chapter 9 In: Antisense Research and Applications, Crooke et al., eds., CRC Press, Boca Raton, 1993, pp. 171-172).

Another 2′ modification is the introduction of a methoxy (MO) group at this position. Like 2′ME- and 2′F-modified oligonucleotides, this modification prevents the action of RNase H on duplexes formed from such oligonucleotides and their target RNA molecules, but enhances the affinity of an oligonucleotide for its target RNA molecule. ISIS 12914 and 12915 comprise sequences complementary to the 5′ untranslated region of alternative hB7-1 mRNA molecules, which arise from alternative splicing events of the primary hB7-1 transcript. These oligonucleotides include 2′ methoxy modifications, and the enhanced target affinity resulting therefrom may allow for greater activity against alternatively spliced B7-1 mRNA molecules which may be present in low abundance in some tissues (Inobe et al., J. Immun., 1996, 157, 582). Similarly, ISIS 13498 and 13499, which comprise antisense sequences to other alternative hB7-1 mRNAs, include 2′ methoxyethoxy modifications in order to enhance their affinity for their target molecules, and 2′ methoxyethoxy or 2′methoxy substitutions are incorporated into the hB7-2 oligonucleotides ISIS 12912, 12913, 13496 and 13497. These oligonucleotides are tested for their ability to modulate hB7-1 essentially according to the methods of Example 2 or hB7-2 according to the methods of Examples 3, 4, 7 and 8, with the exception that, when necessary, the target cells are transfected with a cDNA clone corresponding to the appropriate alternatively spliced B7 transcript.

### Example 6

### Specificity of Antisense Modulation

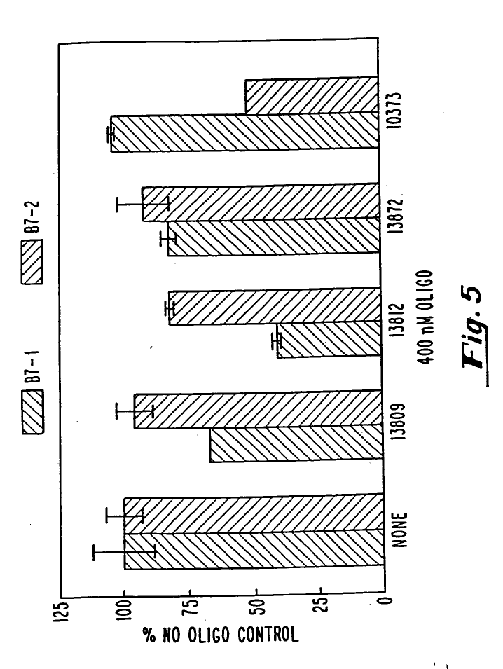

Several oligonucleotides of the invention were evaluated in a cell surface expression flow cytometry assay to determine the specificity of the oligonucleotides for B7-1 as contrasted with activity against B7-2. The oligonucleotides tested in this assay included ISIS 13812, an inhibitor of B7-1 expression (FIG. 1; Example 2) and ISIS 10373, an inhibitor of B7-2 expression (FIG. 3; Example 3). The results of this assay are shown in FIG. 5. ISIS 13812 inhibits B7-1 expression with little or no effect on B7-2 expression. As is also seen in FIG. 5, ISIS 10373 inhibits B7-2 expression with little or no effect on B7-1 expression. ISIS 13872 (SEQ ID NO: 37, AGT-CCT-ACT-ACC-AGC-CGC-CT), a scrambled control of ISIS 13812, and ISIS 13809 (SEQ ID NO: 51) were included in these assays and demonstrated essentially no activity against either B7-1 or B7-2.

### Example 7

### Modulation of hB7-2 Expression by Oligonucleotides in Antigen Presenting Cells

The ability of ISIS 10373 to inhibit expression from the native B7-2 gene in antigen presenting cells (APCs) was evaluated as follows.

Methods:

Monocytes were cultured and treated with oligonucleotides as follows. For dendritic cells, EDTA-treated blood was layered onto Polymorphprep™ (1.113 g/mL; Nycomed, Oslo, Norway) and sedimented at 500×g for 30 minutes at 20° C. Mononuclear cells were harvested from the interface. Cells were washed with PBS, with serum-free RPMI media (Moore et al., N.Y. J. Med., 1968, 68, 2054) and then with RPMI containing 5% fetal bovine serum (FBS). Monocytes were selected by adherence to plastic cell culture cell culture dishes for 1 h at 37° C. After adherence, cells were treated with oligonucleotides in serum-free RPMI containing Lipofectin™ (8 μg/mL). After 4 hours, the cells were washed. Then RPMI containing 5% FBS and oligonucleotide was added to cells along with interleukin-4 (IL-4; R&D Systems, Minneapolis, Minn.) (66 ng/mL) and granulocyte-macrophage colony-stimulating factor (GM-CSF; R&D Systems, Minneapolis, Minn.) (66 ng/mL) to stimulate differentiation (Romani et al., J. Exp. Med., 1994, 180, 83, 1994). Cells were incubated for 48 hours, after which cell surface expression of various molecules was measured by flow cytometry.

Mononuclear cells isolated from fresh blood were treated with oligonucleotide in the presence of cationic lipid to promote cellular uptake. As a control oligonucleotide, ISIS 2302.(an inhibitor of ICAM-1 expression; SEQ ID NO: 17) was also administered to the cells. Expression of B7-2 protein was measured by flow cytometry according to the methods of Example 2. Monoclonal antibodies not described in the previous Examples included anti-hCD3 (Ancell, Bayport, MN) and anti-HLA-DR (Becton Dickinson, San Jose, Calif.).

Results:

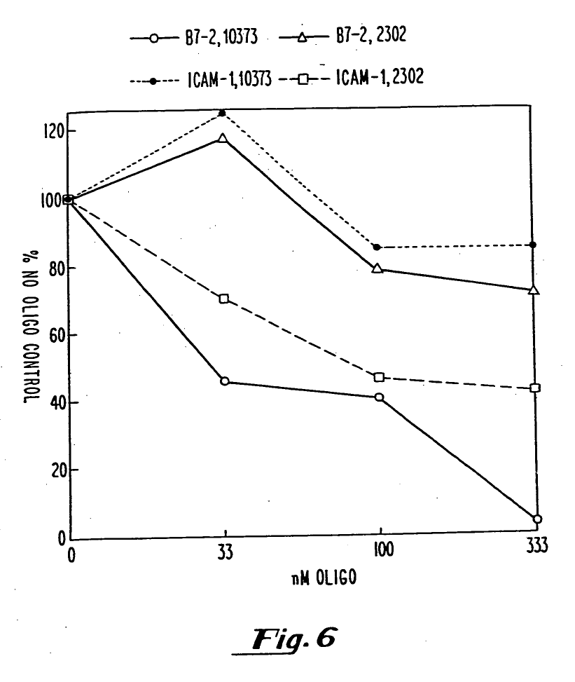

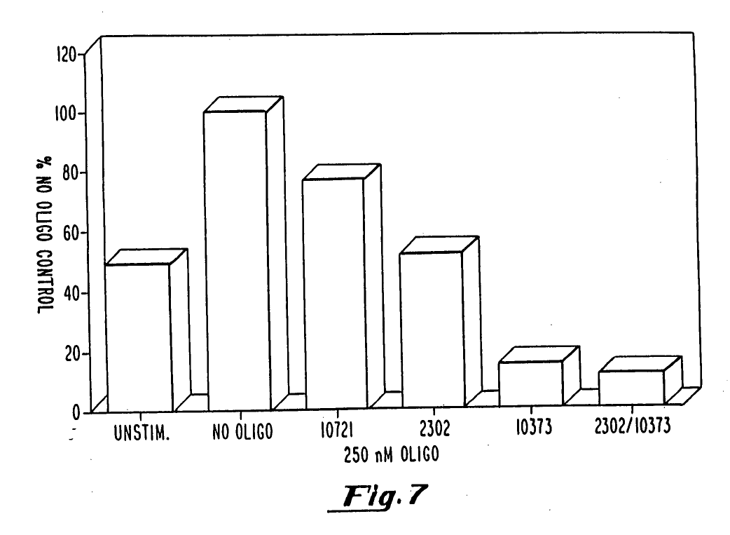

As shown in FIG. 6, ISIS 10373 has a significant inhibitory effect on B7-2 expression with an IC₅₀ of approximately 250 nM. ISIS 10373 had only a slight effect on ICAM-1 expression even at a dose of 1 μM. ISIS 2302 (SEQ ID NO: 17), a control oligonucleotide which has been shown to inhibit ICAM-1 expression, had no effect on B7-2 expression, but significantly decreased ICAM-1 levels with an IC₅₀ of approximately 250 nM. Under similar conditions, ISIS 10373 did not affect the cell surface expression of B7-1, HLA-DR or CD3 as measured by flow cytometry.

### Example 8

### Modulation of T Cell Proliferation by Oligonucleotides

The ability of ISIS 2302 and ISIS 10373 to inhibit T cell proliferation was evaluated as follows. Monocytes treated with oligonucleotide and cytokines (as in Example 6) were used as antigen presenting cells in a T cell proliferation assay. The differentiated monocytes were combined with CD4+ T cells from a separate donor. After 48 hours, proliferation was measured by [³H] thymidine incorporation.

Methods:

For T cell proliferation assays, cells were isolated from EDTA-treated whole blood as described above, except that a faster migrating band containing the lymphocytes was harvested from just below the interface. Cells were washed as described in-Example 6 after which erythrocytes were removed by NH₄Cl lysis. T cells were purified using a T cell enrichment column (R&D Systems, Minneapolis, Minn.) essentially according to the manufacturer's directions. CD4+ T cells were further enriched from the entire T cell population by depletion of CD8+ cells with anti-CD8-conjugated magnetic beads (AMAC, Inc., Westbrook, Me.) according to the manufacturer's directions. T cells were determined to be>80% CD4+by flow cytometry using Cy-chrome-conjugated anti-CD4 mAb (PharMingen, San Diego, Calif.).

Antigen presenting cells (APCs) were isolated as described in Example 6 and treated with mitomycin C (25 μg/mL) for 1 hour then washed 3 times with PBS. APCs (10⁵ cells) were then combined with 4×10⁴ CD4+ T cells in 350 μL of culture media. Where indicated, purified CD3 mAb was also added at a concentration of 1 μg/mL. During the last 6 hours of the 48 hour incubation period, proliferation was measured by determining uptake of 1.5 uCi of [³H]-thymidine per well. The cells were harvested onto filters and the radioactivity measured by scintillation counting.

Results:

As shown in FIG. 7, mononuclear cells which were not cytokine-treated slightly induced T cell proliferation, presumably due to low levels of costimulatory molecules expressed on the cells. However, when the cells were treated with cytokines and induced to differentiate to dendritic-like cells, expression of both ICAM-1 and B7-2 was strongly upregulated. This resulted in a strong T cell proliferative response which could be blocked with either anti-ICAM-1 (ISIS 2302) or anti-B7-2 (ISIS 10373) oligonucleotides prior to induction of the mononuclear cells. The control oligonucleotide (ISIS 10721) had an insignificant effect on T cell proliferation. A combination treatment with both the anti-ICAM-1 (ISIS 2302) and anti-B7-2 (ISIS 10373) oligonucleotides resulted in a further decrease in T cell response.

### Example 9

### Modulation of Murine B7 Genes by Oligonucleotides

Oligonucleotides (see Table 4) capable of inhibiting expression of murine B7-2 transiently expressed in COS-7 cells were identified in the following manner. A series of phosphorothioate oligonucleotides complementary to murine B7-2 (mB7-2) cDNA were screened for their ability to reduce mB7-2 levels (measured by flow cytometry as in Example 2, except that a conjugated anti-mB7-2 antibody (i.e., anti-mCD86-PE, PharMingen, San Diego, Calif.) in COS-7 cells transfected with an mB7-2 cDNA clone. Anti-mB7-2 antibody may also be obtained from the hybridoma deposited at the ATCC under accession No. HB-253. Oligonucleotides (see Table 2) capable of modulating murine B7-1 expression are isolated in like fashion, except that a conjugated anti-mB7-1 antibody is used in conjunction with COS-7 cells transfected with an mB7-1 cDNA clone.

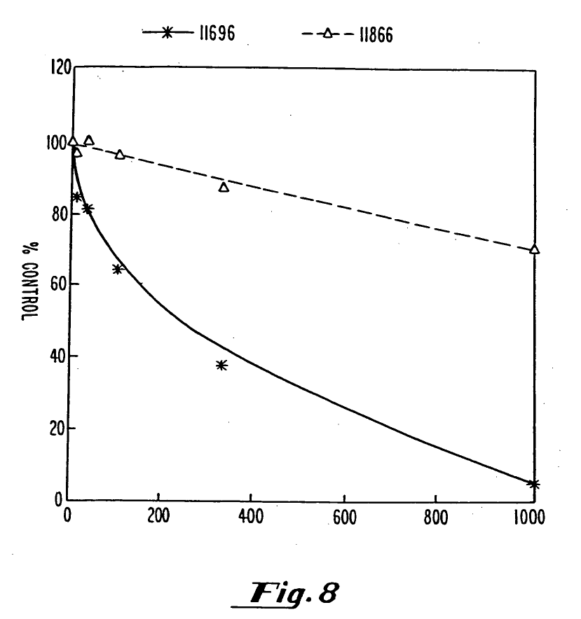

For murine B7-2, the most active oligonucleotide identified was ISIS 11696 (GGA-TTG-CCA-AGC-CCA-TGG-TG, SEQ ID NO: 18), which is complementary to position 96-115 of the cDNA, a site which includes the translation initiation (AUG) codon. FIG. 8 shows a dose-response curve for ISIS 11696 and a scrambled control, ISIS 11866 (CTA-AGT-AGT-GCT-AGC-CGG-GA, SEQ ID NO: 19). ISIS 11696 inhibited cell surface expression of B7-2 in COS-7 cells with an IC₅₀ in the range of 200-300 nM, while ISIS 11866 exhibited less than 20% inhibition at the highest concentration tested (1000 nM).

In order to further evaluate the murine B7-2 antisense oligonucleotides, the IC-21 cell line was used. IC-21 monocyte/macrophage cell line expresses both B7-1 and murine B7-2 (mB7-2) constitutively. A 2-fold induction of expression can be achieved by incubating the cells in the presence of lipopolysaccharide (LPS; GIBCO-BRL, Gaithersburg, Md.) (Hathcock et al., Science, 1993, 262, 905).

IC-21 cells (ATCC; accession No. TIB 186) were seeded at 80% confluency in 12-well plates in DMEM media with 10% FCS. The cells were allowed to adhere to the plate overnight. The following day, the medium was removed and the cells were washed with PBS. Then 500 μL of OPtiMEM™.(GIBCO-BRL, Gaithersburg, Md.) supplemented with 15 μg/mL of Lipofectin™ (GIBCO-BRL, Gaithersburg, Md.) was added to each well. Oligonucleotides were then added directly to the medium at the indicated concentrations. After incubation for 4 hours, the cells were washed with PBS and incubated overnight in culture medium supplemented with 15 μg/mL of LPS. The following day, cells were harvested by scraping, then analyzed for cell surface expression by flow cytometry.

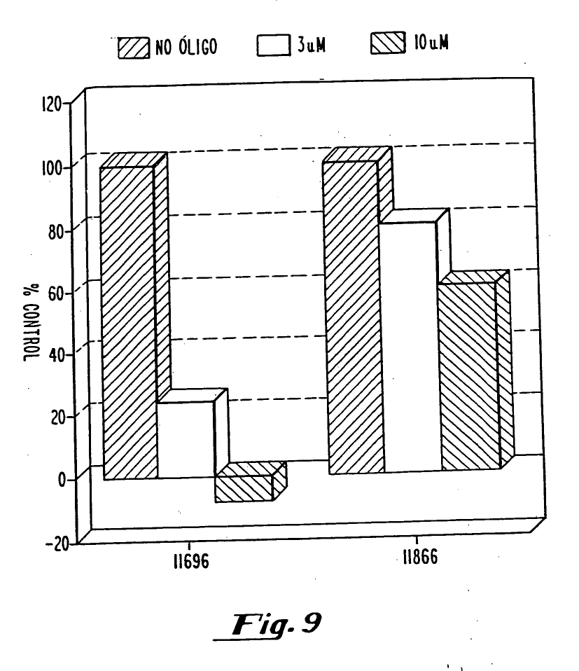

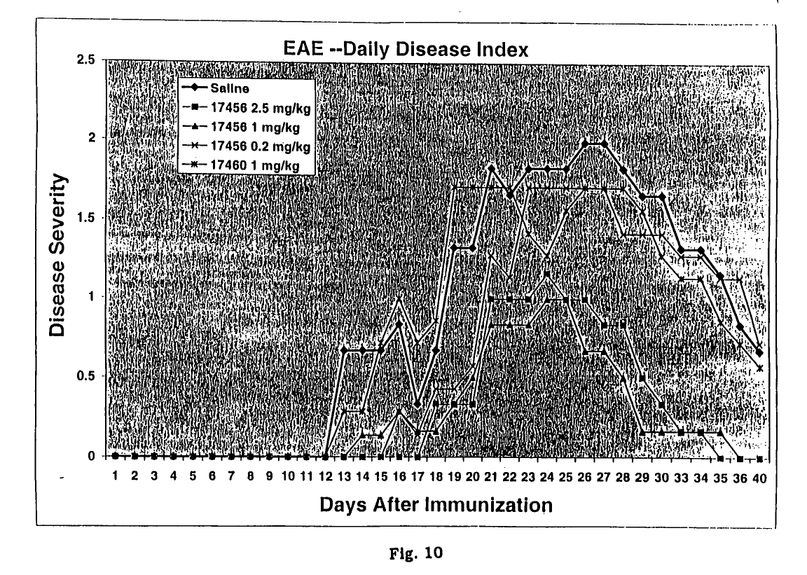

ISIS 11696 and ISIS 11866 were administered to IC-21 cells in the presence of Lipofectin™ (GIBCO-BRL, Gaithersburg, Md.). The results are shown in FIG. 9. At a concentration of 10 uM, ISIS 11696 inhibited mB7-2 expression completely (and decreased mB7-2 levels below the constitutive level of expression), while the scrambled control oligonucleotide, ISIS 11866, produced only a 40% reduction in the level of induced expression. At a concentration of 3 uM, levels of induced expression were greatly reduced by ISIS 11696, while ISIS 11866 had little effect.

Modified oligonucleotides, comprising 2′ substitutions (e.g., 2′ methoxy, 2′ methoxyethoxy) and targeted to alternative transcripts of murine B7-1 (ISIS 12914, 12915, 13498, 13499) or murine B7-2 (ISIS 13100, 13100 and 13102) were prepared. These oligonucleotides are tested for their ability to modulate murine B7 essentially according to the above methods using IC-21 cells or COS-7 transfected with a cDNA clone corresponding to the appropriate alternatively spliced B7 transcript.

### Example 10

### Modulation of Allograft Rejection by Oligonucleotides

A murine model for evaluating compounds for their ability to inhibit heart allograft rejection has been previously described:(Stepkowski et al., J. Immunol., 1994, 153, 5336). This model was used to evaluate the immunosuppressive capacity of antisense oligonucleotides to B7 proteins alone or in combination with antisense oligonucleotides to intercellular adhesion molecule-1 (ICAM-1).

Methods:

Heart allograft rejection studies and oligonucleotide treatments of BALB/c mice were performed essentially as previously described (Stepkowski et al., J. Immunol., 1994, 153, 5336). Antisense oligonucleotides used included ISIS 11696, ISIS 3082 (targeted to ICAM-1) and ISIS 1082 (a control oligonucleotide targeted to the herpes virus UL-13 gene sequence). Dosages used were 1, 2, 2.5, 5 or 10 mg/kg of individual oligonucleotide (as indicated below); when combinations of oligonucleotides were administered, each oligonucleotide was given at a dosage of 1, 5 or 10 mg/kg (total oligonucleotide dosages of 2, 10 and 20 mg/kg, respectively). The survival times of the transplanted hearts and their hosts were monitored and recorded.

Results:

The mean survival time for untreated mice was 8.2±0.8 days (7, 8, 8, 8, 9, 9 days). Treatment of the mice for 7 days with ISIS 1082 (SEQ ID NO: 125, unrelated control oligonucleotide) slightly reduced the mean survival times to 7.1±0.7 days (5 mg/kg/day; 6, 7, 7, 7, 8, 8) or 7.0±0.8 days (10 mg/kg/day; 6, 7, 7, 8). Treatment of the mice for seven days with the murine B7-2 oligonucleotide ISIS 11696 (SEQ ID NO: 108) increased the mean survival time to 9.3 days at two doses (2 mg/kg/day, 9.3±0.6 days, 9, 9, 10; 10 mg/kg/day, 9.3±1.3 days, 8, 9, 9, 11). Treatment of mice for seven days with an ICAM-1 oligonucleotide, ISIS 3082, also increased the mean survival of the mice over several doses Specifically, at 1 mg/kg/day, the mean survival time (MSD) was 11.0±0.0 (11, 11, 11); at 2.5 mg/kg/day, the MSD was 12.0±2.7 (10, 12, 13, 16); at 5 mg/kg/day, the MSD was 14.1±2.7 (10, 12, 12, 13, 16, 16, 17, 17); and, at 10 mg/kg/day, the MSD was 15.3±5.8 (12, 12, 13, 24). Some synergistic effect was seen when the mice were treated for seven days with 1 mg/kg/day each of ISIS 3082 and 11696: the MSD was 13.8±1.0 (13, 13, 14, 15)

### Example 11

### Detection of Nucleic Acids Encoding B7 Proteins

Oligonucleotides are radiolabeled after synthesis by ³²P-labeling at the 5′ end with polynucleotide kinase. Sambrook et al., “Molecular Cloning. A Laboratory Manual,” Cold Spring Harbor Laboratory Press, 1989, Volume 2, pg. 11.31. Radiolabeled oligonucleotide capable of hybridizing to a nucleic acid encoding a B7 protein is contacted with a tissue or cell sample suspected of B7 protein expression under conditions in which specific hybridization can occur, and the sample is washed to remove unbound oligonucleotide. A similar control is maintained wherein the radiolabeled oligonucleotide is contacted with a normal tissue or cell sample under conditions that allow specific hybridization, and the sample is washed to remove unbound oligonucleotide. Radioactivity remaining in the samples indicates bound oligonucleotide and is quantitated using a scintillation counter or other routine means. A greater amount of radioactivity remaining in the samples, as compared to control tissues or cells, indicates increased expression of a B7 gene, whereas a lesser amount of radioactivity in the samples relative to the controls indicates decreased expression of a B7 gene.

Radiolabeled oligonucleotides of the invention are also useful in autoradiography. A section of tissues suspected of expressing a B7 gene is treated with radiolabeled oligonucleotide and washed as described above, then exposed to photographic emulsion according to standard autoradiography procedures. A control of a normal tissue section is also maintained. The emulsion, when developed, yields an image of silver grains over the regions expressing a B7 gene, which is quantitated. The extent of B7 expression is determined by comparison of the silver grains observed with control and test samples.

Analogous assays for fluorescent detection of expression of a B7 gene use oligonucleotides of the invention which are labeled with fluorescein or other fluorescent tags. Labeled oligonucleotides are synthesized on an automated DNA synthesizer (Applied Biosystems, Foster City, Calif.) using standard phosphoramidite chemistry. b-Cyanoethyldiisopropyl phosphoramidites are purchased from Applied Biosystems (Foster City, Calif.). Fluorescein-labeled amidites are purchased from Glen Research (Sterling, Va.). Incubation of oligonucleotide and biological sample is carried out as described above for radiolabeled oligonucleotides except that, instead of a scintillation counter, a fluorescence microscope is used to detect the fluorescence. A greater amount of fluorescence in the: samples, as compared to control tissues or cells, indicates increased expression of a B7 gene, whereas a lesser amount of fluorescence in the samples relative to the controls indicates decreased expression of a B7 gene.

### Example 12

### Chimeric (Deoxy Gapped) Human B7-1 Antisense Oligonucleotides

Additional oligonucleotides targeting human B7-1 were synthesized. Oligonucleotides were synthesized as uniformly phosphorothioate chimeric oligonucleotides having regions of five 2′-O-methoxyethyl (2′-MOE) nucleotides at the wings and a central region of ten deoxynucleotides. Oligonucleotide sequences are shown in Table 6.

Oligonucleotides were screened as described in Example 4. Results are shown in Table 7.

Oligonucleotides 22315 (SEQ ID NO: 128), 22316 (SEQ ID NO: 26), 22317 (SEQ ID NO: 129), 22320 (SEQ ID NO: 132), 22324 (SEQ ID NO: 135), 22325 (SEQ ID NO: 136), 22334 (SEQ ID NO: 145), 22335 (SEQ ID NO: 146), 22337 (SEQ ID NO: 148), and 22338 (SEQ ID NO: 36) resulted in 50% or greater inhibition of B7-1 mRNA in this assay.

TABLE 6

Nucleotide ^{S}equences of Human B7-1 Chimeric
(deoxy gapped) Oligodeoxynucleotides

| | | | | |
|---|---|---|---|---|
| | | | TARGET GENE | |
| | | SEQ | NUCLEOTIDE | GENE |
| ISIS | NUCLEOTIDE SEQUENCE¹ | ID | CO- | TARGET |
| NO. | (5′−>3′) | NO: | ORDINATES² | REGION |

| | | | | |
|---|---|---|---|---|
| 22313 | AGACTCCACTTCTGAGATGT | 126 | 0048-0067 | 5′-UTR |
| 22314 | TGAAGAAAAATTCCACTTTT | 127 | 0094-0113 | 5′-UTR |
| 22315 | TTTAGTTTCACAGCTTGCTG | 128 | 0112-0129 | 5′-UTR |
| 22316 | GCTCACGTAGAAGACCCTCC | 26 | 0193-0212 | 5′-UTR |
| 22317 | TCCCAGGTGCAAAACAGGCA | 129 | 0233-0252 | 5′-UTR |
| 22318 | GTGAAAGCCAACAATTTGGA | 130 | 0274-0293 | 5′-UTR |
| 22319 | CATGGCTTCAGATGCTTAGG | 131 | 0301-0320 | AUG |
| 22320 | TTGAGGTATGGACACTTGGA | 132 | 0351-0370 | coding |
| 22321 | GACCAGCCAGCACCAAGAGC | 31 | 0380-0399 | coding |
| 22322 | GCGTTGCCACTTCTTTCACT | 133 | 0440-0459 | coding |
| 22323 | TTTTGCCAGTAGATGCGAGT | 134 | 0501-0520 | coding |
| 22324 | GGCCATATATTCATGTCCCC | 135 | 0552-0571 | coding |
| 22325 | GCCAGGATCACAATGGAGAG | 136 | 0612-0631 | coding |
| 22326 | GTATGTGCCCTCGTCAGATG | 137 | 0640-0659 | coding |
| 22327 | TTCAGCCAGGTGTTCCCGCT | 138 | 0697-0716 | coding |
| 22328 | GGAAGTCAGCTTTGACTGAT | 139 | 0725-0744 | coding |
| 22329 | CCTCCAGAGGTTGAGCAAAT | 140 | 0798-0817 | coding |
| 22330 | CCAACCAGGAGAGGTGAGGC | 141 | 0827-0846 | coding |
| 22331 | GAAGCTGTGGTTGGTTGTCA | 142 | 0940-0959 | coding |
| 22332 | TTGAAGGTCTGATTCACTCT | 143 | 0987-1006 | coding |
| 22333 | AAGGTAATGGCCCAGGATGG | 144 | 1050-1069 | coding |
| 22334 | AAGCAGTAGGTCAGGCAGCA | 145 | 1098-1117 | coding |
| 22335 | CCTTGCTTCTGCGGACACTG | 146 | 1185-1204 | coding |
| 22336 | AGCCCCTTGCTTCTGCGGAC | 147 | 1189-1208 | coding |
| 22337 | TGACGGAGGCTACCTTCAGA | 148 | 1216-1235 | coding |
| 22338 | GCCTCATGATCCCCACGATC | 36 | 1254-1273 | coding |
| 22339 | GTAAAACAGCTTAAATTTGT | 149 | 1286-1305 | 3′-UTR |
| 22340 | AGAAGAGGTTACATTAAGCA | 150 | 1398-1417 | 3′-UTR |
| 22341 | AGATAATGAATTGGCTGACA | 151 | 1454-1473 | 3′-UTR |
| 24733 | GCGTCATCATCCGCACCATC | 152 | control | |
| 24734 | CGTTGCTTGTGCCGACAGTG | 153 | control | |
| 24735 | GCTCACGAAGAACACCTTCC | 154 | control | |

¹Emboldened residues are 2′-methoxyethoxy residues (others are 2′-deoxy-). All 2′-methoxyethyl cytosines and 2′-deoxy cytosines residues are 5-methyl-cytosines; all linkages are phosphorothioate linkages.
²Co-ordinates from Genbank Accession No. M27533, locus name “HUMIGB7”.

TABLE 7
Inhibition of Human B7-1 mRNA Expression by Chimeric (deoxy
gapped) Phosphorothioate Oligodeoxynucleotides

| | SEQ | GENE | | |
|---|---|---|---|---|
| ISIS | ID | TARGET | % mRNA | % mRNA |
| No: | NO: | REGION | EXPRESSION | INHIBITION |
| basal | — | — | 100% | — |
| 13805 | 30 | AUG | 46% | 54% |
| 13812 | 36 | 3′-UTR | 22% | 78% |
| 22313 | 126 | 5′-UTR | 75% | 25% |
| 22314 | 127 | 5′-UTR | 69% | 31% |
| 22315 | 128 | 5′-UTR | 49% | 51% |
| 22316 | 26 | 5′-UTR | 42% | 58% |
| 22317 | 129 | 5′-UTR | 43% | 57% |
| 22318 | 130 | 5′-UTR | 63% | 37% |
| 22319 | 131 | AUG | 68% | 32% |
| 22320 | 132 | coding | 45% | 55% |
| 22321 | 31 | coding | 57% | 43% |
| 22324 | 135 | coding | 46% | 54% |
| 22325 | 136 | coding | 46% | 54% |
| 22326 | 137 | coding | 62% | 38% |
| 22328 | 139 | coding | 64% | 36% |
| 22329 | 140 | coding | 59% | 41% |
| 22330 | 141 | coding | 54% | 46% |
| 22331 | 142 | coding | 62% | 38% |
| 22332 | 143 | coding | 67% | 33% |
| 22333 | 144 | coding | 73% | 27% |
| 22334 | 145 | coding | 43% | 57% |
| 22335 | 146 | 3′-UTR | 43% | 57% |
| 22336 | 147 | 3′-UTR | 55% | 45% |
| 22337 | 148 | 3′-UTR | 42% | 58% |
| 22338 | 36 | 3′-UTR | 40% | 60% |
| 22339 | 149 | 3′-UTR | 69% | 31% |
| 22340 | 150 | 3′-UTR | 71% | 29% |
| 22341 | 151 | 3′-UTR | 59% | 41% |

Dose response experiments were performed on several of the more active oligonucleotides. The oligonucleotides were screened as described in Example 4 except that the concentration of oligonucleotide was varied as shown in Table 8. Mismatch control oligonucleotides were included. Results are shown in Table 8.

All antisense oligonucleotides tested showed a dose response effect with inhibition of mRNA approximately 60% or greater.

TABLE 8
Dose Response of COS-7 Cells to B7-1 Chimeric (deoxy
gapped) Antisense Oligonucleotides

| ISIS | SEQ ID | ASO Gene | | % mRNA | % mRNA |
|---|---|---|---|---|---|
| # | NO: | Target | Dose | Expression | Inhibition |
| basal | — | — | — | 100% | — |
| 22316 | 26 | 5′-UTR | 10 nM | 99% | 1% |
| ″ | ″ | ″ | 30 nM | 73% | 27% |
| ″ | ″ | ″ | 100 nM | 58% | 42% |
| ″ | ″ | ″ | 300 nM | 33% | 67% |
| 24735 | 154 | control | 10 nM | 100% | — |
| ″ | ″ | ″ | 30 nM | 95% | 5% |
| ″ | ″ | ″ | 100 nM | 81% | 19% |
| ″ | ″ | ″ | 300 nM | 75% | 25% |
| 22335 | 146 | 3′-UTR | 10 nM | 81% | 19% |
| ″ | ″ | ″ | 30 nM | 63% | 37% |
| ″ | ″ | ″ | 100 nM | 43% | 57% |
| ″ | ″ | ″ | 300 nM | 35% | 65% |
| 24734 | 153 | control | 10 nM | 94% | 6% |
| ″ | ″ | ″ | 30 nM | 96% | 4% |
| ″ | ″ | ″ | 100 nM | 94% | 6% |
| ″ | ″ | ″ | 300 nM | 84% | 16% |
| 22338 | 36 | 3′-UTR | 10 nM | 68% | 32% |
| ″ | ″ | ″ | 30 nM | 60% | 40% |
| ″ | ″ | ″ | 100 nM | 53% | 47% |
| ″ | ″ | ″ | 300 nM | 41% | 59% |
| 24733 | 152 | control | 10 nM | 90% | 10% |
| ″ | ″ | ″ | 30 nM | 91% | 9% |
| ″ | ″ | ″ | 100 nM | 90% | 10% |
| ″ | ″ | ″ | 300 nM | 80% | 20% |

### Example 13

### Chimeric (Deoxy Gapped) Mouse B7-1 Antisense Oligonucleotides

Additional oligonucleotides targeting mouse B7-1 were synthesized. Oligonucleotides were synthesized as uniformly phosphorothioate chimeric oligonucleotides having regions of five 2′-O-methoxyethyl (2′-MOE) nucleotides at the wings and a central region of ten deoxynucleotides. Oligonucleotide sequences are shown in Table 9.

Oligonucleotides were screened as described in Example 4. Results are shown in Table 10. Oligonucleotides 18105 (SEQ ID NO: 156), 18106 (SEQ ID NO: 157), 18109 (SEQ ID NO: 160), 18110 (SEQ ID NO: 161), 18111 (SEQ ID NO: 162), 18112 (SEQ ID NO: 163), 18113 (SEQ ID NO: 164), 18114 (SEQ ID NO: 165), 18115. (SEQ ID NO: 166), 1.8117 (SEQ ID NO: 168), 18118 (SEQ ID NO: 169), 18119 (SEQ ID NO: 170), 18120 (SEQ ID NO: 171), 18122 (SEQ ID NO: 173), and 18123 (SEQ ID NO: 174) resulted in greater than approximately 50% inhibition of B7-1 mRNA in this assay.

TABLE 9

Nucleotide Sequences of Mouse B7-1 Chimeric (deoxy
gapped) Oligodeoxynucleotides

| | | | | |
|---|---|---|---|---|
| | | | TARGET GENE | |
| | | SEQ | NUCLEOTIDE | GENE |
| ISIS | NUCLEOTIDE SEQUENCE¹ | ID | CO- | TARGET |
| NO. | (5′−>3′) | NO: | ORDINATES² | REGION |

| | | | | |
|---|---|---|---|---|
| 18104 | AGAGAAACTAGTAAGAGTCT | 155 | 0018-0037 | 5′-UTR |
| 18105 | TGGCATCCACCCGGCAGATG | 156 | 0110-0129 | 5′-UTR |
| 18106 | TCGAGAAACAGAGATGTAGA | 157 | 0144-0163 | 5′-UTR |
| 18107 | TGGAGCTTAGGCACCTCCTA | 158 | 0176-0195 | 5′-UTR |
| 18108 | TGGGGAAAGCCAGGAATCTA | 159 | 0203-0222 | 5′-UTR |
| 18109 | CAGCACAAAGAGAAGAATGA | 160 | 0310-0329 | coding |
| 18110 | ATGAGGAGAGTTGTAACGGC | 161 | 0409-0428 | coding |
| 18111 | AAGTCCGGTTCTTATACTCG | 162 | 0515-0534 | coding |
| 18112 | GCAGGTAATCCTTTTAGTGT | 163 | 0724-0743 | coding |
| 18113 | GTGAAGTCCTCTGACACGTG | 1640 | 927-0946 | coding |
| 18114 | CGAATCCTGCCCCAAAGAGC | 165 | 0995-1014 | coding |
| 18115 | ACTGCGCCGAATCCTGCCCC | 166 | 1002-1021 | coding |
| 18116 | TTGATGATGACAACGATGAC | 167 | 1035-1054 | coding |
| 18117 | CTGTTGTTTGTTTCTCTGCT | 168 | 1098-1117 | coding |
| 18118 | TGTTCAGCTAATGCTTCTTC | 169 | 1134-1153 | coding |
| 18119 | GTTAACTCTATCTTGTGTCA | 170 | 1263-1282 | 3′-UTR |
| 18120 | TCCACTTCAGTCATCAAGCA | 171 | 1355-1374 | 3′-UTR |
| 18121 | TGCTCAATACTCTCTTTTTA | 172 | 1680-1699 | 3′-UTR |
| 18122 | AGGCCCAGCAAACTTGCCCG | 173 | 1330-1349 | 3′-UTR |
| 18123 | AACGGCAAGGCAGCAATACC | 174 | 0395-0414 | coding |

¹Emboldened residues are 2′-methoxyethoxy residues (others are 2′-deoxy-). All 2′-methoxyethyl cytosines and 2′-deoxy cytosines residues are 5-methyl-cytosines; all linkages are phosphorothioate linkages.
²Co-ordinates from Genbank Accession No. X60958, locus name “MMB7BLAA”.

TABLE 10
Inhibition of Mouse B7-1 mRNA Expression by Chimeric (deoxy
gapped) Phosphorothioate Oligodeoxynucleotides

| | SEQ | GENE | | |
|---|---|---|---|---|
| ISIS | ID | TARGET | % mRNA | % mRNA |
| No: | NO: | REGION | EXPRESSION | INHIBITION |
| basal | — | — | 100.0% | — |
| 18104 | 155 | 5′-UTR | 60.0% | 40.0% |
| 18105 | 156 | 5′-UTR | 32.0% | 68.0% |
| 18106 | 157 | 5′-UTR | 51.0% | 49.0% |
| 18107 | 158 | 5′-UTR | 58.0% | 42.0% |
| 18108 | 159 | 5′-UTR | 82.0% | 18.0% |
| 18109 | 160 | coding | 45.5% | 54.5% |
| 18110 | 161 | coding | 21.0% | 79.0% |
| 18111 | 162 | coding | 38.0% | 62.0% |
| 18112 | 163 | coding | 42.0% | 58.0% |
| 18113 | 164 | coding | 24.6% | 75.4% |
| 18114 | 165 | coding | 25.6% | 74.4% |
| 18115 | 166 | coding | 33.5% | 66.5% |
| 18116 | 167 | coding | 65.6% | 34.4% |
| 18117 | 168 | coding | 46.7% | 53.3% |
| 18118 | 169 | coding | 31.7% | 68.3% |
| 18119 | 170 | 3′-UTR | 24.0% | 76.0% |
| 18120 | 171 | 3′-UTR | 25.7% | 73.3% |
| 18121 | 172 | 3′-UTR | 114.0% | — |
| 18122 | 173 | 3′-UTR | 42.0% | 58.0% |
| 18123 | 174 | coding | 42.0% | 58.0% |

### Example 14

### Chimeric (Deoxy Gapped) Human B7-2 Antisense Oligonucleotides

Additional oligonucleotides targeting human B7-2 were synthesized. Oligonucleotides were synthesized as uniformly phosphorothioate chimeric oligonucleotides having regions of five 2′-O-methoxyethyl (2′-MOE) nucleotides at the wings and a central region of ten deoxynucleotides. Oligonucleotide sequences are shown in Table 11.

Oligonucleotides were screened as described in Example 4. Results are shown in Table 12. Oligonucleotides 22284 (SEQ ID NO: 16), 22286 (SEQ ID NO: 176), 22287 (SEQ ID NO: 177), 22288 (SEQ ID NO: 178), 22289 (SEQ ID NO: 179), 22290 (SEQ ID NO: 180), 22291 (SEQ ID NO: 181), 22292 (SEQ ID NO: 182), 22293 (SEQ ID NO: 183), 22294 (SEQ ID NO: 184), 22296 (SEQ ID NO: 186), 22299 (SEQ ID NO: 189), 22300 (SEQ ID NO: 190), 22301 (SEQ ID NO: 191), 22302 (SEQ ID NO: 192), 22303 (SEQ ID NO: 193), 22304 (SEQ ID NO: 194), 22306 (SEQ ID NO: 196), 22307 (SEQ ID NO: 197), 22308 (SEQ ID NO: 198), 22309 (SEQ ID NO: 199), 22310 (SEQ ID NO: 200), and 22311 (SEQ ID NO: 201) resulted in greater than 50% inhibition of B7-2 mRNA in this assay.

TABLE 11

Nucleotide Sequences of Human B7-2 Chimeric (deoxy
gapped) Oligodeoxynucleotides

| | | | | |
|---|---|---|---|---|
| | | | TARGET GENE | |
| | | SEQ | NUCLEOTIDE | GENE |
| ISIS | NUCLEOTIDE SEQUENCE¹ | ID | CO- | TARGET |
| NO. | (5′−>3′) | NO: | ORDINATES² | REGION |

| | | | | |
|---|---|---|---|---|
| 22284 | TGCGAGCTCCCCGTACCTCC | 16 | 0011-0030 | 5′-UTR |
| 22285 | CAGAAGCAAGGTGGTAAGAA | 175 | 0049-0068 | 5′-UTR |
| 22286 | GCCTGTCCACTGTAGCTCCA | 176 | 0113-0132 | 5′-UTR |
| 22287 | AGAATGTTACTCAGTCCCAT | 177 | 0148-0167 | AUG |
| 22288 | TCAGAGGAGCAGCACCAGAG | 178 | 0189-0208 | coding |
| 22289 | TGGCATGGCAGGTCTGCAGT | 179 | 0232-0251 | coding |
| 22290 | AGCTCACTCAGGCTTTGGTT | 180 | 0268-0287 | coding |
| 22291 | TGCCTAAGTATACCTCATTC | 181 | 0324-0343 | coding |
| 22292 | CTGTCAAATTTCTCTTTGCC | 182 | 0340-0359 | coding |
| 22293 | CATATACTTGGAATGAACAC | 183 | 0359-0378 | coding |
| 22294 | GGTCCAACTGTCCGAATCAA | 184 | 0392-0411 | coding |
| 22295 | TGATCTGAAGATTGTGAAGT | 185 | 0417-0436 | coding |
| 22296 | AAGCCCTTGTCCTTGATCTG | 186 | 0430-0449 | coding |
| 22297 | TGTGATGGATGATACATTGA | 187 | 0453-0472 | coding |
| 22298 | TCAGGTTGACTGAAGTTAGC | 188 | 0529-0548 | coding |
| 22299 | GTGTATAGATGAGCAGGTCA | 189 | 0593-0612 | coding |
| 22300 | TCTGTGACATTATCTTGAGA | 190 | 0694-0713 | coding |
| 22301 | AAGATAAAAGCCGCGTCTTG | 191 | 0798-0817 | coding |
| 22302 | AGAAAACCATCACACATATA | 192 | 0900-0919 | coding |
| 22303 | AGAGTTGCGAGGCCGCTTCT | 193 | 0947-0968 | coding |
| 22304 | TCCCTCTCCATTGTGTTGGT | 194 | 0979-0998 | coding |
| 22305 | CATCAGATCTTTCAGGTATA | 195 | 1035-1054 | coding |
| 22306 | GGCTTTACTCTTTAATTAAA | 196 | 1115-1134 | stop |
| 22307 | GAAATCAAAAAGGTTGCCCA | 197 | 1178-1197 | 3′-UTR |
| 22308 | GGAGTCCTGGAGCCCCCTTA | 198 | 1231-1250 | 3′-UTR |
| 22309 | TTGGCATACGGAGCAGAGCT | 199 | 1281-1300 | 3′-UTR |
| 22310 | TGTGCTCTGAAGTGAAAAGA | 200 | 1327-1346 | 3′-UTR |
| 22311 | GGCTTGGCCCATAAGTGTGC | 201 | 1342-1361 | 3′-UTR |
| 22312 | CCTAAATTTTATTTCCAGGT | 202 | 1379-1398 | 3′-UTR |
| 24736 | GCTCCAAGTGTCCCAATGAA | 203 | control | |
| 24737 | AGTATGTTTCTCACTCCGAT | 204 | control | |
| 24738 | TGCCAGCACCCGGTACGTCC | 205 | control | |

¹Emboldened residues are 2′-methoxyethoxy residues (others are 2′-deoxy-). All 2′-methoxyethyl cytosines and 2′-deoxy cytosines residues are 5-methyl-cytosines; all linkages are phosphorothioate linkages.
²Co-ordinates from Genbank Accession No. 1104343 locus name “HSU04343”.

TABLE 12
Inhibition of Human B7-2 mRNA Expression by Chimeric (deoxy
gapped) Phosphorothioate Oligodeoxynucleotides

| | SEQ | GENE | | |
|---|---|---|---|---|
| ISIS | ID | TARGET | % mRNA | % mRNA |
| No: | NO: | REGION | EXPRESSION | INHIBITION |
| basal | — | — | 100% | 0% |
| 10373 | 16 | 5′-UTR | 24% | 76% |
| 22284 | 16 | 5′-UTR | 30% | 70% |
| 22285 | 175 | 5′-UTR | 74% | 26% |
| 22286 | 176 | 5′-UTR | 39% | 61% |
| 22287 | 177 | AUG | 27% | 73% |
| 22288 | 178 | coding | 38% | 62% |
| 22289 | 179 | coding | 41% | 59% |
| 22290 | 180 | coding | 42% | 58% |
| 22291 | 181 | coding | 41% | 59% |
| 22292 | 182 | coding | 39% | 61% |
| 22293 | 183 | coding | 43% | 57% |
| 22294 | 184 | coding | 21% | 79% |
| 22295 | 185 | coding | 66% | 34% |
| 22296 | 186 | coding | 42% | 58% |
| 22297 | 187 | coding | 54% | 46% |
| 22298 | 188 | coding | 53% | 47% |
| 22299 | 189 | coding | 46% | 54% |
| 22300 | 190 | coding | 39% | 61% |
| 22301 | 191 | coding | 51% | 49% |
| 22302 | 192 | coding | 41% | 59% |
| 22303 | 193 | coding | 46% | 54% |
| 22304 | 194 | coding | 41% | 59% |
| 22305 | 195 | coding | 57% | 43% |
| 22306 | 196 | stop | 44% | 56% |
| 22307 | 197 | 3′-UTR | 45% | 55% |
| 22308 | 198 | 3′-UTR | 40% | 60% |
| 22309 | 199 | 3′-UTR | 42% | 58% |
| 22310 | 200 | 3′-UTR | 41% | 59% |
| 22311 | 201 | 3′-UTR | 49% | 51% |
| 22312 | 202 | 3′-UTR | 83% | 17% |

Dose response experiments were performed on several of the more active oligonucleotides. The oligonucleotides were screened as described in Example 4 except that the concentration of oligonucleotide was varied as shown in Table 13. Mismatch control oligonucleotides were included. Results are shown in Table 13.

All antisense oligonucleotides tested showed a dose response effect with maximum inhibition of mRNA approximately 50% or greater.

TABLE 13
Dose Response of COS-7 Cells to B7-2 Chimeric (deoxy
gapped) Antisense Oligonucleotides

| ISIS | SEQ ID | ASO Gene | | % mRNA | % mRNA |
|---|---|---|---|---|---|
| # | NO: | Target | Dose | Expression | Inhibition |
| basal | — | — | — | 100% | — |
| 22284 | 16 | 5′-UTR | 10 nM | 92% | 8% |
| ″ | ″ | ″ | 30 nM | 72% | 28% |
| ″ | ″ | ″ | 100 nM | 59% | 41% |
| ″ | ″ | ″ | 300 nM | 48% | 52% |
| 24738 | 205 | control | 10 nM | 81% | 19% |
| ″ | ″ | ″ | 30 nM | 92% | 8% |
| ″ | ″ | ″ | 100 nM | 101% | — |
| ″ | ″ | ″ | 300 nM | 124% | — |
| 22287 | 177 | AUG | 10 nM | 93% | 7% |
| ″ | ″ | ″ | 30 nM | 79% | 21% |
| ″ | ″ | ″ | 100 nM | 66% | 34% |
| ″ | ″ | ″ | 300 nM | 45% | 55% |
| 24737 | 204 | control | 10 nM | 85% | 15% |
| ″ | ″ | ″ | 30 nM | 95% | 5% |
| ″ | ″ | ″ | 100 nM | 87% | 13% |
| ″ | ″ | ″ | 300 nM | 99% | 1% |
| 22294 | 184 | coding | 10 nM | 93% | 7% |
| ″ | ″ | ″ | 30 nM | 95% | 5% |
| ″ | ″ | ″ | 100 nM | 58% | 42% |
| ″ | ″ | ″ | 300 nM | 45% | 55% |
| 24736 | 203 | control | 10 nM | 102% | — |
| ″ | ″ | ″ | 30 nM | 101% | — |
| ″ | ″ | ″ | 100 nM | 100% | — |
| ″ | ″ | ″ | 300 nM | 107% | — |

### Example 15

### Chimeric (deoxy gapped) Mouse B7-2 Antisense Oligonucleotides

Additional oligonucleotides targeting mouse B7-2 were synthesized. Oligonucleotides were synthesized as uniformly phosphorothioate chimeric oligonucleotides having regions of five 2′-O-methoxyethyl (2′-MOE) nucleotides at the wings and a central region of ten deoxynucleotides. Oligonucleotide sequences are shown in Table 14.

Oligonucleotides were screened as described in Example 4. Results are shown in Table 15.

Oligonucleotides 18084 (SEQ ID NO: 206), 18085 (SEQ ID NO: 207), 18086 (SEQ ID NO: 208), 18087 (SEQ ID NO: 209), 18089 (SEQ ID NO: 211), 18090 (SEQ ID NO: 212), 18091 (SEQ ID NO: 213), 18093 (SEQ ID NO: 215), 18095 (SEQ ID NO: 217), 18096 (SEQ ID NO: 218), 18097 (SEQ ID NO: 219), 18098 (SEQ ID NO: 108), 18102 (SEQ ID NO: 223), and 18103 (SEQ ID NO: 224) resulted in 50% or greater inhibition of B7-2 mRNA expression in this assay.

TABLE 14

Nucleotide Sequences of Mouse B7-2 Chimeric (deoxy
gapped) oligodeoxynucleotides

| | | | | |
|---|---|---|---|---|
| | | | TARGET GENE | |
| | | SEQ | NUCLEOTIDE | GENE |
| ISIS | NUCLEOTIDE SEQUENCE¹ | ID | CO- | TARGET |
| NO. | (5′−>3′) | NO: | ORDINATES² | REGION |

| | | | | |
|---|---|---|---|---|
| 18084 | GCTGCCTACAGGAGCCACTC | 206 | 0003-0022 | 5′-UTR |
| 18085 | TCAAGTCCGTGCTGCCTACA | 207 | 0013-0032 | 5′-UTR |
| 18086 | GTCTACAGGAGTCTGGTTGT | 208 | 0033-0052 | 5′-UTR |
| 18087 | AGCTTGCGTCTCCACGGAAA | 209 | 0152-0171 | coding |
| 18088 | TCACACTATCAAGTTTCTCT | 210 | 0297-0316 | coding |
| 18089 | GTCAAAGCTCGTGCGGCCCA | 211 | 0329-0348 | coding |
| 18090 | GTGAAGTCGTACAGTCCAGT | 212 | 0356-0375 | coding |
| 18091 | GTGACCTTGCTTAGACGTGC | 213 | 0551-0570 | coding |
| 18092 | CATCTTCTTAGGTTTCGGGT | 214 | 0569-0588 | coding |
| 18093 | GGCTGTTGGAGATACTGAAC | 215 | 0663-0682 | coding |
| 18094 | GGGAATGAAAGAGAGAGGCT | 216 | 0679-0698 | coding |
| 18095 | ACATACAATGATGAGCAGCA | 217 | 0854-0873 | coding |
| 18096 | GTCTCTCTGTCAGCGTTACT | 218 | 0934-0953 | coding |
| 18097 | TGCCAAGCCCATGGTGCATC | 219 | 0092-0111 | AUG |
| 18098 | GGATTGCCAAGCCCATGGTG | 108 | 0096-0115 | AUG |
| 18099 | GCAATTTGGGGTTCAAGTTC | 220 | 0967-0986 | coding |
| 18100 | CAATCAGCTGAGAACATTTT | 221 | 1087-1106 | 3′-UTR |
| 18101 | TTTTGTATAAAACAATCATA | 222 | 0403-0422 | coding |
| 18102 | CCTTCACTCTGCATTTGGTT | 223 | 0995-1014 | stop |
| 18103 | TGCATGTTATCACCATACTC | 224 | 0616-0635 | coding |

¹Emboldened residues are 2′-methoxyethoxy residues (others are 2′-deoxy-). All 2′-methoxyethyl cytosines and 2′-deoxy cytosines residues are 5-methyl-cytosines; all linkages are phosphorothioate linkages.
²Co-ordinatesfrom Genbank Accession No. S70108 locus name “S70108”.

TABLE 15
Inhibition of Mouse B7-2 mRNA Expression by Chimeric (deoxy
gapped) Phosphorothioate Oligodeoxynucleotides

| | SEQ | GENE | | |
|---|---|---|---|---|
| ISIS | ID | TARGET | % mRNA | % mRNA |
| No: | NO: | REGION | EXPRESSION | INHIBITION |
| basal | — | — | 100.0% | 0.0% |
| 18084 | 206 | 5′-UTR | 36.4% | 63.6% |
| 18085 | 207 | 5′-UTR | 35.0% | 65.0% |
| 18086 | 208 | 5′-UTR | 40.1% | 59.9% |
| 18087 | 209 | coding | 42.1% | 57.9% |
| 18088 | 210 | coding | 52.3% | 47.7% |
| 18089 | 211 | coding | 20.9% | 79.1% |
| 18090 | 212 | coding | 36.6% | 63.4% |
| 18091 | 213 | coding | 37.1% | 62.9% |
| 18092 | 214 | coding | 58.9% | 41.1% |
| 18093 | 215 | coding | 32.7% | 67.3% |
| 18094 | 216 | coding | 63.8% | 36.2% |
| 18095 | 217 | coding | 34.3% | 65.7% |
| 18096 | 218 | coding | 32.3% | 67.7% |
| 18097 | 219 | AUG | 24.5% | 75.5% |
| 18098 | 108 | AUG | 32.2% | 67.8% |
| 18099 | 220 | coding | 66.8% | 33.2% |
| 18100 | 221 | 3′-UTR | 67.2% | 32.8% |
| 18101 | 222 | coding | 88.9% | 11.1% |
| 18102 | 223 | stop | 33.8% | 66.2% |
| 18103 | 224 | coding | 30.2% | 69.8% |

### Example 16

### Effect of B7 Antisense Oligonucleotides on Cell Surface Expression

B7 antisense oligonucleotides were tested for their effect on cell surface expression of both B7-1 and B7-2. Cell surface expression was measured as described in Example 2. Experiments were done for both human B7 and mouse B7. Results for human B7 are shown in Table 16. Results for mouse B7 are shown in Table 17.

In both species, B7-1 antisense oligonucleotides were able to specifically reduce the cell surface expression of B7-1. B7-2 antisense oligonucleotides were specific for the B7-2 family member. These oligonucleotides were also specific for their effect on B7-1 and B7-2 mRNA levels.

TABLE 16
Inhibition of Human B7 Cell Surface Expression by Chimeric
(deoxy gapped) Phosphorothioate Oligodeoxynucleotides

| | SEQ | | | |
|---|---|---|---|---|
| ISIS | ID | GENE | % B7-1 | % B7-2 |
| No: | NO: | TARGET | EXPRESSION | EXPRESSION |
| basal | — | — | 100% | 0% |
| 22316 | 26 | B7-1 | 31% | 100% |
| 22317 | 129 | B7-1 | 28% | 91% |
| 22320 | 132 | B7-1 | 37% | 86% |
| 22324 | 135 | B7-1 | 37% | 91% |
| 22325 | 136 | B7-1 | 32% | 89% |
| 22334 | 145 | B7-1 | 28% | 92% |
| 22335 | 146 | B7-1 | 23% | 95% |
| 22337 | 148 | B7-1 | 48% | 101% |
| 22338 | 36 | B7-1 | 22% | 96% |
| 22284 | 16 | B7-2 | 88% | 32% |
| 22287 | 177 | B7-2 | 92% | 35% |
| 22294 | 184 | B7-2 | 77% | 28% |

TABLE 17
Inhibition of Mouse B7 Cell Surface Expression by Chimeric
(deoxy gapped) Phosphorothioate Oligodeoxynucleotides

| | SEQ | GENE | | |
|---|---|---|---|---|
| ISIS | ID | TARGET | % B7-1 | % B7-2 |
| No: | NO: | REGION | EXPRESSION | EXPRESSION |
| basal | — | — | 100% | 0% |
| 18089 | 211 | B7-2 | 85% | 36% |
| 18097 | 219 | B7-2 | 87% | 28% |
| 18110 | 161 | B7-1 | 31% | 93% |
| 18113 | 164 | B7-1 | 25% | 91% |
| 18119 | 170 | B7-1 | 27% | 98% |

Dose response experiments were performed on several of the more active human B7-1 antisense oligonucleotides. The oligonucleotides were screened as described in Example 2 except that the concentration of oligonucleotide was varied as shown in Table 18. Results are shown Table 18.

All antisense oligonucleotides tested showed a dose response effect with inhibition of cell surface expression approximately 60% or greater.

TABLE 18
Dose Response of COS-7 Cells to B7-1
Chimeric (deoxy gapped) Antisense Oligonucleotides

| ISIS | SEQ ID | ASO Gene | | % Surface | % Surface |
|---|---|---|---|---|---|
| # | NO: | Target | Dose | Expression | Inhibition |
| basal | — | — | — | 100% | — |
| 22316 | 26 | 5′-UTR | 10 nM | 74% | 26% |
| ″ | ″ | ″ | 30 nM | 74% | 26% |
| ″ | ″ | ″ | 100 nM | 47% | 53% |
| ″ | ″ | ″ | 300 nM | 34% | 66% |
| 22335 | 146 | 3′-UTR | 10 nM | 81% | 19% |
| ″ | ″ | ″ | 30 nM | 69% | 31% |
| ″ | ″ | ″ | 100 nM | 47% | 53% |
| ″ | ″ | ″ | 300 nM | 38% | 62% |
| 22338 | 36 | 3′-UTR | 10 nM | 78% | 22% |
| ″ | ″ | ″ | 30 nM | 65% | 35% |
| ″ | ″ | ″ | 100 nM | 50% | 50% |
| ″ | ″ | ″ | 300 nM | 40% | 60% |

Dose response experiments were performed on several of the more active human B7-2 antisense oligonucleotides. The oligonucleotides were screened as described in Example 2 except that the concentration of oligonucleotide was varied as shown in Table 19. Results are shown in Table 19.

All antisense oligonucleotides tested showed a dose response effect with maximum inhibition of cell surface expression 85% or greater.

TABLE 19
Dose Response of COS-7 Cells to B7-2
Chimeric (deoxy gapped) Antisense Oligonucleotides

| ISIS | SEQ ID | ASO Gene | | % Surface | % Surface |
|---|---|---|---|---|---|
| # | NO: | Target | Dose | Expression | Inhibition |
| basal | — | — | — | 100% | — |
| 22284 | 16 | 5′-UTR | 10 nM | 63% | 37% |
| ″ | ″ | ″ | 30 nM | 60% | 40% |
| ″ | ″ | ″ | 100 nM | 37% | 63% |
| ″ | ″ | ″ | 300 nM | 15% | 85% |
| 22287 | 177 | AUG | 10 nM | 93% | 7% |
| ″ | ″ | ″ | 30 nM | 60% | 40% |
| ″ | ″ | ″ | 100 nM | 32% | 68% |
| ″ | ″ | ″ | 300 nM | 15% | 85% |
| 22294 | 184 | coding | 10 nM | 89% | 11% |
| ″ | ″ | ″ | 30 nM | 62% | 38% |
| ″ | ″ | ″ | 100 nM | 29% | 71% |
| ″ | ″ | ″ | 300 nM | 12% | 88% |

### Example 17

### Effect of B7-1 Antisense Oligonucleotides in a Murine Model for Rheumatoid Arthritis

Collagen-induced arthritis (CIA) was used as a murine model for arthritis (Mussener, A., et al., Clin. Exp. Immunol., 1997, 107, 485-493). Female DBA/1LacJ mice (Jackson Laboratories, Bar Harbor, Me.) between the ages of 6 and 8 weeks were used to assess the activity of B7-1 antisense oligonucleotides.

On day 0, the mice were immunized at the base of the tail with 100 μg of bovine type II collagen which is emulsified in Complete Freund's Adjuvant (CFA). On day 7, a second booster dose of collagen was administered by the same route. On day 14, the mice were injected subcutaneously with 100 μg of LPS. Oligonucleotide was administered intraperitoneally daily (10 mg/kg bolus) starting on day −3 (three days before day 0) and continuing for the duration of the study. Oligonucleotide 17456 (SEQ ID NO. 173) is a fully phosphorothioated analog of 18122.

Weights were recorded weekly. Mice were inspected daily for the onset of CIA. Paw widths are rear ankle widths of affected and unaffected joints were measured three times a week using a constant tension caliper. Limbs were clinically evaluated and graded on a scale from 0-4 (with 4 being the highest).

Results are shown in Table 20. Treatment with B7-1 and B7-2 antisense oligonucleotides was able to reduce the incidence of the disease, but had modest effects on severity. The combination of 17456 (SEQ ID NO. 173) and 11696 (SEQ ID NO. 108) was able to significantly reduce the incidence of the disease and its severity.

TABLE 20
Effect of B7 antisense oligonucleotide on CIA

| | SEQ | Dose | % | | |
|---|---|---|---|---|---|
| ISIS #(s) | ID NO | mg/kg | Incidence | Peak day¹ | Severity² |
| control | | — | 70% | 6.7 ± 2.9 | 3.2 ± 1.1 |
| 17456 (B7-1) | 173 | 10 | 50% | 12.1 ± 4.6 | 2.7 ± 1.3 |
| 11696 (B7-2) | 108 | 10 | 37.5% | 11.6 ± 4.5 | 3.4 ± 1.8 |
| 17456/11696 | | 10 | 30% | 1.0 ± 0.6 | 0.7 ± 0.4 |
| 18110 (B7-1 | 161 | 10 | 55.6% | 2.0 ± 0.8 | 2.0 ± 1.3 |
| 18089 (B7-2 | 211 | 10 | 44.4% | 6.8 ± 2.2 | 2.3 ± 1.3 |
| 18110/18089 | | 10 | 60% | 11.6 ± 0.7 | 4.5 ± 1.7 |

¹Peak day is the day from onset of maximum swelling for each joint measure.
²Severity is the total clinical score divided by the total number of mice in the group.

### Example 18

### Effect of B7-1 Antisense Oligonucleotides in a Murine Model for Multiple Sclerosis

Experimental autoimmune encephalomyelitis (EAE) is a commonly accepted murine model for multiple sclerosis (Myers, K. J., et al., J. Neuroimmunol., 1992, 41, 1-8). SJL/H, PL/J, (SJLxPL/J)F1, (SJLxBalb/c)F1 and Balb/c female mice between the ages of 6 and 12 weeks are used to test the activity of a B7-1 antisense oligonucleotide.

The mice are immunized in the two rear foot pads and base of the tail with an emulsion consisting of encephalitogenic protein or peptide (according to Myers, K. J., et al., J. of Immunol., 1993, 151, 2252-2260) in Complete Freund's Adjuvant supplemented with heat killed Mycobacterium tuberculosis. Two days later, the mice receive an intravenous injection of 500 ng Bordetella pertussis toxin and additional adjuvant.

Alternatively, the disease may also be induced by the adoptive transfer of T-cells. T-cells are obtained from the draining of the lymph nodes of mice immunized with encephalitogenic protein or peptide in CFA. The T cells are grown in tissue culture for several days and then injected intravenously into naive syngeneic recipients.

Mice are monitored and scored daily on a 0-5 scale for signals of the disease, including loss of tail muscle tone., wobbly gait, and various degrees of paralysis.

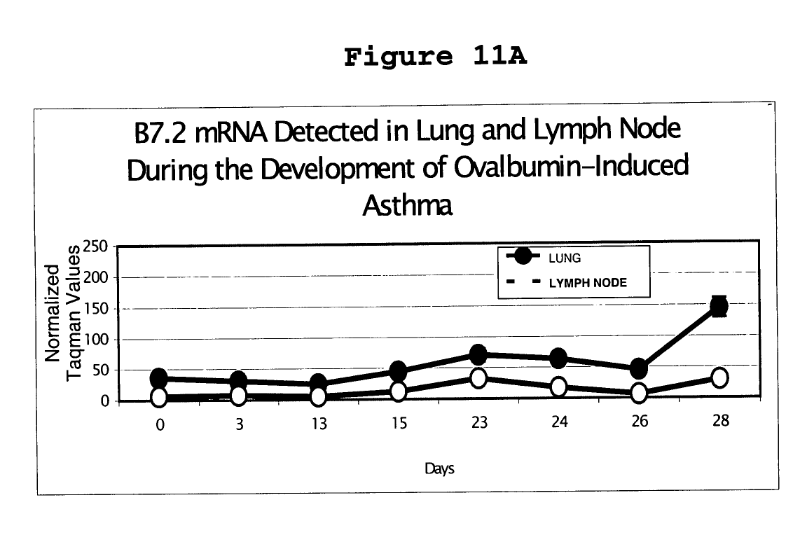

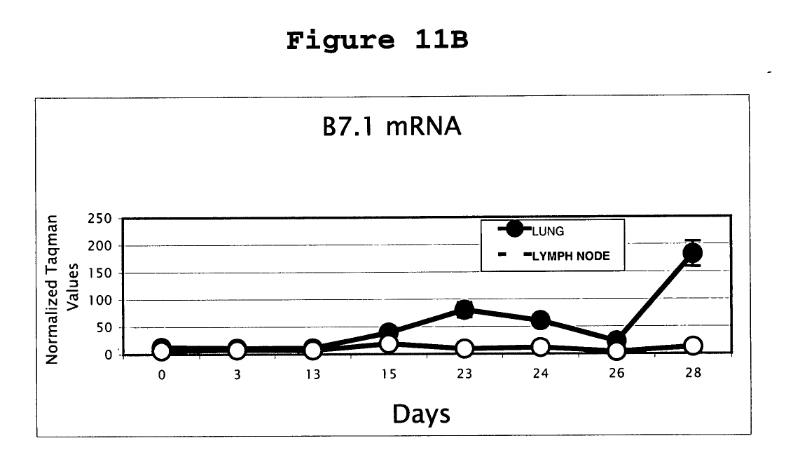

Oligonucleotide 17456 (SEQ ID NO. 173), a fully phosphorothioated analog of 18122, was compared to a saline control and a fully phosphorothioated oligonucleotide of random sequence (Oligonucleotide 17460). Results of this experiment are shown in FIG. 11.

As shown in FIG. 11, for all doses of oligonucleotide 17456 tested, there is a protective effect, i.e. a reduction of disease severity. At 0.2 mg/kg, this protective effect is greatly reduced after day 20, but at the higher doses tested, the protective effect remains throughout the course of the experiment (day 40). The control oligonucleotide gave results similar to that obtained with the saline control.

### Example 19

### Additional Antisense Oligonucleotides Targeted to Human B7-1

Additional oligonucleotides targeting human B7-1 were synthesized. Oligonucleotides were synthesized as uniformly phosphorothioate chimeric oligonucleotides having regions of five 2′-O-methoxyethyl (2′-MOE) nucleotides at the wings and a central region of ten deoxynucleotides. Oligonucleotide sequences are shown in Table 21.

The human promonocytic leukaemia cell line, THP-1 (American Type Culture Collection, Manassas, Va.) was maintained in RPMI 1640 growth media supplemented with 10% fetal calf serum (FCS; Life Technologies, Rockville, Md.). A total of 1×10 cells were electroporated at an oligonucleotide concentration of 10 micromolar in 2 mm cuvettes, using an Electrocell Manipulator 600 instrument (Biotechnologies and Experimental Research, Inc.) employing 200 V, 1000 μF. Electroporated cells were then transferred to petri dishes and allowed to recover for 16 hrs. Cells were then induced with LPS at a final concentration of 1 μg/ml for 16 hours. RNA was isolated and processed as described in previous examples. Results are shown in Table 22.

Oligonucleotides 113492, 113495, 113498, 113499, 113501, 113502, 113504, 113505, 113507, 113510, 113511, 113513 and 113514 (SEQ ID NO: 228, 231, 234, 235, 237, 238, 240, 241, 243, 246, 247, 249 and 250) resulted in 50% or greater inhibition of B7-1 mRNA expression in this assay.

TABLE 21

Nucleotide Sequences of Human B7-1 Chimeric (deoxy
gapped) Oligodeoxynucleotides

| | | | | |
|---|---|---|---|---|
| | | | TARGET | |
| | | | GENE | |
| | | SEQ | NUCLEOTIDE | GENE |
| ISIS | NUCLEOTIDE SEQUENCE¹ | ID | CO- | TARGET |
| NO. | (5′−>3′) | NO. | ORDINATES² | REGION |

| | | | | |
|---|---|---|---|---|
| 113489 | CCCTCCAGTGATGTTTACAA | 225 | 179 | 5′ UTR |
| 113490 | GAAGACCCTCCAGTGATGTT | 226 | 184 | 5′ UTR |
| 113491 | CGTAGAAGACCCTCCAGTGA | 227 | 188 | 5′ UTR |
| 113492 | TTCCCAGGTGCAAAACAGGC | 228 | 234 | 5′ UTR |
| 113493 | TGCCTTCAGATGCTTAGGGT | 229 | 299 | 5′ UTR |
| 113494 | CCTCCGTGTGTGGCCCATGG | 230 | 316 | AUG |
| 113495 | GGTGATGTTCCCTGCCTCCG | 231 | 330 | Coding |
| 113496 | GATCGTGATGTTCCCTGCCT | 232 | 333 | Coding |
| 113497 | AGGTATGGACACTTGGATGG | 233 | 348 | Coding |
| 113498 | GAAAGACCAGCCAGCACCAA | 234 | 384 | Coding |
| 113499 | CAGCGTTGCCACTTCTTTCA | 235 | 442 | Coding |
| 113500 | GTGACCACAGGACAGCGTTG | 236 | 454 | Coding |
| 113501 | AGATGCGAGTTTGTGCCAGC | 237 | 491 | Coding |
| 113502 | CCTTTTGCCAGTAGATGCGA | 238 | 503 | Coding |
| 113503 | CGGTTCTTGTACTCGGGCCA | 239 | 567 | Coding |
| 11350 | CGCAGAGCCAGGATCACAAT | 240 | 618 | Coding |
| 113505 | CTTCAGCCAGGTGTTCCCGC | 241 | 698 | Coding |
| 113506 | TAACGTCACTTCAGCCAGGT | 242 | 706 | Coding |
| 113507 | TTCTCCATTTTCCAACCAGG | 243 | 838 | Coding |
| 113508 | CTGTTGTGTTGATGGCATTT | 244 | 863 | Coding |
| 113509 | CATGAAGCTGTGGTTGGTTG | 245 | 943 | Coding |
| 113510 | AGGAAAATGCTCTTGCTTGG | 246 | 1018 | Coding |
| 113511 | TGGGAGCAGGTTATCAGGAA | 247 | 1033 | Coding |
| 113512 | TAAGGTAATGGCCCAGGATG | 248 | 1051 | Coding |
| 113513 | GGTCAGGCAGCATATCACAA | 249 | 1090 | Coding |
| 113514 | GCCCCTTGCTTCTGCGGACA | 250 | 1188 | 3′ UTR |
| 113515 | AGATCTTTTCAGCCCCTTGC | 251 | 1199 | 3′ UTR |
| 113516 | TTTGTTAAGGGAAGAATGCC | 252 | 1271 | 3′ UTR |
| 113517 | AAACGAGAGGGATGCCAGCC | 253 | 1362 | 3′ UTR |
| 113518 | CAAGACAATTCAAGATGGCA | 254 | 1436 | 3′ UTR |

¹Emboldened residUes are 2′-methoxyethoxy residues (others are 2′-deoxy-). All 2′-methoxyethyl cytosines and 2′-deoxy cytosines residues are 5-methyl-cytosines; all linkages are phosphorothioate linkages.
²Co-ordinates from Genbank Accession No. M27533 to which the oligonucleotides are targeted.

TABLE 22
Inhibition of Human B7-1 mRNA Expression by Chimeric (deoxy
gapped) Phosphorothioate Oligodeoxynucleotides

| | SEQ | GENE | | |
|---|---|---|---|---|
| ISIS | ID | TARGET | % mRNA | % mRNA |
| No: | NO: | REGION | EXPRESSION | INHIBITION |
| 113489 | 225 | 5′ UTR | 122 | — |
| 113490 | 226 | 5′ UTR | 183 | — |
| 113491 | 227 | 5′ UTR | 179 | — |
| 113492 | 228 | 5′ UTR | 27 | 73 |
| 113493 | 229 | 5′ UTR | 488 | — |
| 113494 | 230 | AUG | 77 | 23 |
| 113495 | 231 | Coding | 43 | 57 |
| 113496 | 232 | Coding | 71 | 29 |
| 113497 | 233 | Coding | 78 | 22 |
| 113498 | 234 | Coding | 37 | 63 |
| 113499 | 235 | Coding | 25 | 75 |
| 113500 | 236 | Coding | 83 | 17 |
| 113501 | 237 | Coding | 36 | 64 |
| 113502 | 238 | Coding | 26 | 74 |
| 113503 | 239 | Coding | 65 | 35 |
| 113504 | 240 | Coding | 46 | 54 |
| 113505 | 241 | Coding | 40 | 60 |
| 113506 | 242 | Coding | 105 | — |
| 113507 | 243 | Coding | 36 | 64 |
| 113508 | 244 | Coding | 117 | — |
| 113509 | 245 | Coding | 62 | 38 |
| 113510 | 246 | Coding | 43 | 57 |
| 113511 | 247 | Coding | 48 | 52 |
| 113512 | 248 | Coding | 73 | 27 |
| 113513 | 249 | Coding | 48 | 52 |
| 113514 | 250 | 3′ UTR | 35 | 65 |
| 113515 | 251 | 3′ UTR | 184 | — |
| 113516 | 252 | 3′ UTR | 83 | 17 |
| 113517 | 253 | 3′ UTR | 201 | — |
| 113518 | 254 | 3′ UTR | 97 | 03 |

### Example 20

### Additional Antisense Oligonucleotides Targeted to Human B7-2

Additional oligonucleotides targeting human B7-2 were synthesized. Oligonucleotides were synthesized as uniformly phosphorothioate chimeric oligonucleotides having regions of five 2′-O-methoxyethyl (2′-MOE) nucleotides at the wings and a central region of ten deoxynucleotides. Oligonucleotide sequences are shown in Table 23.

The human promonocytic leukaemia cell line, THP-1 (American Type Culture Collection, Manassas, Va.) was maintained in RPMI 1640 growth media supplemented with 10% fetal calf serum (FCS; Life Technologies, Rockville, Md.). A total of 1×10⁷ cells were electroporated at an oligonucleotide concentration of 10 micromolar in 2 mm cuvettes, using an Electrocell Manipulator 600 instrument (Biotechnologies and Experimental Research, Inc.) employing 200 V, 1000 μF. Electroporated cells were then transferred to petri dishes and allowed to recover for 16 hrs Cells were then induced with LPS and dibutyryl cAMP (500 μM) for 16 hours. RNA was isolated and processed as described in previous examples. Results are shown in Table 24.

Oligonucleotides ISIS 113131, 113132, 113134, 113138, 113142, 113144, 113145, 113146, 113147, 113148, 113149, 113150, 113153, 113155, 113157, 113158, 113159 and 113160 (SEQ ID NO: 255, 256, 258, 262, 266, 268, 269, 270, 271, 272, 273, 274, 277, 279, 281, 282, 283 and 284) resulted in 50% or greater inhibition of B7-2 mRNA expression in this assay.

TABLE 23

Nucleotide Sequences of Human B7-2 Chimeric (deoxy
gapped) Oligodeoxynucleotides

| | | | | |
|---|---|---|---|---|
| | | | TARGET | |
| | | | GENE | |
| | | SEQ | NUCLEOTIDE | GENE |
| ISIS | NUCLEOTIDE SEQUENCE¹ | ID | CO | TARGET |
| NO. | (5′−>3′) | NO: | ORDINATES² | REGION |

| | | | | |
|---|---|---|---|---|
| 113131 | CGTGTGTCTGTGCTAGTCCC | 255 | 38 | 5′ UTR |
| 113132 | GCTGCTTCTGCTGTGACCTA | 256 | 83 | 5′ UTR |
| 113133 | TATTTGCGAGCTCCCCGTAC | 257 | 15 | 5′ UTR |
| 113134 | GCATAAGCACAGCAGCATTC | 258 | 79 | 5′ UTR |
| 113135 | TCCAAAAAGAGACCAGATGC | 259 | 97 | 5′ UTR |
| 113136 | AAATGCCTGTCCACTGTAGC | 260 | 117 | 5′ UTR |
| 113137 | CTTCAGAGGAGCAGCACCAG | 261 | 191 | Coding |
| 113138 | GAATCTTCAGAGGAGCAGCA | 262 | 195 | Coding |
| 113139 | CAAATTGGCATGGCAGGTCT | 263 | 237 | coding |
| 113140 | GCTTTGGTTTTGAGAGTTTG | 264 | 257 | Coding |
| 113141 | AGGCTTTGGTTTTGAGAGTT | 265 | 259 | Coding |
| 113142 | GCTCACTCAGGCTTTCGTTT | 266 | 267 | Coding |
| 113143 | GGTCCTGCCAAAATACTACT | 267 | 288. | Coding |
| 113144 | AGCCCTTGTCCTTGATCTGA | 268 | 429 | Coding |
| 113145 | TCTGGCCTTTTTGTGATGGA | 269 | 464 | Coding |
| 113146 | AATCATTCCTGTCGGCTTTT | 270 | 473 | Coding |
| 113147 | CCGTGTATACATGAGCAGGT | 271 | 595 | Coding |
| 113148 | ACCGTGTATAGATGAGCAGG | 272 | 596 | Coding |
| 113149 | TCATCTTCTTAGGTTCTGGG | 273 | 618 | Coding |
| 113150 | ACAAGCTGATGGAAACCTCG | 274 | 720 | Coding |
| 113151 | TGCTCGTAACATCAGGGAAT | 275 | 747 | Coding |
| 113152 | AAGATGGTCATATTGCTCGT | 276 | 760 | Coding |
| 113153 | CGCGTCTTGTCAGTTTCCAG | 277 | 787 | Coding |
| 113154 | CAGCTGTAATCCAAGGAATG | 278 | 864 | Coding |
| 113155 | GGGCTTCATCACATCTTTCA | 279 | 1041 | Coding |
| 113156 | CATGTATCACTTTTGTCGCA | 280 | 1093 | Coding |
| 113157 | AGCCCCCTTATTACTCATGG | 281 | 1221 | 3′ UTR |
| 113158 | GGAGTTACAGGGAGGCTATT | 282 | 1261 | 3′ UTR |
| 113159 | AGTCTCCTCTTGGCATACGG | 283 | 1290 | 3′ UTR |
| 113160 | CCCATAAGTGTGCTCTGAAG | 284 | 1335 | 3′ UTR |

¹Emboldened residues are 2′-methoxyethoxy residues (others are 2′-deoxy-). All 2′-methoxyethyl cytosines and 2′-deoxy cytosines residues are 5-methyl-cytosines; all linkages are phosphorothioate linkages.
²For ISIS# 113131 and 113132, co-ordinates are from Genbank Accession No. L25259, locus name “HUMB72A”. For remaining oigonucleotides, co-ordinates are from Genbank Accession No.U04343, locus name “HSU04343”.

TABLE 24
Inhibition of Human B7-2 mRNA Expression by Chimeric (deoxy
gapped) Phosphorothioate Oligodeoxynucleotides

| | SEQ | GENE | | |
|---|---|---|---|---|
| ISIS | ID | TARGET | % mRNA | % mRNA |
| No: | NO: | REGION | EXPRESSION | INHIBITION |
| 113131 | 255 | 5′ UTR | 13 | 87 |
| 113132 | 256 | 5′ UTR | 17 | 83 |
| 113133 | 257 | 5′ UTR | 214 | — |
| 113134 | 258 | 5′ UTR | 27 | 73 |
| 113135 | 259 | 5′ UTR | 66 | 34 |
| 113136 | 260 | 5′ UTR | 81 | 19 |
| 113137 | 261 | Coding | 57 | 43 |
| 113138 | 262 | Coding | 12 | 88 |
| 113140 | 264 | Coding | 214 | — |
| 113141 | 265 | Coding | 126 | — |
| 113142 | 266 | Coding | 35 | 65 |
| 113143 | 267 | Coding | 118 | — |
| 113144 | 268 | Coding | 41 | 59 |
| 113145 | 269 | Coding | 46 | 54 |
| 113146 | 270 | Coding | 32 | 68 |
| 113147 | 271 | Coding | 35 | 65 |
| 113148 | 272 | Coding | 23 | 77 |
| 113149 | 273 | Coding | 29 | 71 |
| 113150 | 274 | Coding | 19 | 81 |
| 113151 | 275 | Coding | 208 | — |
| 113152 | 276 | Coding | 89 | 11 |
| 113153 | 277 | Coding | 19 | 81 |
| 113154 | 278 | Coding | 63 | 37 |
| 113155 | 279 | Coding | 13 | 87 |
| 113156 | 280 | Coding | 83 | 17 |
| 113157 | 281 | 3′ UTR | 13 | 87 |
| 113158 | 282 | 3′ UTR | 20 | 80 |
| 113159 | 283 | 3′ UTR | 43 | 57 |
| 113160 | 284 | 3′ UTR | 09 | 91 |

### Example 21

### Human Skin Psoriasis Model

Animal models of psoriasis based on xenotransplantation of human skin from psoriatic patients are advantageous because they involve the direct study of affected human tissue. Psoriasis is solely a disease of the skin and consequently, engraftment of human psoriatic skin onto SCID mice allows psoriasis to be created with a high degree of fidelity in mice.

BALB/cByJSmn-Prkdcscid/J SCID mice (4-6 weeks old) of either sex (Jackson Laboratory, Bar Harbor, Me.) were maintained in a pathogen free environment. At 6-8 weeks of age, mice were anesthetized by intraperitoneal injection of 30 mg/kg body weight ketamine-HCl and 1 mg/kg body weight acepromazine. After anesthesia, mice were prepared for transplantation by shaving the hair from the dorsal skin, 2 cm away from the head. The area was then sterilized and cleaned with povidone iodide and alcohol. Graft beds of about 1 cm×1 cm were created on the shaved areas by removing full thickness skin down to the fascia. Partial thickness human skin was then orthotopically transferred onto the graft bed. The transplants were held in place by gluing the human skin to mouse-to-mouse skin with Nexband liquid, a veterinary bandage (Veterinary Products Laboratories, Phoenix, Ariz.). Finally, the transplant and the wounds were covered with a thick layer of antibiotic ointment. After 4 weeks of transplantation, a 2 mm punch biopsy was obtained to confirm the acceptance of the graft, and the origin of the skin in the transplant area. Only mice whose grafts did not show signs of infection were used for the study. Normal human skin was obtained from elective plastic surgeries and psoriatic plaques were obtained from shave biopsies from psoriatic volunteers. Partial thickness skin was prepared by dermatome shaving of the skin and transplanted to the mouse as described above for the psoriatic skin.

Animals (n=5) were topically treated with 2.5% (w/w) of each antisense oligonucleotide in a cream formulation comprising 10% isopropyl mylistate, 10% glyceryl moriooleate, 3% cetostearyl alcohol, 10% polyoxyl-20-cetyl ether, 6% poloxamer 407, 2.5% phenoxyethanol, 0.5% methylparaben, 0.5% propylparaben and water (final pH about 7.5).

The following oligonucleotides were used: human B7-1 (5′-TTCCCAGGTGCAAAACAGGC-3′; SEQ ID NO: 228) (ISIS 113492) and human B7-2 (5′-CGTGTGTCTGTGCTAGTCCC-3′; SEQ ID NO: 255) (ISIS 113131). Both sequences contained only phosphorothioate linkages and had 2′-MOE modifications at nucleotides 1-5 and 16-20.

Plaques from the same patients were also transplanted onto control mice (n=5) and treated only with the vehicle of the active cream preparation. Both groups received the topical preparation twice a day for 4 weeks. Within 3-4 weeks the animals were sacrificed and 4 mm punch biopsies were taken from each xenograft. Biopsies were fixed in formalin for paraffin embedding and/or transferred to cryotubes and snap-frozen in liquid nitrogen and stored at −80° C.

Significant histological improvement marked by reduction of hyperkeratosis, acanthosis and lymphonuclear cellular infiltrates was observed in mice treated with the antisense oligonucleotides. Rete pegs, finger-like projections of the epidermis into the dermis, were also measured. These are phenotypic markers for psoriasis which lengthen as the disease progresses. The shortening of these rete pegs are a good measure of anti-psoriatic activity. In mice treated with the active agent, the rete pegs changed from 238.56±98.3 μm to 168.4±96.62 μm (p<0.05), whereas in the control group the rete pegs before and after treatment were 279.93±40.56 μm and 294.65±45.64 μm, respectively (p>0.1). HLA-DR positive lymphocytic infiltrates and intraepidermal CD8 positive lymphocytes were significantly reduced in the transplanted plaques treated with the antisense oligonucleotide cream. These results show that antisense oligonucleotides to B7 inhibit psoriasis-induced inflammation and have therapeutic efficacy in the treatment of psoriasis.

### Example 22

### Mouse Model of Allergic Inflammation

In the mouse model of allergic inflammation, mice were sensitized and challenged with aerosolized chicken ovalbumin (OVA). Airway responsiveness was assessed by inducing airflow obstruction with a methacholine aerosol using a noninvasive method. This methodology utilized unrestrained conscious mice that are placed into the main chamber of a plthysmograph (Buxco Electronics, Inc., Troy, N.Y.). Pressure differences between this chamber and a reference chamber were used to extrapolate minute volume, breathing frequency and enhanced pause (Penh). Penh is a dimensionless parameter that is a function of total pulmonary airflow in mice (i.e., the sum of the airflow in the upper and lower respiratory tracts) during the respiratory cycle of the animal. The lower the PENH, the greater the airflow. This parameter closely correlates with lung resistance as measured by traditional invasive techniques using ventilated animals (Hamelmann . . . . Gelfand, 1997). Dose-response data were plotted as raw Penh values to increasing concentrations of methacholine. This system was used to test the efficacy of antisense oligonucleotides targeted to human B7-1 and B7-2.

There are several important features common to human asthma and the mouse model of allergic inflammation. One of these is pulmonary inflammation, in which cytokine expression and Th2 profile is dominant. Another is goblet cell hyperplasia with increased mucus production. Lastly, airway hyperresponsiveness (AHR), occurs resulting in increased sensitivity to cholinergic receptor agonists such as acetylcholine or methacholine. The compositions and methods of the present invention may be used to treat AHR and pulmonary inflammation.

Ovalbumin-Induced Allergic Inflammation

Female Balb/c mice (Charles Rivers Laboratory, Taconic Farms, NY) were maintained in micro-isolator cages housed in a specific pathogen-free (SPF) facility. The sentinel cages within the animal colony surveyed negative for viral antibodies and the presence of known mouse pathogens. Mice were sensitized and challenged with aerosolized chicken OVA. Briefly, 20 μg alum-precipitated OVA was injected intraperitoneally on days 0 and 14. On day 24, 25 and 26, the animals were exposed for 20 minutes to 1.0% OVA (in saline) by nebulization. The challenge was conducted using an ultrasonic nebulizer (PulmoSonic, The DeVilbiss Co., Somerset, PA). Animals were analyzed about 24 hours following the last nebulization using the Buxco electronics Biosystem. Lung function (Penh), lung histology (cell infiltration and mucus production), target mRNA reduction in the lung, inflammation (BAL cell type & number, cytokine levels), spleen weight and serum AST/ALT were determined.

This method has been used to show that prophylactic treatment with an anti-B7.2 monoclinal antibody continued throughout the sensitization and challenge periods decreases OVA-specific serum IgE and IgE levels, IL-4 and IFN-γ levels in bronchoalveolar lavage (BAL) fluid, airway eosinophilia and airway hyperresponsiveness (Haczku et al., Am. J. Respir. Crit. Care Med. 159:1638-1643, 1999). Treatment during antigen challenge with both anti-B7.1 and anti-B7.2 mAbs is effective; however, either mAb alone is only partially active (Mathur et al., 21:498-509, 1999). However, the anti-B7.2 mAb had no activity when administered after the OVA challenge. The anti-B7.1 monoclonal antibody had no effect, either prophylactically or post-antigen challenge. Thus, there is a need for an effective B7 inhibitor which can be administered after antigen challenge, and which will reduce airway hyperresponsiveness and pulmonary inflammation. As described below, the antisense oligonucleotides of the present inventors fit this description.

Oligonucleotide Administration

Antisense oligonucleotides (ASOs) were dissolved in saline and used to intratracheally dose mice every day, four times per day, from days 15-26 of the OVA sensitization and challenge protocol. Specifically, the mice were anesthetized with isofluorane and placed on a board with the front teeth hung from a line. The nose was covered and the animal's tongue was extended with forceps and 25 μl of various doses of ASO, or an equivalent volume of saline (control) was placed at the back of the tongue until inhaled into the lung. The deposition pattern of an ASO in the lung, ISIS 13920 (5′-TCCGTCATCGCTCCTCAGGG-3′; SEQ ID NO:285) was also examined by immunohistochemical staining using a monoclonal antibody to the oligonucleotide, and showed that the ASO is taken up; throughout the lung, most strongly by antigen presenting cells (APCs) and alveolar epithelium.

The B7 oligonucleotides used were:

(ISIS 121844; SEQ ID NO: 286)

| | |
|---|---|
| B7-1: | 5′-GCTCAGCCTTTCCACTTCAG-3′ |

(ISIS 121874; SEQ ID NO: 287)

| | |
|---|---|
| B7-2: | 5′-GCTCAGCCTTTCCACTTCAG-3′ |

Both of these oligonucleotides are phosphorothioates with 2′-MOE modifications on nucleotides 1-5 and 16-20, and 2′-deoxy at positions 6-15. These ASOs were identified by mouse-targeted ASO screening by target mRNA reduction in mouse cell lines. For B7-2, 19 mouse-targeted ASOs were screened by target mRNA reduction (Northern analysis) in IC-21 macrophages. Dose-response confirmation led to selection of ISIS 121874 (>70% reduction at 25 nM). For B7-1, 22 mouse-targeted ASOs were screened by target mRNA reduction (RT-PCR) in L-929 fibroblasts. Dose-response confirmation led to selection of ISIS 121844 (>70% reduction at 100 nM). No cross hybridization was predicted, and no cross-target reduction was detected in transfected cells.

RT-PCR Analysis

RNA was harvested from experimental lungs removed on day 28 of the OVA protocol. B7.2 and B7.1 levels were measured by quantitative RT-PCR using the Applied Biosystems PRISM 7700 Sequence Detection System (Applied Biosystems, Foster City, Calif.). Primers and probes used for these studies were synthesized by Operon Technologies (Alameda, Calif.). The primer and probe sequences were as follows:

B7-2:

| | | |
|---|---|---|
| B7-2: | | |
| forward: | 5′-GGCCCTCCTCCTTGTGATG-3′ | (SEQ ID NO: 288) |
| probe: | 5′-/56- | (SEQ ID NO: 289) |
| | FAM/TGCTCATCATTGTATGTCACAAGAAGCCG/36-TAMTph/-3′ | |
| reverse: | 5′-CTGGGCCTGCTAGGCTGAT-3′ | (SEQ ID NO: 290) |
| B7-1: | | |
| forward: | 5′-CAGCAAGCTACGGGCAAGTT-3′ | (SEQ ID NO: 291) |
| probe: | 5′-/56- | (SEQ ID NO: 292) |
| | FAM/TGGGCCTTTGATTGCTTGATGACTGAA/36-TAMTph/-3′ | |
| reverse: | 5′-GTGGGCTCAGCCTTTCCA-3′ | (SEQ ID NO: 293) |

Collection of Bronchial Alveolar Lavage (BAL) Fluid and Blood Serum for the Determination of Cytokine and Chemokine Levels

Animals were injected with a lethal dose of ketamine, the trachea was exposed and a cannula was inserted and secured by sutures. The lungs were lavaged twice with 0.5 ml aliquots of ice cold PBS with 0.2% FCS.

The recovered BAL fluid was centrifuged at 1,000 rpm for 10 min at 4° C., frozen on dry ice and stored at −80° C. until used. Luminex was used to measure cytokine levels in BAL fluid and serum.

BAL Cell Counts and Differentials

Cytospins of cells recovered from BAL fluid were prepared using a Shandon Cytospin 3 (Shandon Scientific LTD, Cheshire, England). Cell differentials were performed from slides stained with Leukostat (Fisher Scientific, Pittsburgh, Pa.). Total cell counts were quantified by hemocytometer and, together with the percent type bty differential, were used to calculate specific cell number.

Tissue Histology

Before resection, lungs were inflated with 0.5 ml of 10% phosphate-buffered formalin and fixed overnight at 4° C. The lung samples were washed free of formalin with 1×PBS and subsequently dehydrated through an ethanol series prior to equilibration in xylene and embedded in paraffin. Sections (6μ) were mounted on slides and stained with hematoxylin/eosin, massons trichome and periodic acid-schiff (PAS) reagent. Parasagittal sections were analyzed by bright-field microscopy. Mucus cell content was assessed as the airway epithelium staining with PAS. Relative comparisons of mucus content were made between cohorts of animals by counting the number of PAS-positive airways.

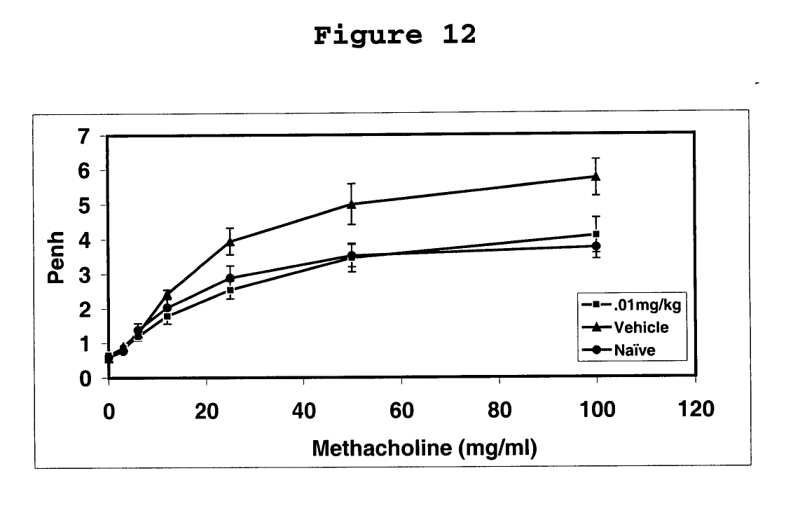

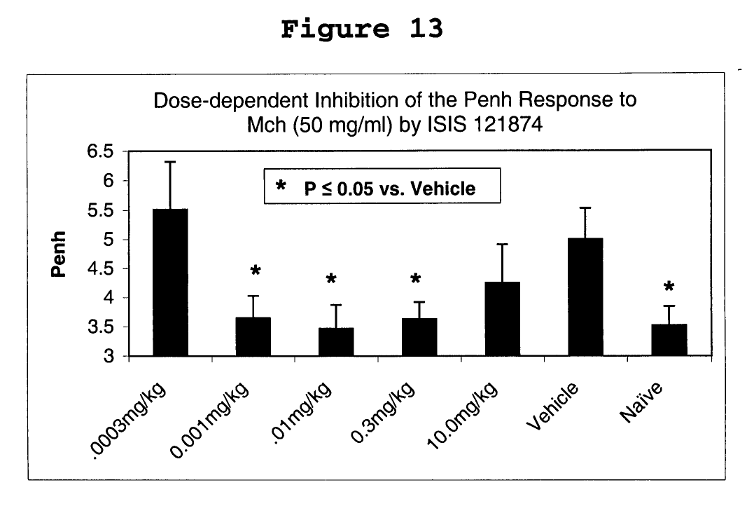

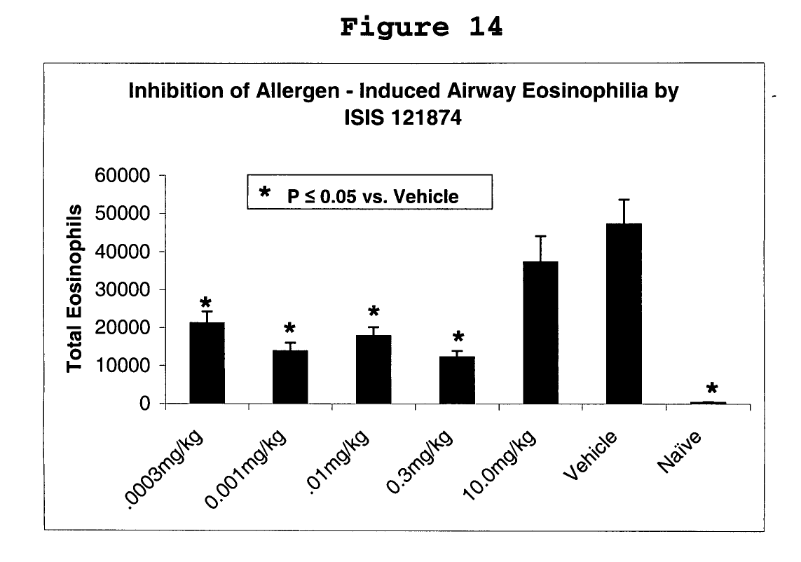

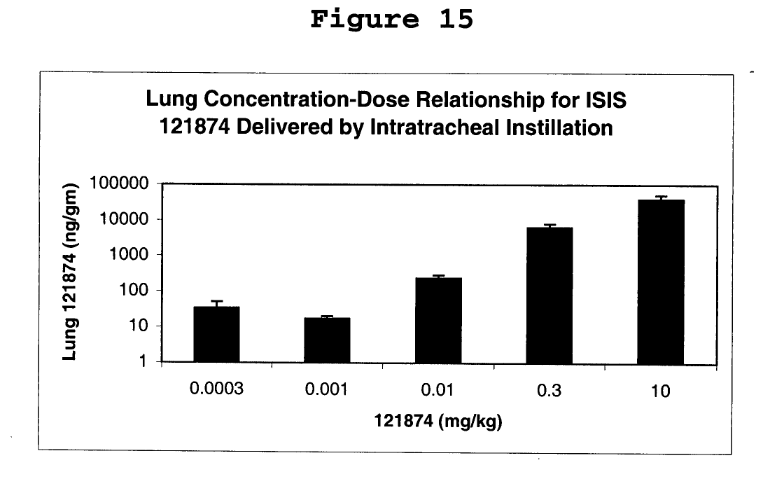

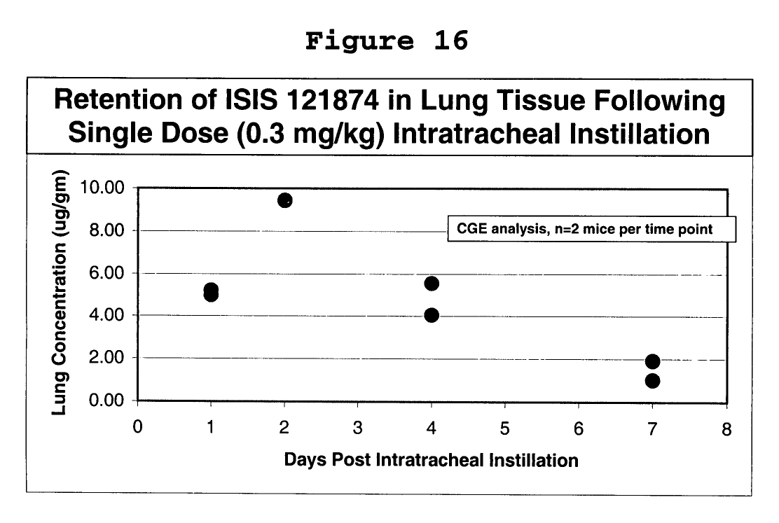

As shown in FIGS. 11A-11B, B7.2 mRNA (FIG. 11A) and B7.1 mRNA (FIG. 11B) were detected in mouse lung and lymph node during the development of ovalbumin-induced asthma. Treatment with ISIS 121874 following allergen challenge reduces the airway response to methacholine (FIG. 12). The Penh value in B7.2 ASO-treated mice was about 40% lower than vehicle-treated mice, and was statistically the same as naive mice which were not sensitized with the allergen or treated with the ASO. This shows that B7.2 ASO-treated mice had significantly better airflow, and less inflammation, than mice which were not treated with the ASO. The dose-dependent inhibition of the Penh response to methacholine by ISIS 121874 is shown in FIG. 13. The inhibition of allergen-induced eosinophilia by ISIS 121874 is shown in FIG. 14. ISIS 121874 at 0.3 mg/kg reduced the total number of eosinophils by about 75% compared to vehicle-treated mice. Since increased numbers of eosinophils result from inflammation, this provides further support for the anti-inflammatory properties of the B7.2 ASO. In addition, daily intratracheal delivery of ISIS 121874 does not induce splenomegaly, the concentration of ISIS 121874 achieved in lung tissue via daily intratracheal administration is proportional to the dose delivered (FIG. 15) and ISIS 121874 is retained in lung tissue for at least one week following single dose (0.3 mg/kg) intratracheal administration as determined by capillary gel electrophoresis (CGE) analysis (FIG. 16).

### Example 23

Support for an Antisense Mechanism of Action for ISIS 121874

Two variants of ISIS 121874 were synthesized: a 7 base mismatch 5′-TCAAGTCCTTCCACACCCAA-3′ (ISIS 306058; SEQ ID NO: 294) and a gap ablated oligonucleotide ISIS 306058 having the same sequence as ISIS 121'874, but with 2′-MOE modifications at nucleotides 1, 2, 3, 6, 9, 13, 16, 18, 19 and 20. Because of the presence of 2′-MOE in the gap, this oligonucleotide is no longer an RNase H substrate and will not recruit RNase H to the RNA-DNA hybrid which is formed.

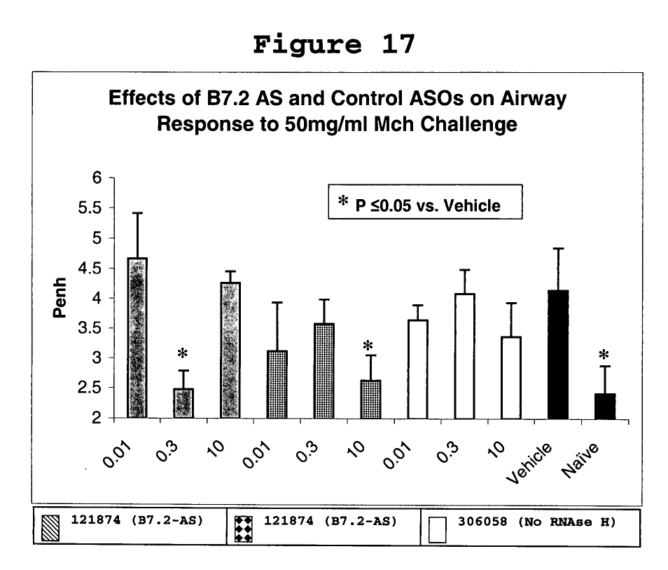

The results (FIG. 17) show that at 0.3 mg/kg, only ISIS 121874, and not the mismatch and gap ablated controls, significantly lowered Penh, which supports that ISIS 121874 is working by an antisense mechanism.

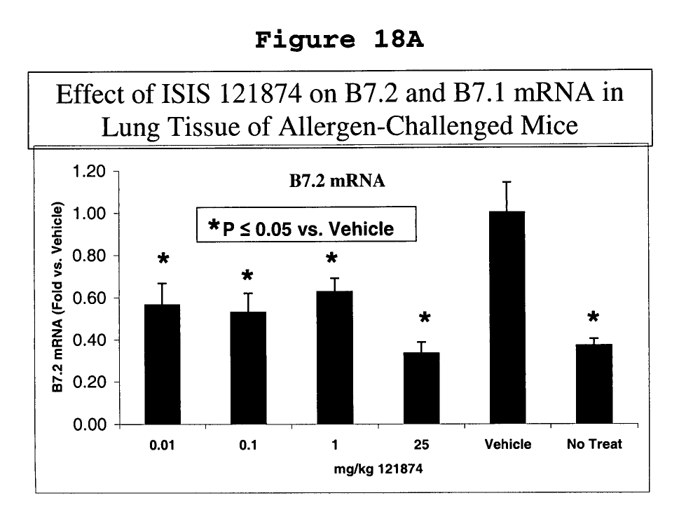

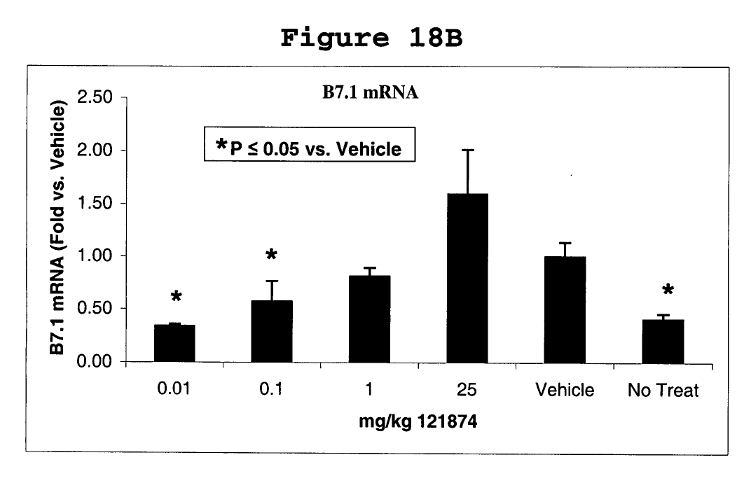

The effects of ISIS 121874 and the control oligonucleotides on airway mucus production in the ovalbumin-induced model were also tested. The results (FIG. 18) show that only ISIS 121874 significantly inhibited mucus production.

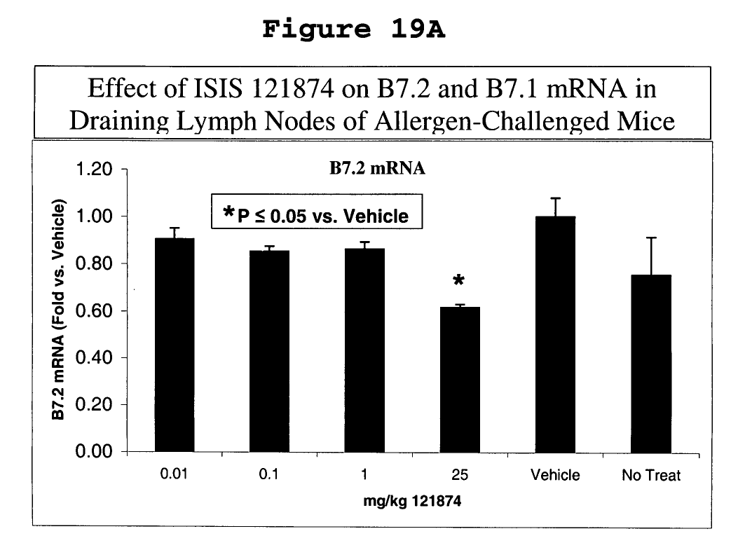

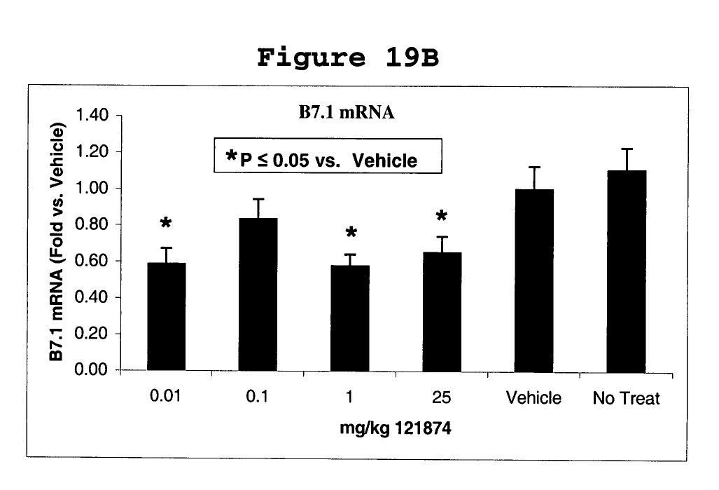

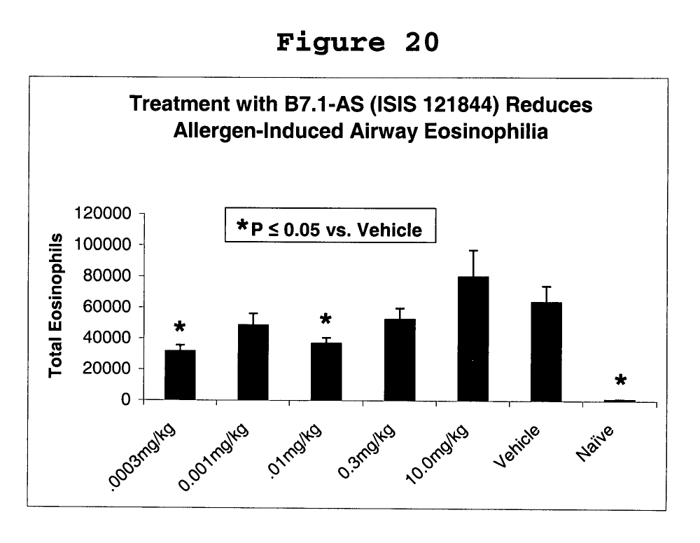

The effect of ISIS 121874 on B7.2 and B7.1 mRNA in lung tissue of allergen-challenged mice is shown in FIGS. 19A and 19B, respectively. The effect of ISIS 121874 on B7.2 and B7.1 mRNA in draining lymph nodes of allergen-challenged mice is shown in FIGS. 20A and 20B, respectively. This shows that ISIS 121874 reduces both B7.2 and B7.1 mRNA (greater in lung vs. node).

In summary, ISIS 121874 resulted in a dose-dependent inhibition of airway hypersensitivity, inhibited eosinophilia and reduced B7.1 and B7.2 expression in the lung and lymph nodes. In addition, ISIS 121874 reduced levels of the following inflammatory molecules: IQE mRNA in the lung and IgE protein in the serum; reduced IL-5 mRNA in the lung and IL-5 protein in the BAL fluid; and reduced the serum level of macrophage chemokine (KC).

In the aerosolized ISIS 121874 study, treatment with 0.001, 0.01, 0.1 or 1.0 mg/kg estimated inhaled dose was delivered by nose-only inhalation of an aerosol solution, four times per day, on days 15-26 (n=8 mice per group). The airway response to methacholine was reduced to the level seen in naive mice at 0.001 mg/kg dose (estimated inhaled dose=1 μg/kg). No gross adverse effects were seen.

### Example 24

### B7.1 ASO in Ovalbumin Model of Asthma

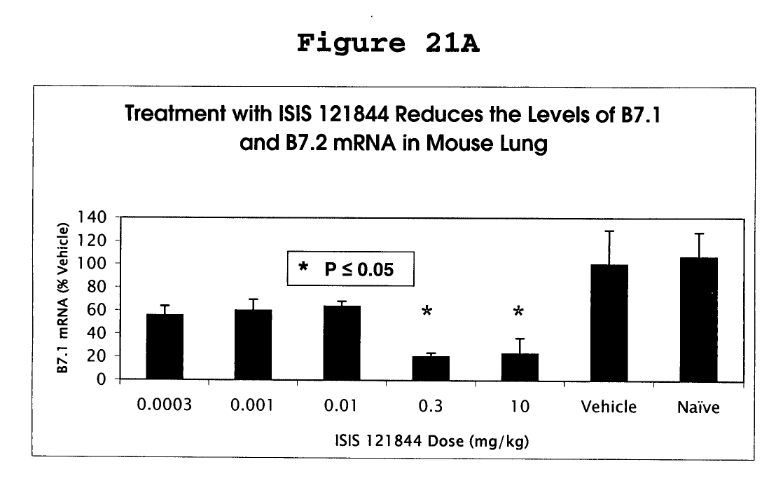

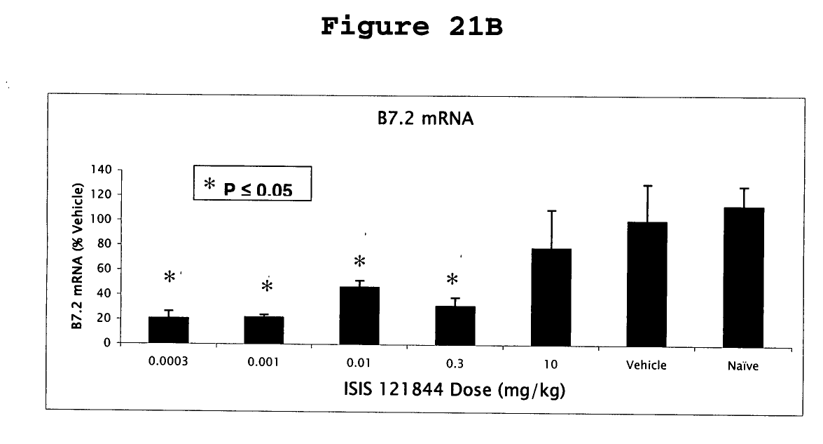

The same protocols described above for the B7.2 ASOs were used to test the effect of the B7.1 ASO ISIS 121844 (SEQ ID NO: 286). In contrast to the B7.2 ASO, ISIS 121844 had no effect on the Penh response in mice challenged with methacholine. Although there was no effect on Penh, ISIS 121844 reduced allergen-induced airway eosinophilia (FIG. 21) and reduced the levels of B7.1 and B7.2 in the mouse lung. (FIGS. 22A-B). Thus, treatment with B7.1 ASO produced anti-inflammatory effects, but did not prevent airway hyper-responsiveness. There was no effect of ISIS 121844 on the Penh response despite achieving an 80% reduction of B7.2 mRNA in the lung (FIG. 21B). Treatment with ISIS 121844 reduced eosinophil and PMN numbers in BAL fluid. This effect was associated with a reduction in lung B7.2 (not B7.1) mRNA.

The combined use of B7.1 or B7.2 with one or more conventional asthma medications including, but not limited to, montelukast sodium (Singulair™), albuterol, beclomethasone dipropionate, triamcinolone acetonide, ipratropium bromide (Atrovent™); flunisolide, fluticasone propionate (Flovent™) and other steroids is also contemplated. The combined use of oligonucleotides which target both B7.1 and B7.2 for the treatment of asthma is also within the scope of the present invention. B7.1 and B7.2 may also be combined with one or more conventional asthma medications as described above for B7.1 or B7.2 alone.

### Example 25

Design and Screening of Duplexed Antisense Compounds Targeting B7.1 or B7.2

In accordance with the present invention, a series of nucleic acid duplexes comprising the antisense compounds of the present invention and their complements can be designed to target B7.1 or B7.2. The nucleobase sequence of the antisense strand of the duplex comprises at least a portion of an oligonucleotide to B7.1 or B7.2 as described herein. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini. For example, a duplex comprising an antisense strand having the sequence CGAGAGGCGGACGGGACCG and having a two-nucleobase overhang of deoxythymidine (dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5×solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate B7.1 or B7.2 expression according to the protocols described herein.

### Example 26

Design of Phenotypic Assays and In Vivo Studies for the Use of B7.1 or B7.2 Inhibitors

Phenotypic Assays

Once B7.1 or B7.2 inhibitors have been identified by the methods disclosed herein, the compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of B7.1 or B7.2 in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.) cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays. (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with B7.1 or B7.2 inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the genotype of the cell (measurement of the expression of one or more of the genes of the cell) after treatment is also used as an indicator of the efficacy or potency of the B7.1 or B7.2 inhibitors. Hallmark genes, or those genes suspected to be associated with a specific disease state, condition, or phenotype, are measured in both treated and untreated cells.

### Example 27

Antisense Inhibition of Human B7.2 Expression by Chimeric Phosphorothioate Oligonucleotides Having 2′-MOE Wings and a Deoxy Gap

In accordance with the present invention, an additional series of antisense compounds were designed to target different regions of the human B7.2 RNA, using published sequences (GenBark accession number U04343.1, incorporated herein as SEQ ID NO: 295, GenBank accession number BC040261.1, incorporated herein as SEQ ID NO: 296 and GenBank accession number NT_{—}005543.12, a portion of which is incorporated herein as SEQ ID NO: 297). The compounds are shown in Table 25. “Target site” indicates the first (5′-most) nucleotide number on the particular target sequence to which the compound binds. All compounds in Table 25 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on human B7.2 mRNA levels in THP-1 cells by quantitative real-time PCR as described in other examples herein. Data are averages from three experiments. If present, “N.D.” indicates “no data”.

TABLE 25

Inhibition of human B7.2 mRNA levels by chimeric
phosphorothioate oligonucleotides having 2′-MOE wings and a
deoxy gap

| | | | | | |
|---|---|---|---|---|---|
| | | SEQ | | Genbank | |
| Isis | Sequence | ID | % | Accession | Target |
| Number | 5′ to 3′ | NO: | Targt | Site | Region |

| | | | | | | |
|---|---|---|---|---|---|---|
| 322216 | ACCAAAAGGAGTATTTGCG | 298 | N.D. | U04343.1 | 26 | 5′UTR |
| 322217 | CATTCCCAAGGAACACAGA | 299 | N.D. | U04343.1 | 64 | 5′UTR |
| 322218A | CTGTAGCTCCAAAAAGAG | 300 | N.D. | U04343.1 | 105 | 5′UTR |
| 322219 | CTGTCACAAATGCCTGTCC | 301 | N.D. | U04343.1 | 124 | 5′UTR |
| 322220 | TCAGTCCCATAGTGCTGTC | 302 | N.D. | U04343.1 | 138 | START |
| 322221 | CTGTTACAGCAGCAGAGAA | 303 | N.D. | BC040261.1 | 29 | 5′UTR |
| 322222 | TCCCTGTTACAGCAGCAGA | 304 | N.D. | BC040261.1 | 32 | 5′UTR |
| 322223 | ATCTGCAAATGACCCCACT | 305 | N.D. | BC040261.1 | 71 | 5′UTR |
| 322224 | GTGACCTAATATCTGGAAA | 306 | N.D. | BC040261.1 | 81 | 5′UTR |
| 322225 | CATTTTTGCTGCTTCTGCT | 307 | N.D. | BC040261.1 | 100 | START |
| 322226 | GGAACTTACAAAGGAAAGG | 308 | N.D. | BC040261.1 | 1145 | 3′UTR |
| 322227 | AAAAAGGTTGCCCAGGAAC | 309 | N.D. | BC040261.1 | 1159 | 3′UTR |
| 322228 | TGCCTTCTGGAAGAAATCA | 310 | N.D. | BC040261.1 | 1177 | 3′UTR |
| 322229 | TTTTTGCCTTCTGGAAGAA | 311 | N.D. | BC040261.1 | 1181 | 3′UTR |
| 322230 | CTATTCCACTTAGAGGGAG | 312 | N.D. | BC040261.1 | 1233 | 3′UTR |
| 322231 | TCTGATCTGGAGGAGGTAT | 313 | N.D. | BC040261.1 | 1389 | 3′UTR |
| 322232 | AGAAATTGAGAGGTCTATT | 314 | N.D. | BC040261.1 | 1444 | 3′UTR |
| 322233 | CACCAGCTTAGAATTCTGG | 315 | N.D. | BC040261.1 | 1484 | 3′UTR |
| 322234 | AGGTAGTTCTTTAGTCACA | 316 | N.D. | BC040261.1 | 1524 | 3′UTR |
| 322235 | CCAGACTGAGGAGGTAGTT | 317 | N.D. | BC040261.1 | 1535 | 3′UTR |
| 322236 | CAGTACATAGATCTCTATG | 318 | N.D. | BC040261.1 | 1599 | 3′UTR |
| 322237 | TTACAGTACATACATCTCT | 319 | M.D. | BC040261.1 | 1602 | 3′UTR |
| 322238 | GATGAGAACTCCTTAGCAG | 320 | N.D. | BC040261.1 | 1657 | 3′UTR |
| 322239 | TAGCAACAGCCCAGATAGA | 321 | N.D. | BC040261.1 | 1787 | 3′UTR |
| 322240 | TCTGTTGCTTGTTTCAAGA | 322 | N.D. | BC040261.1 | 2043 | 3′UTR |
| 322241 | TCCATTTGGACAGACTATC | 323 | N.D. | BC040261.1 | 2064 | 3′UTR |
| 322242 | GGGAAACTGCTGTCTGTCT | 324 | N.D. | BC040261.1 | 2087 | 3′UTR |
| 322243 | TGCTTCCAGGAAGATGACA | 325 | N.D. | BC040261.1 | 2149 | 3′UTR |
| 322244 | ATTCATCCCATTATCAAGG | 326 | N.D. | BC040261.1 | 2191 | 3′UTR |
| 322245 | ACCCAGGAGTCGAAAGTCC | 327 | N.D. | BC040261.1 | 2223 | 3′UTR |
| 322246 | CTTCCTAATTCCGTTGCAG | 328 | N.D. | BC040261.1 | 2255 | 3′UTR |
| 322247 | CATCTGTAGGCTAAGTAAG | 329 | N.D. | BC040261.1 | 2297 | 3′UTR |
| 322248 | CCCGTAGGACATCTGTAGG | 330 | N.D. | BC040261.1 | 2306 | 3′UTR |
| 322249 | GCCCTATCCTGGGCCAGCC | 331 | N.D. | BC040261.1 | 2331 | 3′UTR |
| 322250 | GTCTCTGTATGCAAGTTTC | 332 | N.D. | BC040261.1 | 2396 | 3′UTR |
| 322251 | CCAGTATATCTGTCTCTGT | 333 | N.D. | BC040261.1 | 2407 | 3′UTR |
| 322252 | CCAGGTTTTCAAAGTCATT | 334 | N.D. | BC040261.1 | 2430 | 3′UTR |
| 322253 | AGCCAGGTTTTCAAAGTCA | 335 | N.D. | BC040261.1 | 2432 | 3′UTR |
| 322254 | CCCTTAGTGATCCCACCTT | 336 | N.D. | BC040261.1 | 2453 | 3′UTR |
| 322255 | CTGCCCCATCCCTTAGTGA | 337 | N.D. | BC040261.1 | 2462 | 3′UTR |
| 322256 | TTTATGTTTGGGCAGAGAC | 338 | N.D. | BC040261.1 | 2480 | 3′UTR |
| 322257 | CATGCCAGTCTATAACCCT | 339 | N.D. | BC040261.1 | 2556 | 3′UTR |
| 322258 | TAGCATGGCAGTCTATAAC | 340 | N.D. | BC040261.1 | 2559 | 3′UTR |
| 322259 | TCTAGCATGGCAGTCTATA | 341 | N.D. | BC040261.1 | 2561 | 3′UTR |
| 322260 | TTGTCTAGCATGGCAGTCT | 342 | N.D. | BC040261.1 | 2564 | 3′UTR |
| 322261 | AAGCTTGTCTAGCATGGCA | 343 | N.D. | BC040261.1 | 2568 | 3′UTR |
| 322262 | ACATGGACAAGCTTGTCTA | 344 | N.D. | BC040261.1 | 2576 | 3′UTR |
| 322263 | TTACATGGACAAGCTTGTC | 345 | N.D. | BC040261.1 | 2578 | 3′UTR |
| 322264 | GAATATTACATGGACAAGC | 346 | N.D. | BC040261.1 | 2583 | 3′UTR |
| 322265 | AACTAGCCAGGTGCTAGGA | 347 | N.D. | BC040261.1 | 2636 | 3′UTR |
| 322266 | AATTATTACTCACCACTGC | 348 | N.D. | NT_005543.12 | 1124 | genomic |
| 322267 | TAATATTTAGGCAAGCATG | 349 | N.D. | NT_005543.12 | 13890 | genomic |
| 322268 | GGACCCTGGGCCAGTTATT | 350 | N.D. | NT_005543.12 | 22504 | genomic |
| 322269 | CAAACATACCTGTCACAAA | 351 | N.D. | NT_005543.12 | 23662 | genomic |
| 322270 | CTCATATCAATTGATGGCA | 352 | N.D. | NT_005543.12 | 29265 | genomic |
| 322271 | TGCTACATCTACTCAGTGT | 353 | N.D. | NT_005543.12 | 31796 | genomic |
| 322272 | TGGAAACTCTTGCCTTCGG | 354 | N.D. | NT_005543.12 | 32971 | genomic |
| 322273 | CCATCCACATTGTAGCATG | 355 | N.D. | NT_005543.12 | 34646 | genomic |
| 322274 | TCAGGATGGTATGGCCATA | 356 | N.D. | NT_005543.12 | 36251 | genomic |
| 322275 | TCCCATAGTGCTAGAGTCG | 357 | N.D. | NT_005543.12 | 37218 | genomic |
| 322276 | AGGTTCTTACCAGAGAGCA | 358 | N.D. | NT_005543.12. | 37268 | genomic |
| 322277 | CAGAGGAGCACCACCTAAA | 359 | N.D. | NT_005543.12 | 49133 | genomic |
| 322278 | GACCACATACCAAGCACTG | 360 | N.D. | NT_005543.12 | 49465 | genomic |
| 322279 | ATCTTTCAGAAACCCAAGC | 361 | N.D. | NT_005543.12 | 51347 | genomic |
| 322280 | GAGTCACCAAAGATTTACA | 362 | N.D. | NT_005543.12 | 51542 | genomic |
| 322281 | CTGAAGTTAGCTGAAAGCA | 363 | N.D. | NT_005543.12 | 51815 | genomic |
| 322282 | ACAGCTTTACCTATAGACA | 364 | N.D. | NT_005543.12 | 52118 | genomic |
| 322283 | TCCTCAAGCTCTACAAATG | 365 | N.D. | NT_005543.12 | 54882 | genornic |
| 322284 | GACTCACTCACCACATTTA | 366 | N.D. | NT_005543.12 | 55027 | genomic |
| 322285 | AGTGATACCAAGGCTTCTC | 367 | N.D. | NT_005543.12 | 56816 | genomic |
| 322286 | CTTGGAGAGAATGGTTATC | 368 | N.D. | NT_005543.12 | 61044 | genomic |
| 322287 | GAAGATGTTGATGCCTAAA | 369 | N.D. | NT_005543.12 | 63271 | genomic |
| 322288 | GTGTTGGTTCCTGAAAGAC | 370 | N.D. | NT_005543.12 | 63665 | genomic |
| 322289 | CAGGATTTACCTTTTCTTC | 371 | N.D. | NT_005543.12 | 63711 | genomic |
| 322290 | AGGGCAGAATAGAGGTTGC | 372 | N.D. | NT_005543.12 | 64973 | Genomic |
| 322291 | TTTTTCTCTGGAGAAATAG | 373 | N.D. | NT_005543.12 | 65052 | genomic |
| 323624 | GTTACTCAGTCCCATAGTG | 374 | 59 | U04343.1 | 143 | START |
| 323625 | CAAAGAGAATGTTACTCAG | 375 | 21 | U04343.1 | 153 | Coding |
| 323626 | CCATCACAAAGAGAATGTT | 376 | 32 | U04343.1 | 159 | Coding |
| 323627 | GGAAGGCCATCACAAAGAG | 377 | 54 | U04343.1 | 165 | Coding |
| 323628 | GAGCAGGAAGGCCATCACA | 378 | 44 | U04343.1 | 170 | Coding |
| 323629 | CCAGAGAGCAGGAAGGCCA | 379 | 36 | U04343.1 | 175 | Coding |
| 323630 | AAATAAGCTTGAATCTTCA | 380 | 22 | U04343.1 | 205 | Coding |
| 323631 | AGTCTCATTGAAATAAGCT | 381 | 56 | U04343.1 | 215 | Coding |
| 323632 | AGGTCTGCAGTCTCATTGA | 382 | 41 | U04343.1 | 223 | Coding |
| 323633 | CTACTAGCTCACTCAGGCT | 383 | 50 | U04343.1 | 273 | Coding |
| 323634 | AAATACTACTAGCTCACTC | 384 | 30 | U04343.1 | 278 | Coding |
| 323635 | CTGCCAAAATACTACTAGC | 385 | 24 | U04343.1 | 284 | Coding |
| 323636 | TTCAGAACCAAGTTTTCCT | 386 | 23 | U04343.1 | 307 | Coding |
| 323637 | CCTCATTCAGAACCAAGTT | 387 | 19 | U04343.1 | 312 | Coding |
| 323638 | GTATACCTCATTCAGAACC | 388 | 20 | U04343.1 | 317 | Coding |
| 323639 | GCCTAAGTATACCTCATTC | 389 | 55 | U04343.1 | 323 | Coding |
| 323640 | CTCTTTGCCTAAGTATACC | 390 | 28 | U04343.1 | 329 | Coding |
| 323641 | CCCATATACTTGGAATGAA | 391 | 88 | U04343.1 | 361 | Coding |
| 323642 | CTTGTGCGGCCCATATACT | 392 | 27 | U04343.1 | 370 | Coding |
| 323643 | ATCAAAACTTGTGCGGCCC | 393 | 80 | U04343.1 | 377 | Coding |
| 323644 | CCCTTGTCCTTGATCTGAA | 394 | 71 | U0.4343.1 | 427 | Coding |
| 323645 | ACAAGCCCTTGTCCTTGAT | 395 | 56 | U04343.1 | 432 | Coding |
| 323646 | TTGATACAAGCCCTTGTCC | 396 | 33 | U04343.1 | 437 | Coding |
| 323647 | ATACATTGATACAAGCCCT | 397 | 41 | U04343.1 | 442 | Coding |
| 323648 | TGGATGATACATTGATACA | 398 | 31 | U04343.1 | 448 | Coding |
| 323649 | GAATTCATCTGGTGGATGC | 399 | 81 | U04343.1 | 493 | Coding |
| 323650 | GTTCACAATTCATCTGGTG | 400 | 92 | U04343.1 | 498 | Coding |
| 323651 | TGACAGTTCAGAATTCATC | 401 | 64 | U04343.1 | 503 | Coding |
| 323652 | AGCACTGACAGTTCAGAAT | 402 | 87 | U04343.1 | 508 | Coding |
| 323653 | TAGCAAGCACTGACAGTTC | 403 | 96 | U04343.1 | 513 | Coding |
| 323654 | TGAAGTTAGCAAGCACTGA | 404 | 87 | U04343.1 | 519 | Coding |
| 323655 | TTGACTGAAGTTAGCAAGC | 405 | 65 | U04343.1 | 524 | Coding |
| 323656 | CTATTTCAGGTTGACTGAA | 406 | 76 | U04343.1 | 534 | Coding |
| 323657 | TCTGTTATATTAGAAATTG | 407 | 43 | U04343.1 | 556 | Coding |
| 323658 | GCAGGTCAAATTTATGTAC | 408 | 36 | U04343.1 | 581 | Coding |
| 323659 | GTATAGATGAGCAGGTCAA | 409 | 56 | U04343.1 | 591 | Coding |
| 323660 | GGGTAACCGTGTATAGATG | 410 | 71 | U04343.1 | 601 | Coding |
| 323661 | AGGTTCTGGGTAACCGTGT | 411 | 68 | U04343.1 | 608 | Coding |
| 323662T | AGCAAAACACTCATCTTC | 412 | 22 | U04343.1 | 629 | Coding |
| 323663 | GTTCTTAGCAAAACACTCA | 413 | 23 | U04343.1 | 634 | Coding |
| 323664 | ATTCTTGGTTCTTAGCAAA | 414 | 35 | U04343.1 | 641 | Coding |
| 323665 | GATAdTTGAATTCTTGGTT | 415 | 43 | U04343.1 | 650 | Coding |
| 323666 | ACCATCATACTCGATAGTT | 416 | 71 | U04343.1 | 662 | Coding |
| 323667 | ATCTTGAGATTTCTGCATA | 417 | 52 | U04343.1 | 683 | Coding |
| 323668A | CATTATCTTCAGATTTCT | 418 | 39 | U04343.1 | 688 | Coding |
| 323669 | CGTACACTTCTGTGACATT | 419 | 68 | U04343.1 | 702 | Coding |
| 323670 | AGACAAGCTGATGGAAACG | 420 | 19 | U04343.1 | 722 | Coding |
| 323671 | GAAACAGACAAGCTGATGG | 421 | 26 | U04343.1 | 727 | Coding |
| 323672 | GGAATGAAACAGACAAGCT | 422 | 33 | U043431 | 732 | Coding |
| 323673 | CATCAGGGAATGAAACAGA | 423 | 38 | U04343.1 | 738 | Coding |
| 323674 | CGTAACATCAGGGAATGAA | 424 | 47 | U04343.1 | 743 | Coding |
| 323675 | AGCTCTATAGAGAAAGGTG | 425 | 77 | U04343.1 | 817 | Coding |
| 323676 | CCTCAAGCTCTATAGAGAA | 426 | 24 | U04343.1 | 822 | Coding |
| 323677 | GGAGGCTGAGGGTCCTCAA | 427 | 55 | U04343.1 | 835 | Coding |
| 323678 | AGTACAGCTGTAATCCAAG | 428 | 23 | U04343.1 | 868 | Coding |
| 323679 | TTGGAAGTACAGCTGTAAT | 429 | 60 | U04343.1 | 873 | Coding |
| 323680 | ATAATAACTGTTGGAAGTA | 430 | 51 | U04343.1 | 883 | Coding |
| 323681 | CATCACACATATAATAACT | 431 | 8 | U04343.1 | 893 | Coding |
| 323682 | TCCATTTCCATAGAATTAG | 432 | 35 | U04343.1 | 921 | Coding |
| 323683 | TCTTCTTCCATTTCCATAG | 433 | 16 | U04343.1 | 927 | Coding |
| 323684 | ATTTATAACAGTTGCGAGG | 434 | 32 | U04343.1 | 954 | Coding |
| 323685 | TTGGTTCCACATTTATAAG | 435 | 18 | U04343.1 | 964 | Coding |
| 323686 | CTCTCCATTGTGTTGGTTC | 436 | 53 | U04343.1 | 976 | Coding |
| 323687 | CTTCCCTCTCCATTGTGTT | 437 | 19 | U04343.1 | 981 | Coding |
| 323688 | TGGTCTGTTCACTCTCTTC | 438 | 58 | U04343.1 | 996 | Coding |
| 323689 | TTCATCAGATCTTTCAGGT | 439 | 43 | U04343.1 | 1037 | Coding |
| 323690 | ATCACTTTTGTCGCATGAA | 440 | 82 | U04343.1 | 1088 | Coding |
| 323691 | GCTTTACTCTTTAATTAAA | 441 | 40 | U04343.1 | 1114 | STOP |
| 323692 | GTATGOGCTTTACTCTTTA | 442 | 5 | 7 U04343.1 | 1120 | 3′UTR |
| 323693 | ATACTTGTATGOCCTTTAC | 443 | 62 | U04343.1 | 1126 | 3′UTR |
| 323694 | AATGAATACTTGTATGGGC | 444 | 71 | U04343.1 | 1131 | 3′UTR |

What is claimed is:

1. A method for treating airway hyperresponsiveness or pulmonary inflammation in an individual in need thereof, comprising administering to said individual an antisense compound 8 to 30 nucleobases in length targeted to a nucleic acid molecule encoding a human B7 protein to said individual.

2. The method of claim 1, wherein said antisense compound is an antisense oligonucleotide.

3. The method of claim 2, wherein at least one covalent linkage of said antisense compound is a modified covalent linkage.

4. The method of claim 2, wherein at least one nucleotide of said antisense compound has a modified sugar moiety.

5. The method of claim 2, wherein at least one nucleotide of said antisense compound has a modified nucleobase.

6. The method of claim 1, wherein said human B7 protein is human B7-1 protein.

7. The method of claim 1, wherein said human B7 protein is human B7-2 protein.

8. The method of claim 1, wherein said human B7 protein is both human B7.1 and human B7.2 protein.

9. The method of claim 1, further comprising administering an anti-asthma medication to said individual.

10. The method of claim 1 wherein said antisense compound comprises at least one lipophilic moiety which oligonucleotide is aerosolized and inhaled by said individual.

12. The method of claim 1, wherein said oligonucleotide is administered intranasally, intrapulmonarily or intratracheally.

13. The method of claim 1, wherein said airway hyperresponsiveness or pulmonary inflammation is associated with asthma.

14. A pharmaceutical composition comprising an antisense oligonucleotide targeted to nucleic acid encoding human B7.1 or B7.2 in a formulation suitable for intranasal, intrapulmonary or intratracheal administration.

15. The pharmaceutical composition of claim 14, wherein said composition is in a metered dose inhaler or nebulizer.

16. An RNA compound 8 to 80 nucleobases in length targeted to a nucleic acid molecule encoding a human B7 protein, wherein said compound specifically hybridizes with said nucleic acid molecule encoding human B7 protein and inhibits the expression of human B7 protein.

17. The RNA compound of claim 16 wherein said B7 protein is B7.1.

18. The RNA compound of claim 16 wherein said B7 protein is B7.2.
